(19) United States
(12) Patent Application Publication
(10) Pub. No.: US 2009/0311237 A1
(43) Pub. Date: Dec. 17, 2009

(54) Combination therapy using a soluble hyaluronidase and a bisphosphonate

(76) Inventor: Gregory I. Frost, Del Mar, CA (US)

Correspondence Address: K&L Gates LLP, 3580 Carmel Mountain Road, Suite 200, San Diego, CA, 92130, US

(21) Appl. No.: 12/386,249

(22) Filed: Apr. 14, 2009

Related U.S. Application Data
(60) Provisional application No. 61/124,277, filed on Apr. 14, 2008.
(60) Provisional application No. 61/124,330, filed on Apr. 15, 2008.

(51) Int. Cl. A61K 38/47

(52) U.S. Cl. 424/94.62

(57) ABSTRACT

Provided are combinations, compositions and kits containing a bisphosphonate composition and a soluble hyaluronidase composition formulated for subcutaneous administration. Such products can be used in methods of treating bisphosphonate-treatable diseases or conditions. Also provided are methods for subcutaneous administration of a bisphosphonate compound whereby the dosing regimen is substantially the same as for intravenous administration of the same dosage for treatment of the same bisphosphonate-treatable disease or condition.

### RELATED APPLICATIONS

Benefit of priority is claimed under 35 U.S.C. §119(e) to U.S. Provisional Application Ser. No. 61/124,277, filed Apr. 14, 2008, entitled “COMBINATION THERAPY USING A SOLUBLE HYALURONIDASE AND A BISPHOSPHONATE” and to U.S. Provisional Application Ser. No. 61/124,330, filed Apr. 15, 2008, entitled “COMBINATION THERAPY USING A SOLUBLE HYALURONIDASE AND A BISPHOSPHONATE,” each to Gregory Frost.

This application is related to International Application No. (Attorney Dkt. No. 0119374-00099/3062PC), filed Apr. 14, 2009, entitled “COMBINATION THERAPY USING A SOLUBLE HYALURONIDASE AND A BISPHOSPHONATE,” which also claims priority to U.S. Provisional Application Ser. Nos. 61/124,277 and 61/124,330.

The subject matter of each of the above-referenced applications is incorporated by reference in its entirety.

### Incorporation by Reference of Sequence Listing Provided on Compact Discs

An electronic version on compact disc (CD-R) of the Sequence Listing is filed herewith in duplicate (labeled Copy #1 and Copy #2), the contents of which are incorporated by reference in their entirety. The computer-readable file on each of the aforementioned compact discs, created on Apr. 14, 2009 is identical, 799 kilobytes in size, and titled 3062SEQ.001.txt.

### FIELD OF THE INVENTION

Provided are combinations, compositions and kits containing a bisphosphonate composition and a soluble hyaluronidase composition formulated for subcutaneous administration. Such products can be used in methods of treating bisphosphonate-treatable diseases or conditions. Also provided are methods for subcutaneous administration of a bisphosphonate compound whereby the dosing regimen is substantially the same as for intravenous administration of the same dosage for treatment of the same bisphosphonate-treatable disease or condition.

### BACKGROUND

Osteoporosis affects an estimated 75 million people in Europe, USA and Japan. One in three women over the age of 50 will experience osteoporotic fractures, as will one in five men. Studies have shown that, depending on the drug and the patient population, treatment reduces the risk of vertebral fracture by between 30-65% and of nonvertebral fractures by between 16-53%. Typical treatments for bone disorder, including osteoporosis, involve oral or intravenous (IV) administration of bisphosphonates. Oral administration of bisphosphonates is often associated with irritation of the esophagus (e.g., esophagitis, ulcerative esophagitis, Barrett's esophagus, esophageal disorder, erosive esophagitis, esophageal stenosis and reflux esophagitis), heartburn and dyspepsia (i.e., stomach upset). The pills containing bisphosphonates must be ingested according to a strict protocol in order to ensure absorption. For example, patients must take bisphosphonate pills on an empty stomach, while sitting or standing straight up, and must maintain an upright position for at least 30 minutes following administration. Studies have shown that patients often skip pills and do not take them according to instructions. Further, due to difficulties of IV administration of bisphosphonates, such as patient comfort and time requirements (i.e., intravenous infusions that sometimes require 15 minutes to several hours to perform), there are issues with patient compliance. IV administration also can cause fever, flu-like symptoms, fatigues, gastrointestinal effects, injection site reactions and anemia (Body et al. (2004) Seminars in Oncology 31:73-78). Poor compliance by patients with drug therapies for osteoporosis over a year leaves them at risk for fractures and higher healthcare costs.

Subcutaneous (SC) administration of bisphosphonates is an alternative to oral or intravenous administration. Compared to oral and IV infusions, SC administration of bisphosphonates has several advantages. For example, compared to IV administration, SC administration would reduce the incidence of systemic reactions, does not require sometimes-difficult IV access, improves trough levels, and gives patients more independence. Furthermore, compared to oral administration, SC administration would reduce the incidence of gastrointestinal irritation and provide significant improvement in bioavailability of the drug. SC administration of bisphosphonates is not currently prescribed due to difficulties with skin toxicity at the injection site and poor absorption of the drug. Hence, there is a need for alternative methods for administering bisphosphonates via SC administration.

### SUMMARY

Provided are methods and uses for treating a bisphosphonate-treatable or preventable disease or condition in a subject in need of such treatment. The methods and uses include a step of subcutaneously administering a bisphosphonate in combination with a hyaluronidase, particularly a soluble hyaluronidase, such as any of the animal or bacterial hyaluronidases or human hyaluronidases. Exemplary of such is the soluble human hyaluronidase and preparations thereof described in co-pending U.S. patent application Ser. No. 10/795,095, published as US 2004/0268425, U.S. patent application Ser. No. 11/065,716, published as US 2005/0260186, U.S. patent application Ser. No. 11/238,171, published as US 2006-0104968, particularly the preparation designated rHuPH20, and also described herein. Bisphosphonates include, but are not limited to, nitrogenous bisphosphonates, such as alendronate, cimadronate, ibandronate, neridronate, olpandronate, risedronate, piridronate, pamidronate, zoledronate, and non nitrogenous bisphosphonates, such as etidronate, clodronate, tiludronate, pharmaceutically acceptable salts or esters thereof, any hydrate thereof and combinations thereof. Exemplary of such bisphosphonates are zoledronate, ibandronate or pamidronate. The methods herein, are advantageously employed with the more potent bisphosphonates, such as the nitrogenous bisphosphonates.

Provided herein are compositions containing a soluble hyaluronidase for use for treating a bisphosphonate-treatable or preventable disease or condition. Such composition contain a soluble hyaluronidase formulated for subcutaneous administration in an amount effective to prevent an ISR when formulated for administration subcutaneously with the bisphosphonate.

Also provided herein are pharmaceutical compositions and combinations containing the soluble hyaluronidase and bisphosphonate.

Also provided herein are uses of a hyaluronidase for the formulation of a medicament for use in combination with a bisphosphonate for treating bisphosphonate-treatable or preventable disease or condition. For such uses, the soluble hyaluronidase is generally formulated for subcutaneous administration in an amount effective to prevent an injection site reaction (ISR) when formulated for administration subcutaneously with the bisphosphonate.

Provided are uses of and methods of using a soluble hyaluronidase for the formulation of a medicament for preventing an injection site reaction when administered in combination with a bisphosphonate, which is administered for treating bisphosphonate-treatable or preventable disease or conditions. Also provided are compositions that contain the soluble hyaluronidase and a bisphosphonate. For the uses, methods and compositions, the soluble hyaluronidase is formulated for subcutaneous administration in an amount effective to prevent an injection site reaction (ISR) when formulated for administration subcutaneously in combination with the bisphosphonate. The soluble hyaluronidase and bisphosphonate can be administered as separate compositions or in a single composition. The compositions and methods can contain/administer more than bisphosphonate.

Injection of bisphosphonates, particularly subcutaneously, without a soluble hyaluronidase, such as rHuPH20, results in injection site reactions characterized by erythema, induration, and ulceration in a concentration dependent manner. As shown herein, the maximal concentration of bisphosphonates that can be administered without producing ISRs can be increased by administering them with a soluble hyaluronidase, such a rHuPH20. The amount of bisphosphonate administered typically can be typically 3-5 fold when co-administered with rHuPH20. Absolute bioavailability by subcutaneous (SC) injection with, for example, rHuPH20 is at least comparable to IV infusion.

The amount of bisphosphonate administered typically is the amount and regimen used for treatment of a particular disease for which it has been employed. For purposes herein, it is co-administered (either separately, where the compositions are administered simultaneously or sequentially within a predetermined time, or as a single composition) subcutaneously with an amount of the soluble hyaluronidase sufficient to prevent or substantially reduce (i.e. to patient tolerable level), the ISR from the bisphosphonate. The amount of soluble hyaluronidase depends upon the particular soluble hyaluronidase and the bisphosphonate and amount administered as well as the volume and time of administration. Typical amounts are in the range of about or at 100 Units to 100,000 Units; 100 Units to at or about 1000, 3000, 5000, 10,000, 20,000, 50,000, 80,000 or 100,000 Units; at or about 1000 Units to 1000, 3000, 5000, 10,000, 20,000, 50,000, 80,000 or 100,000 Units; at or about 3000 Units to 1000, 3000, 5000, 10,000, 20,000, 50,000, 80,000 or 100,000 Units; at or about 5000 Units to 1000, 3000, 5000, 10,000, 20,000, 50,000, 80,000 or 100,000 Units; at or about 10,000 Units to 1000, 3000, 5000, 10,000, 20,000, 50,000, 80,000 or 100,000 Units; at or about 1000 Units to 50,000 Units; at or about 1000 Units to 24,000 Units; at or about 1000 Units to 10,000 Units; at or about 3000 Units to 10,000 Units, at or about 3000 Units to 24,000 or 25,000 Units, at or about 5000 Units to 30,000 Units or other amount sufficient to prevent or reduce the ISR.

Exemplary concentrations of soluble hyaluronidase in the compositions, include, but are not limited to, soluble hyaluronidase in the composition for administration is at or about 10 Units/ml to 5,000,000 Units/ml, 500,000 Units/ml, 100 Units/ml to 100,000 Units/ml, 500 Units/ml to 50,000 Units/ml, 1000 Units/ml to 10,000 Units/ml, 5000 Units/ml to 7500 Units/ml, 5000 Units/ml to 50,000 Units/ml, 1,000 Units/ml to 10,000 Units/ml, or 100 Units/ml to 1000 Units/ml. Bisphosphonate-treatable or preventable disease or condition, include, but are not limited to, osteoporosis, Paget's Disease, abnormally increased bone turnover, periodontal disease, tooth loss, bone fractures, rheumatoid arthritis, periprosthetic osteolysis, osteogenesis imperfecta, metastatic bone disease, bone metastases, hypercalcemia of malignancy and multiple myeloma.

The amounts of the bisphosphonate depend, for example, on the particular bisphosphonate, the disease or condition treated, the patient and other such parameters. Typical amounts include, but are not limited to, is or is about 0.5 milligrams (mg), about or 1 mg, about or 3 mg, about or 5 mg, about or 10 mg, about or 20 mg, about or 30 mg, about or 40 mg, about or 50 mg, about or 60 mg, about or 70 mg, about or 80 mg, about or 90 mg, about or 100 mg.

For example, where the bisphosphonate is zoledronate or ibandronate, the amounts can be at or about 0.5 milligrams (mg), about or 1 mg, about or 1.5 mg, about or 2 mg, about or 2.5 mg, about or 3 mg, about or 3.5 mg, about or 4 mg, about or 4.5 mg, about or 5 mg, about or 5.5 mg, about or 6 mg, about or 6.5 mg, about or 7 mg, about or 7.5 mg, about or 8 mg, about or 8.5 mg, about or 9 mg, about or 9.5 mg, or about or 10 mg. For example, in one exemplary embodiment, the amount of ibandronate in the composition is or is about 3 milligrams in a liquid formulation, and the volume of the formulation is or is about 1 milliliter to 5 milliliters. For example, where the bisphosphonate is zoledronate, it can be provided in amount of 5 milligrams (or any desired amount, such as noted above) in a liquid formulation, wherein the volume of the formulation is or is about 25 milliliters to 400 milliliters.

For example, where the bisphosphonate is pamidronate, the amount of pamidronate in the composition can be about or 10 mg, about or 20 mg, about or 30 mg, about or 40 mg, about or 50 mg, about or 60 mg, about or 70 mg, about or 80 mg, about or 90 mg, or about or 100 mg. The volume can be, for example, 100 milliliter to 200 milliliters.

Also provided are combinations of the soluble hyaluronidase and bisphosphonate that contain:
(a) a first composition comprising a bisphosphonate formulated for single dosage subcutaneous administration at a dosage frequency of no greater than once per week in an amount sufficient for treating the disease or condition; and
(b) a second composition comprising an amount of a soluble hyaluronidase formulated for single dosage subcutaneous administration at a dosage frequency of no greater than once per week, wherein the amount of soluble hyaluronidase is at or about 100 Units to 100,000 Units.

The first and second compositions can be provided separately or can be mixed to form a single composition for subcutaneous administration. The combinations can be provided as kits, that optionally include, for example, instructions for use and other reagents and devices for administration amounts, concentrations, volumes and types of soluble hyaluronidase and types, volumes, concentrations and amounts of bisphosphonate are as described above and below for the compositions, uses and methods. For example in some embodiments, the amount of bisphosphonate in the first composition is sufficient to treat the disease or condition for a period of at least one week, two weeks, three weeks, four weeks, one month, two months, three months, four months, five months, six months seven months, eight months, nine months, ten months, eleven months, twelve months, eighteen months or twenty four months without need for additional bisphosphonate administration to the subject during the period; and
(b) the amount of soluble hyaluronidase supplied in the preparation is such that, following subcutaneous administration of the bisphosphonate and hyaluronidase dosages over a desired length of time for completing such administration, the incidence of injection site reactions is eliminated or substantially reduced compared to subcutaneous administration of the same amount of bisphosphonate administered in the absence of the hyaluronidase over the same length of time.

In other exemplary embodiments:
(a) the amount of bisphosphonate supplied in the first composition is sufficient to treat the disease or condition for a period of at least one week, two weeks, three weeks, four weeks, one month, two months, three months, four months, five months, six months seven months, eight months, nine months, ten months, eleven months, twelve months, eighteen months or twenty four months without need for additional bisphosphonate administration to the subject during the period; and
(b) the amount of bisphosphonate in the first composition is such that, following subcutaneous administration, the bisphosphonate causes the same or substantially no greater degree or severity of injection site reactions compared to subcutaneous administration of about one third to one fifth the amount of bisphosphonate, administered at the same rate, in the absence of hyaluronidase.

The soluble hyaluronidase can be provided in the combination/kits, for example, in the form of a dry powder or a liquid. Volumes, include, but are not limited to, 1 ml, 5 ml, 10 ml, 25 ml, 50 ml, 100 ml, 150 ml, 200 ml, 300 ml, 400 ml, 500 ml, 600 ml and 700 ml or more.

The bisphosphonate administered in the methods herein can be administered at or about 0.5 milligrams (mg), at or about 1 mg, at or about 3 mg, at or about 5 mg, at or about 10 mg, at or about 20 mg, at or about 30 mg, at or about 40 mg, at or about 50 mg, at or about 60 mg, at or about 70 mg, at or about 80 mg, at or about 90 mg, at or about 100 mg. Where the bisphosphonate is zoledronate, the bisphosphonate can be administered at or about 0.5 milligrams (mg), at or about 1 mg, at or about 1.5 mg, at or about 2 mg, at or about 2.5 mg, at or about 3 mg, at or about 3.5 mg, at or about 4 mg, at or about 4.5 mg, at or about 5 mg, at or about 5.5 mg, at or about 6 mg, at or about 6.5 mg, at or about 7 mg, at or about 7.5 mg, at or about 8 mg, at or about 8.5 mg, at or about 9 mg, at or about 9.5 mg, or at or about 10 mg. For example, the 5 mg of zoledronate can be administered once yearly. The zoledronate can be provided in a liquid formulation, wherein the volume of the formulation is or is about 25 milliliters to 400 milliliters. In such a method, a soluble hyaluronidase can be administered with the zoledronate in the liquid formulation is 100 Units/ml to 1000 Units/ml of soluble hyaluronidase in a volume of the liquid formulation is or is about 25 milliliters to 200 milliliters.

In another example, where the bisphosphonate is ibandronate, the ibandronate can be administered at or about 0.5 milligrams (mg), at or about 1 mg, at or about 1.5 mg, at or about 2 mg, at or about 2.5 mg, at or about 3 mg, at or about 3.5 mg, at or about 4 mg, at or about 4.5 mg, at or about 5 mg, at or about 5.5 mg, at or about 6 mg, at or about 6.5 mg, at or about 7 mg, at or about 7.5 mg, at or about 8 mg, at or about 8.5 mg, at or about 9 mg, at or about 9.5 mg, or at or about 10. For example, the ibandronate can be administered at 3 mg once every three months. In another example, the ibandronate is administered at or about 2 mg to 5 mg in a liquid formulation wherein the volume of the formulation is or is about 1 milliliter to 5 milliliter. In such methods, a soluble hyaluronidase can be administered with the ibandronate in the liquid formulation at or at about 100 Units/ml to 1000 Units/ml of soluble hyaluronidase.

In an additional example, the bisphosphonate is pamidronate, and the pamidronate can be administered at or about 10 mg, at or about 20 mg, at or about 30 mg, at or about 40 mg, at or about 50 mg, at or about 60 mg, at or about 70 mg, at or about 80 mg, at or about 90 mg, or at or about 100 mg of pamidronate is administered. For example, the pamidronate is administered at or at about 90 mg, such as in a liquid formulation, wherein the volume of the formulation is or is about 100 milliliters to 200 milliliters. In such methods, a soluble hyaluronidase can be administered with the pamidronate in the liquid formulation at or at about 100 Units/ml to 1000 Units/ml of soluble hyaluronidase.

Generally, in any of the methods herein, the soluble hyaluronidase is administered at a ratio of Units hyaluronidase/milligrams of bisphosphonate that is at or about 10 U/milligram (mg); at or about 25 U/mg; at or about 100 U/mg; at or about 1000 U/mg; at or about 2500 U/mg; at or about 5000 U/mg; at or about 10,000 U/mg; at or about 20,000 U/mg; at or about 100,000 U/mg; at or about 200,000 U/mg; at or about 1,000,000 U/mg; or at or about 2,000,000 U/mg. For example, the hyaluronidase can be administered at a ratio (Units hyaluronidase/milligrams of bisphosphonate) at or about 200 U/mg; or at or about 25,000 U/mg.

In any of the compositions, uses and methods herein, the bisphosphonate-treatable or preventable disease or condition includes, for example osteoporosis, Paget's Disease, abnormally increased bone turnover, periodontal disease, tooth loss, bone fractures, rheumatoid arthritis, periprosthetic osteolysis, osteogenesis imperfecta, metastatic bone disease, bone metastases, hypercalcemia of malignancy and multiple myeloma. In some examples, administration of soluble hyaluronidase and bisphosphonate results in an increase in bone density in the subject or a decrease in the rate of bone degradation in the subject following treatment.

The soluble hyaluronidase in the methods, uses, compositions or combinations herein a neutral active soluble hyaluronidase, such as a soluble form of PH20. The PH20 can be any species, for example, ovine, mouse, monkey, bovine or human PH20, so long as it is soluble. For example, where the PH20 is a human PH20, it can be rendered soluble by removal or elimination of all or a portion of a C-terminal glycosylphosphatidylinositol attachment site, for example, using standard recombinant DNA techniques or other methods known to one of skill in the art. Exemplary of such truncated human PH20 are any having a sequence of amino acids set forth in any of SEQ ID NOS:4-9 and 47-48, and allelic variants, species variants and other variants thereof. For example, where the PH20 includes other variants the other variants include polypeptides having at least 60, 65, 70, 75, 80, 85, 86, 87, 88, 89, 90, 91, 92, 93, 94, 95, 96, 97, 98, 99% or more sequence identity along their full length to a contiguous sequence of amino acids set forth in SEQ ID NO:1. Exemplary of a human soluble PH20 include one encoded by a sequence of nucleic acids that encodes a sequence of amino acids set forth in SEQ ID NO:3 or 4. For example, a human soluble PH20 includes a polypeptide encoded by a sequence of nucleic acids set forth in SEQ ID NO:49.

For example, the soluble hyaluronidase is a soluble human PH20 that lacks a C-terminal glycosylphosphatidylinositol attachment site, such as among polypeptides containing a sequence of amino acids set forth in any of SEQ ID NOS: 3, 4-9 and 48, and allelic variants, species variants and other variants thereof that retain hyaluronidase activity, such as variants selected from among polypeptides having at least 60, 65, 70, 75, 80, 85, 88, 90, 91, 92, 93, 94, 95, 96, 97, 98, 99% or more sequence identity along their full length to a contiguous sequence of amino acids set forth in SEQ ID NO:1. In some embodiments the soluble hyaluronidase is an rHuPH20, which is produced by expression of amino acids 36-482 of SEQ ID NO:1 in a CHO cell. The soluble

Any of the soluble hyaluronidases provided herein can be produced in any by any method, such as, for example, by production in a mammalian cell line (e.g. CHO cells). Generally, such soluble hyaluronidases are glycosylated. For example, such an exemplary soluble hyaluronidase includes rHuPH20. Further, any of the soluble hyaluronidases provided herein can be modified by conjugation to a polymer that increases half-life. Exemplary of such modification include, but are not limited to, PEGylation, salivation or conjugation to DEXTRAN.

The bisphosphonate in the methods, compositions and combinations herein include an N-bisphosphonate or a pharmaceutically acceptable salt or ester thereof or any hydrate thereof. For example, bisphosphonates include, but are not limited to, nitrogenous bisphosphonates, such as alendronate, cimadronate, ibandronate, neridronate, olpandronate, risedronate, piridronate, pamidronate, zoledronate, and non nitrogenous bisphosphonates, such as etidronate, clodronate, tiludronate, pharmaceutically acceptable salts or esters thereof, any hydrate thereof and combinations thereof. Exemplary of such bisphosphonates are zoledronate, ibandronate or pamidronate.

The bisphosphonates and/or soluble hyaluronidase, can be provided, such as in the combinations herein, in the form of a dry powder or a liquid and/or the soluble hyaluronidase is provided in the form of a dry powder or a liquid.

When administered, the bisphosphonate and soluble hyaluronidase are a liquid. The volume of liquid of the hyaluronidase and bisphosphonate composition, separately or in a single composition, is any suitable volume for subcutaneous administration and is typically at or about 1 ml, 5 ml, 10 ml, 25 ml, 50 ml, 100 ml, 150 ml, 200 ml, 300 ml, 400 ml, 500 ml, 600 ml or 700 ml or more. The volume depends upon the dosage of bisphosphonate, the particular bisphosphonate, patient, disease or condition and other such parameters.

Bisphosphonate in the methods and uses provided can be co-administered with hyaluronidase subcutaneously, in combination with other agents used in the treatment of bisphosphonate-treatable diseases and conditions. For example, additional agents that can be administered include, but are not limited to, vitamin and mineral supplements, such as calcium and Vitamin D or an analog thereof or anticancer agents.

Provided are methods for treating a bisphosphonate-treatable or preventable disease or condition in a subject in need of such treatment. In the methods, compositions/combinations of a bisphosphonate and soluble hyaluronidase are administered. The bisphosphonate, soluble hyaluronidase and compositions and combinations are as described above.

In practicing the methods, (a) an amount of a soluble hyaluronidase and (b) a bisphosphonate, such as a nitrogenous bisphosphonate, including, for example, zoledronate, ibandronate and/or pamidronate, in an amount sufficient for treating the disease or condition is administered to the subject. The soluble hyaluronidase is administered, for example, at a concentration of at or about 10 Units/ml to 1000 Units/ml in an amount such that the incidence of injection site reactions in the subject is eliminated or substantially reduced compared to subcutaneous administration of the same amount of bisphosphonate in the absence of the hyaluronidase. The amounts and volumes are as described above, such as at or about 100 Units/ml to 1000 Units/ml in at or about 1 ml to 500 ml, 600 ml, 700 ml or more. Exemplary amounts of the soluble hyaluronidase administered is at or about 100 Units to 100,000 Units; at or about 1000 Units to 100,000 Units; at or about 3000 Units to 100,000 Units; at or about 5000 Units to 100,000 Units; at or about 10,000 Units to 100,000 Units; at or about 1000 Units to 50,000 Units; at or about 1000 Units to 24,000 Units; at or about 1000 Units to 10,000 Units; or at or about 3000 Units to 10,000 Units. The frequency of administration of the bisphosphonate is substantially the same as for intravenous administration of the same amount of bisphosphonate for the same disease or condition. The compositions can be administered, sequentially, simultaneously in the same composition or in separate compositions, or intermittently.

In exemplary methods, a soluble hyaluronidase is administered in an amount to reduce the incidence of an injection site reaction caused by subcutaneous administration of a bisphosphonate. In one method, a soluble hyaluronidase and a bisphosphonate are subcutaneously administered for treating a bisphosphonate-treatable or preventable disease or condition in a subject in need of such treatment where the bisphosphonate in an amount sufficient for treating the disease or condition and the soluble hyaluronidase is administered in an amount such that the incidence of injection site reactions in the subject is eliminated or substantially reduced compared to subcutaneous administration of the same amount of bisphosphonate in the absence of the hyaluronidase. In such examples, the soluble hyaluronidase is generally administered at a concentration of at or about 10 Units/ml to 1000 Units/ml.

In another exemplary method, a soluble hyaluronidase and a bisphosphonate are subcutaneously administered over a predetermined length of time to a subject in an amount for treating the disease or condition. In such an example, the amount of soluble hyaluronidase is such that, following subcutaneous administration of the amount of bisphosphonate over a predetermined length of time to complete such administration, the incidence of injection site reactions in the subject is eliminated or substantially reduced compared to subcutaneous administration of the same amount of bisphosphonate, administered over the same length of time, in the absence of the hyaluronidase.

In another example of the method, a soluble hyaluronidase and a bisphosphonate are administered in an amount for treating the disease or condition at a predetermined rate of administration. In such an example of the method, the amount of bisphosphonate administered is such that, following subcutaneous administration at a predetermined rate of administration, the bisphosphonate causes the same or substantially no greater degree or severity of injection site reactions compared to subcutaneous administration of about one third to one fifth the amount of bisphosphonate, administered at the same rate, in the absence of hyaluronidase.

In a further example of the method, a soluble hyaluronidase and a bisphosphonate are subcutaneously administered to the subject in an amount effective for treating the disease or condition. In such an example, the quantify of bisphosphonate and a dosing frequency for successive administrations of a bisphosphonate to the subject are selected such that the therapeutic effect of the subcutaneous bisphosphonate administration upon the subject is at least substantially equivalent to intravenous administration of the bisphosphonate to the subject using the same dosing regimen.

In each of the above methods of subcutaneously administering a soluble hyaluronidase and a bisphosphonate, the frequency of administration of the bisphosphonate is the same as for intravenous administration of the same amount of bisphosphonate for the same disease or condition. In another example, the frequency of administration of the bisphosphonate is less than for intravenous administration of the same amount of bisphosphonate for the same disease or condition.

In an another example of the method, a soluble hyaluronidase and bisphosphonate are subcutaneously administered to the subject in an amount for treating the disease or condition. In such an example, the amount of the bisphosphonate administered and the frequency of administration is substantially the same as for intravenous administration of the same amount for the same disease or condition.

In each and all of the methods for treating a bisphosphonate-treatable or preventable disease or condition herein, the subject can be a human subject.

In the methods, one or more bisphosphonates can be administered. The bisphosphonates can be administered for the same length of time required to complete administration as for intravenous administration of the same amount of bisphosphonate for the same disease of condition. In an additional example, the bisphosphonate is administered for a shorter length of time required to complete administration as for intravenous administration of the same amount of bisphosphonate for the same disease or condition.

In the methods herein, administration of a bisphosphonate in combination with a soluble hyaluronidase permits bioavailability of the administered bisphosphonate to at least or about 90% of the bioavailability of the same dosage administered via intravenous administration. Generally, in the methods, the amount of bisphosphonate administered is sufficient to treat the subject for a period of up to one week, two weeks, three weeks, four weeks, one month, two months, three months, four months, five months, six months, seven months, eight months, nine months, ten months, eleven months, twelve months, eighteen months or twenty-four months without need for additional bisphosphonate administration to the subject during the period.

In the methods provided herein, the bisphosphonate and hyaluronidase can be administered as a single subcutaneous injection, or as a series of subcutaneous injections. The soluble hyaluronidase that is administered in the methods herein is administered in an amount that is sufficient to effect subcutaneous administration of the bisphosphonate at a dosage administered no more than once per week. Typically, the frequency of the dosage regimen comprises administration of bisphosphonate and soluble hyaluronidase once every week, once every two weeks, once every three weeks, once every four weeks, once every month, once every two months, once every three months, once every four months, once every five months, once every six months, once every seven months, once every eight months, once every nine months, once every ten months, once every eleven months, once every twelve months, once every twelve months or once every two years. In the methods herein, the time interval between two successive treatments is greater than the time interval between treatments for administration of the same amount of bisphosphonate via intravenous administration.

In one example, the bisphosphonate and hyaluronidase are administered together in the same subcutaneous injection. In another example, the bisphosphonate and hyaluronidase are administered separately. When administered separately, the bisphosphonate and hyaluronidase are administered simultaneously, sequentially, or intermittently, using selected and prescribed solution injection volumes. For example, the hyaluronidase can be administered prior to administration of the bisphosphonate (i.e., “leading edge” administration of the hyaluronidase). The hyaluronidase can be administered 0.5 minutes, 1 minute, 2 minutes, 3 minutes, 4 minutes, 5 minutes, 6 minutes, 7 minutes, 8 minutes, 9 minutes, 10 minutes, 20 minutes or 30 minutes prior to administration of the bisphosphonate. In a further example, the bisphosphonate and hyaluronidase are formulated together as a single composition.

In the methods herein, the bisphosphonate is in liquid formulation, and the time required to subcutaneously administer the dosage of bisphosphonate is determined based on the concentration of the bisphosphonate in the liquid dose formulation and at a desired rate of infusion of the liquid formulation. In such a method, the rate of infusion is controlled by a pump, by gravity or controlled dispersion from a syringe or other known administration device over a period of time.

In certain embodiments, the bisphosphonate is administered for the same length of time required to complete administration as for intravenous administration of the same amount of bisphosphonate for the same disease of condition. In others, the bisphosphonate is administered for a shorter length of time required to complete administration as for intravenous administration of the same amount of bisphosphonate for the same disease or condition. In these methods, the bioavailability of the subcutaneously administered bisphosphonate can be at least about 90% of the bioavailability of the same dosage administered via intravenous administration.

In the methods, the amount of bisphosphonate administered can be is sufficient to treat the subject for a period of one week, two weeks, three weeks, four weeks, one month, two months, three months, four months, five months, six months, seven months, eight months, nine months, ten months, eleven months, twelve months, eighteen months or twenty-four months without need for additional bisphosphonate administration to the subject during the period.

The amount of soluble hyaluronidase administered can be sufficient to effect subcutaneous administration of the bisphosphonate at a dosage administered no more than once per week. The frequency of the dosage regimen can include administration of bisphosphonate and soluble hyaluronidase once every week, once every two weeks, once every three weeks, once every four weeks, once every month, once every two months, once every three months, once every four months, once every five months, once every six months, once every seven months, once every eight months, once every nine months, once every ten months, once every eleven months, once every twelve months, once every twelve months or once every two years. The time interval between two successive treatments can be greater than the time interval between treatments for administration of the same amount of bisphosphonate via intravenous administration. The soluble hyaluronidase includes any described above or below, such as a PH20, such as a soluble form of ovine, mouse, monkey, bovine or human PH20 or a truncated form thereof, including the rHuPH20 preparation.

In the methods, the bisphosphonate and hyaluronidase, for example, can be administered as a single subcutaneous injection; they can be administered, administered separately, together, simultaneously, sequentially or intermittently, in any order, such as administration of the hyaluronidase prior to administration of the bisphosphonate.

### DETAILED DESCRIPTION

### A. Definitions

### B. Subcutaneous Administration of Bisphosphonates

### C. Bisphosphonates

### D. Hyaluronan Degrading Enzymes

1. Hyaluronidases
a. Mammalian-type hyaluronidases
   i. PH20
b. Bacterial hyaluronidases
c. Hyaluronidases from leeches, other parasites and crustaceans

2. Other hyaluronan degrading enzymes

3. Soluble hyaluronan degrading enzymes
a. Soluble Human PH20
b. HuPH20

4. Glycosylation of hyaluronan degrading enzymes

E. Methods of Producing Nucleic Acids encoding a soluble Hyaluronidase and Polypeptides Thereof

1. Vectors and cells

2. Expression
a. Prokaryotic Cells
b. Yeast Cells
c. Insect Cells
d. Mammalian Cells
e. Plants

3. Purification Techniques

### F. Preparation, Formulation and Administration of Bisphosphonates and Soluble Hyaluronidase Polypeptides

1. Formulations
a. Lyophilized powder

2. Dosage and Administration

### G. Methods of Assessing Activity, Bioavailability and Pharmacokinetics

1. Pharmacokinetics and tolerability

2. Biological activity
a. Bisphosphonate
b. Hyaluronidase
   H. Therapeutic uses

1. Non-malignant bone disorders
a. Osteoporosis
b. Glucocorticoid-induced osteoporosis
c. Paget's Disease of Bone
d. Osteogenesis imperfecta

2. Cancer-related bone disorders
a. Hypercalcemia of malignancy
b. Metastatic bone disease
c. Multiple myeloma
   I. Articles of manufacture and kits

### J. EXAMPLES

### A. Definitions

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, a “bisphosphonate” refers to any member of the class of compounds characterized by two PO₃ (phosphate) groups covalently linked to carbon and can inhibit osteoclast-mediated bone resorption. The term bisphosphonate encompasses bisphosphonate free acid, bisphosphonate salts, and bisphosphonate esters, bisphosphonate hydrate, diphosphonates, diphosphonic acids, as well as any salts, derivatives or mixtures thereof. Examples include, but are not limited to, alendronate, risedronate, etidronate, clodronate, pamidronate, tiludronate, ibandronate, zoledronate, incadronate, olpadronate, neridronate, or amidronate.

As used herein, bisphosphonate-treatable or preventable diseases or conditions refer to any disease or condition for which bisphosphonate preparations are used. Such diseases and conditions, include, but are not limited to, osteoporosis, Paget's disease, abnormally increased bone turnover, periodontal disease, tooth loss, bone fractures, rheumatoid arthritis, periprosthetic osteolysis, osteogenesis imperfecta (e.g., brittle bones), metastatic bone disease, heterotopic ossification, fibrous dysplasia, primary hyperparathyroidism, bone metastases, hypercalcemia of malignancy, and multiple myeloma.

As used herein, “bone resorption inhibiting” refers to preventing bone resorption by the direct or indirect alteration of osteoclast formation or activity. Inhibition of bone resorption refers to prevention of bone loss, especially the inhibition of removal of existing bone either from the mineral phase and/or the organic matrix phase, through direct or indirect alteration of osteoclast formation or activity.

As used herein, “preventing an injection site reaction” means that one or more symptoms exhibited at the site of an injection in a subject, such as a site of subcutaneous injection of a bisphosphonate, is partially or totally alleviated. An injection site reaction is characterized by inflammation in or damage to the tissue surrounding where a drug was injected. Such injection site reactions include for example erythema, induration, and ulceration of the skin surrounding the injection site and can cause redness, tenderness, warmth, itching, pain, blistering and/or skin damage in the subject. Injection site reactions are commonly observed in patients receiving intravenous administration of bisphosphonates. The ISRs, including those described herein, are partially or totally alleviated when the bisphosphonate is administered in combination with a soluble hyaluronidase provided herein.

As used herein, dosing regime refers to the amount of bisphosphonate administered and the frequency of administration. The dosing regime is a function of the disease or condition to be treated, and thus can vary.

As used herein, “substantially the same as an intravenous bisphosphonate dosing regime refers to” a regimen in which the dose and/or frequency is within an amount that is effective for treating a particular disease or condition, typically is at or about 10% of the IV dose or frequency. Amounts of a bisphosphonate that are effective for treating a particular disease or condition are known or can empirically determined by one of skill in the art. For example, as exemplified below, 5 mg is the typical yearly dose of a bisphosphonate, such as zoledronate, administered to patients intravenously having osteoporosis, Paget's disease of the bone; and 4 mg is the yearly dose administered to patients intravenously for treatment of hypercalcemia of malignancy and bone metastases. In another example, 1 mg of a bisphosphonate, such as ibandronate, is administered to patients intravenously having osteoporosis, Paget's disease of the bone. In another example, 30-90 mg of a bisphosphonate, such as pamidronate is administered intravenously for the treatment of hypercalcemia of malignancy and bone metastases. Hence, bisphosphonate, when administered in combination with hyaluronidase, is administered subcutaneously at doses that are the same as the intravenous dose for a particular bisphosphonate.

As used herein, frequency of administration refers to the time between successive doses of a bisphosphonate. For example, frequency can be one, two, three, four weeks, and is function of the particular disease or condition treated. Generally, frequency is a least every two or three weeks, and typically no more than once a month.

As used herein, the phrases “administered in combination with” or “administered with” when referring to a bisphosphonate administered in combination with a hyaluronidase, such a soluble hyaluronidase, mead that the bisphosphonate and the hyaluronidase can be administered together (i.e. simultaneously), separately, intermittently, in the same composition, or in separate compositions. When administered separately, the bisphosphonate and the hyaluronidase can be administered in combination sequentially, for example, the bisphosphonate can be immediately administered following administration of the hyaluronidase or can be administered at a selected time interval following administration of the hyaluronidase, such as for example, 1 minute, 2 minute, 3 minute, 4 minute, 5 minute, 6 minute, 7 minutes, 8 minutes, 9 minutes, 10 minutes, 20 minutes or 30 minutes following administration of the hyaluronidase.

As used herein, hyaluronidase refers to an enzyme that degrades hyaluronic acid. Hyaluronidases include bacterial hyaluronidases (EC 4.2.99.1), hyaluronidases from leeches, other parasites, and crustaceans (EC 3.2.1.36), and mammalian-type hyaluronidases (EC 3.2.1.35). Hyaluronidases also include any of non-human origin including, but not limited to, murine, canine, feline, leporine, avian, bovine, ovine, porcine, equine, piscine, ranine, bacterial, and any from leeches, other parasites, and crustaceans. Exemplary non-human hyaluronidases include, hyaluronidases from cows (SEQ ID NO:10, 11, and 64), sheep (SEQ ID NO: 63), yellow jacket wasp (SEQ ID NOS:12 and 13), honey bee (SEQ ID NO:14), white-face hornet (SEQ ID NO:15), paper wasp (SEQ ID NO:16), mouse (SEQ ID NOS:17-19, 31), pig (SEQ ID NOS:20-21), rat (SEQ ID NOS:22-24, 30), rabbit (SEQ ID NO:25), sheep (SEQ ID NO:26 and 27), orangutan (SEQ ID NO:28), cynomolgus monkey (SEQ ID NO:29), guinea pig (SEQ ID NO:32), Staphylococcus aureus (SEQ ID NO:33), Streptococcus pyogenes (SEQ ID NO:34), and Clostridium perfringens (SEQ ID NO:35). Hyaluronidases also include those of human origin. Exemplary human hyaluronidases include HYAL1 (SEQ ID NO:36), HYAL2 (SEQ ID NO:37), HYAL3 (SEQ ID NO:38), HYAL4 (SEQ ID NO:39), and PH20 (SEQ ID NO:1). Also included amongst hyaluronidases are soluble hyaluronidases, including, ovine and bovine PH20, soluble human PH20 and soluble rHuPH20.

Reference to hyaluronidases includes precursor hyaluronidase polypeptides and mature hyaluronidase polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the precursor polypeptides set forth in SEQ ID NOS: 1 and 10-39, or the mature form thereof. For example, reference to hyaluronidase also includes the human PH20 precursor polypeptide variants set forth in SEQ ID NOS:50-51. Hyaluronidases also include those that contain chemical or posttranslational modifications and those that do not contain chemical or posttranslational modifications, including modifications that improve the half-life of the polypeptide. Such modifications include, but are not limited to, pegylation, albumination, glycosylation, farnysylation, carboxylation, hydroxylation, phosphorylation, and other polypeptide modifications known in the art.

As used herein, a soluble hyaluronidase refers to a hyaluronidase that is characterized by its solubility under physiologic conditions. Soluble hyaluronidases can be distinguished, for example, by its partitioning into the aqueous phase of a Triton X-114 solution warmed to 37° C. (Bordier et al., (1981) J. Biol. Chem., 256:1604-7). Membrane-anchored, such as lipid anchored hyaluronidases, will partition into the detergent rich phase, but will partition into the detergent-poor or aqueous phase following treatment with Phospholipase-C. Included among soluble hyaluronidases are membrane anchored hyaluronidases in which one or more regions associated with anchoring of the hyaluronidase to the membrane has been removed or modified, where the soluble form retains hyaluronidase activity. Soluble hyaluronidases include recombinant soluble hyaluronidases and those contained in or purified from natural sources, such as, for example, testes extracts from sheep or cows. Exemplary of such soluble hyaluronidases are soluble human PH20. Other soluble hyaluronidases include ovine (SEQ ID NO:27) and bovine (SEQ ID NO:11) PH20.

As used herein, soluble human PH20 or sHuPH20 include mature polypeptides lacking all or a portion of the glycosylphosphatidylinositol (GPI) attachment site at the C-terminus such that upon expression, the polypeptides are soluble. Exemplary sHuPH20 polypeptides include mature polypeptides having an amino acid sequence set forth in any one of SEQ ID NOS:4-9 and 47-48. The precursor polypeptides for such exemplary sHuPH20 polypeptides include a signal sequence. Exemplary of the precursors are those set forth in SEQ ID NOS:3 and 40-46, each of which contains a 35 amino acid signal sequence at amino acid positions 1-35. Soluble HuPH20 polypeptides also include those degraded during or after the production and purification methods described herein.

As used herein, soluble recombinant human PH20 (rHuPH20) refers to a soluble form of human PH20 that is recombinantly expressed in Chinese Hamster Ovary (CHO) cells. Soluble rHuPH20 is encoded by nucleic acid that includes the signal sequence and is set forth in SEQ ID NO:49. Also included are DNA molecules that are allelic variants thereof and other soluble variants. The nucleic acid encoding soluble rHuPH20 is expressed in CHO cells which secrete the mature polypeptide. As produced in the culture medium there is heterogeneity at the C-terminus so that the product includes a mixture of species that can include any one or more of SEQ ID NOS:4-9 in various abundance. Corresponding allelic variants and other variants also are included, including those corresponding to the precursor human PH20 polypeptides set forth in SEQ ID NOS:50-51. Other variants can have 60%, 70%, 80%, 85%, 90%, 91%, 92%, 93%, 94%, 95%, 96%, 97%, 98%, 99% or more sequence identity with any of SEQ ID NOS. 4-9 and 47-48 as long they retain a hyaluronidase activity and are soluble.

As used herein, activity refers to a functional activity or activities of a polypeptide or portion thereof associated with a full-length (complete) protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, hyaluronidase activity refers to the ability of hyaluronidase to cleave hyaluronic acid. In vitro assays to determine the hyaluronidase activity of hyaluronidases, such as soluble rHuPH20, are know in the art and described herein. Exemplary assays include the microturbidity assay described below (see e.g., Example 5) that measures cleavage of hyaluronic acid by hyaluronidase indirectly by detecting the insoluble precipitate formed when the uncleaved hyaluronic acid binds with serum albumin.

As used herein, the residues of naturally occurring α-amino acids are the residues of those 20 α-amino acids found in nature which are incorporated into protein by the specific recognition of the charged tRNA molecule with its cognate mRNA codon in humans.

As used herein, nucleic acids include DNA, RNA and analogs thereof, including peptide nucleic acids (PNA) and mixtures thereof. Nucleic acids can be single or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous nucleotides of sequence complementary to or identical to a gene of interest. Probes and primers can be 10, 20, 30, 50, 100 or more nucleic acids long.

As used herein, a peptide refers to a polypeptide that is from 2 to 40 amino acids in length.

As used herein, the amino acids which occur in the various sequences of amino acids provided herein are identified according to their known, three-letter or one-letter abbreviations (Table 1a). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem. (1968) 243:3557-3559, and §§ 1.821-1.822, abbreviations for amino acid residues are adopted 37 C.F.R. shown in Table 1a:

TABLE 1a
Table of Correspondence

| SYMBOL | | |
|---|---|---|
| 1-Letter | 3-Letter | AMINO ACID |
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glu and/or Gln |
| W | Trp | Tryptophan |
| R | Arg | Arginine |
| D | Asp | Aspartic acid |
| N | Asn | Asparagine |
| B | Asx | Asn and/or Asp |
| C | Cys | Cysteine |
| X | Xaa | Unknown or other |

It is noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence (Table 1a) and modified and unusual amino acids, such as those referred to in 37 C.F.R. §§ 1.821-1.822, and incorporated herein by reference. Furthermore, it is noted that a dash at the beginning or end of an amino acid residue sequence indicates a peptide bond to a further sequence of one or more amino acid residues, to an amino-terminal group such as NH₂ or to a carboxyl-terminal group such as COOH.

As used herein, “naturally occurring amino acids” refer to the 20 L-amino acids that occur in polypeptides.

As used herein, “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally-occurring amino acids and include, but are not limited to, the D-isostereomers of amino acids. Exemplary non-natural amino acids are described herein and are known to those of skill in the art.

As used herein, a DNA construct is a single or double stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule can not be paired. Such unpaired ends will, in general, not exceed 20 nucleotides in length.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

“Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exists a number of methods to measure identity between two polynucleotide or polypeptides, the term “identity” is well known to skilled artisans (Carillo, H. & Lipton, D., SIAM J Applied Math 48:1073 (1988)).

As used herein, homologous (with respect to nucleic acid and/or amino acid sequences) means about greater than or equal to 25% sequence homology, typically greater than or equal to 25%, 40%, 50%, 60%, 70%, 80%, 85%, 90% or 95% sequence homology; the precise percentage can be specified if necessary. For purposes herein the terms “homology” and “identity” are often used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence homology, the number of conserved amino acids is determined by standard alignment algorithms programs, and can be used with default gap penalties established by each supplier. Substantially homologous nucleic acid molecules typically hybridize at moderate stringency or at high stringency all along the length of the nucleic acid of interest. Also contemplated are nucleic acid molecules that contain degenerate codons in place of codons in the hybridizing nucleic acid molecule.

Whether any two molecules have nucleotide sequences or amino acid sequences that are at least 60%, 70%, 80%, 85%, 90%, 95%, 96%, 97%, 98% or 99% “identical” or “homologous” can be determined using known computer algorithms such as the “FASTA” program, using for example, the default parameters as in Pearson et al. (1988) Proc. Natl. Acad. Sci. USA 85:2444 (other programs include the GCG program package (Devereux, J. et al. (1984) Nuc. Acids Res. 12(I):387), BLASTP, BLASTN, FASTA (Atschul, S. F. et al. (1990) J. Mol. Biol. 215:403); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. As used herein, the term at least “90% identical to” refers to percent identities from 90 to 99.99 relative to the reference nucleic acid or amino acid sequence of the polypeptide. Identity at a level of 90% or more is indicative of the fact that, assuming for exemplification purposes a test and reference polypeptide length of 100 amino acids are compared. No more than 10% (i.e., 10 out of 100) of the amino acids in the test polypeptide differs from that of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result is independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that a certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g., by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application. Complementary, when referring to two nucleotide sequences, means that the two sequences of nucleotides are capable of hybridizing, typically with less than 25%, 15% or 5% mismatches between opposed nucleotides. If necessary, the percentage of complementarity will be specified. Typically the two molecules are selected such that they will hybridize under conditions of high stringency.

As used herein, substantially identical to a product means sufficiently similar so that the property of interest is sufficiently unchanged so that the substantially identical product can be used in place of the product.

As used herein, it also is understood that the terms “substantially identical” or “similar” varies with the context as understood by those skilled in the relevant art.

As used herein, an allelic variant or allelic variation references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide. Reference to an allelic variant herein generally refers to variations n proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include substitutions, deletions and insertions of nucleotides. An allele of a gene also can be a form of a gene containing a mutation.

As used herein, species variants refer to variants in polypeptides among different species, including different mammalian species, such as mouse and human.

As used herein, a splice variant refers to a variant produced by differential processing of a primary transcript of genomic DNA that results in more than one type of mRNA.

As used herein, modification is in reference to modification of a sequence of amino acids of a polypeptide or a sequence of nucleotides in a nucleic acid molecule and includes deletions, insertions, and replacements of amino acids and nucleotides, respectively. Methods of modifying a polypeptide are routine to those of skill in the art, such as by using recombinant DNA methodologies.

As used herein, the term promoter means a portion of a gene containing DNA sequences that provide for the binding of RNA polymerase and initiation of transcription. Promoter sequences are commonly, but not always, found in the 5′ non-coding region of genes.

As used herein, isolated or purified polypeptide or protein or biologically-active portion thereof is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification does not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

The term substantially free of cellular material includes preparations of proteins in which the protein is separated from cellular components of the cells from which it is isolated or recombinantly-produced. In one embodiment, the term substantially free of cellular material includes preparations of enzyme proteins having less that about 30% (by dry weight) of non-enzyme proteins (also referred to herein as a contaminating protein), generally less than about 20% of non-enzyme proteins or 10% of non-enzyme proteins or less that about 5% of non-enzyme proteins. When the enzyme protein is recombinantly produced, it also is substantially free of culture medium, i.e., culture medium represents less than at or about 20%, 10% or 5% of the volume of the enzyme protein preparation.

As used herein, the term substantially free of chemical precursors or other chemicals includes preparations of enzyme proteins in which the protein is separated from chemical precursors or other chemicals that are involved in the synthesis of the protein. The term includes preparations of enzyme proteins having less than about 30% (by dry weight) 20%, 10%, 5% or less of chemical precursors or non-enzyme chemicals or components.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant means by using recombinant DNA methods means the use of the well known methods of molecular biology for expressing proteins encoded by cloned DNA.

As used herein, vector (or plasmid) refers to discrete elements that are used to introduce a heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, vector also includes “virus vectors” or “viral vectors.” Viral vectors are engineered viruses that are operatively linked to exogenous genes to transfer (as vehicles or shuttles) the exogenous genes into cells.

As used herein, operably or operatively linked when referring to DNA segments means that the segments are arranged so that they function in concert for their intended purposes, e.g., transcription initiates in the promoter and proceeds through the coding segment to the terminator.

As used herein the term assessing is intended to include quantitative and qualitative determination in the sense of obtaining an absolute value for the activity of a protease, or a domain thereof, present in the sample, and also of obtaining an index, ratio, percentage, visual or other value indicative of the level of the activity. Assessment can be direct or indirect and the chemical species actually detected need not of course be the proteolysis product itself but can for example be a derivative thereof or some further substance. For example, detection of a cleavage product of a complement protein, such as by SDS-PAGE and protein staining with Coomasie blue.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities. Thus, for purposes herein a biological activity of a protease is its catalytic activity in which a polypeptide is hydrolyzed.

As used herein equivalent, when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with only amino acid substitutions that do not substantially alter the activity or function of the protein or peptide. When equivalent refers to a property, the property does not need to be present to the same extent (e.g., two peptides can exhibit different rates of the same type of enzymatic activity), but the activities are usually substantially the same.

As used herein, “modulate” and “modulation” or “alter” refer to a change of an activity of a molecule, such as a protein. Exemplary activities include, but are not limited to, biological activities, such as signal transduction. Modulation can include an increase in the activity (i.e., up-regulation or agonist activity) a decrease in activity (i.e., down-regulation or inhibition) or any other alteration in an activity (such as a change in periodicity, frequency, duration, kinetics or other parameter). Modulation can be context dependent and typically modulation is compared to a designated state, for example, the wildtype protein, the protein in a constitutive state, or the protein as expressed in a designated cell type or condition.

As used herein, a composition refers to any mixture. It can be a solution, suspension, liquid, powder, paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a kit is a packaged combination that optionally includes other elements, such as additional reagents and instructions for use of the combination or elements thereof.

As used herein, “disease or disorder” refers to a pathological condition in an organism resulting from cause or condition including, but not limited to, infections, acquired conditions, genetic conditions, and characterized by identifiable symptoms. Diseases and disorders of interest herein are those involving components of the ECM.

As used herein, “treating” a subject with a disease or condition means that the subject's symptoms are partially or totally alleviated, or remain static following treatment. Hence treatment encompasses prophylaxis, therapy and/or cure. Prophylaxis refers to prevention of a potential disease and/or a prevention of worsening of symptoms or progression of a disease. Treatment also encompasses any pharmaceutical use of a modified interferon and compositions provided herein.

As used herein, a pharmaceutically effective agent includes any therapeutic agent or bioactive agents, including, but not limited to, for example, anesthetics, vasoconstrictors, dispersing agents, conventional therapeutic drugs, including small molecule drugs and therapeutic proteins.

As used herein, treatment means any manner in which the symptoms of a condition, disorder or disease or other indication, are ameliorated or otherwise beneficially altered.

As used herein therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, the term “subject” refers to an animal, including a mammal, such as a human being.

As used herein, a patient refers to a human subject.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, prevention or prophylaxis refers to methods in which the risk of developing disease or condition is reduced.

As used herein, a “therapeutically effective amount” or a “therapeutically effective dose” refers to the quantity of an agent, compound, material, or composition containing a compound that is at least sufficient to produce a therapeutic effect. Hence, it is the quantity necessary for preventing, curing, ameliorating, arresting or partially arresting a symptom of a disease or disorder.

As used herein, unit dose form refers to physically discrete units suitable for human and animal subjects and packaged individually as is known in the art.

As used herein, a single dosage formulation refers to a formulation for direct administration.

As used herein, an “article of manufacture” is a product that is made and sold. As used throughout this application, the term is intended to encompass bisphosphonate and hyaluronidase compositions contained in articles of packaging.

As used herein, fluid refers to any composition that can flow. Fluids thus encompass compositions that are in the form of semi-solids, pastes, solutions, aqueous mixtures, gels, lotions, creams and other such compositions.

As used herein, a “kit” refers to a combination of compositions provided herein and another item for a purpose including, but not limited to, activation, administration, diagnosis, and assessment of a biological activity or property. Kits optionally include instructions for use.

As used herein, a cellular extract or lysate refers to a preparation or fraction which is made from a lysed or disrupted cell.

As used herein, animal includes any animal, such as, but are not limited to primates including humans, gorillas and monkeys; rodents, such as mice and rats; fowl, such as chickens; ruminants, such as goats, cows, deer, sheep; ovine, such as pigs and other animals. Non-human animals exclude humans as the contemplated animal. The enzymes provided herein are from any source, animal, plant, prokaryotic and fungal. Most enzymes are of animal origin, including mammalian origin.

As used herein, a control refers to a sample that is substantially identical to the test sample, except that it is not treated with a test parameter, or, if it is a plasma sample, it can be from a normal volunteer not affected with the condition of interest. A control also can be an internal control.

As used herein, the singular forms “a,” “an” and “the” include plural referents unless the context clearly dictates otherwise. Thus, for example, reference to a compound, comprising “an extracellular domain” includes compounds with one or a plurality of extracellular domains. Reference to a composition containing “a soluble hyaluronidase” is intended to encompass composition containing one or more soluble hyaluronidases. Likewise, reference to composition containing “a bisphosphonate” is intended to encompass a composition containing one or more bisphosphonates.

As used herein, ranges and amounts can be expressed as “about” a particular value or range. About also includes the exact amount. Hence “about 5 bases” means “about 5 bases” and also “5 bases.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, C₁-Cₓ includes C₁-C₂, C₁-C₃. C₁-Cₓ.

The term “alkyl” refers to straight or branched chain substituted or unsubstituted hydrocarbon groups, in one embodiment 1 to 40 carbon atoms, in another embodiment, 1 to 20 carbon atoms, in another embodiment, 1 to 10 carbon atoms. The expression “lower alkyl” refers to an alkyl group of 1 to 6 carbon atoms. An alkyl group can be a “saturated alkyl,” meaning that it does not contain any alkene or alkyne groups and in certain embodiments, alkyl groups are optionally substituted. An alkyl group can be an “unsaturated alkyl,” meaning that it contains at least one alkene or alkyne group. An alkyl group that includes at least one carbon-carbon double bond (C═C) also is referred to by the term “alkenyl,” and in certain embodiments, alkenyl groups are optionally substituted. An alkyl group that includes at least one carbon-carbon triple bond (C≡C) also is referred to by the term “alkynyl,” and in certain embodiments, alkynyl groups are optionally substituted.

In certain embodiments, an alkyl contains 1 to 20 carbon atoms (whenever it appears herein, a numerical range such as “1 to 20” refers to each integer in the given range; e.g., “1 to 20 carbon atoms” means that an alkyl group can contain only 1 carbon atom, 2 carbon atoms, 3 carbon atoms, etc., up to and including 20 carbon atoms, although the term “alkyl” also includes instances where no numerical range of carbon atoms is designated). An alkyl can be designated as “C₁-C₄ alkyl” or by similar designations. By way of example only, “C₁-C₄ alkyl” indicates an alkyl having one, two, three, or four carbon atoms, i.e., the alkyl is selected from among methyl, ethyl, propyl, iso-propyl, n-butyl, iso-butyl, sec-butyl and t-butyl. Thus “C₁-C₄” includes C₁-C₂, C₁-C₃, C₂-C₃ and C₂-C₄ alkyl. Alkyls include, but are not limited to, methyl, ethyl, propyl, isopropyl, butyl, isobutyl, tertiary butyl, pentyl, hexyl, ethenyl, propenyl, butenyl, hexenyl, ethynyl, propynyl, butynyl and hexynyl.

As used herein, “halogen” or “halide” or “halo” refers to F, Cl, Br or I and includes pseudohalo or pseudohalides. As used herein, pseudohalo or pseudohalides are compounds that behave substantially similar to halides. Such compounds can be used in the same manner and treated in the same manner as halides (X-, in which X is a halogen, such as Cl, F or Br). Pseudohalos and pseudohalides include, but are not limited to, cyanide, cyanate, thiocyanate, selenocyanate, trifluoromethoxy, trifluoromethyl and azide.

As used herein, “cycloalkyl” refers to a saturated mono- or multicyclic ring system where each of the atoms forming a ring is a carbon atom. Cycloalkyls can be formed by three, four, five, six, seven, eight, nine, or more than nine carbon atoms. In one embodiment, the ring system includes 3 to 12 carbon atoms. In another embodiment, they ring system includes 3 to 6 carbon atoms. The term “cycloalkyl” includes rings that contain one or more unsaturated bonds. As used herein, the terms “cycloalkenyl” and “cycloalkynyl” are unsaturated cycloalkyl ring system. Cycloalkyls can be optionally substituted. In certain embodiments, a cycloalkyl contains one or more unsaturated bonds. Examples of cycloalkyls include, but are not limited to, cyclopropane, cyclobutane, cyclopentane, cyclopentene, cyclopentadiene, cyclohexane, cyclohexene, 1,3-cyclohexadiene, 1,4-cyclohexadiene, cycloheptane and cycloheptene.

As used herein, the term “cycloalkenyl” refers to mono- or multicyclic ring systems that includes at least one carbon-carbon double bond (C═C).

As used herein, the term “cycloalkynyl” refers to mono- or multicyclic ring systems that includes at least one carbon-carbon triple bond (C≡C).

Cycloalkenyl and cycloalkynyl groups include ring systems that include 3 to 12 carbon atoms. In some embodiments, the cycloalkenyl groups include 4 to 7 carbon atoms. In some embodiment, the cycloalkynyl groups include 8 to 10 carbon atoms. The ring systems of the cycloalkyl, cycloalkenyl and cycloalkynyl groups can be composed of one ring or two or more rings that can be joined together in a fused, bridged or spiro-connected fashion, and can be optionally substituted with one or more alkyl group substituents.

As used herein, the term “heterocycle” refers to a ring wherein at least one atom forming the ring is a carbon atom and at least one atom forming the ring is a heteroatom. Heterocyclic rings can be formed by three, four, five, six, seven, eight, nine, or more than nine atoms. Any number of those atoms can be heteroatoms (i.e., a heterocyclic ring can contain one, two, three, four, five, six, seven, eight, nine, or more than nine heteroatoms, provided that at least one atom in the ring is a carbon atom). Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocyclic ring will have additional heteroatoms in the ring. Designations such as “4-6 membered heterocycle” refer to the total number of atoms that comprise the ring (i.e., a four, five, or six membered ring, in which at least one atom is a carbon atom, at least one atom is a heteroatom and the remaining two to four atoms are either carbon atoms or heteroatoms). In heterocycles containing two or more heteroatoms, those two or more heteroatoms can be the same or different from one another. In one embodiment, the heterocycle includes 3-12 members. In other embodiments, the heterocycle includes 4, 5, 6, 7 or 8 members. The heterocycle can be optionally substituted with one or more substituents. In some embodiments, the substituents of the heterocyclic group are selected from among hydroxy, amino, alkoxy containing 1 to 4 carbon atoms, halo lower alkyl, including trihalomethyl, such as trifluoromethyl, and halogen. As used herein, the term heterocycle can include reference to heteroaryl. Binding to a heterocycle can be at a heteroatom or via a carbon atom. Examples of heterocycles include, but are not limited to, the following:

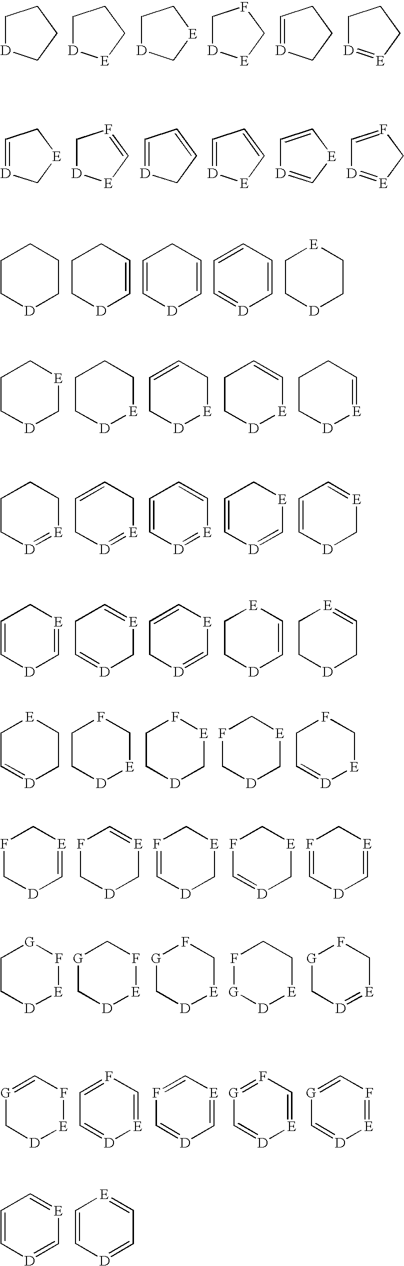

where D, E, F and G independently represent a heteroatom. Each of D, E, F and G can be the same or different from one another.

As used herein, the term “bicyclic ring” refers to two rings, wherein the two rings are fused. Bicyclic rings include, for example, decaline, pentalene, naphthalene, azulene, heptalene, isobenzofuran, chromene, indolizine, isoindole, indole, purine, indoline, indene, quinolizine, isoquinoline, quinoline, phthalazine, naphthyrididine, quinoxaline, cinnoline, pteridine, isochroman, chroman and various hydrogenated derivatives thereof. Bicyclic rings can be optionally substituted. Each ring is independently aromatic or non-aromatic. In certain embodiments, both rings are aromatic. In certain embodiments, both rings are non-aromatic. In certain embodiments, one ring is aromatic and one ring is non-aromatic.

As used herein, the term “aromatic” refers to a planar ring having a delocalized π-electron system containing 4n+2π electrons, where n is an integer. Aromatic rings can be formed by five, six, seven, eight, nine, or more than nine atoms. Aromatics can be optionally substituted. Examples of aromatic groups include, but are not limited to phenyl, naphthalenyl, phenanthrenyl, anthracenyl, tetralinyl, fluorenyl, indenyl and indanyl. The term aromatic includes, for example, benzenoid groups, connected via one of the ring-forming carbon atoms, and optionally carrying one or more substituents selected from an aryl, a heteroaryl, a cycloalkyl, a non-aromatic heterocycle, a halo, a hydroxy, an amino, a cyano, a nitro, an alkylamido, an acyl, a C₁₋₆ alkoxy, a C₁₋₆ alkyl, a C₁₋₆ hydroxyalkyl, a C₁₋₆ aminoalkyl, a C₁₋₆ alkylamino, an alkylsulfenyl, an alkylsulfinyl, an alkylsulfonyl, an sulfamoyl, or a trifluoromethyl. In certain embodiments, an aromatic group is substituted at one or more of the para, meta, and/or ortho positions. Examples of aromatic groups containing substitutions include, but are not limited to, phenyl, 3-halophenyl, 4-halo-phenyl, 3-hydroxyphenyl, 4-hydroxy-phenyl, 3-aminophenyl, 4-aminophenyl, 3-methyl-phenyl, 4-methylphenyl, 3-methoxyphenyl, 4-methoxyphenyl, alkoxyphenyl, 4-trifluoro-methoxyphenyl, 3-cyano-phenyl, 4-cyanophenyl, dimethylphenyl, naphthyl, hydroxy-naphthyl, hydroxy-methylphenyl, (trifluoromethyl)phenyl, 4-morpholin-4-ylphenyl, 4-pyrazolylphenyl, 4-pyrrolidin-1-ylphenyl, 4-triazolylphenyl and 4-(2-oxopyrrolidin-1-yl)phenyl.

As used herein, the term “aryl” refers to a monocyclic, bicyclic or tricyclic aromatic system that contains no ring heteroatoms. Where the systems are not monocyclic, the term aryl includes for each additional ring the saturated form (perhydro form) or the partially unsaturated form (for example the dihydro form or tetrahydro form) or the maximally unsaturated (nonaromatic) form. In some embodiments, the term aryl refers to bicyclic radicals in which the two rings are aromatic and bicyclic radicals in which only one ring is aromatic. Examples of aryl include phenyl, naphthyl, anthracyl, indanyl, indenyl, 1,2-dihydronaphthyl, 1,4-dihydronaphthyl, 1,4-naphthoquinonyl and 1,2,3,4-tetrahydronaphthyl.

Aryl rings can be formed by three, four, five, six, seven, eight, nine, or more than nine carbon atoms. In some embodiments, aryl refers to a 3-, 4-, 5-, 6-, 7-, 8-, 9-, 10-, 11-, 12-, 13- or 14-membered, aromatic mono-, bi- or tricyclic system. In some embodiments, aryl refers to an aromatic C₃-C₉ ring. In some embodiments, aryl refers to an aromatic C₄-C₈ ring. Aryl groups can be optionally substituted.

As used herein, the term “heteroaryl” refers to an aromatic ring in which at least one atom forming the aromatic ring is a heteroatom. Heteroaryl rings can be formed by three, four, five, six, seven, eight, nine and more than nine atoms. Heteroaryl groups can be optionally substituted. Examples of heteroaryl groups include, but are not limited to, aromatic C₃₋₈ heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl) pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (for example the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-iso-quinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydro-benzo[g]-isoquinolinyl, 1,2,3,4-tetrahydro-benzo[g]isoquinolinyl, chromanyl, isochromanonyl, 2,3-dihydrochromonyl, 1,4-benzodioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydrobenzoxazolyl, 1,4-naphthoquinolyl, 5,6,7,8-tetrahydro-quinolinyl, 5,6,7,8-tetrahydroisoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diazanaphthalen-2-onyl, 1,3-dihydroimidizolo-[4,5]-pyridin-2-onyl, 2,3-dihydro-1,4-dinaphthoquinonyl, 2,3-dihydro-1H-pyrrol[3,4-b]quinolinyl, 1,2,3,4-tetrahydrobenzo[b][1,7]naphthyridinyl, 1,2,3,4-tetrahydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo[3,4-b]indolyl, 1H-2,3,4,5-tetrahydro-azepino[3,4-b]indolyl, 1H-2,3,4,5-tetrahydroazepino[4,3-b]indolyl, 1H-2,3,4,5-tetrahydro-azepino[4,5-b]indolyl, 5,6,7,8-tetrahydro[1,7]napthyridinyl, 1,2,3,4-tetrahydro[2,7]-naphthyridyl, 2,3-dihydro-[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo[4,5-c]pyridyl, 6,7-dihydro-[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro[1,8]napthyridinyl or 1,2,3,4-tetrahydro[2,6]napthyridinyl.

In certain embodiments, heteroaryl groups are optionally substituted. In one embodiment, the one or more substituents are each independently selected from among halo, hydroxy, amino, cyano, nitro, alkylamido, acyl, C₁₋₆-alkoxy, C₁₋₆-alkyl, C₁₋₆-halo-alkyl, C₁₋₆-hydroxy-alkyl, C₁₋₆-aminoalkyl, C₁₋₆-alkylamino, alkylsulfenyl, alkylsulfinyl, alkylsulfonyl, sulfamoyl, or trifluoromethyl. Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O—C₁₋₆-alkyl, C₁₋₆-alkyl, hydroxy-C₁₋₆-alkyl and amino-C₁₋₆-alkyl.

As used herein, the term “non-aromatic ring” refers to a ring that does not have a delocalized 4n+2 π-electron system.

As used herein, the term “non-aromatic heterocycle” refers to a non-aromatic ring wherein one or more atoms forming the ring is a heteroatom. Non-aromatic heterocyclic rings can be formed by three, four, five, six, seven, eight, nine, or more than nine atoms. Non-aromatic heterocycles can be optionally substituted. In certain embodiments, non-aromatic heterocycles contain one or more carbonyl or thiocarbonyl groups such as, for example, oxo- and thio-containing groups. Examples of non-aromatic heterocycles include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine and 1,3-oxathiolane.

Unless otherwise indicated, the term “optionally substituted,” refers to a group in which none, one, or more than one of the hydrogen atoms has been replaced with one or more group(s) individually and independently selected from among alkyl, cycloalkyl, heteroalkyl, aryl, heteroaryl, non-aromatic heterocycle, hydroxy, alkoxy, aryloxy, mercapto, alkylthio, arylthio, cyano, halo, carbonyl, thiocarbonyl, O-carbamyl, N carbamyl, O thiocarbamyl, N thiocarbamyl, C amido, N amido, S-sulfonamido, N sulfonamido, C carboxy, O carboxy, isocyanato, thiocyanato, isothiocyanato, nitro, silyl, trihalomethane-sulfonyl, and amino, including mono- and di-substituted amino groups, and the protected derivatives of amino groups. Such protective derivatives (and protecting groups that can form such protective derivatives) are known to those of skill in the art and can be found in references such as Greene and Wuts, above. In embodiments in which two or more hydrogen atoms have been substituted, the substituent groups can together form a ring.

Throughout the specification, groups and substituents thereof can be chosen by one skilled in the field to provide stable moieties and compounds.

As used herein, “pharmaceutically acceptable salts” include, but are not limited to, amine salts, such as but not limited to chloroprocaine, choline, N,N′-dibenzyl-ethylenediamine, ammonia, diethanolamine and other hydroxyalkylamines, ethylenediamine, N-methylglucamine, procaine, N-benzyl-phenethylamine, 1-para-chloro-benzyl-2-pyrrolidin-1′-ylmethyl-benzimidazole, diethylamine and other alkylamines, piperazine and tris(hydroxymethyl)-aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

As used herein, “solvates” and “hydrates” are complexes of a compound with one or more solvent or water molecules, or 1 to about 100, or 1 to about 10, or one to about 2, 3 or 4, solvent or water molecules.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochemistry. 11:1726).

### B. Subcutaneous Administration of Bisphosphonates

Provided herein are methods and uses of treating bisphosphonate-treatable diseases and conditions by subcutaneously administering a bisphosphonate in combination with a soluble hyaluronidase. Hence, also provided are combinations of a bisphosphonate and a soluble hyaluronidase. By virtue of the ability of hyaluronidase to break down hyaluronic acid in the extracellular matrix, hyaluronidase facilitates subcutaneous infusions of therapeutic agents, such as bisphosphonates. A bisphosphonate is a therapeutic that is primarily administered by intravenous or oral routes to treat individuals with bone disorders that are treatable by inhibition of bone resorption. Subcutaneous injection of bisphosphonates alone causes injection site reactions characterized by erythema, induration, and ulceration of the skin surrounding the injection site in a concentration dependent manner. Injection site reactions also are commonly observed in patients receiving intravenous administration of bisphosphonates (Body (2004) Seminars in Oncology 31:73-78).

It is discovered herein that the incidence degree of injection site reactions is substantially reduced when a bisphosphonate (e.g., zoledronate or ibandronate) is administered in the presence of a hyaluronidase (see Examples 1 and 2). Maximal concentration of bisphosphonates that can be administered without producing ISRs is increased typically 3-5 fold when co-administered with rHuPH20. Further, the bioavailability of a bisphosphonate in the presence of hyaluronidase is greater than 90% of the bioavailability of the bisphosphonate following IV treatment (see Example 3). Hence, in the methods and uses provided herein, the combination therapy of hyaluronidase and a bisphosphonate permits the subcutaneous administration of a bisphosphonate at dosages and frequencies that are similar to IV treatment.

In the methods and uses provided herein, a bisphosphonate, when administered subcutaneously in the presence of a soluble hyaluronidase, can be administered at prevailing IV doses for the particular indication. Further, because hyaluronidase acts to open flow channels in the skin, infusion rates can be increased. Hence, methods of subcutaneously administering a bisphosphonate co-formulated and/or co-administered with hyaluronidase increases infusion rates of the therapeutic agent and thereby can decrease time of delivery of bisphosphonate therapy.

The SC space is formed by a collagen network filled with a gel-like substance, hyaluronic acid. Hyaluronic acid is largely responsible for the resistance to fluid flow through the tissues. High levels of hyaluronic acid are normally maintained in the SC space by rapid synthesis, which is balanced by high turnover rate (i.e., hyaluronic acid in SC tissues is replaced with a half-life of approximately 5 hours). Hyaluronidase temporarily digests the hyaluronic acid, thereby facilitating infusions into the subcutaneous space. The hyaluronic acid is restored within 24 hours, leaving no observable changes. Thus, due to the ability of hyaluronidase to open channels in the interstitial space through degradation of glycosaminoglycans, administration of a soluble hyaluronidase permits the diffusion of molecules, thereby improving the bioavailability, pharmacokinetics and/or pharmacodynamic characteristics of such co-formulated or co-administered molecules.

In some examples, the bioavailability of a bisphosphonate co-administered subcutaneously with hyaluronidase is 70%, 80%, 90%, 91%, 92%, 93%, 94%, 95%, 96%, 97%, 98%, 99%, or 100% of the bioavailability of the bisphosphonate administered intravenously. Typically, the bioavailability is greater than 90%.

Further, subcutaneous co-administration of a bisphosphonate in the presence of a soluble hyaluronidase permits infusion of large volumes at a single subcutaneous site. For example, volumes up to 600 ml or greater of a bisphosphonate can be administered at a single site in a single sitting, for example 200 ml, 300 ml, 400 ml, 500, ml, 600 ml or more can be administered at a single site in a single administration.

Smaller volumes, such as, for example, less than 1 ml, less than less than 2 ml less than 3 ml, less than 4 ml, less than 5 ml, less than 6 ml, less than 7 ml, less than 8 ml, less than 9 ml, less than 10 ml, less than 20 ml, less than 30 ml less than 40 ml less than 50 less than 60 less than 70 less than 80 less than 90 less than 100 or less than 200 ml of a bisphosphonate in the presence of a hyaluronidase also can be administered. Because hyaluronidase can eliminate or reduce injection site toxicity induced by subcutaneous administration of bisphosphonates, higher concentrations of bisphosphonates can be administered subcutaneously and at higher infusion rates.

A bisphosphonate preparation can be co-administered subcutaneously with a soluble hyaluronidase at dosages equivalent to IV doses, for example, at or about 0.5 milligrams (mg), at or about 1 mg, at or about 3 mg, at or about 5 mg, at or about 10 mg, at or about 20 mg, at or about 30 mg, at or about 40 mg, at or about 50 mg, at or about 60 mg, at or about 70 mg, at or about 80 mg, at or about 90 mg, at or about 100 mg or more of a bisphosphonate. Hence, by administering a bisphosphonate subcutaneously in the presence of a soluble hyaluronidase, one, or more, or all of the considerations and problems associated with subcutaneous administration of a bisphosphonate are addressed. Thus, by virtue of the dispersion properties of hyaluronidase, it is concluded herein that subcutaneously administering a bisphosphonate in the presence of a soluble hyaluronidase permits administration of doses typically used for IV administration at frequencies that are the same as for IV administration or less frequently, while maintaining bisphosphonate bioavailability and reducing injection site toxicity.

The following sections describe exemplary bisphosphonates and soluble hyaluronidases in the combinations herein, methods of making them, and using them to treat a bisphosphonate-treatable diseases and conditions.

### C. Bisphosphonates

Bisphosphonates are specific inhibitors of osteoclast-mediated bone resorption. An osteoclast is a type of bone cell characterized by multiple nuclei and formed from differentiated macrophages. Osteoclasts are nondividing, motile cells that reabsorb surplus or inferior bone matrix in a process known as bone resorption or osteolysis. During bone resorption, osteoclasts secrete acid. The lowering of pH causes the decalcification of the bone's surface layer. Acid hydrolases, collagenases and other proteolytic enzymes secreted by the osteoclasts then break down the organic portion of the bone matrix. The organic and inorganic degradation products are reabsorbed by the osteoclasts and sequentially released into the capillaries where they are recycled to other locations. During the growth and continual remodeling of bone, osteoclasts and osteoblasts, which form bone, work together in the balance of bone resorption and formation.

Bisphosphonates attach to bone due to their affinity for hydroxyapatite, which is part of the bone mineral matrix. Following attachment, the bisphosphonates are taken up by osteoclasts. Non-nitrogenous bisphosphonates (e.g., etidronate, clodronate, tiludronate) are metabolized in the cell into compounds that replace the terminal pyrophosphate moiety of adenosine triphosphate (ATP), forming a nonfunctional molecule that competes with ATP in cellular energy metabolism. As a result, the osteoclast undergoes apoptosis, leading to an overall decrease in the breakdown of bone. Nitrogenous bisphosphonates (e.g., pamidronate, neridronate, olpadronate, alendronate, ibandronate, risedronate and zoledronate) affect bone metabolism by binding and blocking the enzyme farnesyl diphosphate synthase (FPPS) in the HMG-CoA reductase pathway (i.e., mevalonate pathway). Disruption of the HMG CoA-reductase pathway prevents the formation of two metabolites, farnesol and geranylgeraniol, that are essential for attachment of proteins to the inner cell membrane (i.e., prenylation). Inhibition of prenylation can affect osteoclastogenesis, cell survival, and cytoskeletal dynamics, and can result in breakdown of the osteoclast cell membrane “ruffled border” that is required for contact between a resorbing osteoclast and a bone surface. (see H. Fleisch, Bisphosphonates In Bone Disease, From The Laboratory To The Patient, 3rd Edition, Parthenon Publishing (1997); D. E. Hughes et al. (1995) Journal of Bone and Mineral Research 10(10):1478-1487).

Bisphosphonates are used clinically for the treatment of bone disorders including, osteoporosis, osteitis deformans (Paget's disease of the bone), bone metastasis (with or without hypercalcaemia), multiple myeloma, osteogenesis imperfecta and other conditions that are characterized by bone fragility. Bisphosphonate therapy reduces elevated bone turnover that occurs in these disorders, leading to a net gain in bone mass. Commercial bisphosphonates available for the treatment of such diseases and disorders, and can be used in the methods and uses provided, include alendronate/alendronic acid (FOSAMAX), clodronate/clodronic acid (BONEFOS), etidronate/etidronic acid (DIDRONEL), ibandronate.ibadronic acid (BONIVA, BONDRONAT, BONVIVA), pamidronate/pamidronic acid (AREDIA), risendronate/risendronic acid (ACTONEL), tiludronate/tiludronic acid (SKELID) and zoledronate/zoledronic acid (ZOMETA, ZOMERA, ACLASTA, RECLAST).

The methods and uses provided herein for the prevention or treatment of a bisphosphonate-treatable disease or condition involve administration of a bisphosphonate in the presence of a soluble hyaluronidase. Further, the compositions and combinations provided contain a bisphosphonate and a soluble hyaluronidase. Exemplary bisphosphonates that can be used in a combination with a soluble hyaluronidase provided herein include, but are not limited to, bisphosphonates corresponding to the chemical formula

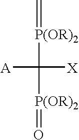

wherein X is selected from among H, OH, halogen, NH₂, NHR′, NR′R′, C₁-C₃₀ alkyl, OR′, CO₂R′, SH, SR′, C₃-C₃₀ cycloalkyl, C₃-C₃₀ heterocycloalkyl, C₅-C₁₄ aryl and C₅-C₁₄ heteroaryl, wherein the alkyl, cycloalkyl, heterocycloalkyl, aryl and heteroaryl groups are optionally substituted with a substituent selected from among OH, halogen, NH₂, NHR′, NR′R′, C₁-C₃₀ alkyl, OR′, SH, SR′, C₃-C₃₀ cycloalkyl, C₅-C₁₄ heterocycloalkyl, C₅-C₁₄ aryl and C₅-C₁₄ heteroaryl; each R′ is independently selected from among halogen, C₁-C₁₀ alkyl and C₅-C₁₄ aryl, wherein the alkyl and aryl groups are optionally substituted with a halogen substituent; each R is independently selected from among hydrogen and C₁-C₈ alkyl; A is selected from among H, OH, halogen, NH₂, NHR′, NR′R′, C₁-C₃₀ alkyl, OR′, CO₂R′, SH, SR′, C₃-C₃₀ cycloalkyl, C₃-C₃₀ heterocycloalkyl, C₅-C₁₄ aryl and C₅-C₁₄ heteroaryl wherein the alkyl, cycloalkyl, heterocycloalkyl, aryl and heteroaryl groups are optionally substituted with a substituent selected from among OH, halogen, NH₂, NHR′, NR′R′, C₁-C₃₀ alkyl, OR′, SH, SR′, C₃-C₃₀ cycloalkyl, C₅-C₁₄ heterocycloalkyl, C₅-C₁₄ aryl and C₅-C₁₄ heteroaryl; and pharmaceutically acceptable salts or esters thereof or any hydrate thereof.

Bisphosphonic acids or their physiologically acceptable salts as pharmaceutical active substances are described for example in U.S. Pat. Nos. 4,666,895; 4,719,203; 4,777,163; 5,002,937; and 4,971,958; and European Patent Nos. EP252,504 and EP252,505. Methods for the preparation of bisphosphonic acids may be found in, e.g., U.S. Pat. Nos. 3,962,432; 4,054,598; 4,267,108; 4,327,039; 4,407,761; 4,621,077; 4,624,947; 4,746,654; 4,922,077; 4,970,335; 5,019,651; 4,761,406; and 4,876,248; and J. Org. Chem. 32:4111 (1967); EP1296689 and EP252,504.

Exemplary bisphosphonates for use in the combinations, compositions and methods provided herein include, but are not limited to, 1-hydroxy-2-(1H-imidazol-1-yl)ethylidene-1,1-bisphosphonic acid (zoledronate; CAS No. 118072-93-8); 1-hydroxy-3-(N-methyl-N-pentylamino)propylidene-1,1-bisphosphonic acid (ibandronate; described in U.S. Pat. No. 4,927,814; CAS No. 114084-78-5); 3-amino-1-hydroxypropylidene-1,1-bisphosphonic acid (pamidronate; CAS No. 40391-99-9); (1-hydroxyl-1-phosphono-ethyl)phosphonic acid (etidronate; CAS 2809-21-4); 4-amino-1-hydroxy-1 (hydroxyl-oxido-phosphoryl)-butyl]monosodium trihydrate (alendronate; described in U.S. Pat. Nos. 4,922,007 and 5,019,651; CAS No. 121268-17-5); 1-hydroxy-2-(3-pyridinyl)-ethylidene-1,1-bisphosphonic acid (risedronate; CAS No. 105462-24-6); (dichloro-phosphono-methyl)phosphonic acid (clodronate; CAS No. 10596-23-3); {[(4-chlorophenyl)thio]methylene}bis(phosphonic acid) (tiludronate; CAS No. 89987-06-4); cycloheptylaminomethylene-1,1-bisphosphonic acid (cimadronate: described in U.S. Pat. No. 4,970,335; CAS No. 138330-18-4); 1-hydroxy-3-(1-pyrrolidinyl)-propylidene-1,1-bisphosphonic acid (EB-1053; CAS No. 125946-92-1); 6-amino-1-hydroxyhexylidene-1,1-bisphosphonic acid (neridronate; CAS No. 79778-41-9); 3-(dimethylamino)-1-hydroxypropylidene-1,1-bisphosphonic acid (olpadronate; CAS No. 63132-39-8); [2-(2-pyridinyl)ethylidene]-1,1-bisphosphonic acid (piridronate; described in U.S. Pat. No. 4,761,406; CAS No. 75755-07-6); [1-hydroxy-2-imidazo-(1,2-a)pyridin-3-ylethylidene]bisphosphonate (Yamanouchi YH 529; CAS No. 127657-42-5) and mixtures thereof. In particular examples, the bisphosphonate is selected from among pamidronate, ibandronate and zoledronate.

The bisphosphonate can be in crystalline or amorphous form or liquid form, and mixtures of bisphosphonates can be used in the combinations, compositions and methods provided. The bisphosphonate can be in the form of a pharmaceutically acceptable salt, ester, anhydride, carbamate, amide, hydrate, or other analog, derivative or prodrug, or a combination thereof. Salts of bisphosphonic acid compounds can be obtained commercially or can be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, amino acids, and the like, as well as inorganic acids, e.g., hydrochloric acid, hydrobromic acid, sulfuric acid, nitric acid, phosphoric acid, and the like. Basic salts of acid moieties, e.g., phosphonic acid groups, can be prepared using a pharmaceutically acceptable base. Salts formed with the phosphonic acid group include, but are not limited to, alkali metal salts, alkaline earth metal salts and organic base salts. For example, bases such as sodium hydroxide, potassium hydroxide, ammonium hydroxide, calcium hydroxide, magnesium hydroxide, trimethylamine, lysine, arginine, triethanolamine, and the like, can be used. Preparation of Esters Involves Functionalization of Hydroxyl and/or Carboxyl Groups which may be present. These esters are typically acyl-substituted derivatives of free alcohol groups, i.e., moieties which are derived from carboxylic acids of the formula RCOOH where R is alkyl, and preferably is lower alkyl. Pharmaceutically acceptable esters can be prepared using methods known to those skilled in the art and/or described in the art. Anhydrides, carbamates, amides, hydrates, and other analogs, derivatives and prodrugs can be readily prepared as well, using conventional means, and incorporated into the combinations, compositions and methods provided.

### D. Hyaluronan Degrading Enzymes

Provided herein are combinations containing a bisphosphonate and hyaluronan degrading enzymes, in particular soluble hyaluronidases, and methods of using such combinations for subcutaneous administration for the treatment of a bisphosphonate-mediated diseases and conditions. Any such hyaluronan degrading enzyme can be used herein provided the enzyme exhibits enzymatic activity for hyaluronic acid (e.g. hyaluronidase activity).

Hyaluronan, also called hyaluronic acid or hyaluronate, is a non-sulfated glycosaminoglycan that is widely distributed throughout connective, epithelial, and neural tissues. Hyaluronan is an essential component of the extracellular matrix and a major constituent of the interstitial barrier. By catalyzing the hydrolysis of hyaluronan, hyaluronan degrading enzymes lower the viscosity of hyaluronan, thereby increasing tissue permeability and increasing the absorption rate of fluids administered parenterally. As such, hyaluronan degrading enzymes, such as hyaluronidases, have been used, for example, as spreading or dispersing agents in conjunction with other agents, drugs and proteins to enhance their dispersion and delivery.

Hyaluronan degrading enzymes act to degrade hyaluronan by cleaving hyaluronan polymers, which are composed of repeating disaccharides units, D-glucuronic acid (GlcA) and N-acetyl-D-glucosamine (GlcNAc), linked together via alternating β-1→4 and β-1→3 glycosidic bonds. Hyaluronan chains can reach about 25,000 disaccharide repeats or more in length and polymers of hyaluronan can range in size from about 5,000 to 20,000,000 Da in vivo. Accordingly, hyaluronan degrading enzymes for the uses and methods provided include any enzyme having the ability to catalyze the cleavage of a hyaluronan disaccharide chain or polymer. In some examples the hyaluronan degrading enzyme cleaves the β-1→4 glycosidic bond in the hyaluronan chain or polymer. In other examples, the hyaluronan degrading enzyme catalyze the cleavage of the β-1→3 glycosidic bond in the hyaluronan chain or polymer.

Hence, hyaluronan degrading enzymes, such as hyaluronidases, are a family of enzymes that degrade hyaluronic acid, which is an essential component of the extracellular matrix and a major constituent of the interstitial barrier. By catalyzing the hydrolysis of hyaluronic acid, a major constituent of the interstitial barrier, hyaluronan degrading enzymes lower the viscosity of hyaluronic acid, thereby increasing tissue permeability. As such, hyaluronan degrading enzymes, such as hyaluronidases, have been used, for example, as a spreading or dispersing agent in conjunction with other agents, drugs and proteins to enhance their dispersion and delivery. Exemplary of hyaluronan degrading enzymes in the compositions and methods provided herein are soluble hyaluronidases. Other exemplary hyaluronan degrading enzymes include, but are not limited to particular chondroitinases and lyases that have the ability to cleave hyaluronan.

As described below, hyaluronan-degrading enzymes exist in membrane-bound or soluble form. For purposes herein, soluble hyaluronan-degrading enzymes are provided for use in the methods, uses, compositions or combinations herein. Thus, where hyaluronan-degrading enzymes include a glycosylphosphatidylinositol (GPI) anchor and/or are otherwise membrane-anchored or insoluble, hyaluronan-degrading enzymes are provided herein in soluble form. Thus, hyaluronan-degrading enzymes include truncated variants, e.g. truncated to remove all or a portion of a GPI anchor. Hyaluronan-degrading enzymes provide herein also include allelic or species variants or other variants, of a soluble hyaluronan-degrading enzyme. For example, hyaluronan degrading enzymes can contain one or more variations in its primary sequence, such as amino acid substitutions, additions and/or deletions. A variant of a hyaluronan-degrading enzyme generally exhibits at least or about 60%, 70%, 80%, 90%, 91%, 92%, 93%, 94%, 95%, 96%, 97%, 98%, 99% or more sequence identity compared to the hyaluronan-degrading enzyme not containing the variation. Any variation can be included in the hyaluronan degrading enzyme for the purposes herein provided the enzyme retains hyaluronidase activity, such as at least or about 5%, 10%, 15%, 20%, 25%, 30%, 35%, 40%, 45%, 50%, 55%, 60%, 65%, 70%, 75%, 80%, 85%, 90%, 95% or more of the activity of a hyaluronan degrading enzyme not containing the variation (as measured by in vitro and/or in vivo assays well known in the art and described herein).

Where the methods and uses provided herein describe the use of a soluble hyaluronidase, accordingly any hyaluronan degrading enzyme, generally a soluble hyaluronan degrading enzyme, can be used.

1. Hyaluronidases

Hyaluronidases are members of a large family of hyaluronan degrading enzymes. There are three general classes of hyaluronidases: mammalian-type hyaluronidases, bacterial hyaluronidases and hyaluronidases from leeches, other parasites and crustaceans. Such enzymes can be used in the compositions, combinations and methods provided.

a. Mammalian-type hyaluronidases

Mammalian-type hyaluronidases (EC 3.2.1.35) are endo-β-N-acetyl-hexosaminidases that hydrolyze the β-1→4 glycosidic bond of hyaluronan into various oligosaccharide lengths such as tetrasaccharides and hexasaccharides. These enzymes have both hydrolytic and transglycosidase activities, and can degrade hyaluronan and chondroitin sulfates (CS), generally C4-S and C6-S. Hyaluronidases of this type include, but are not limited to, hyaluronidases from cows (bovine) (SEQ ID NOS:10, 11 and 64 and BH55 (U.S. Pat. Nos. 5,747,027 and 5,827,721)), sheep (ovis aries) (SEQ ID NO: 26, 27, 63 and 64), yellow jacket wasp (SEQ ID NOS:12 and 13), honey bee (SEQ ID NO:14), white-face hornet (SEQ ID NO:15), paper wasp (SEQ ID NO:16), mouse (SEQ ID NOS:17-19, 31), pig (SEQ ID NOS:20-21), rat (SEQ ID NOS:22-24, 30), rabbit (SEQ ID NO:25), orangutan (SEQ ID NO:28), cynomolgus monkey (SEQ ID NO:29), guinea pig (SEQ ID NO:32), and human hyaluronidases. Exemplary of hyaluronidases in the compositions, combinations and methods provided herein are soluble hyaluronidases.

Mammalian hyaluronidases can be further subdivided into those that are neutral active, predominantly found in testes extracts, and acid active, predominantly found in organs such as the liver. Exemplary neutral active hyaluronidases include PH20, including but not limited to, PH20 derived from different species such as ovine (SEQ ID NO:27), bovine (SEQ ID NO:11) and human (SEQ ID NO:1). Human PH20 (also known as SPAM1 or sperm surface protein PH20), is generally attached to the plasma membrane via a glycosylphosphatidyl inositol (GPI) anchor. It is naturally involved in sperm-egg adhesion and aids penetration by sperm of the layer of cumulus cells by digesting hyaluronic acid.

Besides human PH20 (also termed SPAM1), five hyaluronidase-like genes have been identified in the human genome, HYAL1, HYAL2, HYAL3, HYAL4 and HYALP1. HYALP1 is a pseudogene, and HYAL3 (SEQ ID NO:38) has not been shown to possess enzyme activity toward any known substrates. HYAL4 (precursor polypeptide set forth in SEQ ID NO:39) is a chondroitinase and exhibits little activity towards hyaluronan. HYAL1 (precursor polypeptide set forth in SEQ ID NO:36) is the prototypical acid-active enzyme and PH20 (precursor polypeptide set forth in SEQ ID NO:1) is the prototypical neutral-active enzyme. Acid-active hyaluronidases, such as HYAL1 and HYAL2 (precursor polypeptide set forth in SEQ ID NO:37) generally lack catalytic activity at neutral pH (i.e. pH 7). For example, HYAL1 has little catalytic activity in vitro over pH 4.5 (Frost et al. (1997) Anal. Biochem. 251:263-269). HYAL2 is an acid-active enzyme with a very low specific activity in vitro. The hyaluronidase-like enzymes also can be characterized by those which are generally attached to the plasma membrane via a glycosylphosphatidyl inositol (GPI) anchor such as human HYAL2 and human PH20 (Danilkovitch-Miagkova, et al. (2003) Proc Natl Acad Sci USA 100(8):4580-5), and those which are generally soluble such as human HYAL1 (Frost et al. (1997) Biochem Biophys Res Commun. 236(1):10-5).

i. PH20

PH20, like other mammalian hyaluronidases, is an endo-β-N-acetyl-hexosaminidase that hydrolyzes the β1→4 glycosidic bond of hyaluronic acid into various oligosaccharide lengths such as tetrasaccharides and hexasaccharides. They have both hydrolytic and transglycosidase activities and can degrade hyaluronic acid and chondroitin sulfates, such as C4-S and C6-S. PH20 is naturally involved in sperm-egg adhesion and aids penetration by sperm of the layer of cumulus cells by digesting hyaluronic acid. PH20 is located on the sperm surface, and in the lysosome-derived acrosome, where it is bound to the inner acrosomal membrane. Plasma membrane PH20 has hyaluronidase activity only at neutral pH, while inner acrosomal membrane PH20 has activity at both neutral and acid pH. In addition to being a hyaluronidase, PH20 also appears to be a receptor for HA-induced cell signaling, and a receptor for the zona pellucida surrounding the oocyte.

Exemplary PH20 proteins include, but are not limited to, human (precursor polypeptide set forth in SEQ ID NO:1, mature polypeptide set forth in SEQ ID NO: 2), chimpanzee (SEQ ID NO:101), Rhesus monkey (SEQ ID NO:102) bovine (SEQ ID NOS: 11 and 64), rabbit (SEQ ID NO: 25), ovine PH20 (SEQ ID NOS: 27, 63 and 65), Cynomolgus monkey (SEQ ID NO: 29), guinea pig (SEQ ID NO: 32), rat (SEQ ID NO: 30) and mouse (SEQ ID NO: 31) PH20 polypeptides.

Bovine PH20 is a 553 amino acid precursor polypeptide (SEQ ID NO:11). Alignment of bovine PH20 with the human PH20 shows only weak homology, with multiple gaps existing from amino acid 470 through to the respective carboxy termini due to the absence of a GPI anchor in the bovine polypeptide (see e.g., Frost G I (2007) Expert Opin. Drug. Deliv. 4: 427-440). In fact, clear GPI anchors are not predicted in many other PH20 species besides humans. Thus, PH20 polypeptides produced from ovine and bovine naturally exist as soluble forms. Though bovine PH20 exists very loosely attached to the plasma membrane, it is not anchored via a phospholipase sensitive anchor (Lalancette et al. (2001) Biol Reprod. 65(2):628-36). This unique feature of bovine hyaluronidase has permitted the use of the soluble bovine testes hyaluronidase enzyme as an extract for clinical use (Wydase®, Hyalase®).

The human PH20 mRNA transcript is normally translated to generate a 509 amino acid precursor polypeptide (SEQ ID NO:1) containing a 35 amino acid signal sequence at the N-terminus (amino acid residue positions 1-35) and a 19 amino acid glycosylphosphatidylinositol (GPI) anchor attachment signal sequence at the C-terminus (amino acid residue positions 491-509). The mature PH20 is, therefore, a 474 amino acid polypeptide set forth in SEQ ID NO:2. Following transport of the precursor polypeptide to the ER and removal of the signal peptide, the C-terminal GPI-attachment signal peptide is cleaved to facilitate covalent attachment of a GPI anchor to the newly-formed C-terminal amino acid at the amino acid position corresponding to position 490 of the precursor polypeptide set forth in SEQ ID NO: 1. Thus, a 474 amino acid GPI-anchored mature polypeptide with an amino acid sequence set forth in SEQ ID NO:2 is produced.

Human PH20 exhibits hyaluronidase activity at both neutral and acid pH. In one aspect, human PH20 is the prototypical neutral-active hyaluronidase that is generally locked to the plasma membrane via a GPI anchor. In another aspect, PH20 is expressed on the inner acrosomal membrane where it has hyaluronidase activity at both neutral and acid pH. It appears that PH20 contains two catalytic sites at distinct regions of the polypeptide: the Peptide 1 and Peptide 3 regions (Cherr et al., (2001) Matrix Biology 20:515-525). Evidence suggests that the Peptide 1 region of PH20, which corresponds to amino acid positions 107-137 of the mature polypeptide set forth in SEQ ID NO:2 and positions 142-172 of the precursor polypeptide set forth in SEQ ID NO:1, is required for enzyme activity at neutral pH. Amino acids at positions 111 and 113 (corresponding to the mature PH20 polypeptide set forth in SEQ ID NO:2) within this region appear to be important for activity, as mutagenesis by amino acid replacement results in PH20 polypeptides with 3% hyalutonidase activity or undetectable hyaluronidase activity, respectively, compared to the wild-type PH20 (Arming et al., (1997) Eur. J. Biochem. 247:810-814).

The Peptide 3 region, which corresponds to amino acid positions 242-262 of the mature polypeptide set forth in SEQ ID NO:2, and positions 277-297 of the precursor polypeptide set forth in SEQ ID NO: 1, appears to be important for enzyme activity at acidic pH. Within this region, amino acids at positions 249 and 252 of the mature PH20 polypeptide appear to be essential for activity, and mutagenesis of either one results in a polypeptide essentially devoid of activity (Arming et al., (1997) Eur. J. Biochem. 247:810-814).

In addition to the catalytic sites, PH20 also contains a hyaluronan-binding site. Experimental evidence suggest that this site is located in the Peptide 2 region, which corresponds to amino acid positions 205-235 of the precursor polypeptide set forth in SEQ ID NO: 1 and positions 170-200 of the mature polypeptide set forth in SEQ ID NO:2. This region is highly conserved among hyaluronidases and is similar to the heparin binding motif. Mutation of the arginine residue at position 176 (corresponding to the mature PH20 polypeptide set forth in SEQ ID NO:2) to a glycine results in a polypeptide with only about 1% of the hyaluronidase activity of the wild type polypeptide (Arming et al., (1997) Eur. J. Biochem. 247:810-814).

There are seven potential N-linked glycosylation sites in human PH20 at N82, N166, N235, N254, N368, N393, N490 of the polypeptide exemplified in SEQ ID NO: 1. Because amino acids 36 to 464 of SEQ ID NO:1 appears to contain the minimally active human PH20 hyaluronidase domain, the N-linked glycosylation site N-490 is not required for proper hyaluronidase activity. There are six disulfide bonds in human PH20. Two disulphide bonds between the cysteine residues C60 and C351 and between C224 and C238 of the polypeptide exemplified in SEQ ID NO: 1 (corresponding to residues C25 and C316, and C189 and C203 of the mature polypeptide set forth in SEQ ID NO:2, respectively). A further four disulphide bonds are formed between the cysteine residues C376 and C387; between C381 and C435; between C437 and C443; and between C458 and C464 of the polypeptide exemplified in SEQ ID NO: 1 (corresponding to residues C341 and C352; between C346 and C400; between C402 and C408; and between C423 and C429 of the mature polypeptide set forth in SEQ ID NO:2, respectively).

b. Bacterial Hyaluronidases

Bacterial hyaluronidases (EC 4.2.2.1 or EC 4.2.99.1) degrade hyaluronan and, to various extents, chondroitin sulfates and dermatan sulfates. Hyaluronan lyases isolated from bacteria differ from hyaluronidases (from other sources, e.g., hyaluronoglucosaminidases, EC 3.2.1.35) by their mode of action. They are endo-β-N-acetylhexosaminidases that catalyze an elimination reaction, rather than hydrolysis, of the β1→4-glycosidic linkage between N-acetyl-beta-D-glucosamine and D-glucuronic acid residues in hyaluronan, yielding 3-(4-deoxy-β-D-gluc-4-enuronosyl)-N-acetyl-D-glucosamine tetra- and hexasaccharides, and disaccharide end products. The reaction results in the formation of oligosaccharides with unsaturated hexuronic acid residues at their nonreducing ends.

Exemplary hyaluronidases from bacteria for use in the compositions, combinations and methods provided include, but are not limited to, hyaluronan degrading enzymes in microorganisms, including strains of Arthrobacter, Bdellovibrio, Clostridium, Micrococcus, Streptococcus, Peptococcus, Propionibacterium, Bacteroides, and Streptomyces. Particular examples of such enzymes include, but are not limited to Arthrobacter sp. (strain FB24) (SEQ ID NO:67), Bdellovibrio bacteriovorus (SEQ ID NO:68), Propionibacterium acnes (SEQ ID NO:69), Streptococcus agalactiae ((SEQ ID NO:70); 18RS21 (SEQ ID NO:71); serotype Ia (SEQ ID NO:72); serotype III (SEQ ID NO:73), Staphylococcus aureus (strain COL (SEQ ID NO:74); strain MRSA252 (SEQ ID NOS:75 and 76); strain MSSA476 (SEQ ID NO:77); strain NCTC 8325 (SEQ ID NO:78); strain bovine RF122 (SEQ ID NOS:79 and 80); strain USA300 (SEQ ID NO:81), Streptococcus pneumoniae ((SEQ ID NO:82); strain ATCC BAA-255/R6 (SEQ ID NO:83); serotype 2, strain D39/NCTC 7466 (SEQ ID NO:84), Streptococcus pyogenes (serotype M1) (SEQ ID NO:85); serotype M2, strain MGAS10270 (SEQ ID NO:86); serotype M4, strain MGAS10750 (SEQ ID NO:87); serotype M6 (SEQ ID NO:88); serotype M12, strain MGAS2096 (SEQ ID NOS:89 and 90); serotype M12, strain MGAS9429 (SEQ ID NO:91); serotype M28 (SEQ ID NO:92); Streptococcus suis (SEQ ID NOS:93-95); Vibrio fischeri (strain ATCC 700601/ES114 (SEQ ID NO:96)), and the Streptomyces hyaluronolyticus hyaluronidase enzyme, which is specific for hyaluronic acid and does not cleave chondroitin or chondroitin sulfate (Ohya, T. and Kaneko, Y. (1970) Biochim. Biophys. Acta 198:607).

c. Hyaluronidases from Leeches, Other Parasites and Crustaceans

Hyaluronidases from leeches, other parasites, and crustaceans (EC 3.2.1.36) are endo-β-glucuronidases that generate tetra- and hexasaccharide end-products. These enzymes catalyze hydrolysis of 1→3-linkages between β-D-glucuronate and N-acetyl-D-glucosamine residues in hyaluronate. Exemplary hyaluronidases from leeches include, but are not limited to, hyaluronidase from Hirudinidae (e.g., Hirudo medicinalis), Erpobdellidae (e.g., Nephelopsis obscura and Erpobdella punctata), Glossiphoniidae (e.g., Desserobdella picta, Helobdella stagnalis, Glossiphonia complanata, Placobdella ornata and Theromyzon sp.) and Haemopidae (Haemopis marmorata) (Hovingh et al. (1999) Comp Biochem Physiol B Biochem Mol Biol. 124(3):319-26). An exemplary hyaluronidase from bacteria that has the same mechanism of action as the leech hyaluronidase is that from the cyanobacteria, Synechococcus sp. (strain RCC307, SEQ ID NO:97).

2. Other Hyaluronan Degrading Enzymes

In addition to the hyaluronidase family, other hyaluronan degrading enzymes can be used in the compositions, combinations and methods provided. For example, enzymes, including particular chondroitinases and lyases, that have the ability to cleave hyaluronan can be employed. Exemplary chondroitinases that can degrade hyaluronan include, but are not limited to, chondroitin ABC lyase (also known as chondroitinase ABC), chondroitin AC lyase (also known as chondroitin sulfate lyase or chondroitin sulfate eliminase) and chondroitin C lyase. Methods for production and purification of such enzymes for use in the compositions, combinations, and methods provided are known in the art (e.g., U.S. Pat. No. 6,054,569; Yamagata, et al. (1968) J. Biol. Chem. 243(7):1523-1535; Yang et al. (1985) J. Biol. Chem. 160(30): 1849-1857).

Chondroitin ABC lyase contains two enzymes, chondroitin-sulfate-ABC endolyase (EC 4.2.2.20) and chondroitin-sulfate-ABC exolyase (EC 4.2.2.21) (Hamai et al. (1997) J Biol Chem. 272(14):9123-30), which degrade a variety of glycosaminoglycans of the chondroitin-sulfate- and dermatan-sulfate type. Chondroitin sulfate, chondroitin-sulfate proteoglycan and demmatan sulfate are the preferred substrates for chondroitin-sulfate-ABC endolyase, but the enzyme also can act on hyaluronan at a lower rate. Chondroitin-sulfate-ABC endolyase degrades a variety of glycosaminoglycans of the chondroitin-sulfate- and dermatan-sulfate type, producing a mixture of Δ4-unsaturated oligosaccharides of different sizes that are ultimately degraded to Δ4-unsaturated tetra- and disaccharides. Chondroitin-sulfate-ABC exolyase has the same substrate specificity but removes disaccharide residues from the non-reducing ends of both polymeric chondroitin sulfates and their oligosaccharide fragments produced by chondroitin-sulfate-ABC endolyase (Hamai, A. et al. (1997) J. Biol. Chem. 272:9123-9130). A exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase is set forth in SEQ ID NO: 98 (Sato et al. (1994) Appl. Microbiol. Biotechnol. 41(1):39-46).

Chondroitin AC lyase (EC 4.2.2.5) is active on chondroitin sulfates A and C, chondroitin and hyaluronic acid, but is not active on dermatan sulfate (chondroitin sulfate B). Exemplary chondroitinase AC enzymes from the bacteria include, but are not limited to, those from Flavobacterium heparinum and Victivallis vadensis, set forth in SEQ ID NOS:99 and 100, respectively, and Arthrobacter aurescens (Tkalec et al. (2000) Applied and Environmental Microbiology 66(1):29-35; Ernst et al. (1995) Critical Reviews in Biochemistry and Molecular Biology 30(5):387-444).

Chondroitinase C cleaves chondroitin sulfate C producing tetrasaccharide plus an unsaturated 6-sulfated disaccharide (delta Di-6S). It also cleaves hyaluronic acid producing unsaturated non-sulfated disaccharide (delta Di-OS). Exemplary chondroitinase C enzymes from the bacteria include, but are not limited to, those from Streptococcus and Flavobacterium (Hibi et al. (1989) FEMS-Microbiol-Lett. 48(2):121-4; Michelacci et al. (1976) J. Biol. Chem. 251:1154-8; Tsuda et al. (1999) Eur. J. Biochem. 262:127-133)

3. Soluble Hyaluronan Degrading Enzymes

Provided in the compositions, combinations and methods herein are soluble hyaluronan degrading enzymes, including soluble hyaluronidases. Soluble hyaluronan degrading enzymes include any hyaluronan degrading enzymes that exist in soluble form, including, but not limited to, soluble hyaluronidases, including non-human soluble hyaluronidases, including non-human animal soluble hyaluronidases, bacterial soluble hyaluronidases and human hyaluronidases, Hyall, bovine PH20 and ovine PH20, allelic variants thereof and other variants thereof. For example, included among soluble hyaluronan degrading enzymes are any hyaluronan degrading enzymes that have been modified to be soluble. For example, hyaluronan degrading enzymes that contain a GPI anchor can be made soluble by truncation of and removal of all or a portion of the GPI anchor. In one example, the human hyaluronidase PH20, which is normally membrane anchored via a GPI anchor, can be made soluble by truncation of and removal of all or a portion of the GPI anchor at the C-terminus.

Soluble hyaluronan degrading enzymes also include neutral active and acid active hyaluronidases. Depending on factors, such as, but not limited to, the desired level of activity of the enzyme following administration and/or site of administration, neutral active and acid active hyaluronidases can be selected. In a particular example, the hyaluronan degrading enzyme for use in the compositions, combinations and methods herein is a soluble neutral active hyaluronidase.

Exemplary of a soluble hyaluronidase is PH20 from any species, such as any set forth in any of SEQ ID NOS: 1, 2, 11, 25, 27, 30, 31, 63-65 and 101-102, or truncated forms thereof lacking all or a portion of the C-terminal GPI anchor, so long as the hyaluronidase is soluble and retains hyaluronidase activity. Also included among soluble hyaluronidases are allelic variants or other variants of any of SEQ ID NOS:1, 2, 11, 25, 27, 30 31, 63-65 and 101-102, or truncated forms thereof. Allelic variants and other variants are known to one of skill in the art, and include polypeptides having 60%, 70%, 80%, 90%, 91%, 92%, 93%, 94%, 95%. 96%. 97%. 98% or more sequence identity to any of SEQ ID NOS: 1, 2, 11, 25, 27, 30 31, 63-65 and 101-102, or truncated forms thereof. Amino acid variants include conservative and non-conservative mutations. It is understood that residues that are important or otherwise required for the activity of a hyaluronidase, such as any described above or known to skill in the art, are generally invariant. These include, for example, active site residues. Thus, for example, amino acid residues 111, 113 and 176 (corresponding to residues in the mature PH20 polypeptide set forth in SEQ ID NO:2) of a human PH20 polypeptide, or soluble form thereof, are generally invariant and are not altered. Other residues that confer glycosylation and formation of disulfide bonds required for proper folding also can be invariant.

In some instances, the soluble hyaluronan degrading enzyme is normally GPI-anchored (such as, for example, human PH20) and is rendered soluble by truncation at the C-terminus. Such truncation can remove of all of the GPI anchor attachment signal sequence, or can remove only some of the GPI anchor attachment signal sequence. The resulting polypeptide, however, is soluble. In instances where the soluble hyaluronan degrading enzyme retains a portion of the GPI anchor attachment signal sequence, 1, 2, 3, 4, 5, 6, 7 or more amino acid residues in the GPI-anchor attachment signal sequence can be retained, provided the polypeptide is soluble. Polypeptides containing one or more amino acids of the GPI anchor are termed extended soluble hyaluronan degrading enzymes. One of skill in the art can determine whether a polypeptide is GPI-anchored using methods well known in the art. Such methods include, but are not limited to, using known algorithms to predict the presence and location of the GPI-anchor attachment signal sequence and co-site, and performing solubility analyses before and after digestion with phosphatidylinositol-specific phospholipase C (PI-PLC) or D (PI-PLD).

Extended soluble hyaluronan degrading enzymes can be produced by making C-terminal truncations to any naturally GPI-anchored hyaluronan degrading enzyme such that the resulting polypeptide is soluble and contains one or more amino acid residues from the GPI-anchor attachment signal sequence. Exemplary extended soluble hyaluronan degrading enzymes that are C-terminally truncated but retain a portion of the GPI anchor attachment signal sequence include, but are not limited to, extended soluble PH20 (esPH20) polypeptides of primate origin, such as, for example, human and chimpanzee esPH20 polypeptides. For example, the esPH20 polypeptides can be made by C-terminal truncation of any of the mature or precursor polypeptides set forth in SEQ ID NOS:1, 2 or 101, or allelic or other variation thereof, including active fragment thereof, wherein the resulting polypeptide is soluble and retains one or more amino acid residues from the GPI-anchor attachment signal sequence. Allelic variants and other variants are known to one of skill in the art, and include polypeptides having 60%, 70%, 80%, 90%, 91%, 92%, 93%, 94%, 95% or more sequence identity to any of SEQ ID NOS: 1 or 2. The esPH20 polypeptides provided herein can be C-terminally truncated by 1, 2, 3, 4, 5, 6, 7, 8, 9, 10 or more amino acids compared to the wild type polypeptide, such as a polypeptide with a sequence set forth in SEQ ID NOS: 1, 2 or 101, provided the resulting esPH20 polypeptide is soluble and retains 1 or more amino acid residues from the GPI-anchor attachment signal sequence.

Typically, for use in the compositions, combinations and methods herein, a soluble human hylauronan degrading enzyme, such as a soluble human PH20, is used. Although hylauronan degrading enzymes, such as PH20, from other animals can be utilized, such preparations are potentially immunogenic, since they are animal proteins. For example, a significant proportion of patients demonstrate prior sensitization secondary to ingested foods, and since these are animal proteins, all patients have a risk of subsequent sensitization. Thus, non-human preparations may not be suitable for chronic use. If non-human preparations are desired, it is contemplated herein that such polypeptides can be prepared to have reduced immunogenicity. Such modifications are within the level of one of skill in the art and can include, for example, removal and/or replacement of one or more antigenic epitopes on the molecule.

Hyaluronan degrading enzymes, including hyaluronidases (e.g., PH20), used in the methods herein can be recombinantly produced or can be purified or partially-purified from natural sources, such as, for example, from testes extracts. Methods for production of recombinant proteins, including recombinant hyaluronan degrading enzymes, are provided elsewhere herein and are well known in the art.

a. Soluble Human PH20

Exemplary of a soluble hyaluronidase is soluble human PH20. Soluble forms of recombinant human PH20 have been produced and can be used in the compositions, combinations and methods described herein. The production of such soluble forms of PH20 is described in U.S. Published Patent Application Nos. US20040268425; US 20050260186 and US20060104968, and in the Examples, below. For example, soluble PH20 polypeptides, include C-terminally truncated variant polypeptides that include a sequence of amino acids in SEQ ID NO:1, or have at least 91%, 92%, 93%, 94%, 95%, 95%, 97%, 98% sequence identity to a sequence of amino acids included in SEQ ID NO:1, retain hyaluronidase activity and are soluble. Included among these polypeptides are soluble PH20 polypeptides that completely lack all or a portion of the GPI-anchor attachment signal sequence. Also included are extended soluble PH20 (esPH20) polypeptides that contain at least one amino acid of the GPI anchor. Thus, instead of having a GPI-anchor covalently attached to the C-terminus of the protein in the ER and being anchored to the extracellular leaflet of the plasma membrane, these polypeptides are secreted and are soluble. C-terminally truncated PH20 polypeptides can be C-terminally truncated by 1, 2, 3, 4, 5, 6, 7, 8, 9, 10, 11, 12, 13, 14, 15, 16, 17, 18, 19, 20, 25, 30, 35, 40, 45, 50, 5, 60 or more amino acids compared to the full length wild type polypeptide, such as a full length wild type polypeptide with a sequence set forth in SEQ ID NOS:1 or 2, or allelic or species variants or other variants thereof.

Exemplary C-terminally truncated human PH20 polypeptides provided herein include any having C-terminal truncations to generate polypeptides containing amino acid 1 to amino acid 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, of the sequence of amino acids set forth in SEQ ID NO: 1, or corresponding positions in an allelic or species variant thereof. When expressed in mammalian cells, the 35 amino acid N-terminal signal sequence is cleaved during processing, and the mature form of the protein is secreted. Thus, exemplary mature C-terminally truncated soluble PH20 polypeptides can contain amino acids 36 to 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497 of the sequence of amino acids set forth in SEQ ID NO: 1 or corresponding positions in an allelic or species variant thereof. Table 1b provides non-limiting examples of exemplary C-terminally truncated PH20 polypeptides, including C-terminally truncated soluble PH20 polypeptides. In Table 1b below, the length (in amino acids) of the precursor and mature polypeptides, and the sequence identifier (SEQ ID NO) in which exemplary amino acid sequences of the precursor and mature polypeptides of the C-terminally truncated PH20 proteins are set forth, are provided. The wild-type PH20 polypeptide also is included in Table 1b for comparison.

TABLE 1b
Exemplary C-terminally truncated PH20 polypeptides

| | Precursor | | | |
|---|---|---|---|---|
| | (amino | Precursor | Mature | Mature |
| Polypeptide | acids) | SEQ ID NO | (amino acids) | SEQ ID NO |
| wildtype | 509 | 1 | 474 | 2 |
| SPAM1-FIVS | 497 | 107 | 462 | 151 |
| SPAM1-MFIV | 496 | 141 | 461 | 185 |
| SPAM1-TMFI | 495 | 108 | 460 | 152 |
| SPAM1-ATMF | 494 | 142 | 459 | 186 |
| SPAM1-SATM | 493 | 109 | 458 | 153 |
| SPAM1-LSAT | 492 | 143 | 457 | 187 |
| SPAM1-TLSA | 491 | 110 | 456 | 154 |
| SPAM1-PSTL | 489 | 111 | 454 | 155 |
| SPAM1-SPST | 488 | 144 | 453 | 188 |
| SPAM1-STLS | 490 | 112 | 455 | 156 |
| SPAM1-ASPS | 487 | 113 | 452 | 157 |
| SPAM1-NASP | 486 | 145 | 451 | 189 |
| SPAM1-YNAS | 485 | 114 | 450 | 158 |
| SPAM1-FYNA | 484 | 115 | 449 | 159 |
| SPAM1-IFYN | 483 | 46 | 448 | 48 |
| SPAM1-QIFY | 482 | 3 | 447 | 4 |
| SPAM1-PQIF | 481 | 45 | 446 | 5 |
| SPAM1-EPQI | 480 | 44 | 445 | 6 |
| SPAM1-EEPQ | 479 | 43 | 444 | 7 |
| SPAM1-TEEP | 478 | 42 | 443 | 8 |
| SPAM1-ETEE | 477 | 41 | 442 | 9 |
| SPAM1-METE | 476 | 116 | 441 | 160 |
| SPAM1-PMET | 475 | 117 | 440 | 161 |
| SPAM1-PPME | 474 | 118 | 439 | 162 |
| SPAM1-KPPM | 473 | 119 | 438 | 163 |
| SPAM1-LKPP | 472 | 120 | 437 | 164 |
| SPAM1-FLKP | 471 | 121 | 436 | 165 |
| SPAM1-AFLK | 470 | 122 | 435 | 166 |
| SPAM1-DAFL | 469 | 123 | 434 | 167 |
| SPAM1-IDAF | 468 | 124 | 433 | 168 |
| SPAM1-CIDA | 467 | 40 | 432 | 47 |
| SPAM1-VCID | 466 | 125 | 431 | 169 |
| SPAM1-GVCI | 465 | 126 | 430 | 170 |

Soluble forms include, but are not limited to, any having C-terminal truncations to generate polypeptides containing amino acids 1 to amino acid 467, 477, 478, 479, 480, 481, 482 and 483 of the sequence of amino acids set forth in SEQ ID NO:1. When expressed in mammalian cells, the 35 amino acid N-terminal signal sequence is cleaved during processing, and the mature form of the protein is secreted. Thus, the mature soluble polypeptides contain amino acids 36 to 467, 477, 478, 479, 480, 481, 482 and 483 of SEQ ID NO:1. Deletion mutants ending at amino acid position 477 to 483 (corresponding to the precursor polypeptide set forth in SEQ ID NO:1) exhibit higher secreted hyaluronidase activity than the full length GPI-anchored form. Hence, exemplary of soluble hyaluronidases soluble human PH20 polypeptides that are 442, 443, 444, 445, 446 or 447 amino acids in length, such as set forth in any of SEQ ID NOS: 4-9, or allelic or species variants or other variants thereof.

Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g. DG44 CHO cells).

b. HuPH20

Recombinant soluble forms of human PH20 have been generated and can be used in the compositions, combinations and methods provided herein. The generation of such soluble forms of recombinant human PH20 are described in U.S. Published Patent Application Nos. US20040268425; US 20050260186 and US20060104968, and in Examples 2-6, below. Exemplary of such polypeptides are those generated from a nucleic acid molecule encoding amino acids 1-482 (set forth in SEQ ID NO:3). Such an exemplary nucleic acid molecule is set forth in SEQ ID NO:49. Post translational processing removes the 35 amino acid signal sequence, leaving a 447 amino acid soluble recombinant human PH20 (SEQ ID NO:4). As produced in the culture medium there is heterogeneity at the C-terminus such that the product, designated rHuPH20, includes a mixture of species that can include any one or more of SEQ ID NOS. 4-9 in various abundance. Typically, rHuPH20 is produced in cells that facilitate correct N-glycosylation to retain activity, such as CHO cells (e.g. DG44 CHO cells).

4. Glycosylation of Hyaluronan Degrading Enzymes

Glycosylation, including N- and O-linked glycosylation, of some hyaluronan degrading enzymes, including hyaluronidases, can be important for their catalytic activity and stability. While altering the type of glycan modifying a glycoprotein can have dramatic affects on a protein's antigenicity, structural folding, solubility, and stability, most enzymes are not thought to require glycosylation for optimal enzyme activity. For some hyaluronidases, removal of N-linked glycosylation can result in near complete inactivation of the hyaluronidase activity. Thus, for such hyaluronidases, the presence of N-linked glycans is critical for generating an active enzyme.

N-linked oligosaccharides fall into several major types (oligomannose, complex, hybrid, sulfated), all of which have (Man) 3-GlcNAc-GlcNAc-cores attached via the amide nitrogen of Asn residues that fall within-Asn-Xaa-Thr/Ser-sequences (where Xaa is not Pro). Glycosylation at an-Asn-Xaa-Cys-site has been reported for coagulation protein C. In some instances, a hyaluronan degrading enzyme, such as a hyaluronidase, can contain both N-glycosidic and O-glycosidic linkages. For example, PH20 has O-linked oligosaccharides as well as N-linked oligosaccharides. There are seven potential N-linked glycosylation sites at N82, N166, N235, N254, N368, N393, N490 of human PH20 exemplified in SEQ ID NO: 1. As noted above, N-linked glycosylation at N490 is not required for hyaluronidase activity.

In some examples, the hyaluronan degrading enzymes for use in the compositions, combinations and/or methods provided are glycosylated at one or all of the glycosylation sites. For example, for human PH20, or a soluble form thereof, 2, 3, 4, 5, or 6 of the N-glycosylation sites corresponding to amino acids N82, N166, N235, N254, N368, and N393 of SEQ ID NO: 1 are glycosylated. In some examples the hyaluronan degrading enzymes are glycosylated at one or more native glycosylation sites. In other examples, the hyaluronan degrading enzymes are modified at one or more non-native glycosylation sites to confer glycosylation of the polypeptide at one or more additional site. In such examples, attachment of additional sugar moieties can enhance the pharmacokinetic properties of the molecule, such as improved half-life and/or improved activity.

In other examples, the hyaluronan degrading enzymes for use in the compositions, combinations and/or methods provided herein are partially deglycosylated (or N-partially glycosylated polypeptides). For example, partially deglycosylated soluble PH20 polypeptides that retain all or a portion of the hyaluronidase activity of a fully glycosylated hyaluronidase can be used in the compositions, combinations and/or methods provided herein. Exemplary partially deglycosylated hyalurodinases include soluble forms of a partially deglycosylated PH20 polypeptides from any species, such as any set forth in any of SEQ ID NOS: 1, 2, 11, 25, 27, 29, 30, 31, 32, 63, 65, 101 and 102, or allelic variants, truncated variants, or other variants thereof. Such variants are known to one of skill in the art, and include polypeptides having 60%, 70%, 80%, 90%, 91%, 92%, 93%, 94%, 95% or more sequence identity to any of SEQ ID NOS: 1, 2, 11, 25, 27, 29, 30, 31, 32, 63, 65, 101 and 102, or truncated forms thereof. The partially deglycosylated hyaluronidases provided herein also include hybrid, fusion and chimeric partially deglycosylated hyaluronidases, and partially deglycosylated hyaluronidase conjugates.

Glycosidases, or glycoside hydrolases, are enzymes that catalyze the hydrolysis of the glycosidic linkage to generate two smaller sugars. The major types of N-glycans in vertebrates include high mannose glycans, hybrid glycans and complex glycans. There are several glycosidases that result in only partial protein deglycosylation, including: EndoF1, which cleaves high mannose and hybrid type glycans; EndoF2, which cleaves biantennary complex type glycans; EndoF3, which cleaves biantennary and more branched complex glycans; and EndoH, which cleaves high mannose and hybrid type glycans. Treatment of a hyaluronan degrading enzyme, such as a soluble hyaluronidase, such as a soluble PH20, with one or all of these glycosidases can result in only partial deglycosylation and, therefore, retention of hyaluronidase activity.

Partially deglycosylated hyaluronan degrading enzymes, such as partially deglycosylated soluble hyaluronidases, can be produced by digestion with one or more glycosidases, generally a glycosidase that does not remove all N-glycans but only partially deglycosylates the protein. For example, treatment of PH20 (e.g. a recombinant PH20 designated rHuPH20) with one or all of the above glycosidases (e.g. EndoF1, EndoF2 and/or EndoF3) results in partial deglycosylation. These partially deglycosylated PH20 polypeptides can exhibit hyaluronidase enzymatic activity that is comparable to the fully glycosylated polypeptides. In contrast, treatment of PH20 with PNGaseF, a glycosidase that cleaves all N-glycans, results in complete removal of all N-glycans and thereby renders PH20 enzymatically inactive. Thus, although all N-linked glycosylation sites (such as, for example, those at amino acids N82, N166, N235, N254, N368, and N393 of human PH20, exemplified in SEQ ID NO: 1) can be glycosylated, treatment with one or more glycosidases can render the extent of glycosylation reduced compared to a hyaluronidase that is not digested with one or more glycosidases.

The partially deglycosylated hyaluronan degrading enzymes, including partially deglycosylated soluble PH20 polypeptides, can have 10%, 20%, 30%, 40%, 50%, 60%, 70% or 80% of the level of glycosylation of a fully glycosylated polypeptide. Typically, the partially deglyclosylated hyaluronan degrading enzymes, including partially deglycosylated soluble PH20 polypeptides, exhibit hyaluronidase activity that is 10%, 20%, 30%, 40%, 50%, 60%, 70%, 80%, 90%, 100%, 110%, 120%, 130%, 140%, 150%, 200%, 300%, 400%, 500%, 1000% or more of the hyaluronidase activity exhibited by the fully glycosylated polypeptide.

### E. Methods of Producing Nucleic Acids Encoding a Soluble Hyaluronidase and Polypeptides Thereof

Polypeptides of a soluble hyaluronidase set forth herein, can be obtained by methods well known in the art for protein purification and recombinant protein expression. Any method known to those of skill in the art for identification of nucleic acids that encode desired genes can be used. Any method available in the art can be used to obtain a full length (i.e., encompassing the entire coding region) cDNA or genomic DNA clone encoding a hyaluronidase, such as from a cell or tissue source. Modified or variant soluble hyaluronidases, can be engineered from a wildtype polypeptide, such as by site-directed mutagenesis.

Polypeptides can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding a desired polypeptide, including for example, polymerase chain reaction (PCR) methods. A nucleic acid containing material can be used as a starting material from which a desired polypeptide-encoding nucleic acid molecule can be isolated. For example, DNA and mRNA preparations, cell extracts, tissue extracts, fluid samples (e.g. blood, serum, saliva), samples from healthy and/or diseased subjects can be used in amplification methods. Nucleic acid libraries also can be used as a source of starting material. Primers can be designed to amplify a desired polypeptide. For example, primers can be designed based on expressed sequences from which a desired polypeptide is generated. Primers can be designed based on back-translation of a polypeptide amino acid sequence. Nucleic acid molecules generated by amplification can be sequenced and confirmed to encode a desired polypeptide.

Additional nucleotide sequences can be joined to a polypeptide-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a polypeptide-encoding nucleic acid molecule. Examples of such sequences include, but are not limited to, promoter sequences designed to facilitate intracellular protein expression, and secretion sequences, for example heterologous signal sequences, designed to facilitate protein secretion. Such sequences are known to those of skill in the art. Additional nucleotide residues sequences such as sequences of bases specifying protein binding regions also can be linked to enzyme-encoding nucleic acid molecules. Such regions include, but are not limited to, sequences of residues that facilitate or encode proteins that facilitate uptake of an enzyme into specific target cells, or otherwise alter pharmacokinetics of a product of a synthetic gene. For example, enzymes can be linked to PEG moieties.

In addition, tags or other moieties can be added, for example, to aid in detection or affinity purification of the polypeptide. For example, additional nucleotide residues sequences such as sequences of bases specifying an epitope tag or other detectable marker also can be linked to enzyme-encoding nucleic acid molecules. Exemplary of such sequences include nucleic acid sequences encoding a His tag (e.g., 6×His, HHHHHH; SEQ ID NO:54) or Flag Tag (DYKDDDDK; SEQ ID NO:55).

The identified and isolated nucleic acids can then be inserted into an appropriate cloning vector. A large number of vector-host systems known in the art can be used. Possible vectors include, but are not limited to, plasmids or modified viruses. The vector system selected is compatible with the host cell used. Such vectors include, but are not limited to, bacteriophages such as lambda derivatives, or plasmids such as pCMV4, pBR322 or pUC plasmid derivatives or the Bluescript vector (Stratagene, La Jolla, Calif.). Other expression vectors include the HZ24 expression vector exemplified herein. The insertion into a cloning vector can, for example, be accomplished by ligating the DNA fragment into a cloning vector which has complementary cohesive termini. Insertion can be effected using TOPO cloning vectors (INVITROGEN, Carlsbad, Calif.). If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and protein gene can be modified by homopolymeric tailing. Recombinant molecules can be introduced into host cells via, for example, transformation, transfection, infection, electroporation and sonoporation, so that many copies of the gene sequence are generated.

In specific embodiments, transformation of host cells with recombinant DNA molecules that incorporate the isolated protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

1. Vectors and Cells

For recombinant expression of one or more of the desired proteins, such as any described herein, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals also can be supplied by the native promoter for enzyme genes, and/or their flanking regions.

Also provided are vectors that contain a nucleic acid encoding the enzyme. Cells containing the vectors also are provided. The cells include eukaryotic and prokaryotic cells, and the vectors are any suitable for use therein.

Prokaryotic and eukaryotic cells, including endothelial cells, containing the vectors are provided. Such cells include bacterial cells, yeast cells, fungal cells, Archaea, plant cells, insect cells and animal cells. The cells are used to produce a protein thereof by growing the above-described cells under conditions whereby the encoded protein is expressed by the cell, and recovering the expressed protein. For purposes herein, for example, the enzyme can be secreted into the medium.

Provided are vectors that contain a sequence of nucleotides that encodes the soluble hyaluronidase polypeptide coupled to the native or heterologous signal sequence, as well as multiple copies thereof. The vectors can be selected for expression of the enzyme protein in the cell or such that the enzyme protein is expressed as a secreted protein.

A variety of host-vector systems can be used to express the protein coding sequence. These include but are not limited to mammalian cell systems infected with virus (e.g. vaccinia virus, adenovirus and other viruses); insect cell systems infected with virus (e.g. baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding protein, or domains, derivatives, fragments or homologs thereof, can be regulated by a second nucleic acid sequence so that the genes or fragments thereof are expressed in a host transformed with the recombinant DNA molecule(s). For example, expression of the proteins can be controlled by any promoter/enhancer known in the art. In a specific embodiment, the promoter is not native to the genes for a desired protein. Promoters which can be used include but are not limited to the SV40 early promoter (Bemoist and Chambon (1981) Nature 290:304-310), the promoter contained in the 3′ long terminal repeat of Rous sarcoma virus (Yamamoto et al. (1980) Cell 22:787-797), the herpes thymidine kinase promoter (Wagner et al. (1981) Proc. Natl. Acad. Sci. USA 78:1441-1445), the regulatory sequences of the metallothionein gene (Brinster et al. (1982) Nature 296:39-42); prokaryotic expression vectors such as the β-lactamase promoter (Jay et al. (1981) Proc. Natl. Acad. Sci. USA 78:5543) or the tac promoter (DeBoer et al. (1983) Proc. Natl. Acad. Sci. USA 80:21-25); see also “Useful Proteins from Recombinant Bacteria”: in Scientific American 242:79-94 (1980)); plant expression vectors containing the nopaline synthetase promoter (Herrara-Estrella et al., Nature 303:209-213 (1984)) or the cauliflower mosaic virus 35S RNA promoter (Garder et al. (1981) Nucleic Acids Res. 9:2871), and the promoter of the photosynthetic enzyme ribulose bisphosphate carboxylase (Herrera-Estrella et al. (1984) Nature 310:115-120); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al. (1984) Cell 38:639-646; Ornitz et al. (1986) Cold Spring Harbor Symp. Quant. Biol. 50:399-409; MacDonald (1987) Hepatology 7:425-515); insulin gene control region which is active in pancreatic beta cells (Hanahan et al. (1985) Nature 315:115-122), immunoglobulin gene control region which is active in lymphoid cells (Grosschedl et al. (1984) Cell 38:647-658; Adams et al. (1985) Nature 318:533-538; Alexander et al. (1987) Mol. Cell. Biol. 7:1436-1444), mouse mammary tumor virus control region which is active in testicular, breast, lymphoid and mast cells (Leder et al. (1986) Cell 45:485-495), albumin gene control region which is active in liver (Pinckert et al. (1987) Genes and Devel. 1:268-276), alpha-fetoprotein gene control region, which is active in liver (Krumlauf et al. (1985) Mol. Cell. Biol. 5:1639-1648; Hammer et al. (1987) Science 235:53-58), alpha-1 antitrypsin gene control region, which is active in liver (Kelsey et al. (1987) Genes and Devel. 1:161-171), beta globin gene control region which is active in myeloid cells (Magram et al. (1985) Nature 315:338-340; Kollias et al. (1986) Cell 46:89-94), myelin basic protein gene control region which is active in oligodendrocyte cells of the brain (Readhead et al. (1987) Cell 48:703-712), myosin light chain-2 gene control region which is active in skeletal muscle (Shani (1985) Nature 314:283-286), and gonadotrophic releasing hormone gene control region which is active in gonadotrophs of the hypothalamus (Mason et al. (1986) Science 234:1372-1378).

In a specific embodiment, a vector is used that contains a promoter operably linked to nucleic acids encoding a desired protein, or a domain, fragment, derivative or homolog, thereof, one or more origins of replication, and optionally, one or more selectable markers (e.g., an antibiotic resistance gene). Exemplary plasmid vectors for transformation of E. coli cells, include, for example, the pQE expression vectors (available from Qiagen, Valencia, Calif.; see also literature published by Qiagen describing the system). pQE vectors have a phage T5 promoter (recognized by E. coli RNA polymerase) and a double lac operator repression module to provide tightly regulated, high-level expression of recombinant proteins in E. coli, a synthetic ribosomal binding site (RBS II) for efficient translation, a 6×His tag coding sequence, t₀ and T1 transcriptional terminators, ColE1 origin of replication, and a beta-lactamase gene for conferring ampicillin resistance. The pQE vectors enable placement of a 6×His tag at either the N- or C-terminus of the recombinant protein. Such plasmids include pQE 32, pQE 30, and pQE 31 which provide multiple cloning sites for all three reading frames and provide for the expression of N-terminally 6×His-tagged proteins. Other exemplary plasmid vectors for transformation of E. coli cells, include, for example, the pET expression vectors (see, U.S. Pat. No. 4,952,496; available from NOVAGEN, Madison, Wis.; see, also literature published by Novagen describing the system). Such plasmids include pET 11a, which contains the T7lac promoter, T7 terminator, the inducible E. coli lac operator, and the lac repressor gene; pET 12a-c, which contains the T7 promoter, T7 terminator, and the E. coli ompT secretion signal; and pET 15b and pET19b (NOVAGEN, Madison, Wis.), which contain a His-Tag™ leader sequence for use in purification with a His column and a thrombin cleavage site that permits cleavage following purification over the column, the T7-lac promoter region and the T7 terminator.

Exemplary of a vector for mammalian cell expression is the HZ24 expression vector. The HZ24 expression vector was derived from the pCI vector backbone (Promega). It contains DNA encoding the Beta-lactamase resistance gene (AmpR), an F1 origin of replication, a Cytomegalovirus immediate-early enhancer/promoter region (CMV), and an SV40 late polyadenylation signal (SV40). The expression vector also has an internal ribosome entry site (IRES) from the ECMV virus (Clontech) and the mouse dihydrofolate reductase (DHFR) gene.

2. Expression

Soluble hyaluronidase polypeptides can be produced by any method known to those of skill in the art including in vivo and in vitro methods. Desired proteins can be expressed in any organism suitable to produce the required amounts and forms of the proteins, such as for example, needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

Soluble hyaluronidase polypeptides also can be utilized or expressed as protein fusions. For example, an enzyme fusion can be generated to add additional functionality to an enzyme. Examples of enzyme fusion proteins include, but are not limited to, fusions of a signal sequence, a tag such as for localization, e.g. a his₆ tag or a myc tag, or a tag for purification, for example, a GST fusion, and a sequence for directing protein secretion and/or membrane association.

a. Prokaryotic Cells

Prokaryotes, especially E. coli, provide a system for producing large amounts of proteins. Transformation of E. coli is simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

Proteins, such as any provided herein, can be expressed in the cytoplasmic environment of E. coli. The cytoplasm is a reducing environment and for some molecules, this can result in the formation of insoluble inclusion bodies. Reducing agents such as dithiothreitol and β-mercaptoethanol and denaturants, such as guanidine-HCl and urea can be used to resolubilize the proteins. An alternative approach is the expression of proteins in the periplasmic space of bacteria which provides an oxidizing environment and chaperonin-like and disulfide isomerases and can lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast Cells

Yeasts such as Saccharomyces cerevisae, Schizosaccharomyces pombe, Yarrowia lipolytica, Kluyveromyces lactis and Pichia pastoris are well known yeast expression hosts that can be used for production of proteins, such as any described herein. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insect Cells

Insect cells, particularly using baculovirus expression, are useful for expressing polypeptides such as hyaluronidase polypeptides. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the Bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schneider 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express proteins including soluble hyaluronidase polypeptides. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. IRES elements also can be added to permit bicistronic expression with another gene, such as a selectable marker. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase (DHFR) and thymidine kinase. For example, expression can be performed in the presence of methotrexate to select for only those cells expressing the DHFR gene. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. Examples include CHO—S cells (Invitrogen, Carlsbad, Calif., cat #11619-012) and the serum free EBNA-1 cell line (Pham et al. (2003) Biotechnol. Bioeng. 84:332-42). Cell lines also are available that are adapted to grow in special mediums optimized for maximal expression. For example, DG44 CHO cells are adapted to grow in suspension culture in a chemically defined, animal product-free medium.

e. Plants

Transgenic plant cells and plants can be used to express proteins such as any described herein. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline syntase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce hyaluronidase polypeptides. Because plants have different glycosylation patterns than mammalian cells, this can influence the choice of protein produced in these hosts.

3. Purification Techniques

Method for purification of polypeptides, including soluble hyaluronidase polypeptides or other proteins, from host cells will depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary, the proteins can be extracted and further purified using standard methods in the art.

Proteins, such as soluble hyaluronidase polypeptides, can be purified using standard protein purification techniques known in the art including but not limited to, SDS-PAGE, size fraction and size exclusion chromatography, ammonium sulfate precipitation and ionic exchange chromatography, such as anion exchange. Affinity purification techniques also can be utilized to improve the efficiency and purity of the preparations. For example, antibodies, receptors and other molecules that bind hyaluronidase enzymes can be used in affinity purification. Expression constructs also can be engineered to add an affinity tag to a protein such as a myc epitope, GST fusion or His₆ and affinity purified with myc antibody, glutathione resin and Ni-resin, respectively. Purity can be assessed by any method known in the art including gel electrophoresis and staining and spectrophotometric techniques.

### F. Preparation, Formulation and Administration of Bisphosphonates and Soluble Hyaluronidase Polypeptides

Pharmaceutical compositions of bisphosphonates and soluble hyaluronidases are provided herein for subcutaneous administration. Soluble hyaluronidases are co-formulated or co-administered with pharmaceutical formulations of a bisphosphonate to reduce injection site toxicity of subcutaneous administration of bisphosphonates and to enhance the delivery of the bisphosphonate to desired sites within the body by increasing the bioavailability of bisphosphonates. For example, co-administration or co-formulation of a bisphosphonate with a hyaluronidase can improve the extent and/or rate of absorption and thus bioavailability of an agent by causing more of it to reach the bloodstream and/or less of it being degraded after administration by more rapid permeation. Increased absorption and bioavailability can be achieved, for example, by accelerating interstitial flow and potentially connective transport following administration by applying hydrostatic pressure associated with the volume injection combined with a reduction in impedance to flow associated with degradation of hyaluronan. Thus, soluble hyaluronidases can be used to achieve elevated and/or more rapidly achieved concentrations of the bisphosphonate following subcutaneous administration compared to conventional methods of subcutaneous administration, to provide, for example, a more potent and/or more rapid response for a given dose. Alternatively, the soluble hyaluronidase can be used to allow a given response to be achieved with a lower dose of administered bisphosphonate. The ability of a soluble hyaluronidase to enhance bulk fluid flow at and near a site of injection or infusion also can improve other aspects of associated pharmacologic delivery. For example, the increase in bulk fluid flow can help to allow the volume of fluid injected to be more readily dispersed from the site of injection (reducing potentially painful or other adverse consequences of injection). This is particularly important for subcutaneous infusions to permit higher doses to be administered. In addition to increased bioavailability, co-administration or co-formulation of a bisphosphonate with soluble hyaluronidase provides for a safer or more convenient route of administration compared to conventional intravenous routes of administration.

Thus, by virtue of the increased bioavailability, bisphosphonates can be administered subcutaneously at dosages and frequencies for which current intravenous (IV) preparations are prepared and administered. The advantages over current formulations of bisphosphonates is that co-administered or co-formulated hyaluronidase/bisphosphonate administered by subcutaneous injection can result in more favorable dosing regimens, for example, shorter dosing times and less frequent dosing. By less frequent or lower dosing, side effects associated with toxicity can be reduced. In addition, subcutaneous infusion can permit infusion by the patient or family as opposed to a skilled nurse; infusion can be achieved at higher rates; there is no requirement for functional veins; and infusion can be performed at home or anywhere. The advantages over current formulations of oral bisphosphonates is that co-administered or co-formulated hyaluronidase/bisphosphonate administered by subcutaneous injection avoids the gastrointestinal disorders and esophageal damage associated with oral bisphosphonate therapy and overcomes the poor bioavailability of oral administration. Generally, the pharmacokinetic and/or pharmacodynamics of bisphosphonate therapy is improved by the methods and uses provided herein.

The compositions can be formulated in lyophilized or liquid form. Where the compositions are provided in lyophilized form they can be reconstituted just prior to use by an appropriate buffer, for example, a sterile saline solution. The compositions can be provided together or separately. For purposes herein, such compositions typically are provided separately. The soluble hyaluronidase and bisphosphonate can be packaged as separate compositions for administration together, sequentially or intermittently. The combinations can be packaged as a kit.

1. Formulations

The compounds can be formulated into any suitable pharmaceutical preparations for subcutaneous administration such as solutions, suspensions, powders, or sustained release formulations. Typically, the compounds are formulated into pharmaceutical compositions using techniques and procedures well known in the art (see e.g., Ansel Introduction to Pharmaceutical Dosage Forms, Fourth Edition, 1985, 126). Pharmaceutically acceptable compositions are prepared in view of approvals for a regulatory agency or other agency prepared in accordance with generally recognized pharmacopeia for use in animals and in humans. The formulation selected is suitable for the mode of administration.

Pharmaceutical compositions can include carriers such as a diluent, adjuvant, excipient, or vehicle with which a hyaluronidase or bisphosphonate is administered. Examples of suitable pharmaceutical carriers are described in “Remington's Pharmaceutical Sciences” by E. W. Martin. Such compositions will contain a therapeutically effective amount of the compound, generally in purified form or partially purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents.

An exemplary standard stabilized formulation of a soluble hyaluronidase domain as provided herein is formulated with one or more of EDTA, NaCl, CaCl₂, histidine, lactose, albumin, Pluronic® F68, TWEEN® and/or other detergent. Generally, a salt, such as NaCl is provided in formulations herein, for example, in an amount that is or is about 100 mM to 150 mM NaCl or more. For example, an exemplary formulation can contain at or about 10 mM histidine and/or at or about 130 mM NaCl. Concentrated formulations of a soluble hyaluronidase are generally diluted in a saline or other salt buffered solution prior to administration in order to maintain the desired salt concentration. Other formulations can contain in addition or alternatively lactose, for example, at or about 13 mg/ml. Additionally, an anti-bacterial or anti-fungal agent, including, but not limited to thiomersal, can be present in the formulation. Formulations can further contain Albumin, Pluronic® F68, TWEEN® and/or other detergent. The formulations are provided at a pH that is or is about 6.0, 6.2, 6.4, 6.5, 6.6, 6.8, 7.0, 7.2, 7.4, 7.6, 7.8 or 8, generally that is or is about pH 6.5. The pH and the osmolarity of the compositions can be adjusted by one of skill in the art to optimize the conditions for the desired activity and stability of the composition.

Pharmaceutically therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared.

Compositions provided herein typically are formulated for administration by subcutaneous route, although other routes of administration are contemplated, such as any route known to those of skill in the art including intramuscular, intravenous, intradermal, intralesional, intraperitoneal injection, epidural, nasal, oral, vaginal, rectal, topical, local, otic, inhalational, buccal (e.g., sublingual), and transdermal administration or any route. Formulations suited for such routes are known to one of skill in the art. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Compositions also can be administered with other biologically active agents, either sequentially, intermittently or in the same composition. Administration also can include controlled release systems including controlled release formulations and device controlled release, such as by means of a pump.

The most suitable route in any given case depends on a variety of factors, such as the nature of the disease, the progress of the disease, the severity of the disease the particular composition which is used. For purposes herein, it is desired that hyaluronidases are administered so that they reach the interstitium of skin or tissues, thereby degrading the interstitial space for subsequent delivery of the bisphosphonate. Thus, direct administration under the skin, such as by subcutaneous administration methods, is contemplated. Thus, in one example, local administration can be achieved by injection, such as from a syringe or other article of manufacture containing a injection device such as a needle. The rate of administration from a syringe can be controlled by controlled pressure over desired period of time to distribute the contents of the syringe. In another example, local administration can be achieved by infusion, which can be facilitated by the use of a pump or other similar device. Other modes of administration also are contemplated. Pharmaceutical composition can be formulated in dosage forms appropriate for each route of administration.

Subcutaneous administration, generally characterized by injection or infusion, is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. The pharmaceutical compositions can contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such compositions is highly dependent on the specific nature thereof, as well as the activity of the compound and the needs of the subject.

Injectables are designed for local and systemic administration. For purposes herein, local administration is desired for direct administration to the affected interstitium. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (i.e., TWEEN 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package. All preparations for parenteral administration are sterile, as is known and practiced in the art.

In one example, pharmaceutical preparation can be in liquid form, for example, solutions, syrups or suspensions. If provided in liquid form, the pharmaceutical preparations can be provided as a concentrated preparation to be diluted to a therapeutically effective concentration before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). In another example, pharmaceutical preparations can be presented in lyophilized form for reconstitution with water or other suitable vehicle before use.

Administration methods can be employed to decrease the exposure of selected compounds to degradative processes, such as proteolytic degradation and immunological intervention via antigenic and immunogenic responses. Examples of such methods include local administration at the site of treatment. Pegylation of therapeutics has been reported to increase resistance to proteolysis, increase plasma half-life, and decrease antigenicity and immunogenicity. Examples of pegylation methodologies are known in the art (see, for example, Lu and Felix (1994) Int. J. Peptide Protein Res. 43:127-138; Lu and Felix (1993) Peptide Res. 6:142-6; Felix et al. (1995) Int. J. Peptide Res. 46:253-64; Benhar et al. (1994) J. Biol. Chem. 269:13398-404; Brumeanu et al. (1995) J. Immunol. 154:3088-95; see also, Caliceti et al. (2003) Adv. Drug Deliv. Rev. 55(10):1261-77 and Molineux (2003) Pharmacotherapy 23 (8 Pt 2):3S-8S). Pegylation also can be used in the delivery of nucleic acid molecules in vivo. For example, pegylation of adenovirus can increase stability and gene transfer (see, e.g., Cheng et al. (2003) Pharm. Res. 20(9):1444-51).

a. Lyophilized Powder

Of interest herein are lyophilized powders, which can be reconstituted for administration as solutions, emulsions and other mixtures. They also can be reconstituted and formulated as solids or gels.

The sterile, lyophilized powder is prepared by dissolving an active compound in a buffer solution. The buffer solution can contain an excipient which improves the stability or other pharmacological component of the powder or reconstituted solution, prepared from the powder. Subsequent sterile filtration of the solution followed by lyophilization under standard conditions known to those of skill in the art provides the desired formulation. Briefly, the lyophilized powder is prepared by dissolving an excipient, such as dextrose, sorbital, fructose, corn syrup, xylitol, glycerin, glucose, sucrose or other suitable agent, in a suitable buffer, such as citrate, sodium or potassium phosphate or other such buffer known to those of skill in the art. Then, a selected enzyme is added to the resulting mixture, and stirred until it dissolves. The resulting mixture is sterile filtered or treated to remove particulates and to insure sterility, and apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with a buffer solution provides a formulation for use in parenteral administration.

2. Dosage and Administration

The soluble hyaluronidase provided herein can be formulated as pharmaceutical compositions, typically for single dosage administration. The selected soluble hyaluronidase is included in an amount sufficient to exert a therapeutically useful effect in the absence of undesirable side effects on the patient treated. For example, the subcutaneous co-administration of a soluble hyaluronidase with a bisphosphonate in the methods and uses provided can substantially reduce or eliminate injection site reactions, such as erythema, edema or ulceration, caused by the bisphosphonate.

The therapeutically effective concentration of a soluble hyaluronidase can be determined empirically by testing the polypeptides in known in vitro and in vivo systems such as by using the assays provided herein or known in the art (see e.g., Taliani et al. (1996) Anal. Biochem. 240:60-67; Filocamo et al. (1997) J. Virol. 71:1417-1427; Sudo et al. (1996) Antiviral Res. 32:9-18; Buffard et al. (1995) Virology 209:52-59; Bianchi et al. (1996) Anal. Biochem. 237:239-244; Hamatake et al. (1996) Intervirology 39:249-258; Steinkuhler et al. (1998) Biochem. 37:8899-8905; D'Souza et al. (1995) J. Gen. Virol. 76:1729-1736; Takeshita et al. (1997) Anal. Biochem. 247:242-246; see also, e.g, Shimizu et al. (1994) J. Virol. 68:8406-8408; Mizutani et al. (1996) J. Virol. 70:7219-7223; Mizutani et al. (1996) Biochem. Biophys. Res. Commun. 227:822-826; Lu et al. (1996) Proc. Natl. Acad. Sci. (USA), 93:1412-1417; Hahm et al. (1996) Virology 226:318-326; Ito et al. (1996) J. Gen. Virol. 77:1043-1054; Mizutani et al. (1995) Biochem. Biophys. Res. Commun. 212:906-911; Cho et al. (1997) J. Virol. Meth. 65:201-207 and then extrapolated therefrom for dosages for humans.

Typically, for therapeutically effective doses of a soluble hyaluronidases, the hyaluronidase is subcutaneously administered in a liquid formulation where the hyaluronidase is at a concentration of about 10-5,000,000 Units/milliliter (U/ml). For example, soluble hyaluronidase can be administered subcutaneously at a concentration at or about 10 U/ml, at or about 100 U/ml, at or about 500 U/ml, at or about 1000 U/ml, at or about 2000 U/ml, at or about 5000 U/ml, at or about 10,000 U/ml, at or about 20,000 U/ml, at or about 30,000 U/ml, at or about 40,000 U/ml, at or about 50,000 U/ml, at or about 60,000 U/ml, at or about 70,000 U/ml, at or about 80,000 U/ml, at or about 90,000 U/ml, at or about 100,000 U/ml or more. In some examples, dosages can be provided as a ratio of units hyaluronidase units to the amount of bisphosphonate administered. For example, hyaluronidase can be administered at or about 10 U/mg to 2,000,000 U/mg or more (hyaluronidase units per milligram bisphosphonates), for example, at or about 10 U/mg, at or about 25 U/mg; at or about 100 U/mg; at or about 1000 U/mg; at or about 2500 U/mg; at or about 5000 U/mg; at or about 10,000 U/mg; at or about 20,000 U/mg; at or about 100,000 U/mg; at or about 200,000 U/mg; at or about 1,000,000 U/mg; or at or about 2,000,000 U/mg or more. Typically, volumes of injections or infusions of hyaluronidase contemplated herein are from at or about 0.5 ml, 1 ml, 2 ml, 3 ml, 4 ml, 5 ml, 6 ml, 7 ml, 8 ml, 9 ml, 10 ml, 15 ml, 20 ml, 30 ml, 40 ml, 50 ml, 100 ml, 150 ml, 200 ml, 300 ml, 400 ml, 500 ml, 600 ml, 700 ml or more. The hyaluronidase can be provided as a stock solution at or about 50 U/ml, 100 U/ml, 150 U/ml, 200 U/ml, 400 U/ml or 500 U/ml or can be provided in a more concentrated form, for example at or about 1000 U/ml, 1500 U/ml, 2000 U/ml, 4000 U/ml, 5000 U/ml, 10000 U/ml or more for use directly or for dilution to the effective concentration prior to use. The soluble hyaluronidase can be provided as a liquid or lyophilized formulation. Lyophilized formulations are ideal for storage of large Units doses of soluble hyaluronidase.

The bisphosphonate preparations provided herein can be formulated as pharmaceutical compositions for single or multiple dose use. Typically, bisphosphonates preparations are formulated for single dose administration in an amount sufficient to provide a dose equivalent to the dosage administered IV. Bisphosphonates preparations also can be provided in lesser amounts for multiple dosage administrations. Typically dosage frequencies for subcutaneous administration of a bisphosphonate preparation with a soluble hyaluronidase are similar to the dosage frequencies for intravenous administration for a particular disease or condition. For example, dosage frequencies can be once a week, once every two weeks, once every three weeks, once every four weeks, once a month, once every two months, once every three months, once every four months, once every five months, once every six months, once every seven months, once every eight months, once every nine months, once every ten months, once every eleven months, once every twelve months, once every twelve months or once every two years. The bisphosphonate preparations can be provided in lyophilized or liquid form as discussed elsewhere herein.

The bisphosphonate is provided in a therapeutically effective dose. Therapeutically effective concentration can be determined empirically by testing the compounds in known in vitro and in vivo systems, such as the assays provided herein. The concentration of a selected bisphosphonate in the composition depends on absorption, inactivation and excretion rates of the complex, the physicochemical characteristics of the complex, the dosage schedule, and amount administered as well as other factors known to those of skill in the art. For example, it is understood that the precise dosage and duration of treatment is a function of the tissue being treated and can be determined empirically using known testing protocols or by extrapolation from in vivo or in vitro test data. It is to be noted that concentrations and dosage values can also vary with the age of the individual treated. It is to be further understood that for any particular subject, specific dosage regimens can be adjusted over time according to the individual need and the professional judgment of the person administering or supervising the administration of the formulations, and that the concentration ranges set forth herein are exemplary only and are not intended to limit the scope thereof. The amount of a selected bisphosphonate preparation to be administered for the treatment of a disease or condition, for example a bisphosphonate-treatable disease or condition, can be determined by standard clinical techniques. In addition, in vitro assays and animal models can be employed to help identify optimal dosage ranges.

Hence, the precise dosage, which can be determined empirically, can depend on the particular bisphosphonate preparation, the regime and dosing schedule with the soluble hyaluronidase, the route of administration, the type of disease to be treated and the seriousness of the disease. Generally, bisphosphonate is provided in an amount that permits subcutaneous administration of a dose equivalent to a once monthly IV dose for the particular indication being treated. The particular once monthly IV dose is a function of the disease to be treated, and thus can vary. Exemplary dosages ranges for subcutaneous administration of a bisphosphonate are from at or about 0.5 milligrams (mg), at or about 1 mg, at or about 3 mg, at or about 5 mg, at or about 10 mg, at or about 20 mg, at or about 30 mg, at or about 40 mg, at or about 50 mg, at or about 60 mg, at or about 70 mg, at or about 80 mg, at or about 90 mg, at or about 100 mg, or more. The particular dosage and formulation thereof depends upon the potency of the bisphosphonate. For example, for administration of ibandronate, dosages can be administered at or about 0.5 milligrams (mg), at or about 1 mg, at or about 1.5 mg, at or about 2 mg, at or about 2.5 mg, at or about 3 mg, at or about 3.5 mg, at or about 4 mg, at or about 4.5 mg, at or about 5 mg, at or about 5.5 mg, at or about 6 mg, at or about 6.5 mg, at or about 7 mg, at or about 7.5 mg, at or about 8 mg, at or about 8.5 mg, at or about 9 mg, at or about 9.5 mg, or at or about 10 mg, or more. In another example, for administration of zoledronate, dosages can be administered at or about 0.5 milligrams (mg), at or about 1 mg, at or about 1.5 mg, at or about 2 mg, at or about 2.5 mg, at or about 3 mg, at or about 3.5 mg, at or about 4 mg, at or about 4.5 mg, at or about 5 mg, at or about 5.5 mg, at or about 6 mg, at or about 6.5 mg, at or about 7 mg, at or about 7.5 mg, at or about 8 mg, at or about 8.5 mg, at or about 9 mg, at or about 9.5 mg, or at or about 10 mg, or more. In another example, for administration of pamidronate, dosages can be administered at or about 10 mg, at or about 20 mg, at or about 30 mg, at or about 40 mg, at or about 50 mg, at or about 60 mg, at or about 70 mg, at or about 80 mg, at or about 90 mg, or at or about 100 mg, or more.

The particular dosage and formulation thereof depends upon the indication and individual. If necessary, dosage can be empirically determined. To achieve such dosages, volumes of bisphosphonate preparations administered subcutaneously can be at or about 1 milliliter (ml), is or is about 5 ml, is or is about 10 ml, is or is about 25 ml, is or is about 50 ml, is or is about 100 ml, is or is about 150 ml, is or is about 200 ml, is or is about 300 ml, is or is about 400 ml, is or is about 500 ml, is or is about 600 ml, is or is about 700 ml, or more.

Where large volumes are administered, administration is typically by infusion. Subjects can be dosed, for example, at rates of infusion at or about 1 ml/kg/BW/h, 2 ml/kg/BW/h1 ml/kg/BW/h, 3 ml/kg/BW/h, 4 ml/kg/BW/h, or 5 ml/kg/BW/h. The infusion rate can be empirically determined, and typically is a function of the tolerability of the subject. If an adverse reaction occurs during the infusion, the rate of infusion can be slowed to the rate immediately below that at which the adverse event occurred. If the adverse event resolves in response to the reduction in rate, the infusion rate can be slowly increased at the discretion of the physician. Subcutaneous bisphosphonate infusion can be facilitated by gravity, pump infusion or injection of the desired dose. Generally, for infusions intravenous infusion pumps can be employed. Infusion rates can be increased during the course of treatment so long as the infusion is tolerated by the patient. Due to the high rate of infusion achieved by subcutaneous administration of bisphosphonate co-formulated and/or co-administered with hyaluronidase, the time of infusion is significantly less than for conventional IV bisphosphonate therapies. Where infusion time exceeds the desired limit, a second infusion site can be started at the physician and subject's discretion.

Techniques for infusion are known to one of skill in the art, and are within the skill of a treating physician. Generally, the appropriate dose of bisphosphonate can be pooled into a standard IV bag. For example, a non-vented infusion set can be used that has a Y-port near its terminus. A 24-gauge subcutaneous infusion needle can be inserted at a site of the subject's preferences, but the abdomen and secondarily the thighs are recommended because of the volume of solution to be infused. The hyaluronidase and bisphosphonate can be provided in the same Y port apparatus. Other articles of manufacture also can be used herein for purposes of infusion by gravity or a pump, and include, but are not limited to tubes, bottles, syringes or other containers.

The soluble hyaluronidase can be administered subsequently, intermittently or simultaneously from the bisphosphonate preparation. Generally, the hyaluronidase is administered prior to administration of the bisphosphonate preparation to permit the hyaluronidase to degrade the hyaluronic acid in the interstitial space. For example, the soluble hyaluronidase can be administered 1 minute, 2 minute, 3 minute, 4 minute, 5 minute, 6 minute, 7 minutes, 8 minutes, 9 minutes, 10 minutes, 20 minutes or 30 minutes prior to administration of the bisphosphonate preparation. In some examples, the hyaluronidase is administered together with the bisphosphonate preparation. As will be appreciated by those of skill in the art, the desired proximity of co-administration depends in significant part n the effect half lives of the agents in the particular tissue setting, and the particular disease being treated, and can be readily optimized by testing the effects of administering the agents at varying times in suitable models, such as in suitable animal models. In some situations, the optimal timing of administration of the hyaluronidase will exceed 60 minutes.

Generally, prior to infusion of bisphosphonate, a soluble hyaluronidase is injected at a rate of at or about 0.2 ml/min, 0.5 ml/min, 1 ml/min, 2 ml/min, 5 ml/min, 10 ml/min or more. For example, the soluble hyaluronidase can be injected through the same Y-port used for subsequent infusion of bisphosphonate. As noted above, the volume of soluble hyaluronidase administered is a function of the dosage required, but can be varied depending on the concentration of a soluble hyaluronidase stock formulation available. For example, it is contemplated herein that soluble hyaluronidase is not administered in volumes greater than about 50 ml, and typically is administered in a volume of 5-30 ml. A syringe pump can be used for the higher volumes, at the discretion of the physician.

In the event that an infusion is not tolerated (e.g., it causes moderate to severe local reactions), a second infusion site can be started so that the subject receives the full dosage.

A bisphosphonate preparation can be administered at once, or can be divided into a number of smaller doses to be administered at intervals of time. Selected bisphosphonate preparations can be administered in one or more doses over the course of a treatment time for example over several hours, days, weeks, or months. In some cases, continuous administration is useful. It is understood that the precise dosage and course of administration depends on the indication and patient's tolerability.

Also, it is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens can be adjusted over time according to the individual need and the professional judgment of the person administering or supervising the administration of the compositions, and that the concentration ranges set forth herein are exemplary only and are not intended to limit the scope or use of compositions and combinations containing them. The compositions can be administered hourly, daily, weekly, monthly, yearly or once. Generally, dosage regimens are chosen to limit toxicity. The attending physician knows how to and when to terminate, interrupt or adjust therapy to lower dosage due to toxicity, or bone marrow, liver or kidney or other tissue dysfunctions. Conversely, the attending physician also knows how to and when to adjust treatment to higher levels if the clinical response is not adequate (precluding toxic side effects).

### G. Methods of Assessing Activity, Bioavailability and Pharmacokinetics

Assays can be used to assess the in vitro and in vivo activities of bisphosphonate alone or in combination with a soluble hyaluronidase. Included among such assays are those that assess the pharmacokinetic properties of subcutaneously-administered bisphosphonates, including bioavailability, and tolerability. The biological activity of both bisphosphonates and hyaluronidase also can be assessed using assays well known in the art. Such assays can be used, for example, to determine appropriate dosages of a bisphosphonate and hyaluronidase, and the frequency of dosing, for treatment.

1. Pharmacokinetics and Tolerability

Pharmacokinetic and tolerability studies, such as those described in the Examples below, can be performed using animal models or can be performed during clinical studies with patients. Animal models include, but are not limited to, mice, pigs, rats, rabbits, dogs, guinea pigs and non-human primate models, such as cynomolgus monkeys or rhesus macaques. In some instances, pharmacokinetic and tolerability studies are performed using healthy animals. In other examples, the studies are performed using animal models of a disease for which therapy with bisphosphonates is considered, such as animal models of any of the diseases and conditions described below.

The pharmacokinetics of subcutaneously administered bisphosphonates can be assessed by measuring such parameters as the maximum (peak) plasma bisphosphonate concentration (Cₘₐₓ), the peak time (i.e. when maximum plasma bisphosphonate concentration occurs; Tₘₐₓ), the minimum plasma bisphosphonate concentration (i.e. the minimum plasma concentration between doses of bisphosphonate; Cₘᵢₙ), the elimination half-life (T_{1/2}) and area under the curve (i.e. the area under the curve generated by plotting time versus plasma bisphosphonate concentration; AUC), following administration. The absolute bioavailability of subcutaneously administered bisphosphonate is determined by comparing the area under the curve of bisphosphonate following subcutaneous delivery (AUC_{sc}) with the AUC of bisphosphonate following intravenous delivery (AUCᵢᵥ). Absolute bioavailability (F), can be calculated using the formula: F=([AUC]_{sc}×dose_{sc})/([AUC]ᵢᵥ×doseᵢᵥ). The concentration of bisphosphonate in the plasma following subcutaneous administration can be measured using any method known in the art suitable for assessing concentrations of bisphosphonate in samples of blood. Exemplary methods include, but are not limited to, liquid chromatography, and tandem mass spectrometry (LC/MS/MS) assays.

A range of doses and different dosing frequency of dosing can be administered in the pharmacokinetic studies to assess the effect of increasing or decreasing concentrations of bisphosphonate and/or hyaluronidase in the dose. Pharmacokinetic properties of subcutaneously administered bisphosphonate, such as bioavailability, also can be assessed with or without co-administration of hyaluronidase. For example, pigs can be administered bisphosphonate subcutaneously in combination with hyaluronidase, or alone. Intravenous doses bisphosphonate also are given to another group of pigs. Blood samples can then be taken at various time points and the amount of bisphosphonate in the plasma can be determined, such as by liquid chromatography, and tandem mass spectrometry (LC/MS/MS) assays. The AUC can then be measured and the bioavailability of subcutaneously administered bisphosphonate administered with or without hyaluronidase can be determined. Such studies can be performed to assess the effect of co-administration with hyaluronidase on pharmacokinetic properties, such as bioavailability, of subcutaneously administered bisphosphonate.

Studies to assess safety and tolerability also are known in the art and can be used herein. Following subcutaneous administration of bisphosphonate, with or without co-administration of hyaluronidase, the development of any adverse reactions can be monitored. Adverse reactions can include, but are not limited to, injection site reactions, such as edema or swelling, headache, fever, fatigue, chills, flushing, dizziness, urticaria, wheezing or chest tightness, nausea, vomiting, rigors, back pain, chest pain, muscle cramps, seizures or convulsions, changes in blood pressure and anaphylactic or severe hypersensitivity responses. Typically, a range of doses and different dosing frequencies are be administered in the safety and tolerability studies to assess the effect of increasing or decreasing concentrations of bisphosphonate and/or hyaluronidase in the dose.

2. Biological Activity

a. Bisphosphonate

The ability of bisphosphonate to act as a therapeutic agent can be assessed in vitro or in vivo. In vitro assays using cell lines or primary osteoclasts generated from human peripheral blood mononuclear cells are available to measure the ability bisphosphonate to induce apoptosis in osteoclasts or inhibit the ability of osteoclasts to absorb bone in a bone pit formation assay (Susa et al. (2004) J Transl Med. 2: 6; Spinola et al. (2006) BMC Musculoskelet Disord. 7: 56).

The clinical efficacy of bisphosphonate therapy in patients receiving bisphosphonate therapy can be monitored by assessing factors such as the incidence of nonvertebral fracture, measuring the bone mineral densities from lumber spine, hip, femoral neck and trochanter (thigh) bone samples. Patients receiving bisphosphonate therapy can be monitored over time and compared to patients receiving placebo or alternative dosages. Histology of bone samples from patients receiving bisphosphonate therapy can also be monitored to ensure no defects in bone mineralization are present.

In vivo studies using animal models also can be performed to assess the therapeutic activity of the bisphosphonate. The therapeutic effect of bisphosphonate can be assessed using animal models of the diseases and conditions for which therapy using bisphosphonate is considered. Such animal models are known in the art, and include, but are not limited to, animal models for osteoporosis and bone metastases (see e.g., Tuner (2001) Eur. Cells and Materials 1:66-81; Jee and Yao (2001) J Musculoskel Neuron Interact 1(3):193-207; Rosol et al. (2003) Cancer 97 (3 Suppl):748-57; Rosol (2004) Cancer Treat Res. 118:47-81; U.S. Pat. No. 7,135,609).

b. Hyaluronidase

Hyaluronidase activity can be assessed using methods well known in the art. In one example, activity is measured using a microturbidity assay. This is based on the formation of an insoluble precipitate when hyaluronic acid binds with serum albumin. The activity is measured by incubating hyaluronidase with sodium hyaluronate (hyaluronic acid) for a set period of time (e.g. 10 minutes) and then precipitating the undigested sodium hyaluronate with the addition of acidified serum albumin. The turbidity of the resulting sample is measured at 640 nm after an additional development period. The decrease in turbidity resulting from hyaluronidase activity on the sodium hyaluronate substrate is a measure of hyaluronidase enzymatic activity. In another example, hyaluronidase activity is measured using a microtiter assay in which residual biotinylated hyaluronic acid is measured following incubation with hyaluronidase (see e.g. Frost and Stern (1997) Anal. Biochem. 251:263-269; U.S. Patent Pub. No. 2005/0260186). The free carboxyl groups on the glucuronic acid residues of hyaluronic acid are biotinylated, and the biotinylated hyaluronic acid substrate is covalently couple to a microtiter plate. Following incubation with hyaluronidase, the residual biotinylated hyaluronic acid substrate is detected using an avidin-peroxidase reaction, and compared to that obtained following reaction with hyaluronidase standards of known activity. Other assays to measure hyaluronidase activity also are known in the art and can be used in the methods herein (see e.g. Delpech et al. (1995) Anal. Biochem. 229:35-41; Takahashi et al. (2003) Anal. Biochem. 322:257-263).

The ability of hyaluronidase to act as a spreading or diffusing agent also can be assessed. For example, trypan blue dye can be injected subcutaneously with or without hyaluronidase into the lateral skin on each side of nude mice. The dye area is then measured, such as with a microcaliper, to determine the ability of hyaluronidase to act as a spreading agent (U.S. Patent Pub. No. 2006/0104968).

### H. Therapeutic Uses

The methods described herein can be used for treatment of any condition for which a bisphosphonate is employed. A bisphosphonate can be administered subcutaneously, in combination with hyaluronidase, to treat any condition that is amendable to treatment with a bisphosphonate. This section provides exemplary therapeutic uses of a bisphosphonate. The therapeutic uses described below are exemplary and do not limit the applications of the methods described herein. Therapeutic uses include, but are not limited to, osteoporosis, Paget's disease, abnormally increased bone turnover, periodontal disease, tooth loss, bone fractures, rheumatoid arthritis, periprosthetic osteolysis, osteogenesis imperfecta (e.g., brittle bones), metastatic bone disease, heterotopic ossification, fibrous dysplasia, primary hyperparathyroidism, bone metastases, hypercalcemia of malignancy and multiple myeloma. It is within the skill of a treating physician to identify such diseases or conditions.

Diseases associated with bone metastasis include cancers that spread from the primary tumor located in one part of the body to another. For example, an individual with prostate cancer can have a metastasis in their bone. Cells that metastasize are typically of the same kind as those in the original tumor, i.e.; if the cancer arose in the lung and metastasized to the bone, the cancer cells growing in the bone are lung cancer cells. Metastatic-associated diseases which may be treated by methods of the invention include, but are not limited to, skin cancer, brain cancer, ovarian cancer, breast cancer, cervical cancer, colorectal cancer, prostate cancer, liver cancer, lung cancer, stomach cancer, bone cancer, and pancreatic cancer.

Bisphosphonates have also been shown to inhibit tumor angiogenesis. Thus, the methods and uses of subcutaneous administration of a hyaluronidase with a bisphosphonate can also be employed in the cancer therapies for the inhibition of tumor growth.

Bisphosphonate can be co-administered with hyaluronidase subcutaneously, in combination with other agents used in the treatment of these diseases and conditions. For example, additional agents that can be administered include, but are not limited to, vitamin and mineral supplements, such as calcium and Vitamin D or an analog, hormones, such as a steroid hormone, e.g. an estrogen; a partial estrogen agonist, or estrogen-gestagen combination; an androgen receptor modulator; a calcitonin or an analogue or derivative thereof, e.g. salmon, eel or human calcitonin parathyroid hormone or analogues thereof, e.g. PTH (1-84), PTH (1-34), PTH (1-36), PTH (1-38), PTH (1-31)NH 2 or PTS 893; a SERM (Selective Estrogen Receptor Modulator) e.g. raloxifene, lasofoxifene, TSE-424, FC1271, Tibolone (Livial®); cathepsin K inhibitor; an inhibitor of osteoclast proton ATPase; an inhibitor of HMG-CoA reductase; an integrin receptor antagonist; selective serotonin reuptake inhibitors (SSRIs); antibodies the prevent bone loss, such as denosumab, which prevents bone removal by inhibition of the RANKL cytokine; and the pharmaceutically acceptable salts and mixtures thereof. Such additional bone active drugs can be administered more frequently than the bisphosphonate.

In some examples, where the disease or condition to be treated is a cancer-related disease of condition, such as for example, hypercalcemia of malignancy, multiple myeloma, or metastatic bone disease, one or more chemotherapeutic agents or anti-cancer treatments can be administered with the bisphosphonate and hyaluronidase. Such agents and treatments are known in the art and include but are not limited to, surgery, radiation therapy, and chemotherapeutic agents, such as, for example, a chemotherapeutic compound, an antibody, a peptide, or a gene therapy vector, virus or DNA. Exemplary chemotherapeutic agents that can be administered after, coincident with or before administration of the bisphosphonate and hyaluronidase include, but not limited to, Acivicins; Aclarubicins; Acodazoles; Acronines; Adozelesins; Aldesleukins; Alemtuzumabs; Alitretinoins (9-Cis-Retinoic Acids); Allopurinols; Altretamines; Alvocidibs; Ambazones; Ambomycins; Ametantrones; Amifostines; Aminoglutethimides; Amsacrines; Anastrozoles; Anaxirones; Ancitabines; Anthramycins; Apaziquones; Argimesnas; Arsenic Trioxides; Asparaginases; Asperlins; Atrimustines; Azacitidines; Azetepas; Azotomycins; Banoxantrones; Batabulins; Batimastats; BCG Live; Benaxibines; Bendamustines; Benzodepas; Bexarotenes; Bevacizumab; Bicalutamides; Bietaserpines; Biricodars; Bisantrenes; Bisantrenes; Bisnafide Dimesylates; Bizelesins; Bleomycins; Bortezomibs; Brequinars; Bropirimines; Budotitanes; Busulfans; Cactinomycins; Calusterones; Canertinibs; Capecitabines; Caracemides; Carbetimers; Carboplatins; Carboquones; Carmofurs; Carmustines with Polifeprosans; Carmustines; Carubicins; Carzelesins; Cedefingols; Celecoxibs; Cemadotins; Chlorambucils; Cioteronels; Cirolemycins; Cisplatins; Cladribines; Clanfenurs; Clofarabines; Crisnatols; Cyclophosphamides; Cytarabine liposomals; Cytarabines; Dacarbazines; Dactinomycins; Darbepoetin Alfas; Daunorubicin liposomals; Daunorubicins/Daunomycins; Daunorubicins; Decitabines; Denileukin Diftitoxes; Dexniguldipines; Dexonnaplatins; Dexrazoxanes; Dezaguanines; Diaziquones; Dibrospidiums; Dienogests; Dinalins; Disermolides; Docetaxels; Dofequidars; Doxifluridines; Doxorubicin liposomals; Doxorubicin HCL; Docorubicin HCL liposome injection; Doxorubicins; Droloxifenes; Dromostanolone Propionates; Duazomycins; Ecomustines; Edatrexates; Edotecarins; Eflomithines; Elacridars; Elinafides; Elliott's B Solutions; Elsamitrucins; Emitefurs; Enloplatins; Enpromates; Enzastaurins; Epipropidines; Epirubicins; Epoetin alfas; Eptaloprosts; Erbulozoles; Esorubicins; Estramustines; Etanidazoles; Etoglucids; Etoposide phosphates; Etoposide VP-16s; Etoposides; Etoprines; Exemestanes; Exisulinds; Fadrozoles; Fazarabines; Fenretinides; Filgrastims; Floxuridines; Fludarabines; Fluorouracils; 5-fluorouracils; Fluoxymesterones; Fluorocitabines; Fosquidones; Fostriecins; Fostriecins; Fotretamines; Fulvestrants; Galarubicins; Galocitabines; Gemcitabines; Gemtuzumabs/Ozogamicins; Geroquinols; Gimatecans; Gimeracils; Gloxazones; Glufosfamides; Goserelin acetates; Hydroxyureas; Ibritumomabs/Tiuxetans; Idarubicins; Ifosfamides; Ilmofosines; Ilomastats; Imatinib mesylates; Imexons; Improsulfans; Indisulams; Inproquones; Interferon alfa-2 as; Interferon alfa-2bs; Interferon Alfas; Interferon Betas; Interferon Gammas; Interferons; Interleukin-2s and other Interleukins (including recombinant Interleukins); Intoplicines; Iobenguanes [131-I]; Iproplatins; Irinotecans; Irsogladines; Ixabepilones; Ketotrexates; L-Alanosines; Lanreotides; Lapatinibs; Ledoxantrones; Letrozoles; Leucovorins; Leuprolides; Leuprorelins (Leuprorelides); Levamisoles; Lexacalcitols; Liarozoles; Lobaplatins; Lometrexols; Lomustines/CCNUs; Lomustines; Lonafamibs; Losoxantrones; Lurtotecans; Mafosfamides; Mannosulfans; Marimastats; Masoprocols; Maytansines; Mechlorethamines; Meclorethamines/Nitrogen mustards; Megestrol acetates; Megestrols; Melengestrols; Melphalans; MelphalanslL-PAMs; Menogarils; Mepitiostanes; Mercaptopurines; 6-Mercaptopurine; Mesnas; Metesinds; Methotrexates; Methoxsalens; Metomidates; Metoprines; Meturedepas; Miboplatins; Miproxifenes; Misonidazoles; Mitindomides; Mitocarcins; Mitocromins; Mitoflaxones; Mitogillins; Mitoguazones; Mitomalcins; Mitomycin Cs; Mitomycins; Mitonafides; Mitoquidones; Mitospers; Mitotanes; Mitoxantrones; Mitozolomides; Mivobulins; Mizoribines; Mofarotenes; Mopidamols; Mubritinibs; Mycophenolic Acids; Nandrolone Phenpropionates; Nedaplatins; Nelzarabines; Nemorubicins; Nitracrines; Nocodazoles; Nofetumomabs; Nogalamycins; Nolatrexeds; Nortopixantrones; Octreotides; Oprelvekins; Ormaplatins; Ortataxels; Oteracils; Oxaliplatins; Oxisurans; Oxophenarsines; Paclitaxels; Patubilones; Pegademases; Pegaspargases; Pegfilgrastims; Peldesines; Peliomycins; Pelitrexols; Pemetrexeds; Pentamustines; Pentostatins; Peplomycins; Perfosfamides; Perifosines; Picoplatins; Pinafides; Pipobromans; Piposulfans; Pirfenidones; Piroxantrones; Pixantrones; Plevitrexeds; Plicamycid Mithramycins; Plicamycins; Plomestanes; Plomestanes; Porfimer sodiums; Porfimers; Porfiromycins; Prednimustines; Procarbazines; Propamidines; Prospidiums; Pumitepas; Puromycins; Pyrazofurins; Quinacrines; Ranimustines; Rasburicases; Riboprines; Ritrosulfans; Rituximabs; Rogletimides; Roquinimexs; Rufocromomycins; Sabarubicins; Safingols; Sargramostims; Satraplatins; Sebriplatins; Semustines; Simtrazenes; Sizofurans; Sobuzoxanes; Sorafenibs; Sparfosates; Sparfosic Acids; Sparsomycins; Spirogermaniums; Spiromustines; Spiroplatins; Spiroplatins; Squalamines; Streptonigrins; Streptovarycins; Streptozocins; Sufosfamides; Sulofenurs; Sunitinib Malate; 6-TG; Tacedinalines; Talcs; Talisomycins; Tallimustines; Tamoxifens; Tariquidars; Tauromustines; Tecogalans; Tegafurs; Teloxantrones; Temoporfins; Temozolomides; Teniposides/VM-26s; Teniposides; Teroxirones; Testolactones; Thiamiprines; Thioguanines; Thiotepas; Tiamiprines; Tiazofurins; Tilomisoles; Tilorones; Timcodars; Timonacics; Tirapazamines; Topixantrones; Topotecans; Toremifenes; Tositumomabs; Trabectedins (Ecteinascidin 743); Trastuzumabs; Trestolones; Tretinoins/ATRA; Triciribines; Trilostanes; Trimetrexates; Triplatin Tetranitrates; Triptorelins; Trofosfamides; Tubulozoles; Ubenimexs; Uracil Mustards; Uredepas; Valrubicins; Valspodars; Vapreotides; Verteporfins; Vinblastines; Vincristines; Vindesines; Vinepidines; Vinflunines; Vinformides; Vinglycinates; Vinleucinols; Vinleurosines; Vinorelbines; Vinrosidines; Vintriptols; Vinzolidines; Vorozoles; Xanthomycin A's (Guamecyclines); Zeniplatins; Zilascorbs [2-H]; Zinostatins; Zorubicins; and Zosuquidars. In particular examples, the chemotherapeutic agent is selected from among doxorubicin, fluorouracil, cyclophosphamide, methotrexate, mitoxantrone, vinblastine, dexamethasone, prednisone, melphalan, vincristine, megesterol, tamoxifen, etoposide, cisplatin, cytarabine, paclitaxel, and aminoglutethimide.

Where an additional agent(s) is administered in combination with the bisphosphonate and hyaluronidase, the additional agent(s) can be administered simultaneously, sequentially or intermittently with the bisphosphonate and hyaluronidase. The agent(s) can be co-administered with the bisphosphonate and hyaluronidase, for example, as part of the same pharmaceutical composition or in a separate composition. The agent(s) can be co-administered with the bisphosphonate and hyaluronidase, for example, by the same method of delivery, or can be administered at the same time as bisphosphonate and hyaluronidase but by a different means of delivery, or can be administered at the same time as either the bisphosphonate or hyaluronidase but by a different means of delivery. The agent(s) also can be administered at a different time than administration of the modified therapeutic antibody, but close enough in time to the administration of the modified therapeutic antibody to have a combined prophylactic or therapeutic effect.

If necessary, a particular dosage and duration and treatment protocol can be empirically determined or extrapolated. For example, exemplary doses of intravenously administered bisphosphonate can be used as a starting point to determine appropriate dosages. Dosage levels can be determined based on a variety of factors, such as body weight of the individual, general health, age, the activity of the specific compound employed, sex, diet, time of administration, rate of excretion, drug combination, the severity and course of the disease, and the patient's disposition to the disease and the judgment of the treating physician. Generally, dosages of bisphosphonates are from or about 0.1 mg per kg body weight (mg/kg BW) to 1.5 mg/kg BW, and dosages of hyaluronidase units per mg bisphosphonate are from or about 10 U/mg to 2,000,000 U/mg or more, for example, at or about 10 U/mg; at or about 25 U/mg; at or about 100 U/mg; at or about 1000 U/mg; at or about 2500 U/mg; at or about 5000 U/mg; at or about 10,000 U/mg; at or about 20,000 U/mg; at or about 100,000 U/mg; at or about 200,000 U/mg; at or about 1,000,000 U/mg; or at or about 2,000,000 U/mg, or more.

Generally, an appropriate amount of bisphosphonate is selected to obtain a bone resorption inhibiting effect, i.e. a bone resorption inhibiting amount of the bisphosphonate is administered. It will be appreciated that the actual unit dose used also will depend upon the potency of the bisphosphonates and the dosing interval employed. Thus, the size of the unit dose is typically lower for more potent bisphosphonates and greater the longer the dosing interval. For example, for more potent bisphosphonates, such as zoledronic acid or ibandronate, a unit dose of from about 0.5 mg up to about 10 mg, for example, at or about 0.5 milligrams (mg), at or about 1 mg, at or about 1.5 mg, at or about 2 mg, at or about 2.5 mg, at or about 3 mg, at or about 3.5 mg, at or about 4 mg, at or about 4.5 mg, at or about 5 mg, at or about 5.5 mg, at or about 6 mg, at or about 6.5 mg, at or about 7 mg, at or about 7.5 mg, at or about 8 mg, at or about 8.5 mg, at or about 9 mg, at or about 9.5 mg, or at or about 10 mg can be used in the methods of administration provided. In a particular example, about 3 mg of ibandronate is administered subcutaneously with 100-1000 Units of a soluble hyaluronidase in a volume of about 3 ml. In another particular example, about 4-5 mg of zoledronate is administered subcutaneously with 100-1000 Units of a soluble hyaluronidase in a volume of about 25-400 ml. Bisphosphonates that are less potent, such as pamidronate, can be administered in a unit dose of from at or about 10 mg up to at or about 90 mg, such as at or about 10 mg, at or about 20 mg, at or about 30 mg, at or about 40 mg, at or about 50 mg, at or about 60 mg, at or about 70 mg, at or about 80 mg, at or about 90 mg, or at or about 100 mg, or more can be administered subcutaneously.

It is also understood that the amount to administer will be a function of the indication treated, and possibly side effects that will be tolerated. Dosages can be empirically determined using recognized models for each disorder.

Upon improvement of a patient's condition, a maintenance dose of a bisphosphonate can be administered subcutaneously in combination with hyaluronidase, if necessary, and the dosage, the dosage form, or frequency of administration, or a combination thereof can be modified. In some cases, a subject can require intermittent treatment on a long-term basis upon any recurrence of disease symptoms.

1. Non-Malignant Bone Disorders

a. Osteoporosis

Osteoporosis is a generalized loss of and thinning of bone that most frequently occurs in women after menopause (i.e., postmenopausal osteoporosis (PMO)) and increases the risk of fractures, especially in the spine, wrist and hip. Osteoporosis is also relatively common in elderly men and may occur in anyone in the presence of particular hormonal disorders and other chronic diseases or as a result of medications, specifically glucocorticoids, when the disease is called steroid- or glucocorticoid-induced osteoporosis (SIOP or GIOP). In osteoporosis, the bone mineral density (BMD) is reduced, bone microarchitecture is disrupted, and the amount and variety of non-collagenous proteins in bone is altered.

For osteoporosis, bisphosphonate drugs are the first-line treatment. The most often prescribed bisphosphonates are presently alendronate (FOSAMAX) 10 mg a day or 70 mg once a week, risedronate (ACTONEL) 5 mg a day or 35 mg once a week and or ibandronate (BONIVA) once a month. Intravenous treatment with bisphosphonates is also prescribed. In patients who had suffered a low-impact hip fracture, annual infusion of 5 mg zoledronic acid reduced risk of any fracture by 35% (from 13.9 to 8.6%), vertebral fracture risk from 3.8% to 1.7% and non-vertebral fracture risk from 10.7% to 7.6%.

Bisphosphonates can be administered subcutaneously to patients in combination with hyaluronidase at an appropriate dose, such as, for example, a dose similar to the dose used to administer selected bisphosphonates intravenously treat patients with osteoporosis. For example, a patient with osteoporosis can be administered about 5 mg of zoledronate or 1-5 mg ibandronate, in combination with hyaluronidase, subcutaneously. The amount of the bisphosphonate can be increased or decreased depending on, for example, the severity of the disease and the clinical response to therapy, which can be readily evaluated by one of skill in the art.

b. Glucocorticoid-Induced Osteoporosis

Glucocorticoid-induced osteoporosis is often caused by the use of glucocorticoids, such as cortisone and prednisone, which are often used to treat rheumatoid arthritis, asthma and a variety of other diseases. The steroids can cause premature death of bone-forming cells and slow their replacement. Therefore, osteoporosis and bone damage are severe long-term side effects of this treatment.

Bisphosphonates can be administered subcutaneously to patients in combination with hyaluronidase at an appropriate dose, such as, for example, a dose similar to the dose used to administer selected bisphosphonates intravenously treat patients with glucocorticoid-induced osteoporosis. For example, a patient with glucocorticoid-induced osteoporosis can be administered about 5 mg of zoledronate or 1-5 mg ibandronate, in combination with hyaluronidase, subcutaneously. The amount of the bisphosphonate can be increased or decreased depending on, for example, the severity of the disease and the clinical response to therapy, which can be readily evaluated by one of skill in the art.

c. Paget's Disease of Bone

Paget's disease of bone, also known as osteitis deformans, is a chronic disorder that typically results in enlargement and deformity of certain bones. Excessive breakdown and formation can cause bone to weaken, which can result in bone pain, arthritis, deformities and fractures. Paget's disease may be caused by a slow virus infection (i.e., paramyxoviruses such as measles and respiratory syncytial virus), present for many years before symptoms appear. There is also a hereditary factor since the disease may appear in more than one family member. Pamidronate is typically administered as an intravenous infusion in dosage of 30 mg over 4 hours on 3 consecutive days or 60 mg over 2-4 hours for 2 or more consecutive or non-consecutive days for the treatment of Paget's disease of the bone. Oral formulations of bisphosphonates, such as etidronate, alendronate, tiludronate also are typically administered for the treatment of Paget's disease of the bone.

Bisphosphonates can be administered subcutaneously to patients in combination with hyaluronidase at an appropriate dose, such as, for example, a dose similar to the dose used to administer selected bisphosphonates intravenously treat patients with Paget's disease. For example, a patient with Paget's disease can be administered about 5 mg of zoledronate or 30-90 mg pamidronate, in combination with hyaluronidase, subcutaneously. The amount of the bisphosphonate can be increased or decreased depending on, for example, the severity of the disease and the clinical response to therapy, which can be readily evaluated by one of skill in the art.

d. Osteogenesis Imperfecta

Osteogenesis imperfecta (OI) is a autosomal dominant genetic disease of the bone that results in brittle and frail bones. Patients with OI are born without the proper protein (collagen), or the ability to make it, usually because of a deficiency of Type-I collagen. Patients with OI either have less collagen than normal or the quality is poorer than normal. Because collagen is an important protein in bone structure, this impairment causes those with the condition to have weak or fragile bones. Bisphosphonates, particularly those containing nitrogen, are administered to increase bone mass and reduce the incidence of fracture. Pamidronate is usually administered as an intravenous infusion, lasting about three hours for the treatment of OI.

Bisphosphonates can be administered subcutaneously to patients in combination with hyaluronidase at an appropriate dose, such as, for example, a dose similar to the dose used to administer selected bisphosphonates intravenously treat patients with osteogenesis imperfecta. For example, a patient with osteogenesis imperfecta can be administered about 4 mg of zoledronate or 90 mg pamidronate, in combination with hyaluronidase, subcutaneously. The amount of the bisphosphonate can be increased or decreased depending on, for example, the severity of the disease and the clinical response to therapy, which can be readily evaluated by one of skill in the art.

2. Cancer-Related Bone Disorders

a. Hypercalcemia of Malignancy

Hypercalcemia of malignancy is a condition in which abnormally high concentrations of calcium are found in the bloodstream of patients with some cancers. Elevations are observed in association with some cancers, particularly those that spread to bone. Hypercalcemia of malignancy is a common complication among cancer patients, affecting approximately 10% to 20% of all patients at some point during the course of their disease and 20% to 40% of patients with advanced cancer. For treatment of hypercalcemia of malignancy, zoledronate (Zometa®) is typically administered as an intravenous infusion in dosage of 4 mg over 15 minutes or longer and pamidronate (Aredia®) is typically administered as an intravenous infusion in dosage of 60 mg over about four hours or 90 mg over about 24 hours.

Bisphosphonates can be administered subcutaneously to patients in combination with hyaluronidase at an appropriate dose, such as, for example, a dose similar to the dose used to administer selected bisphosphonates intravenously treat patients with hypercalcemia of malignancy. For example, a patient with hypercalcemia of malignancy can be administered about 4 mg of zoledronate or 90 mg pamidronate, in combination with hyaluronidase, subcutaneously. The amount of the bisphosphonate can be increased or decreased depending on, for example, the severity of the disease and the clinical response to therapy, which can be readily evaluated by one of skill in the art.

b. Metastatic Bone Disease

Metastatic bone disease involves the spread of cancer cells from their original location to bone. Breast and prostate cancer are the most common of these cancers. For treatment of metastatic bone disease, zoledronate (Zometa®) is typically administered as an intravenous infusion in dosage of 4 mg over 15 minutes or longer and pamidronate (Aredia®) is typically administered as an intravenous infusion in dosage of 90 mg over about 2-4 hours every 3-4 weeks, usually in conjunction with chemotherapy.

Bisphosphonates can be administered subcutaneously to patients in combination with hyaluronidase at an appropriate dose, such as, for example, a dose similar to the dose used to administer selected bisphosphonates intravenously treat patients with metastatic bone disease. For example, a patient with metastatic bone disease can be administered about 4 mg of zoledronate or 90 mg pamidronate, in combination with hyaluronidase, subcutaneously. The amount of the bisphosphonate can be increased or decreased depending on, for example, the severity of the disease and the clinical response to therapy, which can be readily evaluated by one of skill in the art.

c. Multiple Myeloma

Multiple myeloma is a malignant disease of the bone marrow in which certain cells grow out of control and break down bone. Multiple myeloma often causes structural bone damage resulting in painful fractures. Patients with multiple myeloma have abnormally high levels of osteoclasts, which results in breakdown of bone faster than it can be replaced, potentially causing fractures, bone pain, osteoporosis (i.e., thinning of the bones), and hypercalcemia (i.e., high levels of calcium in the blood). Pamidronic acid/pamidronate (AREDIA) and zoledronic acid/zoledronante (ZOMETA) are administered intravenously for treating bone loss from multiple myeloma.

For treatment of multiple myeloma, zoledronate (Zometa®) is typically administered as an intravenous infusion in dosage of 4 mg over 15 minutes or longer and pamidronate (Aredia®) is typically administered as an intravenous infusion in dosage of 90 mg over about 4 hours, usually in conjunction with chemotherapy.

Bisphosphonates can be administered subcutaneously to patients in combination with hyaluronidase at an appropriate dose, such as, for example, a dose similar to the dose used to administer selected bisphosphonates intravenously treat patients with multiple myeloma. For example, a patient with multiple myeloma can be administered about 5 mg of zoledronate or 90 mg pamidronate, in combination with hyaluronidase, subcutaneously. The amount of the bisphosphonate can be increased or decreased depending on, for example, the severity of the disease and the clinical response to therapy, which can be readily evaluated by one of skill in the art.

### I. Articles of Manufacture and Kits

Pharmaceutical compositions of a bisphosphonate and a soluble hyaluronidase, provided together or separately, can be packaged as articles of manufacture containing packaging material, a pharmaceutical composition which is effective for treating a bisphosphonate-treatable disease or condition, and a label that indicates that the composition and combinations are to be used for treating a bisphosphonate-treatable diseases and conditions. Exemplary of articles of manufacture are containers including single chamber and dual chamber containers. The containers include, but are not limited to, tubes, bags, bottles and syringes. The containers can further include a needle for subcutaneous administration.

The articles of manufacture provided herein contain packaging materials. Packaging materials for use in packaging pharmaceutical products are well known to those of skill in the art. See, for example, U.S. Pat. Nos. 5,323,907, 5,033,252 and 5,052,558, each of which is incorporated herein in its entirety. Examples of pharmaceutical packaging materials include, but are not limited to, blister packs, bottles, tubes, inhalers, pumps, bags, vials, containers, syringes, needles, bottles, and any packaging material suitable for a selected formulation and intended mode of administration and treatment. A wide array of formulations of the compounds and compositions provided herein are contemplated as are a variety of treatments for any bisphosphonate-treatable disease or condition.

Compositions of a bisphosphonate and a soluble hyaluronidase, provided together or separately, also can be provided as kits. Kits can include a pharmaceutical composition described herein and an item for administration. For example compositions can be supplied with a device for administration, such as a syringe, including a pre-filled syringe, an inhaler, a dosage cup, a dropper, or an applicator. The kit can, optionally, include instructions for application including dosages, dosing regimens and instructions for modes of administration. Kits also can include a pharmaceutical composition described herein and an item for diagnosis. For example, such kits can include an item for measuring the concentration, amount or activity of the bisphosphonate.

### J. EXAMPLES

The following examples are included for illustrative purposes only and are not intended to limit the scope of the invention.

### Example 1

### Co-administration of Soluble Recombinant Human PH20 (rHuPH20) and Zoledronic acid (ZA) Alleviates ZA-Induced Injection Site Toxicity

Zoledronic Acid (ZA) is a member of the bisphosphonate drug class which inhibits osteoclastic bone resorption. Commercial formulation of ZA (ZOMETA, Novartis) is indicated for prevention of skeletal related events (pathological fractures, spinal compression, radiation or surgery to bone, or tumor-induced hypercalcaemia) in patients with advanced malignancies involving bone and treatment of tumor-induced hypercalcaemia (TIH) (ZOMETA Product insert, Novartis Pharma Stein AG, Stein, Switzerland for Novartis Pharmaceuticals Corporation, East Hanover, N.J. 07936). The recommended administration protocol for ZA is intravenous infusion of 4 mg ZA diluted with 100 ml sterile 0.9% w/v sodium chloride or 5% w/v glucose solution and given in no less than a 15 minute intravenous infusion every 3 to 4 weeks. For osteoporosis and Paget's disease, ZA is available as a 5 mg dose formulated in a 100 mL ready-to-infuse bag (RECLAST). Because ZA is typically administered IV, most doses of Reclast® are given in infusion centers separate from the offices of the primary care physicians who typically treat osteoporosis patients.

Conversion to a subcutaneous (SC) route of administration for ZA would result in increased patient convenience and compliance since dosing of this medication can be conducted in the office of a patient's primary care physician. Development of SC administration of ZA, however, has been inhibited due to ZA-induced skin toxicity. For example, following IV injection, considerable toxicity at the injection site has been observed as skin irritation in 1% of patients receiving Zometa® IV infusion (Drug database (Novartis). Approved by Therapeutic Goods Administration: 13 Dec. 2005; Date of most recent amendment: 16 Feb. 2006) and local irritation at the injection site in rats after single IV administration of 1.6 mg/kg Zometa® (Committee for Medicinal Products for Human Use, European Public Assessment Report (EPAR) for Zometa®, Scientific Discussion, Published Online Jul. 11, 2007).

Hyaluronidase increases the absorption of locally injected drugs by degrading hyaluronan in the skin interstitium or subcutaneous space and has been used as an antidote for treatment of local injuries after oncologic drug extravasations (Bertelli et al. (1994) J. Cancer Res. Clin. Oncol. 120:505-506; Bookbinder L et al. (2006) J. Controlled Release 114:230-241). rHuPH20, a soluble, recombinantly derived form of human hyaluronidase, was employed to study the effects of soluble hyaluronidase on reducing injection site toxicity and increasing adsorption of zoledronic acid.

### A. Rodent Model for Study of Injection Site Toxicity

A rodent model of local injection site injury caused by ZA injection was employed for the study. Because of differences between the rodent and humans in the underlying architecture of the sub-dermal fascia, which facilitates the SC infusion of drugs in the rodent without the use of adjuvants such as rHuPH20, intradermal injections were performed to mimic the effects of SC extravasation in humans (Disa et al. (1998) Plastic and Reconstructive Surgery 101:370-374). Injection into the dermis facilitates external visualization and assessment of the irritant.

1. Materials and Methods Employed in the Study Zoledronic acid (Zometa®: Lots S0065, S0066; MW: 290.11; Mol. Formula: C₅H10N₂O₇P₂·H₂O; 4.264 mg ZA, 4 mg ZA anhydrous, in 5 mL sterile liquid concentrate solution, inactive ingredients: mannitol, USP, as bulking agent, water for injection and sodium citrate, USP, as buffering agent, Novartis) was diluted for intradermal injection using rHuPH20 dilution buffer (each mL contains 8.5 mg sodium chloride, 1.4 mg dibasic sodium phosphate, 1.0 mg albumin human, 0.9 mg edetate disodium, 0.3 mg calcium chloride, with sodium hydroxide added for pH adjustment to 7.4).

Batches 056-133 and 056-122 of rHuPH20 were produced essentially as described in Example 6, except that a 36 L bioreactor (Bellco 1964 series) containing 20 L CD CHO medium supplemented with 800 mL GlutaMAX, 100 mg recombinant human insulin and 300 mg gentamicin sulfate was inoculated with 3 L culture at an initial seeding density of 4.7−4.9×10⁵ cell/mL. Subsequent volumes were adjusted as appropriate. Batch 056-122 also was produced without the use of a viral inactivation step immediately prior to the column chromatography steps. The concentration of the stock solution used was 1,310,100 U/mL rHuPH20. The stock solution was diluted as indicated below for injection.

Nine to ten week old female Sprague Dawley rats, having approximate body weight of 200-250 grams, were employed for the study. One day prior to injection, rats were shaved and hair on the back and flank area were removed by Nair® cream (exposure less than 1 min to depilation cream) followed by deep wash with warm tap water. On the day of dosing, all rats were injected intradermally in flank area (one or two injection spots per side), with 0.1 or 0.2 mL as indicated of zoledronic acid solution, with or without rHuPH20, at each corresponding concentration as shown in study design table. Injection site appearance was observed and quantified by blind assessment each day following the injection until the end of the study, at which time animals were sacrificed and the skin around the dosing area as well as a part of the skin that was not effected by the dosing were collected and fixed in 10% neutral buffered formalin (NBF) and further embedded in paraffin, 5 μm sections were cut for H&E stain and histopathology evaluation.

2. Quantification and Statistical Analysis

Lesion area was calculated by the following formula: W×L×¼π. Quantitative results were analyzed using one-way analysis of variance (ANOVA) method, followed by Dunnett's comparison method (Prizm 4 program). The probability values of less than 0.05 (two-tailed) were used as the critical level of significance for all tests. Groups with a sample size of two were excluded from analysis. If the sample size was 2 for the control group, there were no statistical analyses at that interval for any of the groups. Histopathologic findings were characterized by semi-quantitative analysis of necrosis and infiltration in the skin tissue. The incidences of each finding were compared with control groups by Chi-square test.

### B. Injection Site Reactions Produced by Intra-Dermal Injection of Zoledronic Acid in Sprague Dawley Rats

Prior to ZA intradermal injection, rats were randomized by body weight into 7 groups of 1 or 2 rats per group (Table 2). Rats were injected with either 0.1 mL or 0.2 mL of each corresponding concentration intradermally at two separate locations on each shaved flank side for a total of 4 injections for each animal. Injection sites were observed daily post-injection for four days. Pictures of injection site reaction (ISR) area were captured by digital camera and lesion area was scored for induration, erythema, or ulceration. Two perpendicular diameters (width & length) of injection site lesion were measured by microcaliper. The lesion area was defined as the area bounded by the outer edge of the erythemic zone. Upon completion of the study, the animals were euthanized and skin at the injection site and from an untreated area of the back of the rat were excised, mounted flat onto a piece of cardboard, and placed in 10% NBF.

TABLE 2
Experimental Cohorts for Injections of Zoledronic Acid

| | No. of | Injection Sites | Volume | ZA |
|---|---|---|---|---|
| Group | Animals | per Animal | (mL) | (mg/mL) |
| 1 | 2 | 4 | 0.1/0.2 | 0 |
| 2 | 2 | 4 | 0.1/0.2 | 0.05 |
| 3 | 2 | 4 | 0.1/0.2 | 0.1 |
| 4 | 2 | 4 | 0.1/0.2 | 0.2 |
| 5 | 2 | 4 | 0.1/0.2 | 0.4 |
| 6 | 2 | 4 | 0.1/0.2 | 0.6 |
| 7 | 1 | 2 | 0.1/0.2 | 0.8 |

Injection site reactions to intradermal injection of ZA typically peaked 2- to 4-days after injection (data not shown). Therefore, the 4-day time-point was used as a measure of acute sensitivity to ZA. Injection site reactions were observed at all concentrations of ZA tested (Table 3). For injections at 0.05 to 0.2 mg/mL, erythema was the sole observation occurring in all animals (n=4). At 0.4 mg/mL and above, induration and ulceration were also uniformly observed, becoming more severe at the highest doses.

Quantitation of the area of the injection site lesion was made using erythema to define the lesion area. At lower ZA concentrations (0.05-0.2 mg/ml), the erythema area was flat at ˜35 mm²; however, at dosing levels corresponding with the increase in incidence of induration and ulceration, a clear and statistically significant (r=0.9708) dose-response relationship was evident.

TABLE 3
Incidence results of injection site reactions (ISRs) on day 4 after
injection

| | | ISR Incidence | | |
|---|---|---|---|---|
| | Injection Sites | Induration | Erythema | Ulceration |
| ZA (mg/mL) | per Animal | (%) | (%) | (%) |
| 0.05 | 4 | — | 4/4 (100) | — |
| 0.1 | 4 | — | 4/4 (100) | — |
| 0.2 | 4 | — | 4/4 (100) | — |
| 0.4 | 4 | 4/4 (100)¹ | 4/4 (100) | 4/4 (100)¹ |
| 0.6 | 4 | 4/4 (100)¹ | 4/4 (100) | 4/4 (100)¹ |
| 0.8 | 2 | 2/2 (100)¹ | 2/2 (100) | 2/2 (100)¹ |

¹p < 0.01 compared to ZA 0.05 mg/mL group

Histopathology of skin tissue at the injection site demonstrated increasing severity of local infiltration, transitioning from focal to diffuse between ZA dosing concentrations of 0.05 to 0.1 mg/ml. Necrosis extended from epidermis to into the mid-dermal region with increasing ZA concentrations between 0.05 and 0.6 mg/mL (Table 4). Four days after intradermal administration of 0.1 mL of 0.05 mg/mL ZA, 75% of rats produced epidermal necrosis, while 100% of rats produced epidermal necrosis after 0.1, 0.2, 0.4, 0.6 and 0.8 mg/mL ZA injections. At ZA dosing concentrations more than 0.2 mg/mL, the necrosis extended to the superficial dermis in 100%, 75%, 100% and 100% of rats after injection of 0.2, 0.4, 0.6 and 0.8 mg/mL ZA, respectively. Rats produced 25%, 100% and 100% partial thickness dermal necrosis after injection of 0.4, 0.6 and 0.8 mg/mL ZA.

Inflammatory infiltration was seen in all rats receiving ZA alone. In groups injected with 0.05 mg/mL infiltration was graded focal in 50% of cases and diffuse in the remainder. Diffuse infiltration was seen in all rats injected with more than 0.1 mg/mL ZA. Inflammatory infiltrates were seen in muscle in 75% of rats treated with 0.1 mg/mL ZA, and in all rats injected with concentrations higher than 0.2 mg/mL ZA. Inflammatory infiltrates were identified in all skin layers in all rats receiving injections of 0.2, 0.4, 0.6 and 0.8 mg/mL.

TABLE 4
Semi-quantitative histopathologic analysis 4 days after ID injection of
ZA alone

| | Necrosis (%)¹ | | | | | |
|---|---|---|---|---|---|---|
| | | Superficial | | Infiltration (%)¹ | | |
| ZA (mg/mL) | Epidermis | dermis | Mid-dermis | Focal | Diffuse | Muscular |
| 0.05 | 3/4 (75) | — | — | 2/4 (50) | 2/4 (50) | — |
| 0.1 | 4/4 (100) | — | — | — | 4/4 (100) | 3/4 (75) |
| 0.2 | 4/4 (100) | 4/4 (100) | — | — | 4/4 (100) | 4/4 (100) |
| 0.4 | 4/4 (100) | 3/4 (75) | 1/4 (25) | — | 4/4 (100) | 4/4 (100) |
| 0.6 | 4/4 (100) | 4/4 (100) | 4/4 (100) | — | 4/4 (100) | 4/4 (100) |
| 0.8 | 2/2 (100) | 4/4 (100) | 2/2 (100) | — | 2/2 (100) | 2/2 (100) |

¹Dash indicates no incidence of indicated finding

C. Injection Site Reactions from Intra-Dermal Injection of Zoledronic Acid and Co-administered with a Fixed Dose of rHuPH20 in Sprague Dawley rats

A series of experiments were conducted in which various dosing concentrations of rHuPH20 were tested for the ability to reduce acute injection site lesions. Prior to ZA intra-dermal injection, rats were randomized by body weight into 11 groups of 2 rats per group. Rats were injected intradermally with 0.1 mL of each co-formulated solution as shown in Table 5 and observed daily with ISR development assessed as in Section B. Four days after injection, rats were sacrificed and the skin at the injection site as well as untreated skin from the back was excised, mounted flat onto a piece of cardboard, and placed in 10% NBF.

TABLE 5
Experimental Cohorts for Injections of ZA and With or Without
Maximal Concentrations of rHuPH20

| | Animal # vs. | ZA | rHuPH20 | | Dosing volume |
|---|---|---|---|---|---|
| Group # | injection spots | (mg/mL) | U/mL | Route | (mL) |
| 1 | 2 × 4 | 0.02 | 0 | ID | 0.1 |
| 2 | 2 × 4 | 0.05 | 0 | ID | 0.1 |
| 3 | 2 × 4 | 0.1 | 0 | ID | 0.1 |
| 4 | 2 × 4 | 0.2 | 0 | ID | 0.1 |
| 5 | 2 × 4 | 0.4 | 0 | ID | 0.1 |
| 6 | 2 × 4 | 0.02 | 10,000 | ID | 0.1 |
| 7 | 2 × 4 | 0.05 | 10,000 | ID | 0.1 |
| 8 | 2 × 4 | 0.1 | 10,000 | ID | 0.1 |
| 9 | 2 × 4 | 0.2 | 10,000 | ID | 0.1 |
| 10 | 2 × 4 | 0.4 | 10,000 | ID | 0.1 |
| 11 | 2 × 4 | 0 | 10,000 | ID | 0.1 |

Preliminary experiments established that 10,000 U/mL rHuPH20 has a maximal effect on ISR formation in the rat intradermal (ID) injection model. The effect of co-formulation of 10,000 U/mL with various concentrations of ZA was examined. rHuPH20 was capable of complete suppression of injection site lesion formation resulting from ZA concentrations of up to 0.05 mg/mL. At concentrations of ZA above 0.05 mg/mL, quantifiable injection site lesions were apparent after injection of the rHuPH20 co-formulation. In each case, however, the average injection site lesion area was reduced at each time-point tested for the co-formulation. All comparisons between like groups with and without rHuPH20 were significant (p<0.05) for the reduction in lesion size except 0.4 mg/mL ZA on Day 3. To provide a better means of determining which ZA dosing concentrations give similar lesion areas with or without rHuPH20, a nonlinear regression curve was generated for each time-point. A shift of approximately 2.4, 3.3 and 5.7-fold of half maximal rHuPH20 effect (EC50) can be noted at days 2, 3, or 4 after injection, respectively.

D. Injection Site Reactions from Intra-Dermal Injection of a Fixed Dose of Zoledronic Acid and rHuPH20 Co-Administration in Sprague Dawley Rats

Prior to ZA intra-dermal injection, rats were randomized by body weight into 6 groups of 3 rats per group (Table 6). Rats were intradermally injected with 0.1 mL of each corresponding co-formulated solution at two separate locations, two on each shaved flank side, for a total of 4 injections for each animal. Injection sites were observed daily with ISR development assessed as in Section B for 6 days. Animals were then euthanized and the skin at the injection site as well as untreated skin from the back was excised, mounted flat onto a piece of cardboard, and placed in 10% NBF.

TABLE 6
Experimental Cohorts for Injections of ZA and Escalating
Concentrations of rHuPH20

| | | | | rHuPH20 |
|---|---|---|---|---|
| Group | Animal # | Volume (mL) | ZA (mg/mL) | (U/mL) |
| 1 | 3 | 0.1 | 0 | 10,000 |
| 2 | 3 | 0.1 | X¹ | 0 |
| 3 | 3 | 0.1 | X | 10 |
| 4 | 3 | 0.1 | X | 100 |
| 5 | 3 | 0.1 | X | 1000 |
| 6 | 3 | 0.1 | X | 10,000 |

¹Concentration ZA 0.05, 0.1, and 0.4 mg/mL for each animal, respectively

Doses of rHuPH20 from 1 through 10,000 U/ml were evaluated for the ability to reduce the gross injection site lesion area induced by fixed concentrations of ZA from 0.05 to 0.4 mg/ml. The threshold of rHuPH20 in lesion area reduction was 100 U/mL across all ZA concentrations tested while maximal activity was reached at 1000 U/mL regardless of the ZA concentration tested. In some cases, particularly at the highest ZA dosing concentration, 0.4 mg/mL, there appeared to be a shift toward earlier development of peak lesion area and earlier resolution of the lesion.

In order to provide a single measure of the effectiveness of rHuPH20 over the entire course of lesion development and resolution, the sum of the ISR areas measured on each day for 6-days post injection was used. These are depicted in Table 7. The integrated area is reduced for every group dosed with rHuPH20 at 100 U/mL and above, reaching statistical significance at 1000 U/mL and above.

TABLE 7
Peak Lesion Areas and Accumulated ISR Area over 6-Days after 0.1 mL
Intradermal Injection of 0.05, 0.1 and 0.4 mg/mL ZA with Indicated
Concentration of rHuPH20

| | ZA (mg/mL) | | | | | | | | |
|---|---|---|---|---|---|---|---|---|---|
| | 0.05 | | | 0.1 | | | 0.4 | | |
| rHuPH20 | Peak | AUC¹ | | | AUC¹ | | | AUC¹ | |
| (U/mL) | (mm²) | (mm² · day) | N | Peak (mm²) | (mm² · day) | N | Peak (mm²) | (mm² · day) | N |
| 0 | 28.8 ± 1.8 | 110.8 ± 5.6 | 12 | 32.3 ± 2.7 | 108.3 ± 6.9 | 12 | 62.9 ± 5.1 | 226.5 ± 9.1 | 8 |
| 1 | 23.1 ± 1.9 | 58.5 ± 7.2 | 12 | 29.6 ± 1.2 | 113 ± 4.4 | 12 | 56.0 ± 3.8 | 163.5 ± 9.1 | 8 |
| 10 | 26.9 ± 2.6 | 69.7 ± 6.8 | 12 | 30.3 ± 1.7 | 119.2 ± 6.7 | 12 | 53.4 ± 4.9 | 157.2 ± 7.3 | 8 |
| 100 | 15.3 ± 3.6 | 39.4 ± 6 | 12 | 23.9 ± 2.2 | 78.8 ± 7.4 | 12 | 36.7 ± 1.9 | 91.2 ± 4.8 | 8 |
| 1000 | 5.3 ± 2.5⁽²⁾ | 12.7 ± 4.8 | 12 | 15.9 ± 2.4⁽²⁾ | 55.1 ± 6 | 12 | 25.6 ± 4.4⁽²⁾ | 62.7 ± 6.3 | 8 |
| 10,000 | 4.2 ± 2.5⁽²⁾ | 9.7 ± 5.5 | 12 | 14.9 ± 2.1⁽²⁾ | 34.6 ± 4 | 12 | 21.9 ± 1.7⁽²⁾ | 49.4 ± 4.0 | 8 |

¹Areas under the curves from FIGS 2, 3, and 4 for 6 days post injection
⁽²⁾p < 0.001 by ANOVA and Dunn's test compared to ZA alone

Six days after ID co-administration of 0.05 mg/mL ZA with increasing doses of rHuPH20, histopathology of skin tissue at injection sites demonstrated complete resolution (Table 8) in groups receiving co-injection of 1000 and 10,000 U/mL of rHuPH20. In contrast, rats receiving ZA alone or co-injected with 10 U/mL of rHuPH20 showed focal infiltration (75%, and 25% respectively). Rats receiving ZA with 1 and 100 U/mL rHuPH20 showed epidermal necrosis (25%) and diffuse hypodermal infiltration (25%).

At 0.1 mg/ml ZA a strong trend toward reduction of epidermal necrosis exists above 100 U/ml (Table 9). Inflammatory infiltration in the skin also shows a strong reduction from 100% of animals observed to the 25-50% range above 100 U/ml transitioning from partially diffuse to entirely focal. Infiltration into muscular tissue was also significantly reduced above 100 U/ml with no observations of muscular infiltration at 10,000 U/mL.

At 0.4 mg/mL ZA, similar levels of epidermal necrosis were observed compared with 0.1 mg/mL with a similar dramatic reduction to 13% at rHuPH20 concentrations above 100 U/mL which was statistically significant (Table 10). Higher degrees of inflammatory infiltrates were observed at 0.4 mg/mL ZA compared to 0.1 mg/mL. At levels of rHuPH20 below 100 U/mL, all epidermal tissues showed signs of inflammatory infiltrates in both the skin and muscle. At the highest levels of rHuPH20 tested, 75% of animals showed dermal inflammatory infiltrates and 13% showed infiltration into muscle tissue. The decline in muscular infiltration was statistically significant.

TABLE 8
Semi-quantitative Histopathologic Analysis 4-Days After ID Injection
of 0.05 mg/mL ZA with Increasing Dose of rHuPH20

| | | Infiltration (%)¹ | | |
|---|---|---|---|---|
| rHuPH20 | Necrosis (%)¹ | Skin | | |
| (U/ml) | Epidermis | Focal | Skin Diffuse | Muscle |
| 0 | — | 1/4 (25) | — | — |
| 1 | 1/4 (25) | — | — | — |
| 10 | — | 3/4 (75) | — | — |
| 100 | 1/4 (25) | — | 1/4 (25) | — |
| 1000 | — | — | — | — |
| 10,000 | — | — | — | — |

¹Dash indicates no incidence of indicated finding

TABLE 9
Semi-quantitative histopathologic analysis 4 days after
ID injection of 0.1 mg/mL ZA with increasing dose of rHuPH20

| | Necrosis | Infiltration (%)¹ | | | |
|---|---|---|---|---|---|
| rHuPH20 | (%)¹ | Skin | | | |
| (U/ml) | Epidermis | Focal | Skin Diffuse³ | Muscle | |
| 0 | 4/12 (33) | 4/12 (33) | 8/12 (67) | 9/12 | (75) |
| 1 | 11/12 (92) | 2/12 (17) | 10/12 (83) | 7/12 | (58) |
| 10 | 9/12 (75) | 4/12 (33) | 9/12 (75) | 7/12 | (58) |
| 100 | 8/12 (67) | 3/12 (25) | — | 4/12 | (33)¹ |
| 1000 | 3/12 (25) | 6/12 (50) | — | 1/12 | (8)² |
| 10,000 | 1/11 (9) | 3/11 (27) | — | 0/8 | (0)² |

¹Dash indicates no incidence of indicated finding
²p < 0.05 compared with ZA alone by Chi-square
³p < 0.01 compared with ZA alone by Chi-square

TABLE 10
Semi-quantitative histopathologic analysis 4 days after ID injection of
0.4 mg/mL ZA with increasing dose of rHuPH20

| | Necrosis (%)¹ | | Infiltration (%)¹ | | |
|---|---|---|---|---|---|
| rHuPH20 | | Superficial | Skin | | |
| (U/ml) | Epidermis | dermis² | Focal² | Skin Diffuse | Muscle |
| 0 | 5/7(71) | — | — | 7/7 (100) | 7/7 (100) |
| 1 | 5/8 (63) | — | — | 8/8 (100) | 8/8 (100) |
| 10 | 3/8 (38) | 1/8 (13) | 1/8 (13) | 7/8 (88) | 8/8 (100) |
| 100 | 4/8 (50) | 2/8 (25) | 6/8 (75) | 2/8 (25) | 5/8 (63) |
| 1000 | 1/8 (13)² | — | 5/8 (63) | 3/8 (38) | 3/8 (38) |
| 10,000 | 1/8 (13) | — | 4/8 (50) | 2/8 (25) | 1/8 (13) |

¹Dash indicates no incidence of the indicated observation
²p < 0.05 compared with ZA alone by Chi-square

E. Injection Site Reaction Recovery Study

Sprague Dawley rats were randomized to three groups of 4 rats each. ZA (0.1 ml at 0.1 mg/mL), either alone or supplemented with either 130 U/mL or 100,000 U/mL rHuPH20, was given via intradermal injection. Injection sites were observed and measured daily. Animals were sacrificed at 10 days post-injection and skin from lesion areas were dissected, fixed in 10% buffered formalin, embedded in paraffin and stained with H&E.

To assess histological recovery from acute lesions, a small cohort study was performed to extend the observation time for 10 days after intradermal injection. All rats developed an erythematic lesion at the injection spot. Peak lesion area was typically achieved 3 days after injection. The erythemic area was significant smaller in rats injected with rHuPH20 than those of ZA alone. Complete healing was only observed in rats treated with 0.1 mg/mL ZA with 100,000 U/mL rHuPH20.

Histopathologic analysis revealed that histologically normal skin appeared in all samples from the 100,000 U/mL rHuPH20 dosing group. In contrast, scabbing, epidermal necrosis, infiltration, vessel dilation and hemorrhage were seen in samples from 0.1 mg/mL ZA alone. Epidermal necrosis and mild inflammatory infiltration were seen in samples after 0.1 mg/mL ZA+130 U/mL rHuPH20 injections.

### Example 2

### Co-administration of Soluble Recombinant Human PH20 (rHuPH20) and Ibandronate Alleviates Ibandronate-Induced Injection Site Toxicity

Ibandronate another member of the bisphosphonate drug class which inhibits osteoclastic bone resorption. Commercial formulations of Ibandronate (e.g., BONIVA, BONDRONAT, BONVIVA, Roche/GlaxoSmithKline) are indicated for the treatment of osteoporosis. Oral formulations of Ibandronate are given at a dose of 2.5 mg daily or 150 mg monthly. IV formulation was administered as an infusion of 1 mg every three months. Because injection site toxicity is often associated with members of the bisphosphonate family and inhibits subcutaneous administration, the ability of rHuPH20 to reduce injection site toxicity of Ibandronate was studied.

Employing similar protocols and analytical methods as outlined in Example 1, the effects of soluble hyaluronidase on reducing injection site toxicity induced by administration of Ibandronate was examined.

### A. Injection Site Reactions Produced by Intra-Dermal Injection of Ibandronate in Sprague Dawley Rats

In the first experiment, five dosages of Ibandronate alone were tested (0.08, 0.16, 0.32, 0.64 and 0.9 mg/mL n=8) to examine whether injection site reaction was dose dependent. Similar to what was observed for Zoledronic acid in Example 1B. Injection site reactions were observed at all concentrations of Ibandronate tested.

Quantitation of the area of the injection site lesion was made using erythema to define the lesion area. At lower Ibandronate concentrations (0.08-0.16 mg/mL, the average erythema area was ˜25 mm². At higher dosing levels, a clear and statistically significant (p<0.001 R2=0.9387) dose-response relationship was evident.

B. Injection Site Reactions from Intra-Dermal Injection of Zoledronic Acid and Co-administered with a Fixed Dose of rHuPH20 in Sprague Dawley Rats

A series of experiments were conducted in which various dosing concentrations of rHuPH20 were tested for the ability to reduce acute injection site lesions induced by Ibandronate. Sprague Dawley rats were injected with a 0.1 mL co-formulation of 0.32 mg/mL of Ibandronate and various concentrations of rHuPH20 (0, 10 U/mL, 100 U/mL, 1000 U/mL; n=8 for each dosage). ISR areas were measured daily for 8 days as outlined in Examples 1B and 1D. Data for ISR sizes were plotted against time. The accumulated lesion area after 9 days were demonstrated was 142 mm², 112 mm², 105 mm² and 72 mm² for the rHuPH20 doses of 0, 10 U/mL, 100 U/mL, 1000 U/mL.

### Example 3

### Pharmacokinetic Analysis of Subcutaneous (SC) Co-administration of Human Recombinant Hyaluronidase PH20 (rHuPH20) and Zoledronic Acid

A pharmacokinetic study of zoledronic acid (ZA) co-administered with rHuPH20 in female Yorkshire swines was conducted. The primary objective of the study was to determine the bioavailability of zoledronic acid dosed via subcutaneous (SC) co-administration with human recombinant hyaluronidase PH20 (rHuPH20) and to compare the overall exposure to an equivalent IV dose. Secondary objectives included comparing renal histopathology, if any, following subcutaneous co-administration of ZA with rHuPH20 (ZA+rHuPH20), to that observed following IV administered ZA; and to demonstrate acceptable subcutaneous injection site reactions (ISRs) at feasible subcutaneous ZA+rHuPH20 delivery volumes.

A. Methods of Treatment and Pharmacokinetic Analysis of SC Co-Administration of rHuPH20 and Zoledronic Acid

Thirteen female Yorkshire pigs were assigned to three treatment groups (Groups 1, 2, and 3) as summarized in Table 11. Animals in Study Group 1 were dosed with 0.15 mg/kg ZA given via a 20-minute IV infusion. Animals in Study Groups 2 and 3 were dosed subcutaneously (SC) with 0.15 mg/kg ZA without and with rHuPH20, respectively, in different SC delivery volumes.

Following ZA dose administration, serial blood samples were collected via vascular access ports (VAPs) for serum preparation and determination of ZA concentrations. Blood collection times were at 1, 3, 5, 10, 15, 20, 30, 45, and 60 minutes, and at 1.5, 2, 3, 4, 6, 8, 10, 24, 48, and 72 hours post dose. Urine samples were also collected to compare excretion characteristics between IV and SC routes of administration. Collection times for urine were at 15 minutes before dosing (−15 min.), continuously from dose initiation to 2 hours post dose, and at 4, 6, 8, and 24 hours post dose thereafter.

Serum concentration of ZA was determined using a qualified LC-MS-MS assay after derivatization with diazomethane (Zhu, L S et al. (2006) Rapid Commun. Mass Spectrom. 20: 3421-3426; “Qualification Data of An LC-MS/MS Method for the Determination of Zoledronate in Porcine Serum”, MDS Pharma Services Study No. AA43671-01, Report Date 14 Sep. 2007). The assay range was from 2.0 to 400 ng/mL with a lower limit of quantitation (LLOQ) at 2.0 ng/mL. Urine concentration of ZA was determined by a similar well qualified LC-MS-MS method (“Qualification Data of An LC-MS/MS Method for the Determination of Zoledronate in Porcine Urine”, MDS Pharma Services Study No. AA43671-02, Report Date 14 Sep. 2007) with an assay LLOQ at 10 ng/mL.

Pharmacokinetic (PK) analyses of ZA serum concentration versus time data were conducted using WinNonlin v5.1 (Pharsight, Mountain View, Calif.). Non-compartmental analysis was used for derivation of primary and secondary pharmacokinetic (PK) parameters. AUC, Cmax, and Tmax were compared between dose routes, between co-administration with rHuPH20, and with dose of rHuPH20.

Amount of ZA excreted unchanged in urine was determined for the collection interval from dosing to 2-hour post dose and compared between dose routes and co-administration with rHuPH20.

TABLE 11
Experimental Cohorts for Injections of ZA and With or Without
rHuPH20

| | | | | | | | | | | |
|---|---|---|---|---|---|---|---|---|---|---|
| | | | | | | | | | | 10% |
| | | | | | | | | | Final | Hylenex |
| | Body | Vol/ | # inj. sites | Inf. | Delivery | | | | ZA | or 10% |
| | Wt. | Dose | (needle | Rate | Time | rHuPH20 | ZA | ZA (mg/ | Conc. | Hylenex |
| ID | (kg) | (mL) | insertions) | (mL/min) | (min) | (U/mL) | (mg/kg) | animal) | (mg/mL) | placebo |

Group 1 - Control IV Infusion

| | | | | | | | | | | |
|---|---|---|---|---|---|---|---|---|---|---|
| 67 | 26.4 | 125 | n/a | 6.25 | 20 | 0 | 0.15 | 3.96 | 0.0317 | n/a |
| 61 | 26 | 125 | n/a | 6.25 | 20 | 0 | 0.15 | 3.9 | 0.0312 | n/a |
| 57 | 29.1 | 125 | n/a | 6.25 | 20 | 0 | 0.15 | 4.37 | 0.0349 | n/a |

Group 2 - Control SC Injection

| | | | | | | | | | | |
|---|---|---|---|---|---|---|---|---|---|---|
| 70 | 26 | 10 | 1x | 6.25 | 1.6 | 0 | 0.15 | 3.9 | 0.3900 | Placebo |
| 69 | 30 | 25 | 1x | 6.25 | 4.0 | 0 | 0.15 | 4.50 | 0.1800 | Placebo |
| 62 | 26.8 | 50 | 3x | 6.25 | 8.0 | 0 | 0.15 | 4.02 | 0.0804 | Placebo |
| 60 | 27.3 | 100 | 6x | 6.25 | 16 | 0 | 0.15 | 4.10 | 0.0410 | Placebo |
| 59 | 27.7 | 100 | 8-10x | 6.25 | 16 | 0 | 0.075 | 2.08 | 0.0208 | Placebo |

Group 3 - Co-administration Group

| | | | | | | | | | | |
|---|---|---|---|---|---|---|---|---|---|---|
| 68 | 29 | 10 | 1x | 6.25 | 1.6 | 10,000 | 0.15 | 4.35 | 0.4350 | Hylenex |
| 56 | 26 | 25 | 1x | 6.25 | 4.0 | 10,000 | 0.15 | 3.90 | 0.1560 | Hylenex |
| 58 | 26.4 | 50 | 1x | 6.25 | 8.0 | 10,000 | 0.15 | 3.96 | 0.0792 | Hylenex |
| 64 | 27.3 | 100 | 4x | 6.25 | 16 | 10,000 | 0.15 | 4.10 | 0.0410 | Hylenex |
| 66 | 27.7 | 100 | 8-10x | 6.25 | 16 | 10,000 | 0.075 | 2.08 | 0.0208 | Hylenex |

### B. Pharmacokinetics of ZA by Non-compartmental Analysis Following IV and SC Infusions

Mean and median ZA serum concentrations versus time data for Study Group 1 (0.15 mg/kg ZA given via a 20-minute IV infusion) are listed in Table 12. Pharmacokinetic parameters derived by non-compartmental analysis for this group of animals are presented in Table 13. Individual animal data is shown in Table 18.

Peak ZA serum concentration was attained at the end of the 20-minute IV infusion and declined thereafter. Concentration decline was biphasic and was below the assay limit of quantitation by 4 hours post dose. The early rapid decline (α phase) represented quick distribution from the circulation followed by a slower elimination phase (β phase). The terminal half-life (T½λz) of ZA in pigs was 0.72±0.08 hour. Mean absolute clearance (CL) was 440.12±33.72 mL/h-kg with a volume of distribution of 458.29±75.94 mL/kg. Maximum serum concentration (Cmax) was 575.67±108.56 ng/mL.

The PK of IV zoledronic acid previously has been described as being triphasic in man with a long terminal γ phase (T½γ˜146 hours) defined by very low ZA concentration and attributed to slow equilibrium with distribution to bone tissues and slow release back into the circulation (Prescribing Information for Zometa® concentrate, Novartis Pharmaceuticals Canada Inc., Control No. 113797, Date of Revision: Sep. 14, 2007). The terminal γ phase in this pig study may not have been characterized due to the combination of a low IV dose (0.15 mg/kg) and sensitivity of the quantitation assay.

TABLE 12
Mean Plasma Concentration versus Time Data for Study Group 1
Animals (20-Minute IV Infusion of 0.15 mg/kg ZA)

| | Mean ZA | | Median ZA | |
|---|---|---|---|---|
| Time | Concentration | | Concentration | |
| (h) | (ng/mL) | Std. Dev. | (ng/mL) | n |
| 0.0167 | 222.33 | 55.72 | 254.00 | 3 |
| 0.05 | 310.00 | 126.44 | 288.00 | 3 |
| 0.0833 | 382.00 | 62.02 | 384.00 | 3 |
| 0.167 | 502.33 | 97.59 | 448.00 | 3 |
| 0.25 | 575.67 | 108.56 | 580.00 | 3 |
| 0.33 | 513.33 | 110.37 | 556.00 | 3 |
| 0.50 | 263.00 | 31.19 | 246.00 | 3 |
| 0.75 | 101.57 | 44.92 | 127.00 | 3 |
| 1 | 78.23 | 7.06 | 77.80 | 3 |
| 1.5 | 34.27 | 6.35 | 31.70 | 3 |
| 2 | 16.93 | 1.72 | 16.60 | 3 |
| 3 | 6.19 | 0.44 | 6.10 | 3 |
| 4 | 3.15 | 0.30 | 3.30 | 3 |
| 6 | BLQ | BLQ | BLQ | 3 |
| 8 | BLQ | BLQ | BLQ | 3 |
| 10 | BLQ | BLQ | BLQ | 3 |
| 24 | BLQ | BLQ | BLQ | 3 |
| 48 | BLQ | BLQ | BLQ | 3 |
| 72 | BLQ | BLQ | BLQ | 3 |
| 312 | BLQ | BLQ | BLQ | 3 |

^{a}BLQ = below assay limit of quantification (2.0 ng/mL)

TABLE 13
Pharmacokinetic Parameters^{a} in Pigs Following a Twenty-minute IV
Infusion of 0.15 mg/kg Zoledronic Acid (Study Group 1)

| | | AUC | | AUC | | | | | | |
|---|---|---|---|---|---|---|---|---|---|---|
| | Body | (0-1 h)^{b} | AUC | (0-∞)^{b} | | | | CL^{f} | | |
| | Wt | (ng- | (0-8 h)^{b} | (ng- | Cmax^{c} | Tmax^{d} | T½ λz^{e} | (mL/h- | Vz^{g} | Vss^{h} |
| Pig ID | (kg) | h/mL) | (ng-h/mL | h/mL) | (ng/mL) | (h) | (h) | kg) | (mL/kg) | (mL/kg) |
| 57 | 29.1 | 262.8417 | 317.04 | 316.99 | 465.00 | 0.25 | 0.74 | 473.20 | 506.47 | 130.68 |
| 61 | 26.0 | 303.2708 | 370.55 | 369.64 | 682.00 | 0.25 | 0.63 | 405.81 | 370.76 | 104.41 |
| 67 | 26.4 | 280.3000 | 339.74 | 339.86 | 580.00 | 0.25 | 0.78 | 441.35 | 497.66 | 132.71 |
| Mean | | 282.1 | 342.44 | 342.16 | 575.67 | 0.25 | 0.72 | 440.12 | 458.29 | 122.60 |
| Stdev | | 20.3 | 26.85 | 26.40 | 108.56 | 0.00 | 0.08 | 33.72 | 75.94 | 15.79 |
| Median | | 280.3 | 339.74 | 339.86 | 580.00 | 0.25 | 0.74 | 441.35 | 497.66 | 130.68 |
| n | | 3 | 3 | 3 | 3 | 3 | 3 | 3 | 3 | 3 |

^{a}PK parameters derived by non-compartmental analysis
^{b}AUC = area under the serum concentration versus time curve
^{c}Cmax = maximum serum ZA concentration
^{d}Tmax = time to maximum serum ZA concentration
^{e}T½ λz = Lambda z half-life
^{f}CL = absolute clearance = Dose ÷ AUC
^{g}Vz = volume of distribution
^{h}Vss = volume of distribution at steady state

Selected primary and secondary PK parameters derived from non-compartmental modeling of the SC dose administrations with and without rHuPH20 (Study Groups 2 and 3) are shown in Tables 14 and 15, respectively. The PK parameters from these 2 study groups were used to assess any obvious effect or trend in the experimental variables (volume per dose, infusion/delivery time, and number of injection sites) employed among animals within the study group.

No obvious differences and systematic trending were noted in PK parameters for animals within the same dose groups (Tables 14 and 15). The individual data for each animal within the same dose group were thus pooled for PK analysis. Mean and median serum concentrations versus time data are listed in Table 16 for Study Group 2; and in Table 17 for Study Group 3. ZA concentrations versus time data for individual animals are shown in Tables 19 and 20.

The maximum serum concentrations (Cmax) attained following SC infusion of ZA with and without rHuPH20 were similar between Study Groups 2 and 3. Maximum serum concentration from SC infusion of 0.15 mg/kg ZA was attained between 0.167 to 0.33 hours post dose.

Mean Cmaxvalues were 294.75±34.73 and 296.50±46.92 ng/mL for Study Group 2 and 3, respectively. Decline from peak serum concentration (Cmax=294.75±34.73 ng/mL) was biphasic with a mean half-life of 1.07±0.24 hours. In the comparison to an equivalent IV dose, the peak concentration was ˜ one half of the IV Cmax. There appeared to be a slight difference between values for area under the serum concentration versus time curve from time zero extrapolated to infinite time. AUC_{(0-∞)} was 299.89±51.40 ng-h/mL for Study Group 2 and 331.11±39.93 ng-h/mL for Study Group 3; with slightly greater area when ZA was co-administrated with rHuPH20.

High bioavailability of ZA from SC infusion with rHuPH20 also was observed. SC bioavailability was computed using the 0.15 mg/kg dose since it was given both intravenously and subcutaneously; and area from time 0 to 8-hour post dose did not involve any extrapolation. Overall SC bioavailability for ZA was 87.65±15.02% and 96.00±10.98% when given without and with rHuPH20, respectively. Variability in bioavailability among animals appeared to be less with co-administration of rHuPH20.

Mean time to maximal serum concentration (Tmax) appeared to be shorter for Study Group 3, where rHuPH20 was given simultaneously with ZA SC infusion. Apparent differences in AUC and Tmax cannot be confirmed with statistical analysis due to the small number of observations and the experimental variables in the study design among animals within the same study group.

In summary, co-administration of SC ZA with rHuPH20 appeared to have an effect on the absorption profile of ZA (decreased time to maximum concentration and increased extent of absorption). Maximum serum concentration was comparable to the SC infusion group without co-administration of rHuPH20 and averaged 296.50±46.92 ng/mL. SC bioavailability of ZA+rHuPH20 was 96.00±10.98%.

TABLE 14
Pharmacokinetic Parameters^{a} in Pigs Following a SC Infusion of 0.075
or 0.15 mg/kg Zoledronic Acid (Study Group 2).

| | | | | | AUC^{b} | | | SC | |
|---|---|---|---|---|---|---|---|---|---|
| | Body | ZA | Infusion | Dose | (0-1 h) | (0-8 h) | (0-∞) | Bioavailability^{c} | |
| | Wt | Dose | Time | Vol | (ng- | (ng- | (ng- | (0-1 h) | (0-8 h) |
| Pig ID | (kg) | (mg/kg) | (min) | (mL) | h/mL) | h/mL) | h/mL) | (%) | (%) |
| 59 | 27.7 | 0.075 | 16 | 100 | 85.61 | 152.76 | 156.87 | NC^{j} | NC |
| 60 | 27.3 | 0.15 | 16 | 100 | 156.50 | 358.85 | 358.74 | 55.47 | 104.85 |
| 62 | 26.8 | 0.15 | 8 | 50 | 152.37 | 246.36 | 246.07 | 54.01 | 71.92 |
| 69 | 30.0 | 0.15 | 4 | 25 | 160.43 | 268.78 | 269.51 | 56.86 | 78.77 |
| 70 | 26.0 | 0.15 | 1.6 | 10 | 187.76 | 321.64 | 325.25 | 66.55 | 95.06 |
| Mean | | | | | 164.26 | 298.91 | 299.89 | 58.22 | 87.65 |
| Std Dev | | | | | 16.00 | 50.92 | 51.40 | 5.67 | 15.02 |
| Median | | | | | 158.46 | 295.21 | 297.38 | 56.17 | 86.91 |
| n | | | | | 4 | 4 | 4 | 4 | 4 |

| | Cmax^{d} | Tmax^{e} | T½ λz^{f} | CL/F^{g} | Vz/F^{h} | Vss/Fⁱ |
|---|---|---|---|---|---|---|
| Pig ID | (ng/mL) | (h) | (h) | (mL/h-kg) | (mL/kg) | (mL/kg) |
| 59 | 130.00 | 0.333 | 1.10 | 478.09 | 759.57 | 617.21 |
| 60 | 255.00 | 0.333 | 0.87 | 418.13 | 524.02 | 526.44 |
| 62 | 278.00 | 0.167 | 0.97 | 609.59 | 852.22 | 634.40 |
| 69 | 332.00 | 0.167 | 1.01 | 556.57 | 811.19 | 652.10 |
| 70 | 314.00 | 0.167 | 1.42 | 461.18 | 946.80 | 615.35 |
| Mean | 294.75 | 0.21 | 1.07 | 511.37 | 783.56 | 607.07 |
| Std Dev | 34.73 | 0.08 | 0.24 | 87.37 | 182.10 | 55.81 |
| Median | 296.00 | 0.17 | 0.99 | 508.87 | 831.70 | 624.87 |
| n | 4 | 4 | 4 | 4 | 4 | 4 |

^{a}PK parameters derived by non-compartmental analysis
^{b}AUC = area under the serum concentration versus time curve
^{c}SC Bioavailability calculated by [AUC(0-t)SC ÷ mean AUC(0-t)IV] × 100%
^{d}Cmax = maximum serum ZA concentration
^{e}Tmax = time to maximum serum ZA concentration
^{f}T½ λz = Lambda z half-life
^{g}CL/F = apparent clearance = Dose ÷ AUC
^{h}Vz/F = apparent volume of distribution
^{I}Vss/F = apparent volume of distribution at steady state
^{j}NC = not calculated

TABLE 15
Pharmacokinetic Parameters^{a} in Pigs Following SC Infusion of 0.075
or 0.15 mg/kg Zoledronic Acid Co-administered with rHuPH20 (Study
Group 3).

| | | | | | | | AUC^{c} | | | SC | |
|---|---|---|---|---|---|---|---|---|---|---|---|
| | Body | ZA | Infusion | Infusion | Dose | PH20 | (0-1 h) | (0-8 h) | (0-∞) | Bioavailability^{d} | |
| Pig | Wt | Dose | Time | Vol | Vol | Dose^{b} | (ng- | (ng- | (ng- | (0-1 h) | (0-8 h) |
| ID | (mg/kg) | (mg/kg) | (min) | (mL/min) | (mL) | (U) | h/mL) | h/mL) | h/mL) | (%) | (%) |
| 66 | 27.7 | 0.075 | 16 | 6.25 | 100 | 1000000 | 64.78 | 132.64 | 135.21 | NC^{k} | NC |
| 64 | 27.3 | 0.15 | 16 | 6.25 | 100 | 1000000 | 164.59 | 333.27 | 337.91 | 58.35 | 97.38 |
| 58 | 26.4 | 0.15 | 8 | 6.25 | 50 | 500000 | 191.66 | 309.64 | 309.74 | 67.94 | 90.48 |
| 56 | 26.0 | 0.15 | 4 | 6.25 | 25 | 250000 | 185.46 | 378.93 | 384.07 | 65.74 | 110.73 |
| 68 | 29.0 | 0.15 | 1.6 | 6.25 | 10 | 100000 | 178.96 | 292.25 | 292.70 | 63.44 | 85.40 |

| | | | | | |
|---|---|---|---|---|---|
| Mean | 180.17 | 328.52 | 331.11 | 63.87 | 96.00 |
| Std Dev | 11.61 | 37.57 | 39.93 | 4.11 | 10.98 |
| Median | 182.21 | 321.45 | 323.82 | 64.59 | 93.93 |
| n | 4 | 4 | 4 | 4 | 4 |

| | | | T½ | | | |
|---|---|---|---|---|---|---|
| Pig | Cmax^{e} | Tmax^{f} | λz^{g} | CL/F^{h} | Vz/Fⁱ | Vss/F^{j} |
| ID | (ng/mL) | (h) | (h) | (mL/h-kg) | (mL/kg) | (mL/kg) |
| 66 | 103.00 | 0.33 | 1.02 | 554.70 | 814.79 | 748.02 |
| 64 | 253.00 | 0.33 | 1.24 | 443.91 | 794.17 | 712.95 |
| 58 | 320.00 | 0.17 | 0.93 | 484.28 | 652.67 | 499.77 |
| 56 | 262.00 | 0.17 | 1.35 | 390.55 | 759.95 | 614.50 |
| 68 | 351.00 | 0.08 | 0.97 | 512.47 | 720.01 | 568.52 |
| | 296.50 | 0.19 | 1.12 | 457.80 | 731.70 | 598.93 |
| | 46.92 | 0.10 | 0.20 | 52.93 | 60.78 | 89.44 |
| | 291.00 | 0.17 | 1.11 | 464.09 | 739.98 | 591.51 |
| | 4 | 4 | 4 | 4 | 4 | 4 |

^{a} PK parameters derived by non-compartmental analysis
^{b}PH20 dose (U/animal) determined by “infusion volume (mL/min) × infusion time (min) × rHuPH20 dose concentration (10000 U/mL)”
^{c}AUC = area under the serum concentration versus time curve
^{d}SC Bioavailability calculated by [AUC(0-t)SC ÷ mean AUC(0-t)IV] × 100%
^{e}Cmax = maximum serum ZA concentration
^{f}Tmax = time to maximum serum ZA concentration
^{g}T½ λz = Lambda z half-life
^{h}CL/F = apparent clearance = Dose ÷ AUC
^{I}Vz/F = apparent volume of distribution
^{j}Vss/F = apparent volume of distribution at steady state
^{k}NC = not calculated

TABLE 16
Mean Plasma Concentration versus Time Data for Study Group 2
Animals (Varied Time SC Infusion of 0.15 mg/kg or 0.075 mg/kg ZA)

| ZA Dose 0.15 mg/kg | | | | | ZA Dose 0.075 mg/kg | | |
|---|---|---|---|---|---|---|---|
| | Mean ZA | | Median ZA | | | ZA | |
| Time | Concentration | | Concentration | | Time | Concentration | |
| (h) | (ng/mL) | Std Dev | (ng/mL) | n | (h) | (ng/mL) | n |
| 0.0167 | BLQ^{a} | 6.73 | 7.92 | 4 | 0.0167 | BLQ | 1 |
| 0.05 | 76.75 | 56.41 | 74.55 | 4 | 0.05 | 13.90 | 1 |
| 0.083 | 161.35 | 131.96 | 141.50 | 4 | 0.083 | 32.40 | 1 |
| 0.167 | 215.83 | 113.65 | 228.50 | 4 | 0.167 | 74.30 | 1 |
| 0.25 | 214.50 | 61.17 | 237.50 | 4 | 0.25 | 125.00 | 1 |
| 0.33 | BLQ | 51.92 | 209.00 | 4 | 0.33 | 130.00 | 1 |
| 0.50 | 148.75 | 44.12 | 134.50 | 4 | 0.50 | 120.00 | 1 |
| 0.75 | 94.73 | 14.61 | 94.80 | 4 | 0.75 | 79.30 | 1 |
| 1 | 87.45 | 44.06 | 78.10 | 4 | 1 | 44.60 | 1 |
| 1.5 | 64.43 | 38.43 | 53.05 | 4 | 1.5 | 31.60 | 1 |
| 2 | 39.73 | 24.33 | 30.10 | 4 | 2 | 22.90 | 1 |
| 3 | 18.70 | 6.75 | 17.00 | 4 | 3 | 13.30 | 1 |
| 4 | BLQ | 1.89 | 7.30 | 4 | 4 | 6.48 | 1 |
| 6 | 2.38 | 1.61 | 2.96 | 4 | 6 | BLQ | 1 |
| 8 | BLQ | BLQ | BLQ | 4 | 8 | BLQ | 1 |
| 10 | BLQ | BLQ | BLQ | 4 | 10 | BLQ | 1 |
| 24 | BLQ | BLQ | BLQ | 4 | 24 | BLQ | 1 |
| 48 | BLQ | BLQ | BLQ | 4 | 48 | BLQ | 1 |
| 72 | BLQ | BLQ | BLQ | 4 | 72 | BLQ | 1 |
| 312 | BLQ | BLQ | BLQ | 4 | 312 | BLQ | 1 |

^{a}BLQ = below assay limit of quantification (2.0 ng/mL)

TABLE 17
Mean Plasma Concentration versus Time Data for Study Group 3
Animals (Varied Time SC Infusion of 0.15 mg/kg or 0.075 mg/kg ZA Co-
administered with Varied Doses of rHuPH20)

| ZA Dose 0.15 mg/kg | | | | | ZA Dose 0.075 mg/kg | | |
|---|---|---|---|---|---|---|---|
| | Mean ZA | | Median ZA | | | ZA | |
| Time | Concentration | Std | Concentration | | Time | Concentration | |
| (h) | (ng/mL) | Dev | (ng/mL) | n | (h) | (ng/mL) | n |
| 0.0167 | 42.63 | 44.13 | 28.45 | 4 | 0.0167 | 2.21 | 1 |
| 0.05 | 143.45 | 133.60 | 87.40 | 4 | 0.05 | 12 | 1 |
| 0.083 | 225.50 | 103.61 | 223.00 | 4 | 0.083 | 32.5 | 1 |
| 0.167 | 254.00 | 64.72 | 265.50 | 4 | 0.167 | 66.1 | 1 |
| 0.25 | 252.75 | 38.85 | 247.50 | 4 | 0.25 | 95 | 1 |
| 0.333 | 239.75 | 29.52 | 244.00 | 4 | 0.333 | 103 | 1 |
| 0.50 | 185.75 | 11.35 | 185.50 | 4 | 0.50 | 78.2 | 1 |
| 0.75 | 147.75 | 23.26 | 155.00 | 4 | 0.75 | 52.7 | 1 |
| 1 | 106.65 | 15.76 | 104.00 | 4 | 1 | 53.3 | 1 |
| 1.5 | 64.28 | 9.53 | 67.85 | 4 | 1.5 | 32.3 | 1 |
| 2 | 48.48 | 8.75 | 47.05 | 4 | 2 | 23.2 | 1 |
| 3 | 22.20 | 9.93 | 23.40 | 4 | 3 | 11.4 | 1 |
| 4 | 12.99 | 6.18 | 12.94 | 4 | 4 | 6.39 | 1 |
| 5 | 19.60 | NA^{a} | 19.60 | 1 | 5 | NA | 0 |
| 6 | 4.57 | 2.28 | 4.22 | 4 | 6 | BLQ | 1 |
| 8 | 0.57 | 1.14 | BLQ^{b} | 4 | 8 | BLQ | 1 |
| 10 | BLQ | BLQ | BLQ | 4 | 10 | BLQ | 1 |
| 24 | BLQ | BLQ | BLQ | 4 | 24 | BLQ | 1 |
| 48 | BLQ | BLQ | BLQ | 4 | 48 | BLQ | 1 |
| 72 | BLQ | BLQ | BLQ | 4 | 72 | BLQ | 1 |
| 312 | BLQ | BLQ | BLQ | 4 | 312 | BLQ | 1 |

^{a}NA = not applicable
^{b}BLQ = below assay limit of quantification (2.0 ng/mL)

TABLE 18
Individual Zoledronic Acid Serum Concentration versus Time Data
for Study Group 1

| | | | | | | | | Dose | Inf | Inf | | |
|---|---|---|---|---|---|---|---|---|---|---|---|---|
| Time | Concentration | | Study | Day | | BW | | Vol | time | Rate | rHuPH20 | ZA |
| (h) | (ng/mL) | ID | Group | Nominal | Period | (kg) | Route | (mL) | (min) | (mL/min) | (U/mL) | (mg/kg) |
| 0.016 | 255 | 57 | 1 | 1 | 1 | 29.1 | IV | 125 | 20 | 6.25 | 0 | 0.15 |
| 0.05 | 446 | 57 | 1 | 1 | 1 | 29.1 | IV | 125 | 20 | 6.25 | 0 | 0.15 |
| 0.083 | 443 | 57 | 1 | 1 | 1 | 29.1 | IV | 125 | 20 | 6.25 | 0 | 0.15 |
| 0.167 | 444 | 57 | 1 | 1 | 1 | 29.1 | IV | 125 | 20 | 6.25 | 0 | 0.15 |
| 0.25 | 465 | 57 | 1 | 1 | 1 | 29.1 | IV | 125 | 20 | 6.25 | 0 | 0.15 |
| 0.333 | 388 | 57 | 1 | 1 | 1 | 29.1 | IV | 125 | 20 | 6.25 | 0 | 0.15 |
| 0.50 | 244 | 57 | 1 | 1 | 1 | 29.1 | IV | 125 | 20 | 6.25 | 0 | 0.15 |
| 0.75 | 127 | 57 | 1 | 1 | 1 | 29.1 | IV | 125 | 20 | 6.25 | 0 | 0.15 |
| 1.00 | 71.4 | 57 | 1 | 1 | 1 | 29.1 | IV | 125 | 20 | 6.25 | 0 | 0.15 |
| 1.50 | 29.6 | 57 | 1 | 1 | 1 | 29.1 | IV | 125 | 20 | 6.25 | 0 | 0.15 |
| 2 | 15.4 | 57 | 1 | 1 | 1 | 29.1 | IV | 125 | 20 | 6.25 | 0 | 0.15 |
| 3 | 5.8 | 57 | 1 | 1 | 1 | 29.1 | IV | 125 | 20 | 6.25 | 0 | 0.15 |
| 4 | 2.8 | 57 | 1 | 1 | 1 | 29.1 | IV | 125 | 20 | 6.25 | 0 | 0.15 |
| 6 | BLQ^{a} | 57 | 1 | 1 | 1 | 29.1 | IV | 125 | 20 | 6.25 | 0 | 0.15 |
| 8 | BLQ | 57 | 1 | 1 | 1 | 29.1 | IV | 125 | 20 | 6.25 | 0 | 0.15 |
| 10 | BLQ | 57 | 1 | 1 | 1 | 29.1 | IV | 125 | 20 | 6.25 | 0 | 0.15 |
| 24 | BLQ | 57 | 1 | 2 | 1 | 29.1 | IV | 125 | 20 | 6.25 | 0 | 0.15 |
| 48 | BLQ | 57 | 1 | 3 | 1 | 29.1 | IV | 125 | 20 | 6.25 | 0 | 0.15 |
| 72 | BLQ | 57 | 1 | 4 | 1 | 29.1 | IV | 125 | 20 | 6.25 | 0 | 0.15 |
| 312 | BLQ | 57 | 1 | 14 | 1 | 29.1 | IV | 125 | 20 | 6.25 | 0 | 0.15 |
| 0.016 | 254 | 61 | 1 | 1 | 1 | 26 | IV | 125 | 20 | 6.25 | 0 | 0.15 |
| 0.05 | 196 | 61 | 1 | 1 | 1 | 26 | IV | 125 | 20 | 6.25 | 0 | 0.15 |
| 0.083 | 384 | 61 | 1 | 1 | 1 | 26 | IV | 125 | 20 | 6.25 | 0 | 0.15 |
| 0.167 | 615 | 61 | 1 | 1 | 1 | 26 | IV | 125 | 20 | 6.25 | 0 | 0.15 |
| 0.25 | 682 | 61 | 1 | 1 | 1 | 26 | IV | 125 | 20 | 6.25 | 0 | 0.15 |
| 0.333 | 596 | 61 | 1 | 1 | 1 | 26 | IV | 125 | 20 | 6.25 | 0 | 0.15 |
| 0.5 | 299 | 61 | 1 | 1 | 1 | 26 | IV | 125 | 20 | 6.25 | 0 | 0.15 |
| 0.75 | 49.7 | 61 | 1 | 1 | 1 | 26 | IV | 125 | 20 | 6.25 | 0 | 0.15 |
| 1 | 85.5 | 61 | 1 | 1 | 1 | 26 | IV | 125 | 20 | 6.25 | 0 | 0.15 |
| 1.5 | 41.5 | 61 | 1 | 1 | 1 | 26 | IV | 125 | 20 | 6.25 | 0 | 0.15 |
| 2 | 18.8 | 61 | 1 | 1 | 1 | 26 | IV | 125 | 20 | 6.25 | 0 | 0.15 |
| 3 | 6.1 | 61 | 1 | 1 | 1 | 26 | IV | 125 | 20 | 6.25 | 0 | 0.15 |
| 4 | 3.3 | 61 | 1 | 1 | 1 | 26 | IV | 125 | 20 | 6.25 | 0 | 0.15 |
| 6 | BLQ | 61 | 1 | 1 | 1 | 26 | IV | 125 | 20 | 6.25 | 0 | 0.15 |
| 8 | BLQ | 61 | 1 | 1 | 1 | 26 | IV | 125 | 20 | 6.25 | 0 | 0.15 |
| 10 | BLQ | 61 | 1 | 1 | 1 | 26 | IV | 125 | 20 | 6.25 | 0 | 0.15 |
| 24 | BLQ | 61 | 1 | 2 | 1 | 26 | IV | 125 | 20 | 6.25 | 0 | 0.15 |
| 48 | BLQ | 61 | 1 | 3 | 1 | 26 | IV | 125 | 20 | 6.25 | 0 | 0.15 |
| 72 | BLQ | 61 | 1 | 4 | 1 | 26 | IV | 125 | 20 | 6.25 | 0 | 0.15 |
| 312 | BLQ | 61 | 1 | 14 | 1 | 26 | IV | 125 | 20 | 6.25 | 0 | 0.15 |
| 0.0167 | 158 | 67 | 1 | 1 | 1 | 26.4 | IV | 125 | 20 | 6.25 | 0 | 0.15 |
| 0.05 | 288 | 67 | 1 | 1 | 1 | 26.4 | IV | 125 | 20 | 6.25 | 0 | 0.15 |
| 0.083 | 319 | 67 | 1 | 1 | 1 | 26.4 | IV | 125 | 20 | 6.25 | 0 | 0.15 |
| 0.167 | 448 | 67 | 1 | 1 | 1 | 26.4 | IV | 125 | 20 | 6.25 | 0 | 0.15 |
| 0.25 | 580 | 67 | 1 | 1 | 1 | 26.4 | IV | 125 | 20 | 6.25 | 0 | 0.15 |
| 0.333 | 556 | 67 | 1 | 1 | 1 | 26.4 | IV | 125 | 20 | 6.25 | 0 | 0.15 |
| 0.5 | 246 | 67 | 1 | 1 | 1 | 26.4 | IV | 125 | 20 | 6.25 | 0 | 0.15 |
| 0.75 | 128 | 67 | 1 | 1 | 1 | 26.4 | IV | 125 | 20 | 6.25 | 0 | 0.15 |
| 1 | 77.8 | 67 | 1 | 1 | 1 | 26.4 | IV | 125 | 20 | 6.25 | 0 | 0.15 |
| 1.5 | 31.7 | 67 | 1 | 1 | 1 | 26.4 | IV | 125 | 20 | 6.25 | 0 | 0.15 |
| 2 | 16.6 | 67 | 1 | 1 | 1 | 26.4 | IV | 125 | 20 | 6.25 | 0 | 0.15 |
| 3 | 6.67 | 67 | 1 | 1 | 1 | 26.4 | IV | 125 | 20 | 6.25 | 0 | 0.15 |
| 4 | 3.35 | 67 | 1 | 1 | 1 | 26.4 | IV | 125 | 20 | 6.25 | 0 | 0.15 |
| 6 | BLQ | 67 | 1 | 1 | 1 | 26.4 | IV | 125 | 20 | 6.25 | 0 | 0.15 |
| 8 | BLQ | 67 | 1 | 1 | 1 | 26.4 | IV | 125 | 20 | 6.25 | 0 | 0.15 |
| 10 | BLQ | 67 | 1 | 1 | 1 | 26.4 | IV | 125 | 20 | 6.25 | 0 | 0.15 |
| 24 | BLQ | 67 | 1 | 2 | 1 | 26.4 | IV | 125 | 20 | 6.25 | 0 | 0.15 |
| 48 | BLQ | 67 | 1 | 3 | 1 | 26.4 | IV | 125 | 20 | 6.25 | 0 | 0.15 |
| 72 | BLQ | 67 | 1 | 4 | 1 | 26.4 | IV | 125 | 20 | 6.25 | 0 | 0.15 |
| 312 | BLQ | 67 | 1 | 14 | 1 | 26.4 | IV | 125 | 20 | 6.25 | 0 | 0.15 |

^{a}BLQ = below assay limit of quantification (2.0 ng/mL)

TABLE 19
Individual Zoledronic Acid Serum Concentration versus Time Data
for Study Group 2

| | | | | | | | | Dose | Inf | | | |
|---|---|---|---|---|---|---|---|---|---|---|---|---|
| Time | Concentration | | Study | Day | | BW | | Vol | Time | Inf Rate | rHuPH20 | ZA |
| (h) | (ng/mL) | ID | Group | Nominal | Period | (kg) | Route | (mL) | (min) | (mL/min) | (U/mL) | (mg/kg) |
| 0.0167 | BLQ^{a} | 59 | 2 | 1 | 1 | 27.7 | SC | 100 | 16 | 6.25 | 0 | 0.075 |
| 0.05 | 13.9 | 59 | 2 | 1 | 1 | 27.7 | SC | 100 | 16 | 6.25 | 0 | 0.075 |
| 0.0833 | 32.4 | 59 | 2 | 1 | 1 | 27.7 | SC | 100 | 16 | 6.25 | 0 | 0.075 |
| 0.167 | 74.3 | 59 | 2 | 1 | 1 | 27.7 | SC | 100 | 16 | 6.25 | 0 | 0.075 |
| 0.25 | 125 | 59 | 2 | 1 | 1 | 27.7 | SC | 100 | 16 | 6.25 | 0 | 0.075 |
| 0.333 | 130 | 59 | 2 | 1 | 1 | 27.7 | SC | 100 | 16 | 6.25 | 0 | 0.075 |
| 0.5 | 120 | 59 | 2 | 1 | 1 | 27.7 | SC | 100 | 16 | 6.25 | 0 | 0.075 |
| 0.75 | 79.3 | 59 | 2 | 1 | 1 | 27.7 | SC | 100 | 16 | 6.25 | 0 | 0.075 |
| 1 | 44.6 | 59 | 2 | 1 | 1 | 27.7 | SC | 100 | 16 | 6.25 | 0 | 0.075 |
| 1.5 | 31.6 | 59 | 2 | 1 | 1 | 27.7 | SC | 100 | 16 | 6.25 | 0 | 0.075 |
| 2 | 22.9 | 59 | 2 | 1 | 1 | 27.7 | SC | 100 | 16 | 6.25 | 0 | 0.075 |
| 3 | 13.3 | 59 | 2 | 1 | 1 | 27.7 | SC | 100 | 16 | 6.25 | 0 | 0.075 |
| 4 | 6.48 | 59 | 2 | 1 | 1 | 27.7 | SC | 100 | 16 | 6.25 | 0 | 0.075 |
| 6 | BLQ | 59 | 2 | 1 | 1 | 27.7 | SC | 100 | 16 | 6.25 | 0 | 0.075 |
| 8 | BLQ | 59 | 2 | 1 | 1 | 27.7 | SC | 100 | 16 | 6.25 | 0 | 0.075 |
| 10 | BLQ | 59 | 2 | 1 | 1 | 27.7 | SC | 100 | 16 | 6.25 | 0 | 0.075 |
| 24 | BLQ | 59 | 2 | 2 | 1 | 27.7 | SC | 100 | 16 | 6.25 | 0 | 0.075 |
| 48 | BLQ | 59 | 2 | 3 | 1 | 27.7 | SC | 100 | 16 | 6.25 | 0 | 0.075 |
| 72 | BLQ | 59 | 2 | 4 | 1 | 27.7 | SC | 100 | 16 | 6.25 | 0 | 0.075 |
| 312 | BLQ | 59 | 2 | 14 | 1 | 27.7 | SC | 100 | 16 | 6.25 | 0 | 0.075 |
| 0.0167 | 8.69 | 60 | 2 | 1 | 1 | 27.3 | SC | 100 | 16 | 6.25 | 0 | 0.15 |
| 0.05 | 51.5 | 60 | 2 | 1 | 1 | 27.3 | SC | 100 | 16 | 6.25 | 0 | 0.15 |
| 0.0833 | 85 | 60 | 2 | 1 | 1 | 27.3 | SC | 100 | 16 | 6.25 | 0 | 0.15 |
| 0.167 | 179 | 60 | 2 | 1 | 1 | 27.3 | SC | 100 | 16 | 6.25 | 0 | 0.15 |
| 0.25 | 226 | 60 | 2 | 1 | 1 | 27.3 | SC | 100 | 16 | 6.25 | 0 | 0.15 |
| 0.333 | 255 | 60 | 2 | 1 | 1 | 27.3 | SC | 100 | 16 | 6.25 | 0 | 0.15 |
| 0.5 | 211 | 60 | 2 | 1 | 1 | 27.3 | SC | 100 | 16 | 6.25 | 0 | 0.15 |
| 0.75 | 85.6 | 60 | 2 | 1 | 1 | 27.3 | SC | 100 | 16 | 6.25 | 0 | 0.15 |
| 1 | 149 | 60 | 2 | 1 | 1 | 27.3 | SC | 100 | 16 | 6.25 | 0 | 0.15 |
| 1.5 | 120 | 60 | 2 | 1 | 1 | 27.3 | SC | 100 | 16 | 6.25 | 0 | 0.15 |
| 2 | 75.8 | 60 | 2 | 1 | 1 | 27.3 | SC | 100 | 16 | 6.25 | 0 | 0.15 |
| 3 | 27.5 | 60 | 2 | 1 | 1 | 27.3 | SC | 100 | 16 | 6.25 | 0 | 0.15 |
| 4 | 9.03 | 60 | 2 | 1 | 1 | 27.3 | SC | 100 | 16 | 6.25 | 0 | 0.15 |
| 6 | 3.6 | 60 | 2 | 1 | 1 | 27.3 | SC | 100 | 16 | 6.25 | 0 | 0.15 |
| 8 | BLQ | 60 | 2 | 1 | 1 | 27.3 | SC | 100 | 16 | 6.25 | 0 | 0.15 |
| 10 | BLQ | 60 | 2 | 1 | 1 | 27.3 | SC | 100 | 16 | 6.25 | 0 | 0.15 |
| 24 | BLQ | 60 | 2 | 2 | 1 | 27.3 | SC | 100 | 16 | 6.25 | 0 | 0.15 |
| 48 | BLQ | 60 | 2 | 3 | 1 | 27.3 | SC | 100 | 16 | 6.25 | 0 | 0.15 |
| 72 | BLQ | 60 | 2 | 4 | 1 | 27.3 | SC | 100 | 16 | 6.25 | 0 | 0.15 |
| 312 | BLQ | 60 | 2 | 14 | 1 | 27.3 | SC | 100 | 16 | 6.25 | 0 | 0.15 |
| 0.0167 | 16.4 | 62 | 2 | 1 | 1 | 26.8 | SC | 50 | 8 | 6.25 | 0 | 0.15 |
| 0.05 | 97.6 | 62 | 2 | 1 | 1 | 26.8 | SC | 50 | 8 | 6.25 | 0 | 0.15 |
| 0.083 | 198 | 62 | 2 | 1 | 1 | 26.8 | SC | 50 | 8 | 6.25 | 0 | 0.15 |
| 0.167 | 278 | 62 | 2 | 1 | 1 | 26.8 | SC | 50 | 8 | 6.25 | 0 | 0.15 |
| 0.25 | 249 | 62 | 2 | 1 | 1 | 26.8 | SC | 50 | 8 | 6.25 | 0 | 0.15 |
| 0.333 | 208 | 62 | 2 | 1 | 1 | 26.8 | SC | 50 | 8 | 6.25 | 0 | 0.15 |
| 0.5 | 149 | 62 | 2 | 1 | 1 | 26.8 | SC | 50 | 8 | 6.25 | 0 | 0.15 |
| 0.75 | 104 | 62 | 2 | 1 | 1 | 26.8 | SC | 50 | 8 | 6.25 | 0 | 0.15 |
| 1 | 81.6 | 62 | 2 | 1 | 1 | 26.8 | SC | 50 | 8 | 6.25 | 0 | 0.15 |
| 1.5 | 51.4 | 62 | 2 | 1 | 1 | 26.8 | SC | 50 | 8 | 6.25 | 0 | 0.15 |
| 2 | 28.4 | 62 | 2 | 1 | 1 | 26.8 | SC | 50 | 8 | 6.25 | 0 | 0.15 |
| 3 | 13.5 | 62 | 2 | 1 | 1 | 26.8 | SC | 50 | 8 | 6.25 | 0 | 0.15 |
| 4 | 4.74 | 62 | 2 | 1 | 1 | 26.8 | SC | 50 | 8 | 6.25 | 0 | 0.15 |
| 6 | 2.99 | 62 | 2 | 1 | 1 | 26.8 | SC | 50 | 8 | 6.25 | 0 | 0.15 |
| 8 | BLQ | 62 | 2 | 1 | 1 | 26.8 | SC | 50 | 8 | 6.25 | 0 | 0.15 |
| 10 | BLQ | 62 | 2 | 1 | 1 | 26.8 | SC | 50 | 8 | 6.25 | 0 | 0.15 |
| 24 | BLQ | 62 | 2 | 2 | 1 | 26.8 | SC | 50 | 8 | 6.25 | 0 | 0.15 |
| 48 | BLQ | 62 | 2 | 3 | 1 | 26.8 | SC | 50 | 8 | 6.25 | 0 | 0.15 |
| 72 | BLQ | 62 | 2 | 4 | 1 | 26.8 | SC | 50 | 8 | 6.25 | 0 | 0.15 |
| 312 | BLQ | 62 | 2 | 14 | 1 | 26.8 | SC | 50 | 8 | 6.25 | 0 | 0.15 |
| 0.0167 | 7.15 | 69 | 2 | 1 | 1 | 30 | SC | 25 | 4 | 6.25 | 0 | 0.15 |
| 0.05 | 144 | 69 | 2 | 1 | 1 | 30 | SC | 25 | 4 | 6.25 | 0 | 0.15 |
| 0.083 | 330 | 69 | 2 | 1 | 1 | 30 | SC | 25 | 4 | 6.25 | 0 | 0.15 |
| 0.167 | 332 | 69 | 2 | 1 | 1 | 30 | SC | 25 | 4 | 6.25 | 0 | 0.15 |
| 0.25 | 258 | 69 | 2 | 1 | 1 | 30 | SC | 25 | 4 | 6.25 | 0 | 0.15 |
| 0.333 | 210 | 69 | 2 | 1 | 1 | 30 | SC | 25 | 4 | 6.25 | 0 | 0.15 |
| 0.5 | 115 | 69 | 2 | 1 | 1 | 30 | SC | 25 | 4 | 6.25 | 0 | 0.15 |
| 0.75 | 110 | 69 | 2 | 1 | 1 | 30 | SC | 25 | 4 | 6.25 | 0 | 0.15 |
| 1 | 74.6 | 69 | 2 | 1 | 1 | 30 | SC | 25 | 4 | 6.25 | 0 | 0.15 |
| 1.5 | 54.7 | 69 | 2 | 1 | 1 | 30 | SC | 25 | 4 | 6.25 | 0 | 0.15 |
| 2 | 31.8 | 69 | 2 | 1 | 1 | 30 | SC | 25 | 4 | 6.25 | 0 | 0.15 |
| 3 | 20.5 | 69 | 2 | 1 | 1 | 30 | SC | 25 | 4 | 6.25 | 0 | 0.15 |
| 4 | 8.11 | 69 | 2 | 1 | 1 | 30 | SC | 25 | 4 | 6.25 | 0 | 0.15 |
| 6 | 2.92 | 69 | 2 | 1 | 1 | 30 | SC | 25 | 4 | 6.25 | 0 | 0.15 |
| 8 | BLQ | 69 | 2 | 1 | 1 | 30 | SC | 25 | 4 | 6.25 | 0 | 0.15 |
| 10 | BLQ | 69 | 2 | 1 | 1 | 30 | SC | 25 | 4 | 6.25 | 0 | 0.15 |
| 24 | BLQ | 69 | 2 | 2 | 1 | 30 | SC | 25 | 4 | 6.25 | 0 | 0.15 |
| 48 | BLQ | 69 | 2 | 3 | 1 | 30 | SC | 25 | 4 | 6.25 | 0 | 0.15 |
| 72 | BLQ | 69 | 2 | 4 | 1 | 30 | SC | 25 | 4 | 6.25 | 0 | 0.15 |
| 312 | BLQ | 69 | 2 | 14 | 1 | 30 | SC | 25 | 4 | 6.25 | 0 | 0.15 |
| 0.0167 | 3.58 | 70 | 2 | 1 | 1 | 26 | SC | 10 | 1.6 | 6.25 | 0 | 0.15 |
| 0.05 | 174 | 70 | 2 | 1 | 1 | 26 | SC | 10 | 1.6 | 6.25 | 0 | 0.15 |
| 0.083 | 257 | 70 | 2 | 1 | 1 | 26 | SC | 10 | 1.6 | 6.25 | 0 | 0.15 |
| 0.167 | 314 | 70 | 2 | 1 | 1 | 26 | SC | 10 | 1.6 | 6.25 | 0 | 0.15 |
| 0.25 | 287 | 70 | 2 | 1 | 1 | 26 | SC | 10 | 1.6 | 6.25 | 0 | 0.15 |
| 0.333 | 233 | 70 | 2 | 1 | 1 | 26 | SC | 10 | 1.6 | 6.25 | 0 | 0.15 |
| 0.5 | 191 | 70 | 2 | 1 | 1 | 26 | SC | 10 | 1.6 | 6.25 | 0 | 0.15 |
| 0.75 | 141 | 70 | 2 | 1 | 1 | 26 | SC | 10 | 1.6 | 6.25 | 0 | 0.15 |
| 1 | 101 | 70 | 2 | 1 | 1 | 26 | SC | 10 | 1.6 | 6.25 | 0 | 0.15 |
| 1.5 | 67.4 | 70 | 2 | 1 | 1 | 26 | SC | 10 | 1.6 | 6.25 | 0 | 0.15 |
| 2 | 37.7 | 70 | 2 | 1 | 1 | 26 | SC | 10 | 1.6 | 6.25 | 0 | 0.15 |
| 3 | 22.2 | 70 | 2 | 1 | 1 | 26 | SC | 10 | 1.6 | 6.25 | 0 | 0.15 |
| 4 | 9.47 | 70 | 2 | 1 | 1 | 26 | SC | 10 | 1.6 | 6.25 | 0 | 0.15 |
| 6 | 4.09 | 70 | 2 | 1 | 1 | 26 | SC | 10 | 1.6 | 6.25 | 0 | 0.15 |
| 8 | 2.07 | 70 | 2 | 1 | 1 | 26 | SC | 10 | 1.6 | 6.25 | 0 | 0.15 |
| 10 | BLQ | 70 | 2 | 1 | 1 | 26 | SC | 10 | 1.6 | 6.25 | 0 | 0.15 |
| 24 | BLQ | 70 | 2 | 2 | 1 | 26 | SC | 10 | 1.6 | 6.25 | 0 | 0.15 |
| 48 | BLQ | 70 | 2 | 3 | 1 | 26 | SC | 10 | 1.6 | 6.25 | 0 | 0.15 |
| 72 | BLQ | 70 | 2 | 4 | 1 | 26 | SC | 10 | 1.6 | 6.25 | 0 | 0.15 |
| 312 | BLQ | 70 | 2 | 14 | 1 | 26 | SC | 10 | 1.6 | 6.25 | 0 | 0.15 |

^{a}BLQ = below assay limit of quantification (2.0 ng/mL)

TABLE 20
Individual Zoledronic Acid Serum Concentration versus Time Data
for Study Group 3

| | | | | | | | | Dose | Inf | | | |
|---|---|---|---|---|---|---|---|---|---|---|---|---|
| Time | Concentration | | Study | Day | | BW | | Vol | time | Inf Rate | rHuPH20 | ZA |
| (h) | (ng/mL) | ID | Group | Nominal | Period | (kg) | Route | (mL) | (min) | (mL/min) | (U/mL) | (mg/kg) |
| 0.0167 | 29.2 | 56 | 3 | 1 | 1 | 26 | SC | 25 | 4 | 6.25 | 10000 | 0.15 |
| 0.05 | 101 | 56 | 3 | 1 | 1 | 26 | SC | 25 | 4 | 6.25 | 10000 | 0.15 |
| 0.083 | 254 | 56 | 3 | 1 | 1 | 26 | SC | 25 | 4 | 6.25 | 10000 | 0.15 |
| 0.167 | 262 | 56 | 3 | 1 | 1 | 26 | SC | 25 | 4 | 6.25 | 10000 | 0.15 |
| 0.25 | 248 | 56 | 3 | 1 | 1 | 26 | SC | 25 | 4 | 6.25 | 10000 | 0.15 |
| 0.333 | 235 | 56 | 3 | 1 | 1 | 26 | SC | 25 | 4 | 6.25 | 10000 | 0.15 |
| 0.5 | 194 | 56 | 3 | 1 | 1 | 26 | SC | 25 | 4 | 6.25 | 10000 | 0.15 |
| 0.75 | 153 | 56 | 3 | 1 | 1 | 26 | SC | 25 | 4 | 6.25 | 10000 | 0.15 |
| 1 | 128 | 56 | 3 | 1 | 1 | 26 | SC | 25 | 4 | 6.25 | 10000 | 0.15 |
| 1.5 | 70.4 | 56 | 3 | 1 | 1 | 26 | SC | 25 | 4 | 6.25 | 10000 | 0.15 |
| 2 | 59.7 | 56 | 3 | 1 | 1 | 26 | SC | 25 | 4 | 6.25 | 10000 | 0.15 |
| 3 | 31.2 | 56 | 3 | 1 | 1 | 26 | SC | 25 | 4 | 6.25 | 10000 | 0.15 |
| 4 | 17.6 | 56 | 3 | 1 | 1 | 26 | SC | 25 | 4 | 6.25 | 10000 | 0.15 |
| 5 | 19.6 | 56 | 3 | 1 | 1 | 26 | SC | 25 | 4 | 6.25 | 10000 | 0.15 |
| 6 | 7.21 | 56 | 3 | 1 | 1 | 26 | SC | 25 | 4 | 6.25 | 10000 | 0.15 |
| 8 | 2.28 | 56 | 3 | 1 | 1 | 26 | SC | 25 | 4 | 6.25 | 10000 | 0.15 |
| 10 | BLQ^{a} | 56 | 3 | 1 | 1 | 26 | SC | 25 | 4 | 6.25 | 10000 | 0.15 |
| 24 | BLQ | 56 | 3 | 2 | 1 | 26 | SC | 25 | 4 | 6.25 | 10000 | 0.15 |
| 48 | BLQ | 56 | 3 | 3 | 1 | 26 | SC | 25 | 4 | 6.25 | 10000 | 0.15 |
| 72 | BLQ | 56 | 3 | 4 | 1 | 26 | SC | 25 | 4 | 6.25 | 10000 | 0.15 |
| 312 | BLQ | 56 | 3 | 14 | 1 | 26 | SC | 25 | 4 | 6.25 | 10000 | 0.15 |
| 0.0167 | 6.63 | 58 | 3 | 1 | 1 | 26.4 | SC | 50 | 8 | 6.25 | 10000 | 0.15 |
| 0.05 | 73.8 | 58 | 3 | 1 | 1 | 26.4 | SC | 50 | 8 | 6.25 | 10000 | 0.15 |
| 0.083 | 192 | 58 | 3 | 1 | 1 | 26.4 | SC | 50 | 8 | 6.25 | 10000 | 0.15 |
| 0.167 | 320 | 58 | 3 | 1 | 1 | 26.4 | SC | 50 | 8 | 6.25 | 10000 | 0.15 |
| 0.25 | 305 | 58 | 3 | 1 | 1 | 26.4 | SC | 50 | 8 | 6.25 | 10000 | 0.15 |
| 0.333 | 270 | 58 | 3 | 1 | 1 | 26.4 | SC | 50 | 8 | 6.25 | 10000 | 0.15 |
| 0.5 | 177 | 58 | 3 | 1 | 1 | 26.4 | SC | 50 | 8 | 6.25 | 10000 | 0.15 |
| 0.75 | 167 | 58 | 3 | 1 | 1 | 26.4 | SC | 50 | 8 | 6.25 | 10000 | 0.15 |
| 1 | 107 | 58 | 3 | 1 | 1 | 26.4 | SC | 50 | 8 | 6.25 | 10000 | 0.15 |
| 1.5 | 65.3 | 58 | 3 | 1 | 1 | 26.4 | SC | 50 | 8 | 6.25 | 10000 | 0.15 |
| 2 | 40.1 | 58 | 3 | 1 | 1 | 26.4 | SC | 50 | 8 | 6.25 | 10000 | 0.15 |
| 3 | 10.8 | 58 | 3 | 1 | 1 | 26.4 | SC | 50 | 8 | 6.25 | 10000 | 0.15 |
| 4 | 8.27 | 58 | 3 | 1 | 1 | 26.4 | SC | 50 | 8 | 6.25 | 10000 | 0.15 |
| 6 | 2.65 | 58 | 3 | 1 | 1 | 26.4 | SC | 50 | 8 | 6.25 | 10000 | 0.15 |
| 8 | BLQ | 58 | 3 | 1 | 1 | 26.4 | SC | 50 | 8 | 6.25 | 10000 | 0.15 |
| 10 | BLQ | 58 | 3 | 1 | 1 | 26.4 | SC | 50 | 8 | 6.25 | 10000 | 0.15 |
| 24 | BLQ | 58 | 3 | 2 | 1 | 26.4 | SC | 50 | 8 | 6.25 | 10000 | 0.15 |
| 48 | BLQ | 58 | 3 | 3 | 1 | 26.4 | SC | 50 | 8 | 6.25 | 10000 | 0.15 |
| 72 | BLQ | 58 | 3 | 4 | 1 | 26.4 | SC | 50 | 8 | 6.25 | 10000 | 0.15 |
| 312 | BLQ | 58 | 3 | 14 | 1 | 26.4 | SC | 50 | 8 | 6.25 | 10000 | 0.15 |
| 0.0167 | 27.7 | 64 | 3 | 1 | 1 | 27.3 | SC | 100 | 16 | 6.25 | 10000 | 0.15 |
| 0.05 | 57 | 64 | 3 | 1 | 1 | 27.3 | SC | 100 | 16 | 6.25 | 10000 | 0.15 |
| 0.083 | 105 | 64 | 3 | 1 | 1 | 27.3 | SC | 100 | 16 | 6.25 | 10000 | 0.15 |
| 0.167 | 165 | 64 | 3 | 1 | 1 | 27.3 | SC | 100 | 16 | 6.25 | 10000 | 0.15 |
| 0.25 | 211 | 64 | 3 | 1 | 1 | 27.3 | SC | 100 | 16 | 6.25 | 10000 | 0.15 |
| 0.333 | 253 | 64 | 3 | 1 | 1 | 27.3 | SC | 100 | 16 | 6.25 | 10000 | 0.15 |
| 0.5 | 197 | 64 | 3 | 1 | 1 | 27.3 | SC | 100 | 16 | 6.25 | 10000 | 0.15 |
| 0.75 | 157 | 64 | 3 | 1 | 1 | 27.3 | SC | 100 | 16 | 6.25 | 10000 | 0.15 |
| 1 | 101 | 64 | 3 | 1 | 1 | 27.3 | SC | 100 | 16 | 6.25 | 10000 | 0.15 |
| 1.5 | 70.9 | 64 | 3 | 1 | 1 | 27.3 | SC | 100 | 16 | 6.25 | 10000 | 0.15 |
| 2 | 50.9 | 64 | 3 | 1 | 1 | 27.3 | SC | 100 | 16 | 6.25 | 10000 | 0.15 |
| 3 | 29.8 | 64 | 3 | 1 | 1 | 27.3 | SC | 100 | 16 | 6.25 | 10000 | 0.15 |
| 4 | 19 | 64 | 3 | 1 | 1 | 27.3 | SC | 100 | 16 | 6.25 | 10000 | 0.15 |
| 6 | 5.75 | 64 | 3 | 1 | 1 | 27.3 | SC | 100 | 16 | 6.25 | 10000 | 0.15 |
| 8 | BLQ | 64 | 3 | 1 | 1 | 27.3 | SC | 100 | 16 | 6.25 | 10000 | 0.15 |
| 10 | BLQ | 64 | 3 | 1 | 1 | 27.3 | SC | 100 | 16 | 6.25 | 10000 | 0.15 |
| 24 | BLQ | 64 | 3 | 2 | 1 | 27.3 | SC | 100 | 16 | 6.25 | 10000 | 0.15 |
| 48 | BLQ | 64 | 3 | 3 | 1 | 27.3 | SC | 100 | 16 | 6.25 | 10000 | 0.15 |
| 72 | BLQ | 64 | 3 | 4 | 1 | 27.3 | SC | 100 | 16 | 6.25 | 10000 | 0.15 |
| 312 | BLQ | 64 | 3 | 14 | 1 | 27.3 | SC | 100 | 16 | 6.25 | 10000 | 0.15 |
| 0.0167 | 2.21 | 66 | 3 | 1 | 1 | 27.7 | SC | 100 | 16 | 6.25 | 10000 | 0.075 |
| 0.05 | 12 | 66 | 3 | 1 | 1 | 27.7 | SC | 100 | 16 | 6.25 | 10000 | 0.075 |
| 0.083 | 32.5 | 66 | 3 | 1 | 1 | 27.7 | SC | 100 | 16 | 6.25 | 10000 | 0.075 |
| 0.167 | 66.1 | 66 | 3 | 1 | 1 | 27.7 | SC | 100 | 16 | 6.25 | 10000 | 0.075 |
| 0.25 | 95 | 66 | 3 | 1 | 1 | 27.7 | SC | 100 | 16 | 6.25 | 10000 | 0.075 |
| 0.333 | 103 | 66 | 3 | 1 | 1 | 27.7 | SC | 100 | 16 | 6.25 | 10000 | 0.075 |
| 0.5 | 78.2 | 66 | 3 | 1 | 1 | 27.7 | SC | 100 | 16 | 6.25 | 10000 | 0.075 |
| 0.75 | 52.7 | 66 | 3 | 1 | 1 | 27.7 | SC | 100 | 16 | 6.25 | 10000 | 0.075 |
| 1 | 53.3 | 66 | 3 | 1 | 1 | 27.7 | SC | 100 | 16 | 6.25 | 10000 | 0.075 |
| 1.5 | 32.3 | 66 | 3 | 1 | 1 | 27.7 | SC | 100 | 16 | 6.25 | 10000 | 0.075 |
| 2 | 23.2 | 66 | 3 | 1 | 1 | 27.7 | SC | 100 | 16 | 6.25 | 10000 | 0.075 |
| 3 | 11.4 | 66 | 3 | 1 | 1 | 27.7 | SC | 100 | 16 | 6.25 | 10000 | 0.075 |
| 4 | 6.39 | 66 | 3 | 1 | 1 | 27.7 | SC | 100 | 16 | 6.25 | 10000 | 0.075 |
| 6 | BLQ | 66 | 3 | 1 | 1 | 27.7 | SC | 100 | 16 | 6.25 | 10000 | 0.075 |
| 8 | BLQ | 66 | 3 | 1 | 1 | 27.7 | SC | 100 | 16 | 6.25 | 10000 | 0.075 |
| 10 | BLQ | 66 | 3 | 1 | 1 | 27.7 | SC | 100 | 16 | 6.25 | 10000 | 0.075 |
| 24 | BLQ | 66 | 3 | 2 | 1 | 27.7 | SC | 100 | 16 | 6.25 | 10000 | 0.075 |
| 48 | BLQ | 66 | 3 | 3 | 1 | 27.7 | SC | 100 | 16 | 6.25 | 10000 | 0.075 |
| 72 | BLQ | 66 | 3 | 4 | 1 | 27.7 | SC | 100 | 16 | 6.25 | 10000 | 0.075 |
| 312 | BLQ | 66 | 3 | 14 | 1 | 27.7 | SC | 100 | 16 | 6.25 | 10000 | 0.075 |
| 0.0167 | 107 | 68 | 3 | 1 | 1 | 29 | SC | 10 | 1.6 | 6.25 | 10000 | 0.15 |
| 0.05 | 342 | 68 | 3 | 1 | 1 | 29 | SC | 10 | 1.6 | 6.25 | 10000 | 0.15 |
| 0.083 | 351 | 68 | 3 | 1 | 1 | 29 | SC | 10 | 1.6 | 6.25 | 10000 | 0.15 |
| 0.167 | 269 | 68 | 3 | 1 | 1 | 29 | SC | 10 | 1.6 | 6.25 | 10000 | 0.15 |
| 0.25 | 247 | 68 | 3 | 1 | 1 | 29 | SC | 10 | 1.6 | 6.25 | 10000 | 0.15 |
| 0.333 | 201 | 68 | 3 | 1 | 1 | 29 | SC | 10 | 1.6 | 6.25 | 10000 | 0.15 |
| 0.5 | 175 | 68 | 3 | 1 | 1 | 29 | SC | 10 | 1.6 | 6.25 | 10000 | 0.15 |
| 0.75 | 114 | 68 | 3 | 1 | 1 | 29 | SC | 10 | 1.6 | 6.25 | 10000 | 0.15 |
| 1 | 90.6 | 68 | 3 | 1 | 1 | 29 | SC | 10 | 1.6 | 6.25 | 10000 | 0.15 |
| 1.5 | 50.5 | 68 | 3 | 1 | 1 | 29 | SC | 10 | 1.6 | 6.25 | 10000 | 0.15 |
| 2 | 43.2 | 68 | 3 | 1 | 1 | 29 | SC | 10 | 1.6 | 6.25 | 10000 | 0.15 |
| 3 | 17 | 68 | 3 | 1 | 1 | 29 | SC | 10 | 1.6 | 6.25 | 10000 | 0.15 |
| 4 | 7.09 | 68 | 3 | 1 | 1 | 29 | SC | 10 | 1.6 | 6.25 | 10000 | 0.15 |
| 6 | 2.68 | 68 | 3 | 1 | 1 | 29 | SC | 10 | 1.6 | 6.25 | 10000 | 0.15 |
| 8 | BLQ | 68 | 3 | 1 | 1 | 29 | SC | 10 | 1.6 | 6.25 | 10000 | 0.15 |
| 10 | BLQ | 68 | 3 | 1 | 1 | 29 | SC | 10 | 1.6 | 6.25 | 10000 | 0.15 |
| 24 | BLQ | 68 | 3 | 2 | 1 | 29 | SC | 10 | 1.6 | 6.25 | 10000 | 0.15 |
| 48 | BLQ | 68 | 3 | 3 | 1 | 29 | SC | 10 | 1.6 | 6.25 | 10000 | 0.15 |
| 72 | BLQ | 68 | 3 | 4 | 1 | 29 | SC | 10 | 1.6 | 6.25 | 10000 | 0.15 |
| 312 | BLQ | 68 | 3 | 14 | 1 | 29 | SC | 10 | 1.6 | 6.25 | 10000 | 0.15 |

^{a}BLQ = below assay limit of quantification (2.0 ng/mL)

### C. Urinary Excretion of Zoledronic Acid in Pigs

Urine samples were collected continuously from dosing to 2 hours post dose and at 4, 6, 8, and 24 hours post dose. Urine sample volumes and concentration of ZA in the samples were used to calculate the amount of unchanged ZA excreted via the kidneys. Since continuous collection was halted at 2 hours post dose, the percent of administered ZA dose that were renally excreted was not determined. However, the percent of dose excreted within the 2-hour collection interval was estimated with the relationship that the systemically available SC ZA dose is equal to:

ZA Dose absorbed from SC site (0-2 h interval)(F*Dose)=(AUC₍₀₋₂ₕ₎)×CL

The amount of ZA excreted unchanged and the calculated percent of SC dose excreted are shown in Table 21. Also shown in the table is the urinary excretion profile for the 20-minute IV infusion of ZA. The mean percent dose excreted by the kidneys, from dosing to 2 hours post dose, by animals in Study Groups 1, 2, and 3 were comparable; with higher variability noted for the SC dose groups (Study Groups 2 and 3). The mean percents of dose excreted unchanged for the first 2 hours post dose were 11.40%±1.60%, 9.45%±6.29%, and 10.66%±7.41% for Study Groups 1, 2, and 3 respectively. The excretion characteristics of ZA appeared to be the same regardless of route of administration.

TABLE 21
Percent of ZA Dose Excreted Unchanged in Urine Collected Within A
Time Interval from Dosing to 2 Hours Post Dose

| | | | | | | | Serum | | | |
|---|---|---|---|---|---|---|---|---|---|---|
| | | | | | | | AUC | Serum | Dose | |
| | | Body | | Urine | Urine^{a} | Amount | (0-2 h)^{c} | CL^{d} | Amt | Dose |
| Study | Pig | Wt | ZA Dose | ZA Conc | Volume | Excreted^{b} | (ng- | (mL/h- | 2 hr^{e} | Excreted^{f} |
| Group | ID | (kg) | (mg/kg) | (ug/mL) | (mL) | (ug) | h/mL) | kg) | (mg) | (%) |
| 1 | 57 | 29.1 | 0.15 | 3.85 | 150 | 577.50 | NC^{g} | NC | 4.37 | 13.23 |
| 1 | 61 | 26.0 | 0.15 | 26.10 | 16 | 417.60 | NC | NC | 3.90 | 10.71 |
| 1 | 67 | 26.4 | 0.15 | 8.12 | 50 | 406.00 | NC | NC | 3.96 | 10.25 |
| Mean | | | | | | 467.03 | | | 4.08 | 11.40 |
| Std Dev | | | | | | 95.84 | | | 0.25 | 1.60 |
| Median | | | | | | 417.60 | | | 3.96 | 10.71 |
| n | | | | | | 3 | | | 3 | 3 |
| 2 | 59 | 27.7 | 0.075 | 1.47 | 50 | 73.50 | 118.29 | 440.12 | 1.44 | 5.10 |
| 2 | 60 | 27.3 | 0.15 | 4.41 | 50 | 220.50 | 272.70 | 440.12 | 3.28 | 6.73 |
| 2 | 62 | 26.8 | 0.15 | 9.74 | 50 | 487.00 | 205.57 | 440.12 | 2.42 | 20.08 |
| 2 | 69 | 30.0 | 0.15 | 5.77 | 50 | 288.50 | 214.38 | 440.12 | 2.83 | 10.19 |
| 2 | 70 | 26.0 | 0.15 | 3.03 | 50 | 151.50 | 256.13 | 440.12 | 2.93 | 5.17 |
| Mean | | | | | | 286.88 | 237.20 | | 2.87 | 9.45 |
| Std Dev | | | | | | 144.67 | 32.35 | | 0.35 | 6.29 |
| Median | | | | | | 254.50 | 235.25 | | 2.88 | 6.73 |
| n | | | | | | 4ⁱ | 4ⁱ | | 4ⁱ | 5 |
| 3 | 66 | 27.7 | 0.075 | 6.32 | 35 | 221.20 | 100.06 | 440.12 | 1.22 | 18.13 |
| 3 | 64 | 27.3 | 0.15 | 7.81 | 45 | 351.45 | 238.02 | 440.12 | 2.86 | 12.29 |
| 3 | 58 | 26.4 | 0.15 | 12.50 | 1 | 12.50 | 261.08 | 440.12 | 3.03 | 0.41 |
| 3 | 56 | 26.0 | 0.15 | NS^{h} | NS | NS | 267.58 | 440.12 | 3.06 | NS |
| 3 | 68 | 29.0 | 0.15 | 7.15 | 50 | 357.50 | 237.66 | 440.12 | 3.03 | 11.79 |

| | | | | |
|---|---|---|---|---|
| Mean | 240.48 | 251.08 | 3.00 | 10.66 |
| Std Dev | 197.46 | 15.53 | 0.09 | 7.41 |
| Median | 351.45 | 249.55 | 3.03 | 12.04 |
| n | 3ⁱ | 4ⁱ | 4ⁱ | 4 |

^{a}Urine volume was collected from dose initiation to 2 hours post dose
^{b}Amount excreted (ug) = [urine volume (mL) × urine sample concentration (ng/mL)] ÷ 1000 ng/ug
^{c}Serum AUC(0-2 h) calculated by non-compartmental, trapezoidal method
^{d}Absolute clearance (CL) obtained by non-compartmental analysis of the IV dose (from Table 13)
^{e}Dose amount at 2 hours for Group 1 = Total dose administered via IV infusion = pig body weight × ZA IV dose/kg. Dose amount at 2 hours for Groups 2 and 3 = SC AUC(0-2 h) × CL × pig body weight
^{f}Percent dose excreted unchanged at 2 hours = (Amount excreted ÷ Dose Amount at 2 hours) × 100%
^{g}NC = not calculated
^{h}NS = no sample
ⁱMean, standard deviation, median, and # of observations calculated for the 0.15 mg/kg dose only

### Example 4

### Generation of a Soluble rHuPH20-Expressing Cell Line

The HZ24 plasmid (set forth in SEQ ID NO:52) was used to transfect Chinese Hamster Ovary (CHO cells) (see e.g. U.S. patent application Ser. Nos. 10/795,095, 11/065,716 and 11/238,171). The HZ24 plasmid vector for expression of soluble rHuPH20 contains a pCI vector backbone (Promega), DNA encoding amino acids 1-482 of human PH20 hyaluronidase (SEQ ID NO:49), an internal ribosomal entry site (IRES) from the ECMV virus (Clontech), and the mouse dihydrofolate reductase (DHFR) gene. The pCI vector backbone also includes DNA encoding the Beta-lactamase resistance gene (AmpR), an f1 origin of replication, a Cytomegalovirus immediate-early enhancer/promoter region (CMV), a chimeric intron, and an SV40 late polyadenylation signal (SV40). The DNA encoding the soluble rHuPH20 construct contains an NheI site and a Kozak consensus sequence prior to the DNA encoding the methionine at amino acid position 1 of the native 35 amino acid signal sequence of human PH20, and a stop codon following the DNA encoding the tyrosine corresponding to amino acid position 482 of the human PH20 hyaluronidase set forth in SEQ ID NO:1), followed by a BamHI restriction site. The construct pCI-PH20-IRES-DHFR-SV40pa (HZ24), therefore, results in a single mRNA species driven by the CMV promoter that encodes amino acids 1-482 of human PH20 (set forth in SEQ ID NO:3) and amino acids 1-186 of mouse dihydrofolate reductase (set forth in SEQ ID NO:53), separated by the internal ribosomal entry site (IRES).

Non-transfected DG44 CHO cells growing in GIBCO Modified CD-CHO media for DHFR(−) cells, supplemented with 4 mM Glutamine and 18 ml/L Plurionic F68/L (Gibco), were seeded at 0.5×10⁶ cells/ml in a shaker flask in preparation for transfection. Cells were grown at 37° C. in 5% CO₂ in a humidified incubator, shaking at 120 rpm. Exponentially growing non-transfected DG44 CHO cells were tested for viability prior to transfection.

Sixty million viable cells of the non-transfected DG44 CHO cell culture were pelleted and resuspended to a density of 2×10⁷ cells in 0.7 mL of 2× transfection buffer (2× HeBS: 40 mM Hepes, pH 7.0, 274 mM NaCl, 10 mM KCl, 1.4 mM Na₂HPO₄, 12 mM dextrose). To each aliquot of resuspended cells, 0.09 mL (250 μg) of the linear HZ24 plasmid (linearized by overnight digestion with Cla I (New England Biolabs) was added, and the cell/DNA solutions were transferred into 0.4 cm gap BTX (Gentronics) electroporation cuvettes at room temperature. A negative control electroporation was performed with no plasmid DNA mixed with the cells. The cell/plasmid mixes were electroporated with a capacitor discharge of 330 V and 960 μF or at 350 V and 960 μF.

The cells were removed from the cuvettes after electroporation and transferred into 5 mL of Modified CD-CHO media for DHFR(−) cells, supplemented with 4 mM Glutamine and 18 ml/L Plurionic F68/L (Gibco), and allowed to grow in a well of a 6-well tissue culture plate without selection for 2 days at 37° C. in 5% CO₂ in a humidified incubator.

Two days post-electroporation, 0.5 mL of tissue culture media was removed from each well and tested for the presence of hyaluronidase activity, using the microturbidity assay described in Example 5. Results are shown in Table 22.

TABLE 22
Initial Hyaluronidase Activity of HZ24 Transfected
DG44 CHO cells at 40 hours post-transfection

| | | Activity |
|---|---|---|
| | Dilution | Units/ml |
| Transfection 1 | 1 to 10 | 0.25 |
| 330 V | | |
| Transfection 2 | 1 to 10 | 0.52 |
| 350 V | | |
| Negative Control | 1 to 10 | 0.015 |

Cells from Transfection 2 (350V) were collected from the tissue culture well, counted and diluted to 1×10⁴ to 2×10⁴ viable cells per mL. A 0.1 mL aliquot of the cell suspension was transferred to each well of five, 96 well round bottom tissue culture plates. One hundred microliters of CD-CHO media (GIBCO) containing 4 mM GlutaMAX™-1 supplement (GIBCO™, Invitrogen Corporation) and without hypoxanthine and thymidine supplements were added to the wells containing cells (final volume 0.2 mL). Ten clones were identified from the 5 plates grown without methotrexate (Table 23).

TABLE 23
Hyaluronidase activity of identified clones

| | Relative |
|---|---|
| Plate/Well ID | Hyaluronidase |
| 1C3 | 261 |
| 2C2 | 261 |
| 3D3 | 261 |
| 3E5 | 243 |
| 3C6 | 174 |
| 2G8 | 103 |
| 1B9 | 304 |
| 2D9 | 273 |
| 4D10 | 302 |

Six HZ24 clones were expanded in culture and transferred into shaker flasks as single cell suspensions. Clones 3D3, 3E5, 2G8, 2D9, 1E11, and 4D10 were plated into 96-well round bottom tissue culture plates using a two-dimensional infinite dilution strategy in which cells were diluted 1:2 down the plate, and 1:3 across the plate, starting at 5000 cells in the top left hand well. Diluted clones were grown in a background of 500 non-transfected DG44 CHO cells per well, to provide necessary growth factors for the initial days in culture. Ten plates were made per subclone, with 5 plates containing 50 nM methotrexate and 5 plates without methotrexate.

Clone 3D3 produced 24 visual subclones (13 from the no methotrexate treatment, and 11 from the 50 nM methotrexate treatment. Significant hyaluronidase activity was measured in the supernatants from 8 of the 24 subclones (>50 Units/mL), and these 8 subclones were expanded into T-25 tissue culture flasks. Clones isolated from the methotrexate treatment protocol were expanded in the presence of 50 nM methotrexate. Clone 3D35M was further expanded in 500 nM methotrexate giving rise to clones producing in excess of 1,000 Units/ml in shaker flasks (clone 3D35M; or Gen1 3D35M). A master cell bank (MCB) of the 3D35M cells was then prepared.

### Example 5

### Determination of Hyaluronidase Activity of Soluble rHuPH20

Hyaluronidase activity of soluble rHuPH20 in samples such as cell cultures, purification fractions and purified solutions was determined using a turbidometric assay, which based on the formation of an insoluble precipitate when hyaluronic acid binds with serum albumin. The activity is measured by incubating soluble rHuPH20 with sodium hyaluronate (hyaluronic acid) for a set period of time (10 minutes) and then precipitating the undigested sodium hyaluronate with the addition of acidified serum albumin. The turbidity of the resulting sample is measured at 640 nm after a 30 minute development period. The decrease in turbidity resulting from enzyme activity on the sodium hyaluronate substrate is a measure of the soluble rHuPH20 hyaluronidase activity. The method is performed using a calibration curve generated with dilutions of a soluble rHuPH20 assay working reference standard, and sample activity measurements are made relative to this calibration curve.

Dilutions of the sample were prepared in Enzyme Diluent Solutions. The Enzyme Diluent Solution was prepared by dissolving 33.0±0.05 mg of hydrolyzed gelatin in 25.0 mL of the 50 mM PIPES Reaction Buffer (140 mM NaCl, 50 mM PIPES, pH 5.5) and 25.0 mL of SWFI, and diluting 0.2 mL of 25% Buminate solution into the mixture and vortexing for 30 seconds. This was performed within 2 hours of use and stored on ice until needed. The samples were diluted to an estimated 1-2 U/mL. Generally, the maximum dilution per step did not exceed 1:100 and the initial sample size for the first dilution was not be less than 20 μL. The minimum sample volumes needed to perform the assay were: In-process Samples, FPLC Fractions: 80 μL; Tissue Culture Supernatants: 1 mL; Concentrated Material 80 μL; Purified or Final Step Material: 80 μL. The dilutions were made in triplicate in a Low Protein Binding 96-well plate, and 30 μL of each dilution was transferred to Optilux black/clear bottom plates (BD BioSciences).

Dilutions of known soluble rHuPH20 with a concentration of 2.5 U/mL were prepared in Enzyme Diluent Solution to generate a standard curve and added to the Optilux plate in triplicate. The dilutions included 0 U/mL, 0.25 U/mL, 0.5 U/mL, 1.0 U/mL, 1.5 U/mL, 2.0 U/mL, and 2.5 U/mL. “Reagent blank” wells that contained 60 μL of Enzyme Diluent Solution were included in the plate as a negative control. The plate was then covered and warmed on a heat block for 5 minutes at 37° C. The cover was removed and the plate was shaken for 10 seconds. After shaking, the plate was returned to the plate to the heat block and the MULTIDROP 384 Liquid Handling Device was primed with the warm 0.25 mg/mL sodium hyaluronate solution (prepared by dissolving 100 mg of sodium hyaluronate (LifeCore Biomedical) in 20.0 mL of SWFI. This was mixed by gently rotating and/or rocking at 2-8° C. for 2-4 hours, or until completely dissolved). The reaction plate was transferred to the MULTIDROP 384 and the reaction was initiated by pressing the start key to dispense 30 μL sodium hyaluronate into each well. The plate was then removed from the MULTIDROP 384 and shaken for 10 seconds before being transferred to a heat block with the plate cover replaced. The plate was incubated at 37° C. for 10 minutes

The MULTIDROP 384 was prepared to stop the reaction by priming the machine with Serum Working Solution and changing the volume setting to 240 μL. (25 mL of Serum Stock Solution [1 volume of Horse Serum (Sigma) was diluted with 9 volumes of 500 mM Acetate Buffer Solution and the pH was adjusted to 3.1 with hydrochloric acid] in 75 mL of 500 mM Acetate Buffer Solution). The plate was removed from the heat block and placed onto the MULTIDROP 384 and 240 μL of serum Working Solutions was dispensed into the wells. The plate was removed and shaken on a plate reader for 10 seconds. After a further 15 minutes, the turbidity of the samples was measured at 640 nm and the hyaluronidase activity (in U/mL) of each sample was determined by fitting to the standard curve.

Specific activity (Units/mg) was calculated by dividing the hyaluronidase activity (U/ml) by the protein concentration (mg/mL).

### Example 6

### Production and Purification of Gen1 Human sPH20

### A. 5 L Bioreactor Process

A vial of 3D35M was thawed and expanded from shaker flasks through 1 L spinner flasks in CD-CHO media (Invitrogen, Carlsbad Calif.) supplemented with 100 nM Methotrexate and GlutaMAX™-1 (Invitrogen). Cells were transferred from spinner flasks to a 5 L bioreactor (Braun) at an inoculation density of 4×10⁵ viable cells per ml. Parameters were temperature Setpoint 37° C., pH 7.2 (starting Setpoint), with Dissolved Oxygen Setpoint 25% and an air overlay of 0-100 cc/min. At 168 hours, 250 ml of Feed #1 Medium (CD CHO with 50 g/L Glucose) was added. At 216 hours, 250 ml of Feed #2 Medium (CD CHO with 50 g/L Glucose and 10 mM Sodium Butyrate) was added, and at 264 hours 250 ml of Feed #2 Medium was added. This process resulted in a final productivity of 1600 Units per ml with a maximal cell density of 6×10⁶ cells/ml. The addition of sodium butyrate was to dramatically enhance the production of soluble rHuPH20 in the final stages of production.

Conditioned media from the 3D35M clone was clarified by depth filtration and tangential flow diafiltration into 10 mM Hepes pH 7.0. Soluble rHuPH20 was then purified by sequential chromatography on Q Sepharose (Pharmacia) ion exchange, Phenyl Sepharose (Pharmacia) hydrophobic interaction chromatography, phenyl boronate (Prometics) and Hydroxyapatite Chromatography (Biorad, Richmond, Calif.).

Soluble rHuPH20 bound to Q Sepharose and eluted at 400 mM NaCl in the same buffer. The eluate was diluted with 2M ammonium sulfate to a final concentration of 500 mM ammonium sulfate and passed through a Phenyl Sepharose (low sub) column, followed by binding under the same conditions to a phenyl boronate resin. The soluble rHuPH20 was eluted from the phenyl sepharose resin in Hepes pH 6.9 after washing at pH 9.0 in 50 mM bicine without ammonium sulfate. The eluate was loaded onto a ceramic hydroxyapatite resin at pH 6.9 in 5 mM potassium phosphate and 1 mM CaCl₂ and eluted with 80 mM potassium phosphate, pH 7.4 with 0.1 mM CaCl₂.

The resultant purified soluble rHuPH20 possessed a specific activity in excess of 65,000 USP Units/mg protein by way of the microturbidity assay (Example 5) using the USP reference standard. Purified sPH20 eluted as a single peak from 24 to 26 minutes from a Pharmacia 5RPC styrene divinylbenzene column with a gradient between 0.1% TFA/H₂O and 0.1% TFA/90% acetonitrile/10% H₂O and resolved as a single broad 61 kDa band by SDS electrophoresis that reduced to a sharp 51 kDa band upon treatment with PNGASE-F. N-terminal amino acid sequencing revealed that the leader peptide had been efficiently removed.

B. Upstream Cell Culture Expansion Process into 100 L Bioreactor Cell Culture

A scaled-up process was used to separately purify soluble rHuPH20 from four different vials of 3D35M cell to produce 4 separate batches of sHuPH20; HUA0406C, HUA0410C, HUA0415C and HUA0420C. Each vial was separately expanded and cultured through a 125 L bioreactor, then purified using column chromatography. Samples were taken throughout the process to assess such parameters as enzyme yield. The description of the process provided below sets forth representative specifications for such things as bioreactor starting and feed media volumes, transfer cell densities, and wash and elution volumes. The exact numbers vary slightly with each batch, and are detailed in Tables 24 to 31.

Four vials of 3D35M cells were thawed in a 37° C. water bath, CD CHO containing 100 nM methotrexate and 40 mL/L GlutaMAX was added and the cells were centrifuged. The cells were re-suspended in a 125 mL shake flask with 20 mL of fresh media and placed in a 37° C., 7% CO₂ incubator. The cells were expanded up to 40 mL in the 125 mL shake flask. When the cell density reached 1.5−2.5×10⁶ cells/mL, the culture was expanded into a 125 mL spinner flask in a 100 mL culture volume. The flask was incubated at 37° C., 7% CO₂. When the cell density reached 1.5−2.5×10⁶ cells/mL, the culture was expanded into a 250 mL spinner flask in 200 mL culture volume, and the flask was incubated at 37° C., 7% CO₂. When the cell density reached 1.5−2.5×10⁶ cells/mL, the culture was expanded into a 1 L spinner flask in 800 mL culture volume and incubated at 37° C., 7% CO₂. When the cell density reached 1.5−2.5×10⁶ cells/mL, the culture was expanded into a 6 L spinner flask in 5 L culture volume and incubated at 37° C., 7% CO₂. When the cell density reached 1.5−2.5×10⁶ cells/mL, the culture was expanded into a 36 L spinner flask in 20 L culture volume and incubated at 37° C., 7% CO₂.

A 125 L reactor was sterilized with steam at 121° C., 20 PSI and 65 L of CD CHO media was added. Before use, the reactor was checked for contamination. When the cell density in the 36 L spinner flasks reached 1.8−2.5×10⁶ cells/mL, 20 L cell culture were transferred from the 36 L spinner flasks to the 125 L bioreactor (Braun), resulting a final volume of 85 L and a seeding density of approximately 4×10⁵ cells/mL. Parameters were temperature setpoint, 37° C.; pH: 7.2; Dissolved oxygen: 25%±10%; Impeller Speed 50 rpm; Vessel Pressure 3 psi; Air Sparge 1 L/min.; Air Overlay: 1 L/min. The reactor was sampled daily for cell counts, pH verification, media analysis, protein production and retention. Nutrient feeds were added during the run. At Day 6, 3.4 L of Feed #1 Medium (CD CHO+50 g/L Glucose+40 mL/L GlutaMAX™-1) was added, and culture temperature was changed to 36.5° C. At day 9, 3.5 L of Feed #2 (CD CHO+50 g/L Glucose+40 mL/L GlutaMAX™-1+1.1 g/L Sodium Butyrate) was added, and culture temperature was changed to 36° C. At day 11, 3.7 L of Feed #3 (CD CHO+50 g/L Glucose+40 mL/L GlutaMAX™-1+1.1 g/L Sodium Butyrate) was added, and the culture temperature was changed to 35.5° C. The reactor was harvested at 14 days or when the viability of the cells dropped below 50%. The process resulted in production of soluble rHuPH20 with an enzymatic activity of 1600 Units/ml with a maximal cell density of 8 million cells/mL. At harvest, the culture was sampled for mycoplasma, bioburden, endotoxin, and virus in vitro and in vivo, transmission electron microscopy (TEM) for viral particles, and enzyme activity.

The one hundred liter bioreactor cell culture harvest was filtered through a series of disposable capsule filters having a polyethersulfone medium (Sartorius): first through a 8.0 μm depth capsule, a 0.65 μm depth capsule, a 0.22 μm capsule, and finally through a 0.22 μm Sartopore 2000 cm² filter and into a 100 L sterile storage bag. The culture was concentrated 10× using two TFF with Spiral Polyethersulfone 30 kDa MWCO filters (Millipore), followed by a 6× buffer exchange with 10 mM HEPES, 25 mM Na₂SO₄, pH 7.0 into a 0.22 μm final filter into a 20 L sterile storage bag. Table 24 provides monitoring data related to the cell culture, harvest, concentration and buffer exchange steps.

TABLE 24
Monitoring data for cell culture, harvest, concentration and buffer
exchange steps.

| Parameter | HUA0406C | HUA04010C | HUA0415C | HUA0420C |
|---|---|---|---|---|
| Time from thaw to inoculate | 21 | 19 | 17 | 18 |
| 100 L bioreactor (days) | | | | |
| 100 L inoculation density | 0.45 | 0.33 | 0.44 | 0.46 |
| (×10⁶ cells/mL) | | | | |
| Doubling time in logarithmic | 29.8 | 27.3 | 29.2 | 23.5 |
| growth (hr) | | | | |
| Max. cell density | 5.65 | 8.70 | 6.07 | 9.70 |
| (×10⁶ cells/mL) | | | | |
| Harvest viability (%) | 41 | 48 | 41 | 41 |
| Harvest titer (U/ml) | 1964 | 1670 | 991 | 1319 |
| Time in 100-L bioreactor | 13 | 13 | 12 | 13 |
| (days) | | | | |
| Clarified harvest volume (mL) | 81800 | 93300 | 91800 | 89100 |
| Clarified harvest enzyme assay | 2385 | 1768 | 1039 | 1425 |
| (U/mL) | | | | |
| Concentrate enzyme assay | 22954 | 17091 | 8561 | 17785 |
| (U/mL) | | | | |
| Buffer exchanged concentrate | 15829 | 11649 | 9915 | 8679 |
| enzyme assay (U/mL) | | | | |
| Filtered buffer exchanged | 21550 | 10882 | 9471 | 8527 |
| concentrate enzyme assay | | | | |
| (U/mL) | | | | |
| Buffer exchanged concentrate | 10699 | 13578 | 12727 | 20500 |
| volume (mL) | | | | |
| Ratio enzyme units | 0.87 | 0.96 | 1.32 | 1.4 |
| concentration/harvest | | | | |

A Q Sepharose (Pharmacia) ion exchange column (3 L resin, Height=20 cm, Diameter=14 cm) was prepared. Wash samples were collected for a determination of pH, conductivity and endotoxin (LAL) assay. The column was equilibrated with 5 column volumes of 10 mM Tris, 20 mM Na₂SO₄, pH 7.5. The concentrated, diafiltered harvest was loaded onto the Q column at a flow rate of 100 cm/hr. The column was washed with 5 column volumes of 10 mM Tris, 20 mM Na₂SO₄, pH 7.5 and 10 mM Hepes, 50 mM NaCl, pH 7.0. The protein was eluted with 10 mM Hepes, 400 mM NaCl, pH 7.0 and filtered through a 0.22 μm final filter into a sterile bag.

Phenyl-Sepharose (Pharmacia) hydrophobic interaction chromatography was next performed. A Phenyl-Sepharose (PS) column (9.1 L resin, Height=29 cm, Diameter=20 cm) was prepared. The column was equilibrated with 5 column volumes of 5 mM potassium phosphate, 0.5 M ammonium sulfate, 0.1 mM CaCl₂, pH 7.0. The protein eluate from above was supplemented with 2M ammonium sulfate, 1 M potassium phosphate and 1 M CaCl₂ stock solutions to final concentrations of 5 mM, 0.5 M and 0.1 mM, respectively. The protein was loaded onto the PS column at a flow rate of 100 cm/hr. 5 mM potassium phosphate, 0.5 M ammonium sulfate and 0.1 mM CaCl₂ pH 7.0 was added at 100 cm/hr. The flow through was passed through a 0.22 μm final filter into a sterile bag.

The PS-purified protein was the loaded onto an aminophenyl boronate column (ProMedics) (6.3 L resin, Height=20 cm, Diameter=20 cm) that had been equilibrated with 5 column volumes of 5 mM potassium phosphate, 0.5 M ammonium sulfate. The protein was passed through the column at a flow rate of 100 cm/hr, and the column was washed with 5 mM potassium phosphate, 0.5 M ammonium sulfate, pH 7.0. The column was then washed with 20 mM bicine, 100 mM NaCl, pH 9.0 and the protein eluted with 50 mM Hepes, 100 mM NaCl pH 6.9 through a sterile filter and into a 20 L sterile bag. The eluate was tested for bioburden, protein concentration and enzyme activity.

A hydroxyapatite (HAP) column (BioRad) (1.6 L resin, Height=10 cm, Diameter=14 cm) was equilibrated with 5 mM potassium phosphate, 100 mM NaCl, 0.1 mM CaCl₂ pH 7.0. Wash samples were collected and tested for pH, conductivity and endotoxin (LAL assay. The aminophenyl boronate purified protein was supplemented with potassium phosphate and CaCl₂ to yield final concentrations of 5 mM potassium phosphate and 0.1 mM CaCl₂ and loaded onto the HAP column at a flow rate of 100 cm/hr. The column was washed with 5 mM potassium phosphate pH 7.0, 100 mM NaCl, 0.1 mM CaCl₂, then 10 mM potassium phosphate pH 7.0, 100 mM NaCl, 0.1 mM CaCl₂ pH. The protein was eluted with 70 mM potassium phosphate pH 7.0 and filtered through a 0.22 μm filter into a 5 L sterile storage bag. The eluate was tested for bioburden, protein concentration and enzyme activity.

The HAP-purified protein was then pumped through a 20 nM viral removal filter via a pressure tank. The protein was added to the DV20 pressure tank and filter (Pall Corporation), passing through an Ultipor DV20 Filter with 20 nm pores (Pall Corporation) into a sterile 20 L storage bag. The filtrate was tested for protein concentration, enzyme activity, oligosaccharide, monosaccharide and sialic acid profiling, and process-related impurities. The protein in the filtrate was then concentrated to 1 mg/mL using a 10 kDa molecular weight cut off (MWCO) Sartocon Slice tangential flow filtration (TFF) system (Sartorius). The filter was first prepared by washing with a Hepes/saline solution (10 mM Hepes, 130 mM NaCl, pH 7.0) and the permeate was sampled for pH and conductivity. Following concentration, the concentrated protein was sampled and tested for protein concentration and enzyme activity. A 6× buffer exchange was performed on the concentrated protein into the final buffer: 10 mM Hepes, 130 mM NaCl, pH 7.0. The concentrated protein was passed though a 0.22 μm filter into a 20 L sterile storage bag. The protein was sampled and tested for protein concentration, enzyme activity, free sulflhydryl groups, oligosaccharide profiling and osmolarity.

Tables 25 to 31 provide monitoring data related to each of the purification steps described above, for each 3D35M cell lot.

TABLE 25
Q sepharose column data

| Parameter | HUA0406C | HUA0410C | HUA0415C | HUA0420C |
|---|---|---|---|---|
| Load volume | 10647 | 13524 | 12852 | 20418 |
| (mL) | | | | |
| Load Volume/ | 3.1 | 4.9 | 4.5 | 7.3 |
| Resin Volume | | | | |
| ratio | | | | |
| Column | 2770 | 3840 | 2850 | 2880 |
| Volume (mL) | | | | |
| Eluate volume | 6108 | 5923 | 5759 | 6284 |
| (mL) | | | | |
| Protein Conc. | 2.8 | 3.05 | 2.80 | 2.86 |
| of Eluate | | | | |
| (mg/mL) | | | | |
| Eluate Enzyme | 24493 | 26683 | 18321 | 21052 |
| Assay (U/mL) | | | | |
| Enzyme Yield | 65 | 107 | 87 | 76 |
| (%) | | | | |

TABLE 26
Phenyl Sepharose column data

| Parameter | HUA0406C | HUA0410C | HUA0415C | HUA0420C |
|---|---|---|---|---|
| Volume Before | 5670 | 5015 | 5694 | 6251 |
| Stock Solution | | | | |
| Addition (mL) | | | | |
| Load Volume | 7599 | 6693 | 7631 | 8360 |
| (mL) | | | | |
| Column | 9106 | 9420 | 9340 | 9420 |
| Volume (mL) | | | | |
| Load Volume/ | 0.8 | 0.71 | 0.82 | 0.89 |
| Resin Volume | | | | |
| ratio | | | | |
| Eluate volume | 16144 | 18010 | 16960 | 17328 |
| (mL) | | | | |
| Protein Cone | 0.4 | 0.33 | 0.33 | 0.38 |
| of Eluate | | | | |
| (mg/mL) | | | | |
| Eluate Enzyme | 8806 | 6585 | 4472 | 7509 |
| Assay (U/mL) | | | | |
| Protein Yield | 41 | 40 | 36 | 37 |
| (%) | | | | |
| Enzyme Yield | 102 | 88 | 82 | 96 |
| (%) | | | | |

TABLE 27
Amino Phenyl Boronate column data

| Parameter | HUA0406C | HUA0410C | HUA0415C | HUA0420C |
|---|---|---|---|---|
| Load Volume | 16136 | 17958 | 16931 | 17884 |
| (mL) | | | | |
| Load Volume/ | 2.99 | 3.15 | 3.08 | 2.98 |
| Resin Volume | | | | |
| ratio | | | | |
| Column | 5400 | 5700 | 5500 | 5300 |
| Volume (mL) | | | | |
| Eluate volume | 17595 | 22084 | 20686 | 19145 |
| (mL) | | | | |
| Protein Conc. | 0.0 | 0.03 | 0.03 | 0.04 |
| of Eluate | | | | |
| (mg/mL) | | | | |
| Protein Conc. | not tested | 0.03 | 0.00 | 0.04 |
| of Filtered | | | | |
| Eluate | | | | |
| (mg/mL) | | | | |
| Eluate Enzyme | 4050 | 2410 | 1523 | 4721 |
| Assay (U/mL) | | | | |
| Protein Yield | 0 | 11 | 11 | 12 |
| (%) | | | | |
| Enzyme Yield | not | 41 | 40 | 69 |
| (%) | determined | | | |

TABLE 28
Hydroxyapatite column data

| Parameter | HUA0406C | HUA0410C | HUA0415C | HUA0420C |
|---|---|---|---|---|
| Volume Before | 16345 | 20799 | 20640 | 19103 |
| Stock Solution | | | | |
| Addition (mL) | | | | |
| Load Volume/ | 10.95 | 13.58 | 14.19 | 12.81 |
| Resin Volume | | | | |
| ratio | | | | |
| Column | 1500 | 1540 | 1462 | 1500 |
| Volume (mL) | | | | |
| Load volume | 16429 | 20917 | 20746 | 19213 |
| (mL) | | | | |
| Eluate volume | 4100 | 2415 | 1936 | 2419 |
| (mL) | | | | |
| Protein Conc. | not tested | 0.24 | 0.17 | 0.23 |
| of Eluate | | | | |
| (mg/mL) | | | | |
| Protein Conc. | NA | NA | 0.17 | NA |
| of Filtered | | | | |
| Eluate | | | | |
| (mg/mL) | | | | |
| Eluate Enzyme | 14051 | 29089 | 20424 | 29826 |
| Assay (U/mL) | | | | |
| Protein Yield | Not tested | 93 | 53 | 73 |
| (%) | | | | |
| Enzyme Yield | 87 | 118 | 140 | 104 |
| (%) | | | | |

TABLE 29
DV20 filtration data

| Parameter | HUA0406C | HUA0410C | HUA0415C | HUA0420C |
|---|---|---|---|---|
| Start volume | 4077 | 2233 | 1917 | 2419 |
| (mL) | | | | |
| Filtrate Volume | 4602 | 3334 | 2963 | 3504 |
| (mL) | | | | |
| Protein Conc. | 0.1 | NA | 0.09 | NA |
| of Filtrate | | | | |
| (mg/mL) | | | | |
| Protein Conc. | NA | 0.15 | 0.09 | 0.16 |
| of Filtered | | | | |
| Eluate | | | | |
| (mg/mL) | | | | |
| Protein Yield | not tested | 93 | 82 | 101 |
| (%) | | | | |

TABLE 30
Final concentration data

| Parameter | HUA0406C | HUA0410C | HUA0415C | HUA0420C |
|---|---|---|---|---|
| Start volume | 4575 | 3298 | 2963 | 3492 |
| (mL) | | | | |
| Concentrate | 562 | 407 | 237 | 316 |
| Volume (mL) | | | | |
| Protein Conc. | 0.9 | 1.24 | 1.16 | 1.73 |
| of Concentrate | | | | |
| (mg/mL) | | | | |
| Protein Yield | 111 | 102 | 103 | 98 |
| (%) | | | | |

TABLE 31
Buffer Exchange into Final Formulation data

| Parameter | HUA0406C | HUA0410C | HUA0415C | HUA0420C |
|---|---|---|---|---|
| Start Volume | 562 | 407 | 237 | 316 |
| (mL) | | | | |
| Final Volume | 594 | 516 | 310 | 554 |
| Buffer | | | | |
| Exchanged | | | | |
| Concentrate | | | | |
| (mL) | | | | |
| Protein Conc. | 1.00 | 0.97 | 0.98 | 1.00 |
| of Concentrate | | | | |
| (mg/mL) | | | | |
| Protein Conc. | 0.95 | 0.92 | 0.95 | 1.02 |
| of Filtered | | | | |
| Concentrate | | | | |
| (mg/mL) | | | | |
| Protein Yield | 118 | 99 | 110 | 101 |
| (%) | | | | |

The purified and concentrated soluble rHuPH20 protein was aseptically filled into sterile vials with 5 mL and 1 mL fill volumes. The protein was passed though a 0.22 μm filter to an operator controlled pump that was used to fill the vials using a gravimetric readout. The vials were closed with stoppers and secured with crimped caps. The closed vials were visually inspected for foreign particles and then labeled. Following labeling, the vials were flash-frozen by submersion in liquid nitrogen for no longer than 1 minute and stored at ≦−15° C. (−20±5° C.).

### Example 7

### Production Gen2 Cells Containing Soluble Human PH20 (rHuPH20)

The Gen1 3D35M cell line described in Example 4 was adapted to higher methotrexate levels to produce generation 2 (Gen2) clones. 3D35M cells were seeded from established methotrexate-containing cultures into CD CHO medium containing 4 mM GlutaMAX-1™ and 1.0 μM methotrexate. The cells were adapted to a higher methotrexate level by growing and passaging them 9 times over a period of 46 days in a 37° C., 7% CO₂ humidified incubator. The amplified population of cells was cloned out by limiting dilution in 96-well tissue culture plates containing medium with 2.0 μM methotrexate. After approximately 4 weeks, clones were identified and clone 3E10B was selected for expansion. 3E10B cells were grown in CD CHO medium containing 4 mM GlutaMAX-1™ and 2.0 μM methotrexate for 20 passages. A master cell bank (MCB) of the 3E10B cell line was created and frozen and used for subsequent studies.

Amplification of the cell line continued by culturing 3E10B cells in CD CHO medium containing 4 mM GlutaMAX-1™ and 4.0 μM methotrexate. After the 12^{th} passage, cells were frozen in vials as a research cell bank (RCB). One vial of the RCB was thawed and cultured in medium containing 8.0 μM methotrexate. After 5 days, the methotrexate concentration in the medium was increased to 16.0 μM, then 20.0 μM 18 days later. Cells from the 8^{th} passage in medium containing 20.0 μM methotrexate were cloned out by limiting dilution in 96-well tissue culture plates containing CD CHO medium containing 4 mM GlutaMAX-1™ and 20.0 μM methotrexate. Clones were identified 5-6 weeks later and clone 2B2 was selected for expansion in medium containing 20.0 μM methotrexate. After the 11th passage, 2B2 cells were frozen in vials as a research cell bank (RCB).

The resultant 2B2 cells are dihydrofolate reductase deficient (dhfr-) DG44 CHO cells that express soluble recombinant human PH20 (rHuPH20). The soluble PH20 is present in 2B2 cells at a copy number of approximately 206 copies/cell. Southern blot analysis of Spe I-, Xba I- and BamH I/Hind III-digested genomic 2B2 cell DNA using a rHuPH20-specific probe revealed the following restriction digest profile: one major hybridizing band of ˜7.7 kb and four minor hybridizing bands (˜13.9, ˜6.6, ˜5.7 and ˜4.6 kb) with DNA digested with Spe I; one major hybridizing band of ˜5.0 kb and two minor hybridizing bands (˜13.9 and ˜6.5 kb) with DNA digested with Xba I; and one single hybridizing band of ˜1.4 kb observed using 2B2 DNA digested with BamH I/Hind III. Sequence analysis of the mRNA transcript indicated that the derived cDNA (SEQ ID NO:56) was identical to the reference sequence (SEQ ID NO:47) except for one base pair difference at position 1131, which was observed to be a thymidine (T) instead of the expected cytosine (C). This is a silent mutation, with no effect on the amino acid sequence.

### Example 8

### A. Production of Gen2 Soluble rHuPH20 in 300 L Bioreactor Cell Culture

A vial of HZ24-2B2 was thawed and expanded from shaker flasks through 36 L spinner flasks in CD-CHO media (Invitrogen, Carlsbad, Calif.) supplemented with 20 μM methotrexate and GlutaMAX-1™ (Invitrogen). Briefly, the a vial of cells was thawed in a 37° C. water bath, media was added and the cells were centrifuged. The cells were re-suspended in a 125 mL shake flask with 20 mL of fresh media and placed in a 37° C., 7% CO₂ incubator. The cells were expanded up to 40 mL in the 125 mL shake flask. When the cell density reached greater than 1.5×10⁶ cells/mL, the culture was expanded into a 125 mL spinner flask in a 100 mL culture volume. The flask was incubated at 37° C., 7% CO₂. When the cell density reached greater than 1.5×10⁶ cells/mL, the culture was expanded into a 250 mL spinner flask in 200 mL culture volume, and the flask was incubated at 37° C., 7% CO₂. When the cell density reached greater than 1.5×10⁶ cells/mL, the culture was expanded into a 1 L spinner flask in 800 mL culture volume and incubated at 37° C., 7% CO₂. When the cell density reached greater than 1.5×10⁶ cells/mL the culture was expanded into a 6 L spinner flask in 5000 mL culture volume and incubated at 37° C., 7% CO₂. When the cell density reached greater than 1.5×10⁶ cells/mL the culture was expanded into a 36 L spinner flask in 32 L culture volume and incubated at 37° C., 7% CO₂.

A 400 L reactor was sterilized and 230 mL of CD-CHO media was added. Before use, the reactor was checked for contamination. Approximately 30 L cells were transferred from the 36 L spinner flasks to the 400 L bioreactor (Braun) at an inoculation density of 4.0×10⁵ viable cells per ml and a total volume of 260 L. Parameters were temperature setpoint, 37° C.; Impeller Speed 40-55 RPM; Vessel Pressure: 3 psi; Air Sparge 0.5-1.5 L/Min.; Air Overlay: 3 L/min. The reactor was sampled daily for cell counts, pH verification, media analysis, protein production and retention. Also, during the run nutrient feeds were added. At 120 hrs (day 5), 10.4 L of Feed #1 Medium (4×CD-CHO+33 g/L Glucose+160 mL/L Glutamax-1™+83 mL/L Yeastolate+33 mg/L rHuInsulin) was added. At 168 hours (day 7), 10.8 L of Feed #2 (2×CD-CHO+33 g/L Glucose+80 mL/L Glutamax-1™+167 mL/L Yeastolate+0.92 g/L Sodium Butyrate) was added, and culture temperature was changed to 36.5° C. At 216 hours (day 9), 10.8 L of Feed #3 (1× CD-CHO+50 g/L Glucose+50 mL/L Glutamax-1™+250 mL/L Yeastolate+1.80 g/L Sodium Butyrate) was added, and culture temperature was changed to 36° C. At 264 hours (day 11), 10.8 L of Feed #4 (1×CD-CHO+33 g/L Glucose+33 mL/L Glutamax-1™+250 mL/L Yeastolate+0.92 g/L Sodium Butyrate) was added, and culture temperature was changed to 35.5° C. The addition of the feed media was observed to dramatically enhance the production of soluble rHuPH20 in the final stages of production. The reactor was harvested at 14 or 15 days or when the viability of the cells dropped below 40%. The process resulted in a final productivity of 17,000 Units per ml with a maximal cell density of 12 million cells/mL. At harvest, the culture was sampled for mycoplasma, bioburden, endotoxin and viral in vitro and in vivo, Transmission Electron Microscopy (TEM) and enzyme activity.

The culture was pumped by a peristaltic pump through four Millistak filtration system modules (Millipore) in parallel, each containing a layer of diatomaceous earth graded to 4-8 μm and a layer of diatomaceous earth graded to 1.4-1.1 μm, followed by a cellulose membrane, then through a second single Millistak filtration system (Millipore) containing a layer of diatomaceous earth graded to 0.4-0.11 μm and a layer of diatomaceous earth graded to <0.1 μm, followed by a cellulose membrane, and then through a 0.22 μm final filter into a sterile single use flexible bag with a 350 L capacity. The harvested cell culture fluid was supplemented with 10 mM EDTA and 10 mM Tris to a pH of 7.5. The culture was concentrated 10× with a tangential flow filtration (TFF) apparatus using four Sartoslice TFF 30 kDa molecular weight cut-off (MWCO) polyether sulfone (PES) filter (Sartorious), followed by a 10×buffer exchange with 10 mM Tris, 20 mM Na₂SO₄, pH 7.5 into a 0.22 μm final filter into a 50 L sterile storage bag.

The concentrated, diafiltered harvest was inactivated for virus. Prior to viral inactivation, a solution of 10% Triton X-100, 3% tri (n-butyl) phosphate (TNBP) was prepared. The concentrated, diafiltered harvest was exposed to 1% Triton X-100, 0.3% TNBP for 1 hour in a 36 L glass reaction vessel immediately prior to purification on the Q column.

### B. Purification of Gen2 Soluble rHuPH20

A Q Sepharose (Pharmacia) ion exchange column (9 L resin, H=29 cm, D=20 cm) was prepared. Wash samples were collected for a determination of pH, conductivity and endotoxin (LAL) assay. The column was equilibrated with 5 column volumes of 10 mM Tris, 20 mM Na₂SO₄, pH 7.5. Following viral inactivation, the concentrated, diafiltered harvest was loaded onto the Q column at a flow rate of 100 cm/hr. The column was washed with 5 column volumes of 10 mM Tris, 20 mM Na₂SO₄, pH 7.5 and 10 mM Hepes, 50 mM NaCl, pH 7.0. The protein was eluted with 10 mM Hepes, 400 mM NaCl, pH 7.0 into a 0.22 μm final filter into sterile bag. The eluate sample was tested for bioburden, protein concentration and hyaluronidase activity. A₂₈₀ absorbance reading were taken at the beginning and end of the exchange.

Phenyl-Sepharose (Pharmacia) hydrophobic interaction chromatography was next performed. A Phenyl-Sepharose (PS) column (19-21 L resin, H=29 cm, D=30 cm) was prepared. The wash was collected and sampled for pH, conductivity and endotoxin (LAL assay). The column was equilibrated with 5 column volumes of 5 mM potassium phosphate, 0.5 M ammonium sulfate, 0.1 mM CaCl2, pH 7.0. The protein eluate from the Q sepharose column was supplemented with 2M ammonium sulfate, 1 M potassium phosphate and 1 M CaCl₂ stock solutions to yield final concentrations of 5 mM, 0.5 M and 0.1 mM, respectively. The protein was loaded onto the PS column at a flow rate of 100 cm/hr and the column flow thru collected. The column was washed with 5 mM potassium phosphate, 0.5 M ammonium sulfate and 0.1 mM CaCl2 pH 7.0 at 100 cm/hr and the wash was added to the collected flow thru. Combined with the column wash, the flow through was passed through a 0.22 μm final filter into a sterile bag. The flow through was sampled for bioburden, protein concentration and enzyme activity.

An aminophenyl boronate column (ProMedics) was prepared. The wash was collected and sampled for pH, conductivity and endotoxin (LAL assay). The column was equilibrated with 5 column volumes of 5 mM potassium phosphate, 0.5 M ammonium sulfate. The PS flow through containing purified protein was loaded onto the aminophenyl boronate column at a flow rate of 100 cm/hr. The column was washed with 5 mM potassium phosphate, 0.5 M ammonium sulfate, pH 7.0. The column was washed with 20 mM bicine, 0.5 M ammonium sulfate, pH 9.0. The column was washed with 20 mM bicine, 100 mM sodium chloride, pH 9.0. The protein was eluted with 50 mM Hepes, 100 mM NaCl, pH 6.9 and passed through a sterile filter into a sterile bag. The eluted sample was tested for bioburden, protein concentration and enzyme activity.

The hydroxyapatite (HAP) column (Biorad) was prepared. The wash was collected and test for pH, conductivity and endotoxin (LAL assay). The column was equilibrated with 5 mM potassium phosphate, 100 mM NaCl, 0.1 mM CaCl₂, pH 7.0. The aminophenyl boronate purified protein was supplemented to final concentrations of 5 mM potassium phosphate and 0.1 mM CaCl₂ and loaded onto the HAP column at a flow rate of 100 cm/hr. The column was washed with 5 mM potassium phosphate, pH 7, 100 mM NaCl, 0.1 mM CaCl₂. The column was next washed with 10 mM potassium phosphate, pH 7, 100 mM NaCl, 0.1 mM CaCl₂. The protein was eluted with 70 mM potassium phosphate, pH 7.0 and passed through a 0.22 μm sterile filter into a sterile bag. The eluted sample was tested for bioburden, protein concentration and enzyme activity.

The HAP purified protein was then passed through a viral removal filter. The sterilized Viosart filter (Sartorius) was first prepared by washing with 2 L of 70 mM potassium phosphate, pH 7.0. Before use, the filtered buffer was sampled for pH and conductivity. The HAP purified protein was pumped via a peristaltic pump through the 20 nM viral removal filter. The filtered protein in 70 mM potassium phosphate, pH 7.0 was passed through a 0.22 μm final filter into a sterile bag. The viral filtered sample was tested for protein concentration, enzyme activity, oligosaccharide, monosaccharide and sialic acid profiling. The sample also was tested for process related impurities.

The protein in the filtrate was then concentrated to 10 mg/mL using a 10 kD molecular weight cut off (MWCO) Sartocon Slice tangential flow filtration (TFF) system (Sartorius). The filter was first prepared by washing with 10 mM histidine, 130 mM NaCl, pH 6.0 and the permeate was sampled for pH and conductivity. Following concentration, the concentrated protein was sampled and tested for protein concentration and enzyme activity. A 6× buffer exchange was performed on the concentrated protein into the final buffer: 10 mM histidine, 130 mM NaCl, pH 6.0. Following buffer exchange, the concentrated protein was passed though a 0.22 μm filter into a 20 L sterile storage bag. The protein was sampled and tested for protein concentration, enzyme activity, free sulflhydryl groups, oligosaccharide profiling and osmolarity.

The sterile filtered bulk protein was then aseptically dispensed at 20 mL into 30 mL sterile Teflon vials (Nalgene). The vials were then flash frozen and stored at −20±5° C.

### C. Comparison of Production and Purification of Gen1 Soluble rHuPH20 and Gen2 Soluble rHuPH20

The production and purification of Gen2 soluble rHuPH20 in a 300 L bioreactor cell culture contained some changes in the protocols compared to the production and purification Gen1 soluble rHuPH20 in a 100 L bioreactor cell culture (described in Example 6B). Table 32 sets forth exemplary differences, in addition to simple scale up changes, between the methods.

TABLE 32
Comparison of Gen1 soluble rHuPH20 and Gen2 soluble rHuPH20

| Process Difference | Gen1 soluble rHuPH20 | Gen2 soluble rHuPH20 |
|---|---|---|
| Cell line | 3D35M | 2B2 |
| Media used to expand cell | Contains 0.10 μM methotrexate | Contains 20 μM methotrexate (9 mg/L) |
| inoculum | (0.045 mg/L) | |
| Media in 6 L cultures | Contains 0.10 μM methotrexate | Contains no methotrexate |
| onwards | | |
| 36 L spinner flask | No instrumentation | Equipped with instrumentation that |
| | | monitors and controls pH, dissolved |
| | | oxygen, sparge and overlay gas flow |
| | | rate. |
| | 20 L operating volume. | 32 L operating volume |
| Final operating volume in | Approx. 100 L in a 125 L | Approx. 300 L in a 400 L bioreactor |
| bioreactor | bioreactor (initial culture volume + | (initial culture volume + 260 L) |
| | 65 L) | |
| Culture media in final | No rHuInsulin | 5.0 mg/L rHuInsulin |
| bioreactor | | |
| Media feed volume | Scaled at 4% of the bioreactor | Scaled at 4% of the bioreactor cell |
| | cell culture volume i.e. 3.4, 3.5 | culture volume i.e. 10.4, 10.8, 11.2 |
| | and 3.7 L, resulting in a target | and 11.7 L, resulting in a target |
| | bioreactor volume of ~92 L. | bioreactor volume of ~303 L. |
| Media feed | Feed #1 Medium: CD CHO + 50 g/L | Feed #1 Medium: 4x CD CHO + 33 g/L |
| | Glucose + 8 mM | Glucose + 32 mM Glutamax ™ + |
| | GlutaMAX ™-1 | 16.6 g/L Yeastolate + 33 mg/L |
| | | rHuInsulin |
| | Feed #2 (CD CHO + 50 g/L | Feed #2: 2x CD CHO + 33 g/L |
| | Glucose + 8 mM GlutaMAX + | Glucose + 16 mM Glutamax + 33.4 g/L |
| | 1.1 g/L Sodium Butyrate | Yeastolate + 0.92 g/L Sodium |
| | | Butyrate |
| | Feed #3: CD CHO + 50 g/L | Feed #3: 1x CD CHO + 50 g/L |
| | Glucose + 8 mM GlutaMAX + | Glucose + 10 mM Glutamax + 50 g/L |
| | 1.1 g/L Sodium Butyrate | Yeastolate + 1.80 g/L Sodium |
| | | Butyrate |
| | | Feed #4: 1x CD CHO + 33 g/L |
| | | Glucose + 6.6 mM Glutamax + 50 g/L |
| | | Yeastolate + 0.92 g/L Sodium |
| | | Butyrate |
| Filtration of bioreactor | Four polyethersulfone filters (8.0 μm, | 1^{st} stage - Four modules in parallel, |
| cell culture | 0.65 μm, 0.22 μm and 0.22 μm) | each with a layer of diatomaceous |
| | in series | earth graded to 4-8 μm and a layer |
| | | of diatomaceous earth graded to 1.4-1.1 μm, |
| | | followed by a cellulose |
| | | membrane. |
| | | 2^{nd} stage - single module containing a |
| | | layer of diatomaceous earth graded |
| | | to 0.4-0.11 μm and a layer of |
| | | diatomaceous earth graded to <0.1 μm, |
| | | followed by a cellulose |
| | | membrane. |
| | | 3^{rd} stage - 0.22 μm polyethersulfone |
| | | filter |
| | 100 L storage bag | 300 L storage bag |
| | | Harvested cell culture is |
| | | supplemented with 10 mM EDTA, |
| | | 10 mM Tris to a pH of 7.5. |
| Concentration and buffer | Concentrate with 2 TFF with | Concentrate using four Sartorius |
| exchange prior to | Millipore Spiral | Sartoslice TFF 30K MWCO Filter |
| chromatography | Polyethersulfone 30K MWCO | |
| | Filter | |
| | Buffer Exchange the | Buffer Exchange the Concentrate |
| | Concentrate 6x with 10 mM | 10x with 10 mM Tris, 20 mM |
| | Hepes, 25 mM NaCl, pH 7.0 | Na2SO4, pH 7.5 |
| | 20 L sterile storage bag | 50 L sterile storage bag |
| Viral inactivation prior to | None | Viral inactivation performed with |
| chromatography | | the addition of a 1% Triton X-100, |
| | | 0.3% Tributyl Phosphate, pH 7.5, |
| 1^{st} purification step (Q | No absorbance reading | A280 measurements at the beginning |
| sepharose) | | and end |
| Viral filtration after | Pall DV-20 filter (20 nm) | Sartorius Virosart filter (20 nm) |
| chromatography | | |
| Concentration and buffer | Hepes/saline pH 7.0 buffer | Histidine/saline, pH 6.0 buffer |
| exchange after | Protein concentrated to 1 mg/ml | Protein concentrated to 10 mg/ml |
| chromatography | | |

### Example 9

### Determination of Sialic Acid and Monosaccharide Content

The sialic acid and monosaccharide content of soluble rHuPH20 can be assessed by reverse phase liquid chromatography (RPLC) following hydrolysis with trifluoroacetic acid. In one example, the sialic acid and monosaccharide content of purified hyaluronidase lot #HUB0701E (1.2 mg/mL; produced and purified essentially as described in Example 8) was determined. Briefly, 100 μg sample was hydrolyzed with 40% (v/v) trifluoroacetic acid at 100° C. for 4 hours in duplicate. Following hydrolysis, the samples were dried down and resuspended in 300 μL water. A 45 μL aliquot from each re-suspended sample was transferred to a new tube and dried down, and 10 μL of a 10 mg/mL sodium acetate solution was added to each. The released monosaccharides were fluorescently labeled by the addition of 50 μL of a solution containing 30 mg/mL 2-aminobenzoic acid, 20 mg/mL sodium cyanoborohydride, approximately 40 mg/mL sodium acetate and 20 mg/mL boric acid in methanol. The mixture was incubated for 30 minutes at 80° C. in the dark. The derivatization reaction was quenched by the addition of 440 μL of mobile phase A (0.2% (v/v) n-butylamine, 0.5% (v/v) phosphoric acid, 1% (v/v) tetrahydrofuran). A matrix blank of water also was hydrolyzed and derivatized as described for the hyaluronidase sample as a negative control. The released monosaccharides were separated by RPLC using an Octadecyl (C₁₈) reverse phase column (4.6×250 mm, 5 μm particle size; J. T. Baker) and monitored by fluorescence detection (360 nm excitation, 425 nm emission). Quantitation of the monosaccharide content was made by comparison of the chromatograms from the hyaluronidase sample with chromatograms of monosaccharide standards including N-D-glucosamine (GlcN), N-D-galactosamine (GalN), galactose, fucose and mannose. Table 33 presents the molar ratio of each monosaccharide per hyaluronidase molecule.

TABLE 33
Monosaccharide content of soluble rHuPH20

| Lot | Replicate | GlcN | GalN | Galactose | Mannose | Fucose |
|---|---|---|---|---|---|---|
| HUB0701E | 1 | 14.28 | 0.07* | 6.19 | 25.28 | 2.69 |
| | 2 | 13.66 | 0.08* | 6.00 | 24.34 | 2.61 |
| | Average | 13.97 | 0.08* | 6.10 | 24.81 | 2.65 |

*GalN results were below the limit of detection

### Example 10

### C-terminal Heterogeneity of Soluble rHuPH20 from 3D35M and 2B2 Cells

C-terminal sequencing was performed on two lots of sHuPH20 produced and purified from 3D35M cells in a 100 L bioreactor volume (Lot HUA0505MA) and 2B2 cells in a 300 L bioreactor volume (Lot HUB0701EB). The lots were separately digested with endoproteinase Asp-N, which specifically cleaves peptide bonds N-terminally at aspartic and cysteic acid. This releases the C-terminal portion of the soluble rHuPH20 at the aspartic acid at position 431 of SEQ ID NO:4. The C-terminal fragments were separated and characterized to determine the sequence and abundance of each population in Lot HUA0505MA and Lot HUB0701EB.

It was observed that the soluble rHuPH20 preparations from 3D35M cells and 2B2 cells displayed heterogeneity, and contained polypeptides that differed from one another in their C-terminal sequence (Tables 34 and 35). This heterogeneity is likely the result of C-terminal cleavage of the expressed 447 amino acid polypeptide (SEQ ID NO:4) by peptidases present in the cell culture medium or other solutions during the production and purification process. The polypeptides in the soluble rHuPH20 preparations have amino acid sequences corresponding to amino acids 1-447, 1-446, 1-445, 1-444 and 1-443 of the soluble rHuPH20 sequence set forth SEQ ID NO:4. The full amino acid sequence of each of these polypeptides is forth in SEQ ID NOS: 4 to 8, respectively. As noted in tables 33 and 34, the abundance of each polypeptide in the soluble rHuPH20 preparations from 3D35M cells and 2B2 cells differs.

TABLE 34
Analysis of C-terminal fragments from Lot HUA0505MA

| | | | | | | | |
|---|---|---|---|---|---|---|---|
| | Amino | | | | | | |
| | acid | | | | | | |
| | position | | | | | | |
| | (relative | | | | | | |
| | to SEQ | | Theor. | Exp. | | Elution | |
| Fragment | ID NO: 4) | Sequence | Mass | Mass | Error | time | Abundance |

| | | | | | | | |
|---|---|---|---|---|---|---|---|
| D28a | 431-447 | DAFKLPPMETEEPQIFY | 2053.97 | 2054.42 | 0.45 | 99.87 | 0.2% |
| | | (SEQ ID NO: 57) | | | | | |
| D28b | 431-446 | DAFKLPPMETEEPQIF | 1890.91 | 1891.28 | 0.37 | 97.02 | 18.4% |
| | | (SEQ ID NO: 58) | | | | | |
| D28c | 431-445 | DAFKLPPMETEEPQI | 1743.84 | 1744.17 | 0.33 | 86.4 | 11.8% |
| | | (SEQ ID NO: 59) | | | | | |
| D28d | 431-444 | DAFKLPPMETEEPQ | 1630.70 | 1631.07 | 0.32 | 74.15 | 56.1% |
| | | (SEQ ID NO: 60) | | | | | |
| D28e | 431-443 | DAFKLPPMETEEP | 1502.70 | 1502.98 | 0.28 | 77.36 | 13.6% |
| | | (SEQ ID NO: 61) | | | | | |
| D28f | 431-442 | DAFKLPPMETEE | 1405.64 | ND | N/A | N/A | 0.0% |
| | | (SEQ ID NO: 62) | | | | | |

TABLE 35
Analysis of C-terminal fragments from Lot HUB0701EB

| | Amino | | | | | | |
|---|---|---|---|---|---|---|---|
| | acid | | | | | | |
| | position | | | | | | |
| | (relative | | | | | | |
| | to SEQ | | | | | | |
| | ID NO: | | Theor. | Exp. | | Elution | |
| Fragment | 4) | Sequence | Mass | Mass | Error | time | Abundance |

| | | | | | | | |
|---|---|---|---|---|---|---|---|
| D28a | 431-477 | DAFKLPPMETEEPQIFY | 2053.97 | 2054.42 | 0.45 | 99.89 | 1.9% |
| | | (SEQ ID NO: 57) | | | | | |
| D28b | 431-446 | DAFKLPPMETEEPQIF | 1890.91 | 1891.36 | 0.45 | 96.92 | 46.7% |
| | | (SEQ ID NO: 58) | | | | | |
| D28c | 431-445 | DAFKLPPMETEEPQI | 1743.84 | 1744.24 | 0.40 | 85.98 | 16.7% |
| | | (SEQ ID NO: 59) | | | | | |
| D28d | 431-444 | DAFKLPPMETEEPQ | 1630.70 | 1631.14 | 0.39 | 73.9 | 27.8% |
| | | (SEQ ID NO: 60) | | | | | |
| D28e | 431-443 | DAFKLPPMETEEP | 1502.70 | 1503.03 | 0.33 | 77.02 | 6.9% |
| | | (SEQ ID NO: 61) | | | | | |
| D28f | 431-442 | DAFKLPPMETEE | 1405.64 | ND | N/A | N/A | 0.0% |
| | | (SEQ ID NO: 62) | | | | | |

### Example 11

### Phase 1-Stage 1 Clinical Study in Human Subjects Pharmacokinetic Analysis of Subcutaneous (SC) Co-administration of Human Recombinant Hyaluronidase PH20 (rHuPH20) and Zoledronic Acid

A pharmacokinetic study of zoledronic acid (ZA) co-administered with rHuPH20 in human subjects was conducted. The primary objective of the study was to determine the bioavailability of zoledronic acid dosed via subcutaneous (SC) co-administration with human recombinant hyaluronidase PH20 (rHuPH20).

A. Preparation of rHuPH20/ZA dosing solutions

For Stage 1 of the pharmacokinetic study, dosing solutions were prepared, which contained rHuPH20 (prepared from lots HUA0601 MA, HUA0702MA and HUA0703MA; Halozyme Therapeutics) and commercially prepared zoledronic acid (Zometa®, 0.8 mg/mL ZA; Novartis).

rHuPH20 was prepared from DG44 CHO cells (2B2, see Example 4) according to the Gen1 production method described in Example 6. As described in Example 6, no biologically sourced material was used in the manufacturing process. The media used for growth of the CHO cells is chemically defined (i.e. contains no bovine serum albumin or trypsin). No antibodies or enzymes were employed in the purification process. The specification endpoint parameters for rHuPH20 production were 0.08 to 0.12 mg/ml rHuPH20 with an enzyme specific activity of 9,000 to 15,000 units/ml. Enzymatic activity was determined by microturbidity assay as described in Example 5.

Zometa® Injection (Novartis) is provided as a sterile liquid concentrate solution in which each 5 mL of liquid contains 4.264 mg of zoledronic acid monohydrate, which corresponds to 4 mg zoledronic acid on an anhydrous basis. A vial of Zometa® delivers 4 mg of anhydrous ZA along with additional inactive ingredients including 220 mg mannitol (USP) and 24 mg sodium citrate (USP) per vial. The stock solution of ZA (0.8 mg/ml) was diluted into the dosing solutions as described below before clinical administration as a subcutaneous infusion.

The dosing solutions contained varying amounts of rHuPH20 and ZA as summarized in Table 36. The dosing formulations were designed to neutralize the citrate excipients in the Zometa® product and to account for volume constraints in dosing solutions due to the limit of 0.8 mg/mL ZA present in the Zometa® product.

TABLE 36
Content of Stage 1 Dosing Solutions

| | | | | | Drug |
|---|---|---|---|---|---|
| | | | | | Product |
| | | | | Drug | Volume |
| | ZA (mg) | rHuPH20 | HSA (mg) | Product | (mL) |
| Cohort | in dose | (U) in dose^{†} | in dose | dose (mL) | prepared |
| 1 | 0.25 | 24,000 | 100 | 20* | 25 |
| 2 | 0.5 | 24,000 | 100 | 20* | 25 |
| 3 | 1 | 24,000 | 100 | 20* | 25 |
| 4 | 2 | 24,000 | 100 | 20* | 25 |
| 5 | 5 | 24,000 | 100 | 20* | 25 |
| 6 | 5 | 12,000 | 50 | 20** | 25 |
| 7 | 5 | 6,000 | 25 | 25 | 28 |
| 8 | 5 | 3,000 | 12.5 | 12.5 | 15 |
| 9 | 5 | 1,704 | 7.1 | 7.1 | 10 |
| 10 | 5 | 2,400 | 10 | 10 | 15 |

*Drug product diluted to 100 mL with saline before infusion
**Drug product diluted to 50 mL with saline before injection
^{†}HUA lots employed: HUA0601MA, HUA0702MA and HUA0703MA
HSA = Human Serum albumin

Stock solutions for were first prepared for each of the dosing groups. The composition of each of the stock solutions is summarized in Table 37.

TABLE 37
Composition of Stage 1 Dosing Stock Solutions

| | Prod. Vol. | ZA | HSA | rHuPH20 |
|---|---|---|---|---|
| Cohort | (ml) | (ug/ml) | (mg/ml) | (ug/ml) |
| 1 | 25 | 12.5 | 5 | 10 |
| 2 | 25 | 25 | 5 | 10 |
| 3 | 25 | 50 | 5 | 10 |
| 4 | 25 | 100 | 5 | 10 |
| 5 | 25 | 250 | 5 | 10 |
| 6 | 25 | 250 | 2.5 | 5 |
| 7 | 28 | 200 | 1 | 2 |
| 8 | 15 | 400 | 1 | 2 |
| 9 | 10 | 704 | 1 | 2 |
| 10 | 15 | 500 | 1 | 2 |

Table 38 describes the formulation instructions for Stage 1 dosing stock solutions. rHuPH20 volume additions were based upon the 0.1 mg/ml targeted concentration for the rHuPH20 solution. Given the limited accuracy of pharmacy volume measures (about 0.1 ml increments using syringes), the dose formulations were not adjusted for individual lots of rHuPH20 having nominal differences in concentration. The rHuPH20 solution is a frozen stock, pH 6.5, in 10 mM Histidine and 130 mM sodium chloride.

Each clinical dose was first prepared within a 30 cc vial containing 1 ml of excipients (300 mM NaPO₄ buffer, pH 7.2; 10 mg/ml of human serum albumin). Each component of the dosing solution was added in the order of manufacturing steps depicted in Table 38.

TABLE 38
Process for Generating Stage 1 Dosing Stock Solutions

| Manuf. | | | | | | | | | | | |
|---|---|---|---|---|---|---|---|---|---|---|---|
| steps | Reagent Added | 1 | 2 | 3 | 4 | 5 | 6 | 7 | 8 | 9 | 10 |
| 1 | Excipient Salts | 1 | 1 | 1 | 1 | 1 | 1 | 1 | 1 | 1 | 1 |
| | (ml) | | | | | | | | | | |
| 2 | Saline | 21 | 20.6 | 19.8 | 18.3 | 13.6 | 15.1 | 19 | 6.1 | 0 | 4.2 |
| | solution (ml) | | | | | | | | | | |
| 3 | Zometa ® | 0.1 | 0.8 | 1.6 | 3.1 | 7.8 | 7.8 | 7 | 7.5 | 8.8 | 9.4 |
| | solution (ml) | | | | | | | | | | |
| 4 | HSA solution | 2.3 | 2.3 | 2.3 | 2.3 | 2.3 | 1.0 | 0.4 | 0.1 | 0 | 0.1 |
| | (50 mg/ml; 5% | | | | | | | | | | |
| | USP) | | | | | | | | | | |
| 5 | rHuPH20 | 0.3 | 0.3 | 0.3 | 0.3 | 0.3 | 0.15 | 0.6 | 0.3 | 0.2 | 0.3 |
| | Solution (HUA, | | | | | | | | | | |
| | 1 mg/ml) | | | | | | | | | | |
| | Final Volume | 25 | 25 | 25 | 25 | 25 | 25 | 25 | 25 | 25 | 25 |
| | (ml) | | | | | | | | | | |

For cohorts 7-10 the rHUPH20 was prediluted 1/9 in albumin/buffer/saline solution prior to addition.

For administration to the human subjects, the dosing stock solutions were diluted as required (see Table 39). For cohorts 1-5, 20 ml of the dosing solution was mixed with 80 ml saline to generate the 100 ml dose. For cohort 6, 20 ml of the dosing solution is mixed with 30 ml saline. Cohort dosing solutions 7-10 were not diluted.

TABLE 39
Preparation of Stage 1 Dosing Solutions for Administration and
Amounts of Zoledronate and PH20 Administered to Each Subject

| | Dilution | | Vol. | ZA | [ZA] | [HSA] | [PH20] |
|---|---|---|---|---|---|---|---|
| | Req. | Deliv. | Admin. | Admin. | Admin. | Admin. | Admin. |
| ID # | (Fold) | Mode | (ml) | (mg) | (ug/ml) | (ug/ml) | (ug/ml) |
| 1 | 5 | Infusion | 100 | 0.25 | 2.5 | 1 | 2 |
| | | Bag | | | | | |
| 2 | 5 | Infusion | 100 | 0.5 | 50 | 1 | 2 |
| | | Bag | | | | | |
| 3 | 5 | Infusion | 100 | 1 | 10 | 1 | 2 |
| | | Bag | | | | | |
| 4 | 5 | Infusion | 100 | 2 | 20 | 1 | 2 |
| | | Bag | | | | | |
| 5 | 5 | Infusion | 100 | 5 | 50 | 1 | 2 |
| | | Bag | | | | | |
| 6 | 2.5 | Syringe | 50 | 5 | 100 | 1 | 2 |
| 7 | None | Syringe | 25 | 5 | 200 | 1 | 2 |
| 8 | None | Syringe | 12.5 | 5 | 400 | 1 | 2 |
| 9 | None | Syringe | 7.1 | 5 | 704 | 1 | 2 |
| 10 | None | Syringe | 10 | 5 | 500 | 1 | 2 |

B. Methods of Treatment and Pharmacokinetic Analysis of SC Co-Administration of rHuPH20 and Zoledronic Acid

Human patients were assigned to ten treatment groups (Cohorts 1-10; three subjects per cohort) as summarized in Table 36. The patients were administered the prescribed dosage formulations as follows: Cohorts 1-5 were administered a single subcutaneous infusion in a dosage volume of 100 ml at a rate of 3 mL/min. As described above, the subjects in cohorts 1-5 were administered ZA at a dose of 0.25 mg (Cohort 1), 0.5 mg (Cohort 2), 1 mg (Cohort 3), 2 mg (Cohort 4), or 4 mg (Cohort 5) with 24000 Units of rHuPH20. The dosing solution is administered via needle or catheter into the SC space of the left anterior thigh (midway between the anterior iliac crest and the cephalad border of the patella). For cohorts 1-5, the dosing solutions were administered by a controlled rate infusion pump at approximately 3 mL/minute.

Cohorts 6, 7, 8, 9, and 10 were administered a total dosage volume of 50 ml, 25 ml, 15.5 ml, 7.1 ml, and 10 ml, respectively, subcutaneously via syringe (see Table 39). The dosing solutions were administered by syringe push bolus injection.

Following rHuPH20/ZA dose administration, serial blood samples (2.5 ml) were collected from the patients for serum preparation and determination of ZA concentrations. Blood collection times were at 0 minutes, 10 minutes, 15 minutes, 30 minutes, 45 minutes, 1 hour, 2 hours, 4 hours, 6 hours, 8 hours, 12 hours, 24 hours, and 48 hours post administration of the rHuPH20/ZA dose formulation. The samples were centrifuged at approximately 1800 rpm for 10 minute at 4° C. and stored at −20° C. until analysis of ZA concentrations.

Serum concentration of ZA was determined using a qualified LC-MS-MS assay after derivatization with diazomethane (Zhu, L S et al. (2006) Rapid Commun. Mass Spectrom. 20: 3421-3426). The assay range was from 2.0 to 400 ng/mL with a lower limit of quantitation (LLOQ) at 2.0 ng/mL.

Pharmacokinetic (PK) analyses of ZA serum concentration versus time data were conducted using WinNonlin v5.1 (Pharsight, Mountain View, Calif.). Non-compartmental analysis was used for derivation of primary and secondary pharmacokinetic (PK) parameters. Serum ZA concentrations reported as below the assay's lower limit of quantitation (LLOQ=0.5 ng/mL) were assigned a value of zero prior to PK analysis. Correlations of exposure parameters (Cmax and AUC) with ZA dose were conducted using JMP version 8.0. Summary statistics (mean, standard deviation, and median) were calculated using JMP or Microsoft Excel.

### Results

Zoledronic acid (ZA) concentration data from serum samples obtained in Stage 1 of clinical study were analyzed to determine the pharmacokinetics of ZA administered subcutaneously with co-administration of PH20, Serum ZA concentration versus time data from individual subjects in Cohorts 1 to 5 are listed in Table 40. Mean (±S.D.) and median ZA concentration data are presented in Table 41. Plots of mean PK profiles, median PK profiles, and individual concentration-time profiles were prepared. Derived PK parameters for individual subjects along with summary statistics are shown in Tables 42-44. Correlations of Cmax with ZA dosage, AUC [AUC(0-∞) with ZA dosage, and AUC(0-12h)) with ZA dosage using bivariate fit plot analysis established linear pharmacokinetics over a dose range of 0.25 to 5 mg ZA with co-administration of 24000 Units rHuPH20. Using the data obtained in the experiment, an estimation of subcutaneous absorption time for ZA with 2400 Units rHuPH20 also was calculated (mean absorption time=1.64 hours; see Table 45).

TABLE 40
Serum Zoledronic Acid Concentrations versus Time Data

| | | | | Nominal | |
|---|---|---|---|---|---|
| | | | | Time | |
| | | | | Post | Zoledronic Acid |
| ZA Dose | Body Wt. | Subject | | Infusion | Concentration |
| (mg) | (Kg) | ID | Period | (h) | (ng/mL) |
| 0.25 | 56.2 | 1 | 1 | 0 | 5.04 |
| | | 1 | 1 | 0.1667 | 7.43 |
| | | 1 | 1 | 0.25 | 7.23 |
| | | 1 | 1 | 0.5 | 7.75 |
| | | 1 | 1 | 0.75 | 7.20 |
| | | 1 | 1 | 1 | 7.25 |
| | | 1 | 1 | 2 | 5.42 |
| | | 1 | 1 | 4 | 2.56 |
| | | 1 | 1 | 6 | 1.20 |
| | | 1 | 1 | 8 | 0.56 |
| | | 1 | 1 | 12 | BLQ < (0.500) |
| | | 1 | 1 | 24 | BLQ < (0.500) |
| | | 1 | 1 | 48 | BLQ < (0.500) |
| 0.25 | 76.4 | 8 | 1 | 0 | 4.50 |
| | | 8 | 1 | 0.1667 | 4.97 |
| | | 8 | 1 | 0.25 | 5.15 |
| | | 8 | 1 | 0.5 | 5.17 |
| | | 8 | 1 | 0.75 | 5.28 |
| | | 8 | 1 | 1 | 5.67 |
| | | 8 | 1 | 2 | 4.33 |
| | | 8 | 1 | 4 | 3.45 |
| | | 8 | 1 | 6 | 2.02 |
| | | 8 | 1 | 8 | 1.11 |
| | | 8 | 1 | 12 | BLQ < (0.500) |
| | | 8 | 1 | 24 | BLQ < (0.500) |
| | | 8 | 1 | 48 | BLQ < (0.500) |
| 0.25 | 75.2 | 11 | 1 | 0 | 4.69 |
| | | 11 | 1 | 0.1667 | 4.87 |
| | | 11 | 1 | 0.25 | 6.39 |
| | | 11 | 1 | 0.5 | 6.25 |
| | | 11 | 1 | 0.75 | 6.84 |
| | | 11 | 1 | 1 | 6.60 |
| | | 11 | 1 | 2 | 5.44 |
| | | 11 | 1 | 4 | 3.06 |
| | | 11 | 1 | 6 | 1.35 |
| | | 11 | 1 | 8 | 0.79 |
| | | 11 | 1 | 12 | BLQ < (0.500) |
| | | 11 | 1 | 24 | BLQ < (0.500) |
| | | 11 | 1 | 48 | BLQ < (0.500) |
| 0.5 | 56.7 | 16 | 1 | 0 | 10.80 |
| | | 16 | 1 | 0.1667 | 15.00 |
| | | 16 | 1 | 0.25 | 15.30 |
| | | 16 | 1 | 0.5 | 13.80 |
| | | 16 | 1 | 0.75 | 14.70 |
| | | 16 | 1 | 1 | 14.10 |
| | | 16 | 1 | 2 | 11.10 |
| | | 16 | 1 | 4 | 5.27 |
| | | 16 | 1 | 6 | 2.50 |
| | | 16 | 1 | 8 | 1.19 |
| | | 16 | 1 | 12 | 0.52 |
| | | 16 | 1 | 24 | BLQ < (0.500) |
| | | 16 | 1 | 48 | BLQ < (0.500) |
| 0.5 | 56.1 | 18 | 1 | 0 | 13.60 |
| | | 18 | 1 | 0.1667 | 16.80 |
| | | 18 | 1 | 0.25 | 16.30 |
| | | 18 | 1 | 0.5 | 16.50 |
| | | 18 | 1 | 0.75 | 17.10 |
| | | 18 | 1 | 1 | 15.00 |
| | | 18 | 1 | 2 | 10.60 |
| | | 18 | 1 | 4 | 6.02 |
| | | 18 | 1 | 6 | 3.04 |
| | | 18 | 1 | 8 | 1.34 |
| | | 18 | 1 | 12 | 0.55 |
| | | 18 | 1 | 24 | BLQ < (0.500) |
| | | 18 | 1 | 48 | BLQ < (0.500) |
| 0.5 | 75.2 | 21 | 1 | 0 | 11.10 |
| | | 21 | 1 | 0.1667 | 13.30 |
| | | 21 | 1 | 0.25 | 13.70 |
| | | 21 | 1 | 0.5 | 13.00 |
| | | 21 | 1 | 0.75 | 14.20 |
| | | 21 | 1 | 1 | 13.70 |
| | | 21 | 1 | 2 | 11.50 |
| | | 21 | 1 | 4 | 5.87 |
| | | 21 | 1 | 6 | 2.53 |
| | | 21 | 1 | 8 | 1.42 |
| | | 21 | 1 | 12 | 0.56 |
| | | 21 | 1 | 24 | BLQ < (0.500) |
| | | 21 | 1 | 48 | BLQ < (0.500) |
| 1 | 57.7 | 25 | 1 | 0 | 17.90 |
| | | 25 | 1 | 0.1667 | 24.50 |
| | | 25 | 1 | 0.25 | 23.40 |
| | | 25 | 1 | 0.5 | 28.00 |
| | | 25 | 1 | 0.75 | 30.10 |
| | | 25 | 1 | 1 | 27.20 |
| | | 25 | 1 | 2 | 17.70 |
| | | 25 | 1 | 4 | 7.51 |
| | | 25 | 1 | 6 | 3.76 |
| | | 25 | 1 | 8 | 2.46 |
| | | 25 | 1 | 12 | 0.96 |
| | | 25 | 1 | 24 | 0.50 |
| | | 25 | 1 | 48 | BLQ < (0.500) |
| 1 | 74.1 | 26 | 1 | 0 | 28.40 |
| | | 26 | 1 | 0.1667 | 32.10 |
| | | 26 | 1 | 0.25 | 32.60 |
| | | 26 | 1 | 0.5 | 33.40 |
| | | 26 | 1 | 0.75 | 32.60 |
| | | 26 | 1 | 1 | 29.90 |
| | | 26 | 1 | 2 | 24.00 |
| | | 26 | 1 | 4 | 13.30 |
| | | 26 | 1 | 6 | 5.48 |
| | | 26 | 1 | 8 | 2.44 |
| | | 26 | 1 | 12 | 1.02 |
| | | 26 | 1 | 24 | 0.56 |
| | | 26 | 1 | 48 | BLQ < (0.500) |
| 1 | 51 | 27 | 1 | 0 | 15.30 |
| | | 27 | 1 | 0.1667 | 25.00 |
| | | 27 | 1 | 0.25 | 25.00 |
| | | 27 | 1 | 0.5 | 23.00 |
| | | 27 | 1 | 0.75 | 21.70 |
| | | 27 | 1 | 1 | 19.40 |
| | | 27 | 1 | 2 | 17.00 |
| | | 27 | 1 | 4 | 10.20 |
| | | 27 | 1 | 6 | 5.47 |
| | | 27 | 1 | 8 | 3.17 |
| | | 27 | 1 | 12 | 1.30 |
| | | 27 | 1 | 24 | 0.51 |
| | | 27 | 1 | 48 | BLQ < (0.500) |
| 2 | 60 | 29 | 1 | 0 | 33.30 |
| | | 29 | 1 | 0.1667 | 52.50 |
| | | 29 | 1 | 0.25 | 54.00 |
| | | 29 | 1 | 0.5 | 59.80 |
| | | 29 | 1 | 0.75 | 62.80 |
| | | 29 | 1 | 1 | 57.70 |
| | | 29 | 1 | 2 | 42.50 |
| | | 29 | 1 | 4 | 17.50 |
| | | 29 | 1 | 6 | 6.43 |
| | | 29 | 1 | 8 | 3.15 |
| | | 29 | 1 | 12 | 1.50 |
| | | 29 | 1 | 24 | 0.78 |
| | | 29 | 1 | 48 | BLQ < (0.500) |
| 2 | 65.9 | 31 | 1 | 0 | 39.60 |
| | | 31 | 1 | 0.1667 | 46.30 |
| | | 31 | 1 | 0.25 | 46.10 |
| | | 31 | 1 | 0.5 | 47.30 |
| | | 31 | 1 | 0.75 | 50.30 |
| | | 31 | 1 | 1 | 53.00 |
| | | 31 | 1 | 2 | 40.10 |
| | | 31 | 1 | 4 | 20.30 |
| | | 31 | 1 | 6 | 7.71 |
| | | 31 | 1 | 8 | 3.34 |
| | | 31 | 1 | 12 | 1.64 |
| | | 31 | 1 | 24 | 0.85 |
| | | 31 | 1 | 48 | BLQ < (0.500) |
| 2 | 56.2 | 32 | 1 | 0 | 40.80 |
| | | 32 | 1 | 0.1667 | 46.00 |
| | | 32 | 1 | 0.25 | 47.90 |
| | | 32 | 1 | 0.5 | 48.60 |
| | | 32 | 1 | 0.75 | 58.60 |
| | | 32 | 1 | 1 | 58.70 |
| | | 32 | 1 | 2 | 42.20 |
| | | 32 | 1 | 4 | 23.00 |
| | | 32 | 1 | 6 | 8.93 |
| | | 32 | 1 | 8 | 4.81 |
| | | 32 | 1 | 12 | 2.07 |
| | | 32 | 1 | 24 | 0.98 |
| | | 32 | 1 | 48 | 0.62 |
| 5 | 63.6 | 37 | 1 | 0 | 82.30 |
| | | 37 | 1 | 0.1667 | 121.00 |
| | | 37 | 1 | 0.25 | 131.00 |
| | | 37 | 1 | 0.5 | 130.00 |
| | | 37 | 1 | 0.75 | 126.00 |
| | | 37 | 1 | 1 | 124.00 |
| | | 37 | 1 | 2 | 83.50 |
| | | 37 | 1 | 4 | 45.60 |
| | | 37 | 1 | 6 | 20.30 |
| | | 37 | 1 | 8 | 9.44 |
| | | 37 | 1 | 12 | 3.91 |
| | | 37 | 1 | 24 | 2.15 |
| | | 37 | 1 | 48 | 1.28 |
| 5 | 70.7 | 38 | 1 | 0 | 53.50 |
| | | 38 | 1 | 0.1667 | 76.10 |
| | | 38 | 1 | 0.25 | 97.50 |
| | | 38 | 1 | 0.5 | 112.00 |
| | | 38 | 1 | 0.75 | 117.00 |
| | | 38 | 1 | 1 | 113.00 |
| | | 38 | 1 | 2 | 89.80 |
| | | 38 | 1 | 4 | 42.80 |
| | | 38 | 1 | 6 | 22.30 |
| | | 38 | 1 | 8 | 11.00 |
| | | 38 | 1 | 12 | 4.61 |
| | | 38 | 1 | 24 | 2.98 |
| | | 38 | 1 | 48 | 1.63 |
| 5 | 71.9 | 39 | 1 | 0 | 130.00 |
| | | 39 | 1 | 0.1667 | 132.00 |
| | | 39 | 1 | 0.25 | 147.00 |
| | | 39 | 1 | 0.5 | 132.00 |
| | | 39 | 1 | 0.75 | 116.00 |
| | | 39 | 1 | 1 | 116.00 |
| | | 39 | 1 | 2 | 86.50 |
| | | 39 | 1 | 4 | 61.70 |
| | | 39 | 1 | 6 | 27.80 |
| | | 39 | 1 | 8 | 12.50 |
| | | 39 | 1 | 12 | 5.00 |
| | | 39 | 1 | 24 | 2.23 |
| | | 39 | 1 | 48 | 1.74 |

BLQ = blow limit for quantitation

TABLE 41
Mean (±S.D.) and Median ZA Concentration Data

| Zoledronic | Nominal Time | | | | |
|---|---|---|---|---|---|
| Acid Dose | Post Infusion | Mean Conc. | Std. | Median Conc. | |
| (mg) | (h) | (ng/mL) | Dev. | (ng/mL) | n |
| 0.25 | 0 | 4.74 | 0.27 | 4.69 | 3 |
| 0.25 | 0.1667 | 5.76 | 1.45 | 4.97 | 3 |
| 0.25 | 0.25 | 6.26 | 1.05 | 6.39 | 3 |
| 0.25 | 0.5 | 6.39 | 1.30 | 6.25 | 3 |
| 0.25 | 0.75 | 6.44 | 1.02 | 6.84 | 3 |
| 0.25 | 1 | 6.51 | 0.79 | 6.60 | 3 |
| 0.25 | 2 | 5.06 | 0.64 | 5.42 | 3 |
| 0.25 | 4 | 3.02 | 0.45 | 3.06 | 3 |
| 0.25 | 6 | 1.52 | 0.44 | 1.35 | 3 |
| 0.25 | 8 | 0.82 | 0.27 | 0.79 | 3 |
| 0.25 | 12 | BLQ | NA | BLQ | 3 |
| 0.25 | 24 | BLQ | NA | BLQ | 3 |
| 0.25 | 48 | BLQ | NA | BLQ | 3 |
| 0.5 | 0 | 11.83 | 1.54 | 11.10 | 3 |
| 0.5 | 0.1667 | 15.03 | 1.75 | 15.00 | 3 |
| 0.5 | 0.25 | 15.10 | 1.31 | 15.30 | 3 |
| 0.5 | 0.5 | 14.43 | 1.83 | 13.80 | 3 |
| 0.5 | 0.75 | 15.33 | 1.55 | 14.70 | 3 |
| 0.5 | 1 | 14.27 | 0.67 | 14.10 | 3 |
| 0.5 | 2 | 11.07 | 0.45 | 11.10 | 3 |
| 0.5 | 4 | 5.72 | 0.40 | 5.87 | 3 |
| 0.5 | 6 | 2.69 | 0.30 | 2.53 | 3 |
| 0.5 | 8 | 1.32 | 0.12 | 1.34 | 3 |
| 0.5 | 12 | 0.54 | 0.02 | 0.55 | 3 |
| 0.5 | 24 | BLQ | NA | BLQ | 3 |
| 0.5 | 48 | BLQ | NA | BLQ | 3 |
| 1 | 0 | 20.53 | 6.94 | 17.90 | 3 |
| 1 | 0.1667 | 27.20 | 4.25 | 25.00 | 3 |
| 1 | 0.25 | 27.00 | 4.92 | 25.00 | 3 |
| 1 | 0.5 | 28.13 | 5.20 | 28.00 | 3 |
| 1 | 0.75 | 28.13 | 5.71 | 30.10 | 3 |
| 1 | 1 | 25.50 | 5.45 | 27.20 | 3 |
| 1 | 2 | 19.57 | 3.86 | 17.70 | 3 |
| 1 | 4 | 10.34 | 2.90 | 10.20 | 3 |
| 1 | 6 | 4.90 | 0.99 | 5.47 | 3 |
| 1 | 8 | 2.69 | 0.42 | 2.46 | 3 |
| 1 | 12 | 1.09 | 0.18 | 1.02 | 3 |
| 1 | 24 | 0.52 | 0.03 | 0.51 | 3 |
| 1 | 48 | BLQ | NA | BLQ | 3 |
| 2 | 0 | 37.90 | 4.03 | 39.60 | 3 |
| 2 | 0.1667 | 48.27 | 3.67 | 46.30 | 3 |
| 2 | 0.25 | 49.33 | 4.14 | 47.90 | 3 |
| 2 | 0.5 | 51.90 | 6.87 | 48.60 | 3 |
| 2 | 0.75 | 57.23 | 6.36 | 58.60 | 3 |
| 2 | 1 | 56.47 | 3.04 | 57.70 | 3 |
| 2 | 2 | 41.60 | 1.31 | 42.20 | 3 |
| 2 | 4 | 20.27 | 2.75 | 20.30 | 3 |
| 2 | 6 | 7.69 | 1.25 | 7.71 | 3 |
| 2 | 8 | 3.77 | 0.91 | 3.34 | 3 |
| 2 | 12 | 1.74 | 0.30 | 1.64 | 3 |
| 2 | 24 | 0.87 | 0.10 | 0.85 | 3 |
| 2 | 48 | 0.21 | 0.36 | BLQ | 3 |
| 5 | 0 | 88.60 | 38.64 | 82.30 | 3 |
| 5 | 0.1667 | 109.70 | 29.61 | 121.00 | 3 |
| 5 | 0.25 | 125.17 | 25.26 | 131.00 | 3 |
| 5 | 0.5 | 124.67 | 11.02 | 130.00 | 3 |
| 5 | 0.75 | 119.67 | 5.51 | 117.00 | 3 |
| 5 | 1 | 117.67 | 5.69 | 116.00 | 3 |
| 5 | 2 | 86.60 | 3.15 | 86.50 | 3 |
| 5 | 4 | 50.03 | 10.20 | 45.60 | 3 |
| 5 | 6 | 23.47 | 3.88 | 22.30 | 3 |
| 5 | 8 | 10.98 | 1.53 | 11.00 | 3 |
| 5 | 12 | 4.51 | 0.55 | 4.61 | 3 |
| 5 | 24 | 2.45 | 0.46 | 2.23 | 3 |
| 5 | 48 | 1.55 | 0.24 | 1.63 | 3 |

BLQ = blow limit for quantitation

TABLE 42
PK Parameters Derived by Non-Compartmental Model

| | ZA | | | | | | |
|---|---|---|---|---|---|---|---|
| Subject | Dose | ZA Dose | AUC (0-12h) | AUC (0-∞) | AUC (0-∞)/D | Cmax | Cmax/D |
| No | (mg) | (ug/kg) | (hr*ng/mL) | (hr*ng/mL) | (kg-h/mL) | (ng/mL) | (kg/mL) |
| 1 | 0.25 | 4.448 | 29.30 | 30.00 | 0.00674 | 7.75 | 0.00174 |
| 8 | 0.25 | 3.272 | 29.49 | 31.77 | 0.00971 | 5.67 | 0.00173 |
| 11 | 0.25 | 3.324 | 29.73 | 30.84 | 0.00926 | 6.84 | 0.00205 |
| Mean | | | 29.50 | 30.87 | 0.00857 | 6.75 | 0.00184 |
| S.D. | | | 0.22 | 0.89 | 0.00160 | 1.04 | 0.00018 |
| Median | | | 29.49 | 30.84 | 0.00926 | 6.84 | 0.00174 |
| n | | | 3 | 3 | 3 | 3 | 3 |
| 16 | 0.5 | 8.818 | 60.77 | 62.49 | 0.00709 | 15.30 | 0.00174 |
| 18 | 0.5 | 8.913 | 66.33 | 68.26 | 0.00766 | 17.10 | 0.00192 |
| 21 | 0.5 | 6.649 | 62.43 | 64.36 | 0.00968 | 14.20 | 0.00214 |
| Mean | | | 63.17 | 65.04 | 0.00814 | 15.53 | 0.00193 |
| S.D. | | | 2.85 | 2.95 | 0.00136 | 1.46 | 0.00020 |
| Median | | | 62.43 | 64.36 | 0.00766 | 15.30 | 0.00192 |
| n | | | 3 | 3 | 3 | 3 | 3 |
| 25 | 1 | 17.331 | 102.79 | 121.54 | 0.00701 | 30.10 | 0.00174 |
| 26 | 1 | 13.495 | 137.25 | 158.68 | 0.01176 | 33.40 | 0.00247 |
| 27 | 1 | 19.608 | 104.30 | 122.44 | 0.00624 | 25.00 | 0.00128 |
| Mean | | | 114.78 | 134.22 | 0.00834 | 29.50 | 0.00183 |
| S.D. | | | 19.47 | 21.19 | 0.00299 | 4.23 | 0.00061 |
| Median | | | 104.30 | 122.44 | 0.00701 | 30.10 | 0.00174 |
| n | | | 3 | 3 | 3 | 3 | 3 |
| 29 | 2 | 33.333 | 217.53 | 246.43 | 0.00739 | 62.80 | 0.00188 |
| 31 | 2 | 30.349 | 213.87 | 245.47 | 0.00809 | 53.00 | 0.00175 |
| 32 | 2 | 35.587 | 236.51 | 293.51 | 0.00825 | 58.70 | 0.00165 |
| Mean | | | 222.64 | 261.80 | 0.00791 | 58.17 | 0.00176 |
| S.D. | | | 12.15 | 27.46 | 0.00045 | 4.92 | 0.00012 |
| Median | | | 217.53 | 246.43 | 0.00809 | 58.70 | 0.00175 |
| n | | | 3 | 3 | 3 | 3 | 3 |
| 37 | 5 | 78.616 | 499.14 | 620.17 | 0.00789 | 131.00 | 0.00167 |
| 38 | 5 | 70.721 | 477.36 | 637.47 | 0.00901 | 117.00 | 0.00165 |
| 39 | 5 | 69.541 | 575.89 | 729.31 | 0.01049 | 147.00 | 0.00211 |
| Mean | | | 517.47 | 662.31 | 0.00913 | 131.67 | 0.00181 |
| S.D. | | | 51.76 | 58.66 | 0.00130 | 15.01 | 0.00026 |
| Median | | | 499.14 | 637.47 | 0.00901 | 131.00 | 0.00167 |
| n | | | 3 | 3 | 3 | 3 | 3 |

| Subject | Tmax | T½ₑₗᵢₘ | Vz/F | Vss/F | CL/F | MRT |
|---|---|---|---|---|---|---|
| No | (h) | (h) | (mL/kg) | (mL/kg) | (mL/h-kg) | (h) |
| 1 | 1.06 | 1.88 | 402.13 | 434.92 | 148.27 | 2.93 |
| 8 | 1.56 | 2.44 | 363.27 | 433.43 | 102.99 | 4.21 |
| 11 | 1.31 | 2.10 | 326.69 | 363.78 | 108.04 | 3.37 |
| Mean | 1.31 | 2.14 | 364.03 | 410.71 | 119.77 | 3.50 |
| S.D. | 0.25 | 0.29 | 37.73 | 40.65 | 24.81 | 0.65 |
| Median | 1.31 | 2.10 | 363.27 | 433.43 | 108.04 | 3.37 |
| n | 3 | 3 | 3 | 3 | 3 | 3 |
| 16 | 0.81 | 2.24 | 455.53 | 450.70 | 141.12 | 3.19 |
| 18 | 1.31 | 2.27 | 427.95 | 422.54 | 130.56 | 3.24 |
| 21 | 1.31 | 2.27 | 338.97 | 345.70 | 103.30 | 3.35 |
| Mean | 1.14 | 2.26 | 407.48 | 406.31 | 125.00 | 3.26 |
| S.D. | 0.29 | 0.02 | 60.91 | 54.35 | 19.52 | 0.08 |
| Median | 1.31 | 2.27 | 427.95 | 422.54 | 130.56 | 3.24 |
| n | 3 | 3 | 3 | 3 | 3 | 3 |
| 25 | 1.31 | 12.87 | 2647.09 | 988.08 | 142.60 | 6.93 |
| 26 | 1.06 | 13.95 | 1712.21 | 566.17 | 85.05 | 6.66 |
| 27 | 0.73 | 8.85 | 2045.22 | 1001.59 | 160.15 | 6.25 |
| Mean | 1.03 | 11.89 | 2134.84 | 851.95 | 129.26 | 6.61 |
| S.D. | 0.29 | 2.69 | 473.84 | 247.59 | 39.29 | 0.34 |
| Median | 1.06 | 12.87 | 2045.22 | 988.08 | 142.60 | 6.66 |
| n | 3 | 3 | 3 | 3 | 3 | 3 |
| 29 | 1.31 | 12.72 | 2482.23 | 774.20 | 135.27 | 5.72 |
| 31 | 1.56 | 12.72 | 2269.62 | 765.07 | 123.64 | 6.19 |
| 32 | 1.56 | 22.02 | 3852.64 | 1283.55 | 121.25 | 10.59 |
| Mean | 1.48 | 15.82 | 2868.16 | 940.94 | 126.72 | 7.50 |
| S.D. | 0.14 | 5.37 | 859.18 | 296.74 | 7.50 | 2.68 |
| Median | 1.56 | 12.72 | 2482.23 | 774.20 | 123.64 | 6.19 |
| n | 3 | 3 | 3 | 3 | 3 | 3 |
| 37 | 0.81 | 23.36 | 4271.90 | 1386.59 | 126.77 | 10.94 |
| 38 | 1.31 | 24.45 | 3913.99 | 1531.64 | 110.94 | 13.81 |
| 39 | 0.81 | 26.05 | 3583.42 | 1206.99 | 95.35 | 12.66 |
| Mean | 0.98 | 24.62 | 3923.10 | 1375.07 | 111.02 | 12.47 |
| S.D. | 0.29 | 1.35 | 344.33 | 162.63 | 15.71 | 1.44 |
| Median | 0.81 | 24.45 | 3913.99 | 1386.59 | 110.94 | 12.66 |
| n | 3 | 3 | 3 | 3 | 3 | 3 |

TABLE 43
PK Parameters Derived by Non-Compartmental Model - Ordered by
ZA dosage

| | Nominal | | | | | | |
|---|---|---|---|---|---|---|---|
| | Inf | Infusion | Nominal | PH20 | | | |
| ZA_dose | Time | time | PH20 | dose | | AUC (0-inf) | AUC (0-inf)/D |
| (mg/kg) | (h) | (hr) | dose (U) | (U) | Subject # | (hr * ng/mL) | (hr * kg/mL) |
| 0.0032 | 0.5 | 0.531 | 24000 | 22944 | 8 | 32.62 | 0.010193 |
| 0.0033 | 0.5 | 0.554 | 24000 | 23928 | 11 | 30.76 | 0.009322 |
| 0.0045 | 0.5 | 0.554 | 24000 | 23928 | 1 | 29.87 | 0.006638 |
| 0.0066 | 0.5 | 0.556 | 24000 | 24000 | 21 | 64.34 | 0.009748 |
| 0.0085 | 0.5 | 0.539 | 24000 | 23304 | 18 | 68.08 | 0.008010 |
| 0.0088 | 0.5 | 0.553 | 24000 | 23904 | 16 | 62.45 | 0.007097 |
| 0.0128 | 0.5 | 0.534 | 24000 | 23088 | 26 | 153.34 | 0.011980 |
| 0.0171 | 0.5 | 0.549 | 24000 | 23736 | 25 | 117.32 | 0.006861 |
| 0.0196 | 0.5 | 0.555 | 24000 | 23976 | 27 | 120.39 | 0.006142 |
| 0.0312 | 0.5 | 0.554 | 24000 | 23952 | 31 | 239.13 | 0.007665 |
| 0.0339 | 0.5 | 0.556 | 24000 | 24000 | 29 | 240.52 | 0.007095 |
| 0.0350 | 0.5 | 0.556 | 24000 | 24000 | 32 | 295.44 | 0.008441 |
| 0.0525 | 0.25 | 0.251 | 6000 | 6024 | 51 | 508.45 | 0.009685 |
| 0.0608 | 0.25 | 0.250 | 3000 | 2880 | 68 | 629.51 | 0.010354 |
| 0.0653 | 0.25 | 0.281 | 12000 | 12216 | 41 | 736.25 | 0.011275 |
| 0.0691 | 0.25 | 0.552 | 24000 | 23856 | 39 | 727.04 | 0.010522 |
| 0.0706 | 0.25 | 0.554 | 24000 | 23952 | 38 | 637.31 | 0.009027 |
| 0.0717 | 0.25 | 0.240 | 3000 | 2880 | 67 | 725.36 | 0.010117 |
| 0.0776 | 0.25 | 0.244 | 3000 | 2928 | 70 | 690.49 | 0.008898 |
| 0.0781 | 0.25 | 0.250 | 6000 | 6000 | 71 | 692.98 | 0.008873 |
| 0.0791 | 0.25 | 0.556 | 24000 | 24000 | 37 | 619.98 | 0.007838 |
| 0.0794 | 0.25 | 0.250 | 6000 | 6000 | 74 | 555.36 | 0.006994 |
| 0.0806 | 0.25 | 0.250 | 6000 | 6000 | 77 | 678.04 | 0.008412 |
| 0.0820 | 0.25 | 0.250 | 6000 | 6000 | 63 | 662.32 | 0.008077 |
| 0.0877 | 0.25 | 0.276 | 12000 | 12000 | 46 | 746.89 | 0.008516 |

| | | | | T½ | | | |
|---|---|---|---|---|---|---|---|
| ZA_dose | Cmax | Cmax/D | Tmax | λz | CL | Vz | Vss |
| (mg/kg) | (ng/mL) | (kg/mL) | (hr) | (hr) | (mL/hr/kg) | (mL/kg) | (mL/kg) |
| 0.0032 | 5.67 | 0.001772 | 1.41 | 3.09 | 98.10 | 437.06 | 456.21 |
| 0.0033 | 6.84 | 0.002073 | 1.25 | 2.20 | 107.27 | 339.82 | 364.47 |
| 0.0045 | 7.75 | 0.001722 | 1.02 | 1.88 | 150.66 | 408.61 | 443.18 |
| 0.0066 | 14.20 | 0.002152 | 1.31 | 2.27 | 102.58 | 336.61 | 343.16 |
| 0.0085 | 17.10 | 0.002012 | 1.29 | 2.24 | 124.84 | 404.24 | 402.54 |
| 0.0088 | 15.30 | 0.001739 | 0.80 | 2.21 | 140.91 | 449.19 | 448.93 |
| 0.0128 | 33.40 | 0.002609 | 1.03 | 8.45 | 83.48 | 1017.36 | 426.21 |
| 0.0171 | 30.10 | 0.001760 | 1.30 | 7.82 | 145.76 | 1644.23 | 778.61 |
| 0.0196 | 25.00 | 0.001276 | 0.72 | 6.53 | 162.81 | 1534.48 | 914.22 |
| 0.0312 | 53.00 | 0.001699 | 1.54 | 8.86 | 130.47 | 1668.54 | 670.25 |
| 0.0339 | 62.80 | 0.001853 | 1.29 | 8.70 | 140.94 | 1768.77 | 664.99 |
| 0.0350 | 58.70 | 0.001677 | 1.56 | 22.02 | 118.47 | 3764.29 | 1249.30 |
| 0.0525 | 95.40 | 0.001817 | 0.52 | 20.44 | 103.26 | 3044.13 | 1276.27 |
| 0.0608 | 144.00 | 0.002368 | 1.00 | 27.90 | 96.58 | 3888.14 | 1592.21 |
| 0.0653 | 141.00 | 0.002159 | 1.01 | 42.84 | 88.69 | 5481.66 | 2371.43 |
| 0.0691 | 147.00 | 0.002127 | 0.79 | 26.05 | 95.04 | 3571.83 | 1205.01 |
| 0.0706 | 117.00 | 0.001657 | 1.30 | 24.45 | 110.78 | 3908.22 | 1529.47 |
| 0.0717 | 156.00 | 0.002176 | 0.74 | 18.33 | 98.85 | 2613.40 | 1039.44 |
| 0.0776 | 215.00 | 0.002771 | 0.68 | 19.44 | 112.38 | 3152.69 | 986.73 |
| 0.0781 | 198.00 | 0.002535 | 0.88 | 19.20 | 112.70 | 3122.59 | 874.24 |
| 0.0791 | 131.00 | 0.001656 | 0.81 | 23.36 | 127.59 | 4299.50 | 1395.65 |
| 0.0794 | 120.00 | 0.001511 | 0.75 | 23.70 | 142.97 | 4889.24 | 1842.53 |
| 0.0806 | 147.00 | 0.001824 | 0.75 | 24.59 | 118.87 | 4217.69 | 1608.73 |
| 0.0820 | 167.00 | 0.002037 | 0.50 | 20.53 | 123.81 | 3666.62 | 1473.00 |
| 0.0877 | 213.00 | 0.002429 | 0.78 | 20.76 | 117.42 | 3516.46 | 1096.39 |

TABLE 44
Area Under the Serum Concentration vs Time Curve

| | Nom. | | Nom. | | | | | | | |
|---|---|---|---|---|---|---|---|---|---|---|
| ZA | Inf | Inf | PH20 | PH20 | | AUC | | | | |
| dose | Time | time | dose | dose | | (0-inf) | AUC (0-inf)/D | AUC (0-12 h) | AUC (0-24 h) | AUC (0-48 h) |
| (mg/kg) | (h) | (hr) | (U) | (U) | Subj. # | (hr * ng/mL) | (hr * kg/mL) | (hr * ng/mL) | (hr * ng/mL) | (hr * ng/mL) |
| 0.0032 | 0.5 | 0.531 | 24000 | 22944 | 8 | 32.62 | 0.010193 | 28.77 | 29.42 | 29.42 |
| 0.0033 | 0.5 | 0.554 | 24000 | 23928 | 11 | 30.76 | 0.009322 | 29.45 | 29.87 | 29.87 |
| 0.0045 | 0.5 | 0.554 | 24000 | 23928 | 1 | 29.87 | 0.006638 | 29.16 | 29.44 | 29.44 |
| 0.0066 | 0.5 | 0.556 | 24000 | 24000 | 21 | 64.34 | 0.009748 | 62.40 | 64.74 | 66.11 |
| 0.0085 | 0.5 | 0.539 | 24000 | 23304 | 18 | 68.08 | 0.008010 | 66.20 | 68.45 | 69.80 |
| 0.0088 | 0.5 | 0.553 | 24000 | 23904 | 16 | 62.45 | 0.007097 | 60.77 | 62.92 | 64.20 |
| 0.0128 | 0.5 | 0.534 | 24000 | 23088 | 26 | 153.34 | 0.011980 | 136.91 | 146.69 | 150.39 |
| 0.0171 | 0.5 | 0.549 | 24000 | 23736 | 25 | 117.32 | 0.006861 | 102.70 | 111.80 | 115.14 |
| 0.0196 | 0.5 | 0.555 | 24000 | 23976 | 27 | 120.39 | 0.006142 | 104.27 | 115.62 | 119.00 |
| 0.0312 | 0.5 | 0.554 | 24000 | 23952 | 31 | 239.13 | 0.007665 | 212.93 | 228.36 | 233.98 |
| 0.0339 | 0.5 | 0.556 | 24000 | 24000 | 29 | 240.52 | 0.007095 | 216.76 | 230.88 | 236.02 |
| 0.0350 | 0.5 | 0.556 | 24000 | 24000 | 32 | 295.44 | 0.008441 | 238.45 | 257.44 | 276.83 |
| 0.0525 | 0.25 | 0.251 | 6000 | 6024 | 51 | 508.45 | 0.009685 | 384.47 | 430.19 | 470.90 |
| 0.0608 | 0.25 | 0.250 | 3000 | 2880 | 68 | 629.51 | 0.010354 | 455.81 | 502.57 | 556.98 |
| 0.0653 | 0.25 | 0.281 | 12000 | 12216 | 41 | 736.25 | 0.011275 | 486.53 | 530.79 | 596.59 |
| 0.0691 | 0.25 | 0.552 | 24000 | 23856 | 39 | 727.04 | 0.010522 | 573.76 | 618.86 | 666.80 |
| 0.0706 | 0.25 | 0.554 | 24000 | 23952 | 38 | 637.31 | 0.009027 | 477.24 | 523.91 | 579.99 |
| 0.0717 | 0.25 | 0.240 | 3000 | 2880 | 67 | 725.36 | 0.010117 | 565.15 | 630.35 | 683.35 |
| 0.0776 | 0.25 | 0.244 | 3000 | 2928 | 70 | 690.49 | 0.008898 | 573.29 | 616.09 | 657.66 |
| 0.0781 | 0.25 | 0.250 | 6000 | 6000 | 71 | 692.98 | 0.008873 | 591.54 | 629.18 | 664.94 |
| 0.0791 | 0.25 | 0.556 | 24000 | 24000 | 37 | 619.98 | 0.007838 | 498.97 | 536.50 | 578.16 |
| 0.0794 | 0.25 | 0.250 | 6000 | 6000 | 74 | 555.36 | 0.006994 | 425.73 | 466.81 | 508.93 |
| 0.0806 | 0.25 | 0.250 | 6000 | 6000 | 77 | 678.04 | 0.008412 | 516.11 | 566.05 | 617.66 |
| 0.0820 | 0.25 | 0.250 | 6000 | 6000 | 63 | 662.32 | 0.008077 | 496.89 | 559.09 | 613.30 |
| 0.0877 | 0.25 | 0.276 | 12000 | 12000 | 46 | 746.89 | 0.008516 | 609.55 | 656.09 | 705.53 |

TABLE 45
Estimation of SC Absorption Time for ZA Using a Dose
Volume of 100 mL, Infusion Rate of 3 mL/min and
Co-Administration with 24000 Units of rHuPH20

| ZA Dose | Subject | SC MRT^{a} | IV MRT^{b} | | | Std. | | |
|---|---|---|---|---|---|---|---|---|
| (mg) | No | (h) | (h) | MAT^{c} (h) | Mean^{d} | Dev.^{e} | Median^{f} | n^{g} |
| 0.25 | 1 | 2.9333 | 1.9010 | 1.03 | 1.60 | 0.65 | 1.47 | 3 |
| 0.25 | 8 | 4.2085 | 1.9010 | 2.31 | | | | |
| 0.25 | 11 | 3.3670 | 1.9010 | 1.47 | | | | |
| 0.5 | 16 | 3.1937 | 2.2330 | 0.96 | 1.03 | 0.08 | 1.00 | 3 |
| 0.5 | 18 | 3.2364 | 2.2330 | 1.00 | | | | |
| 0.5 | 21 | 3.3465 | 2.2330 | 1.11 | | | | |
| 1 | 25 | 6.9291 | 5.2494 | 1.68 | 1.36 | 0.34 | 1.41 | 3 |
| 1 | 26 | 6.6571 | 5.2494 | 1.41 | | | | |
| 1 | 27 | 6.2542 | 5.2494 | 1.00 | | | | |
| 2 | 29 | 5.7235 | 5.2494 | 0.47 | 0.93 | 0.46 | 0.94 | 3 |
| 2 | 31 | 6.1881 | 5.2494 | 0.94 | | | | |
| 2 | 32 | 10.5861 | 9.1957 | 1.39 | | | | |
| 5 | 37 | 10.9381 | 9.1957 | 1.74 | 3.27 | 1.44 | 3.46 | 3 |
| 5 | 38 | 13.8059 | 9.1957 | 4.61 | | | | |
| 5 | 39 | 12.6583 | 9.1957 | 3.46 | | | | |
| | | | Mean^{h} | 1.64 | | | | |
| | | | Std Devⁱ | 1.09 | | | | |
| | | | Median^{j} | 1.39 | | | | |
| | | | n^{k} | 15 | | | | |

^{a}Mean residence time (extrapolated to ∞ from t last) for SC 0.56 h infusion obtained from Study HZ2-08-02. Tlast = 8 hours for Subjects 1, 8, and 11. Tlast = 12 hours for Subjects 16, 18, and 21. Tlast = 24 hours for Subjects 25, 26, 27, and 29. Tlast = 48 hours for Subjects 32, 37, 38, and 39.
^{b}Mean residence time (extrapolated to ∞ from t last) for IV 0.25 h infusion was calculated from published ZA PK concentration-time data and adjusted to match the SC doses based on reported dose linearity.
^{c}Mean absorption time = SC MRT − IV MRT
^{d}Mean value calculated for the dose group
^{e}Standard deviation calculated for the dose group
^{f}Median value calculated for the dose group
^{g}Number of determination for the dose group
^{h}Mean value calculated for all dose groups
ⁱStandard deviation calculated for all dose group
^{j}Median value calculated for all dose group
^{k}Number of determination for all dose group

The average mean absorption time (MAT) generally ranged from 1.0 to 1.6 hours for ZA doses of 0.25 to 2 mg/subject. The MAT appeared to be prolonged for the 5 mg dose cohort due to a larger variability among individual subjects in this cohort. The inter-dose group median MAT was 1.4 hours and in good agreement with a median Tmax value of 1.3 hours (mean Tmax=1.2±0.29 hours).

### Example 12

### Phase 1-Stage 2 Clinical Study in Human Subjects Pharmacokinetic Analysis of Subcutaneous (SC) Zoledronic Acid Alone

In order to determine the maximally tolerated subcutaneous dose of zoledronic acid (ZA), a pharmacokinetic analysis is performed in human subjects using increasing doses of ZA alone (referred to herein as Stage 2). The data obtained from this experiment can be used to determine the effects of rHuPH20 on the maximum dosage of ZA and to compare the effects of rHuPH20 on the bioavailability of ZA via subcutaneous route in humans.

For this study, dosing solutions are prepared using commercially available zoledronic acid (Zometa®, 0.8 mg/mL ZA, Novartis; see Example 11). The dosing solutions contain varying amounts of ZA (0.25, 0.5, 1, 2, and 5 mg) in several dosage volumes (100, 50, 25, 12.5, 10, and 7.1 ml) as summarized in Table 44. The dosing formulations are designed to neutralize the citrate excipients in the Zometa® product and to account for volume constraints in dosing solutions due to the limit of 0.8 mg/mL ZA present in the Zometa® product.

TABLE 44
Content of Stage 2 Dosing Solutions

| | | | | | | Drug |
|---|---|---|---|---|---|---|
| | | | | | Drug | Product |
| | ZA | ZA | rHuPH20 | HSA | Product | Volume |
| | (mg) | Conc. | (U) in | (mg) | dose | (mL) |
| Cohort | in dose | (mg/mL) | dose | in dose | (mL) | prepared |
| 1 | 0.25 | 0.0025 | 0 | 100 | 20* | 25 |
| 2 | 0.5 | 0.005 | 0 | 100 | 20* | 25 |
| 3 | 1 | 0.01 | 0 | 100 | 20* | 25 |
| 4 | 2 | 0.02 | 0 | 100 | 20* | 25 |
| 5 | 5 | 0.05 | 0 | 100 | 20* | 25 |
| 6 | 5 | 0.1 | 0 | 50 | 20** | 25 |
| 7 | 5 | 0.2 | 0 | 25 | 25 | 28 |
| 8 | 5 | 0.4 | 0 | 12.5 | 12.5 | 15 |
| 9 | 5 | 0.7 | 0 | 7.1 | 7.1 | 10 |
| 10 | 5 | 0.5 | 0 | 10 | 10 | 15 |

*Drug product is diluted to 100 mL with saline before infusion
**Drug product is diluted to 50 mL with saline before injection

Stock solutions for the experiment are prepared for each of the dosing groups. The composition of each of the stock solutions is summarized in Table 45.

TABLE 45
Composition of Stage 2 Dosing Stock Solutions

| | Prod. Vol. | ZA | HSA | rHuPH20 |
|---|---|---|---|---|
| Cohort | (ml) | (ug/ml) | (mg/ml) | (ug/ml) |
| 1 | 25 | 12.5 | 5 | 0 |
| 2 | 25 | 25 | 5 | 0 |
| 3 | 25 | 50 | 5 | 0 |
| 4 | 25 | 100 | 5 | 0 |
| 5 | 25 | 250 | 5 | 0 |
| 6 | 25 | 250 | 2.5 | 0 |
| 7 | 28 | 200 | 1 | 0 |
| 8 | 15 | 400 | 1 | 0 |
| 9 | 10 | 704 | 1 | 0 |
| 10 | 15 | 500 | 1 | 0 |

Table 46 describes the formulation instructions for preparing the dosing stock solutions. Each clinical dose is first prepared within a 30 cc vial containing 1 ml of excipients. Each component of the dosing solution is added in the order of manufacturing steps as depicted in Table 45.

TABLE 46
Process for Generating Stage 2 Dosing Stock Solutions

| Manuf. | | | | | | | | | | | |
|---|---|---|---|---|---|---|---|---|---|---|---|
| steps | Reagent Added | 1 | 2 | 3 | 4 | 5 | 6 | 7 | 8 | 9 | 10 |
| 1 | Excipient Salts | 1 | 1 | 1 | 1 | 1 | 1 | 1 | 1 | 1 | 1 |
| | (ml) | | | | | | | | | | |
| 2 | Saline | 21.3 | 20.9 | 20.1 | 18.6 | 13.9 | 15.2 | 19.6 | 6.4 | 0.2 | 4.5 |
| | solution (ml) | | | | | | | | | | |
| 3 | Zometa ® | 0.4 | 0.8 | 1.6 | 3.1 | 7.8 | 7.8 | 7 | 7.5 | 8.8 | 9.4 |
| | solution (ml) | | | | | | | | | | |
| 4 | HSA solution | 2.3 | 2.3 | 2.3 | 2.3 | 2.3 | 1.0 | 0.4 | 0.1 | 0 | 0.1 |
| | (50 mg/ml; 5% | | | | | | | | | | |
| | USP) | | | | | | | | | | |
| | rHuPH20 | 25 | 25 | 25 | 25 | 25 | 25 | 25 | 25 | 25 | 25 |
| | Solution (HUA, | | | | | | | | | | |
| | 1 mg/ml) | | | | | | | | | | |

For administration to human subjects, the dosing stock solutions are diluted as required (see Table 47). For cohorts 1-5, 20 ml of the dosing solution is mixed with 80 ml saline to generate the 100 ml dose. For cohort 6, 20 ml of the dosing solution is mixed with 30 ml saline. Cohort dosing solutions 7-10 are not diluted.

TABLE 47
Preparation of Stage 2 Dosing Solutions for Administration

| | Dilution | | Vol. | ZA | [ZA] | [HSA] | [PH20] |
|---|---|---|---|---|---|---|---|
| | Req. | Deliv. | Admin. | Admin. | Admin. | Admin. | Admin. |
| ID # | (Fold) | Mode | (ml) | (mg) | (ug/ml) | (ug/ml) | (ug/ml) |
| 1 | 5 | Infusion | 100 | 0.25 | 2.5 | 1 | 0 |
| | | Bag | | | | | |
| 2 | 5 | Infusion | 100 | 0.5 | 50 | 1 | 0 |
| | | Bag | | | | | |
| 3 | 5 | Infusion | 100 | 1 | 10 | 1 | 0 |
| | | Bag | | | | | |
| 4 | 5 | Infusion | 100 | 2 | 20 | 1 | 0 |
| | | Bag | | | | | |
| 5 | 5 | Infusion | 100 | 5 | 50 | 1 | 0 |
| | | Bag | | | | | |
| 6 | 2.5 | Syringe | 50 | 5 | 100 | 1 | 0 |
| 7 | None | Syringe | 25 | 5 | 200 | 1 | 0 |
| 8 | None | Syringe | 12.5 | 5 | 400 | 1 | 0 |
| 9 | None | Syringe | 7.1 | 5 | 704 | 1 | 0 |
| 10 | None | Syringe | 10 | 5 | 500 | 1 | 0 |

Administration of the dosing solutions, measurement of ZA serum concentrations following administration, and pharmacokinetic analysis are performed as described in Example 11. Before and after completion of study drug administration, the Investigator assesses possible injection site reactions (ISRs) such as erythema, pruritus, edema, ulceration, and necrosis, and the subject makes self-assessments of the level of pain/discomfort. The administration sites also are digitally photographed before and after administration of study drug.

Since modifications will be apparent to those of skill in this art, it is intended that this invention be limited only by the scope of the appended claims.

1. A method for treating a bisphosphonate-treatable or preventable disease or condition in a subject in need of such treatment, comprising:
subcutaneously administering to the subject (a) an amount of a soluble hyaluronidase and (b) a bisphosphonate in an amount sufficient for treating the disease or condition, wherein:
the soluble hyaluronidase is administered at a concentration of at or about 10 Units/ml to 1000 Units/ml in an amount such that the incidence of injection site reactions in the subject is eliminated or substantially reduced compared to subcutaneous administration of the same amount of bisphosphonate in the absence of the hyaluronidase.

2. The method of claim 1, wherein the concentration of the soluble hyaluronidase is at or about 100 Units/ml to 1000 Units/ml.

3. The method of claim 1, wherein the amount is at or about 1 ml to 500 ml.

4. The method of claim 1, wherein the amount of soluble hyaluronidase administered is at or about 100 Units to 100,000 Units; at or about 1000 Units to 100,000 Units; at or about 3000 Units to 100,000 Units; at or about 5000 Units to 100,000 Units; at or about 10,000 Units to 100,000 Units; at or about 1000 Units to 50,000 Units; at or about 1000 Units to 24,000 Units; at or about 1000 Units to 10,000 Units; or at or about 3000 Units to 10,000 Units.

5. The method of claim 1, wherein the frequency of administration of the bisphosphonate is substantially the same as for intravenous administration of the same amount of bisphosphonate for the same disease or condition.

6. The method of claim 1, wherein a soluble hyaluronidase and a bisphosphonate are administered, sequentially, simultaneously in the same composition or in separate compositions, or intermittently.

7. The method of claim 1, wherein the subject is a human.

8. The method of claim 1, wherein one or more different bisphosphonates is administered.

9. The method of claim 1, wherein the bisphosphonate is administered for the same length of time required to complete administration as for intravenous administration of the same amount of bisphosphonate for the same disease or condition.

10. The method of claim 1, wherein the bisphosphonate is administered for a shorter length of time required to complete administration as for intravenous administration of the same amount of bisphosphonate for the same disease or condition.

11. The method of claim 1, wherein bioavailability of the subcutaneously administered bisphosphonate is at least about 90% of the bioavailability of the same dosage administered via intravenous administration.

12. The method of claim 1, wherein the amount of bisphosphonate administered is sufficient to treat the subject for a period of one week, two weeks, three weeks, four weeks, one month, two months, three months, four months, five months, six months, seven months, eight months, nine months, ten months, eleven months, twelve months, eighteen months or twenty-four months without need for additional bisphosphonate administration to the subject during the period.

13. The method of claim 1, wherein the amount of soluble hyaluronidase is sufficient to effect subcutaneous administration of the bisphosphonate at a dosage administered no more than once per week.

14. The method of claim 1, wherein the frequency of the dosage regimen comprises administration of bisphosphonate and soluble hyaluronidase once every week, once every two weeks, once every three weeks, once every four weeks, once every month, once every two months, once every three months, once every four months, once every five months, once every six months, once every seven months, once every eight months, once every nine months, once every ten months, once every eleven months, once every twelve months, once every eighteen months or once every two years.

15. The method of claim 1, wherein the time interval between two successive treatments is greater than the time interval between treatments for administration of the same amount of bisphosphonate via intravenous administration.

16. The method of claim 1, wherein the soluble hyaluronidase comprises a PH20 or a truncated form thereof.

17. The method of claim 16, wherein the soluble hyaluronidase is selected from an ovine, mouse, monkey, bovine or human PH20.

18. The method of claim 16, wherein the soluble hyaluronidase is a soluble PH20 that lacks a C-terminal glycosylphosphatidylinositol attachment site.

19. The method of claim 1, wherein the soluble hyaluronidase is selected from among polypeptides containing a sequence of amino acids set forth in any of SEQ ID NOS:4-9 and 48, and allelic variants, species variants and other variants thereof that retain hyaluronidase activity.

20. The method of claim 19, wherein the soluble hyaluronidase variant is selected from among polypeptides having at least 60, 65, 70, 75, 80, 85, 88, 90, 91, 92, 93, 94, 95, 96, 97, 98, 99% or more sequence identity along their full length to a contiguous sequence of amino acids set forth in SEQ ID NO:1.

21. The method of claim 1, wherein the soluble hyaluronidase comprises a polypeptide encoded by a sequence of nucleic acids that encodes a sequence of amino acids set forth in SEQ ID NO:3 or 4, or comprises a polypeptide encoded by the sequence of nucleotides set forth in SEQ ID NO:49.

22. The method of claim 1, wherein the soluble hyaluronidase comprises rHuPH20.

23. The method of claim 1, wherein the soluble hyaluronidase comprises one or more soluble hyaluronidases.

24. The method of claim 1, wherein the soluble hyaluronidase is glycosylated, pegylated, or sialylated.

25. The method of claim 1, wherein the bisphosphonate is an N-bisphosphonate or a pharmaceutically acceptable salt or ester thereof or any hydrate thereof.

26. The method of claim 1, wherein the bisphosphonate is selected from among alendronate, cimadronate, clodronate, tiludronate, etidronate, ibandronate, neridronate, olpandronate, risedronate, piridronate, pamidronate, zoledronate, pharmaceutically acceptable salts or esters thereof, any hydrate thereof and combinations thereof.

27. The method of claim 1, wherein the bisphosphonate is a nitrogenous bisphosphonate.

28. The method of claim 1, wherein the bisphosphonate is zoledronate, ibandronate or pamidronate.

29. The method of claim 1, wherein the bisphosphonate and hyaluronidase are administered as a single subcutaneous injection.

30. The method of claim 1, wherein the bisphosphonate and hyaluronidase are administered separately.

31. The method of claim 1, wherein the bisphosphonate and hyaluronidase are administered simultaneously or intermittently.

32. The method of claim 1, wherein the hyaluronidase is administered prior to administration of the bisphosphonate.

33. The method of claim 32, wherein hyaluronidase is administered 0.5 minutes, 1 minute, 2 minutes, 3 minutes, 4 minutes, 5 minutes, 6 minutes, 7 minutes, 8 minutes, 9 minutes, 10 minutes, 20 minutes or 30 minutes prior to administration of the bisphosphonate.

34. The method of claim 1, wherein the bisphosphonate is in a liquid formulation, and the time required to subcutaneously administer the dosage of bisphosphonate is determined based on the concentration of the bisphosphonate in the liquid formulation and a desired rate of infusion of the liquid formulation.

35. The method of claim 34, wherein the rate of infusion is controlled by a pump, by gravity or controlled dispersion from a syringe over a period of time.

36. The method of claim 1, wherein the bisphosphonate and hyaluronidase are formulated in a single composition.

37. The method of claim 1, wherein about or 0.5 milligrams (mg), about or 1 mg, about or 3 mg, about or 5 mg, about or 10 mg, about or 20 mg, about or 30 mg, about or 40 mg, about or 50 mg, about or 60 mg, about or 70 mg, about or 80 mg, about or 90 mg, about or 100 mg of bisphosphonate is administered.

38. The method of claim 1, wherein:
(a) the bisphosphonate is zoledronate; and
(b) about or 0.5 milligrams (mg), about or 1 mg, about or 1.5 mg, about or 2 mg, about or 2.5 mg, about or 3 mg, about or 3.5 mg, about or 4 mg, about or 4.5 mg, about or 5 mg, about or 5.5 mg, about or 6 mg, about or 6.5 mg, about or 7 mg, about or 7.5 mg, about or 8 mg, about or 8.5 mg, about or 9 mg, about or 9.5 mg, or about or 10 mg of zoledronate is administered.

39. The method of claim 38, wherein the amount of zoledronate administered subcutaneously is or is about 5 mg and the amount is administered once yearly.

40. The method of claim 38, wherein the amount of zoledronate administered subcutaneously is or is about 5 milligrams in a liquid formulation, wherein the volume of the formulation is or is about 25 milliliters to 400 milliliters.

41. The method of claim 40, wherein the volume of the liquid formulation is or is about 25 milliliters to 200 milliliters.

42. The method of claim 38, wherein the amount of soluble hyaluronidase administered is at or about 100 Units to 100,000 Units; at or about 1000 Units to 100,000 Units; at or about 3000 Units to 100,000 Units; at or about 5000 Units to 100,000 Units; at or about 10,000 Units to 100,000 Units; at or about 1000 Units to 50,000 Units; at or about 1000 Units to 24,000 Units; at or about 1000 Units to 10,000 Units; or at or about 3000 Units to 10,000 Units.

43. The method of claim 1, wherein:
(a) the bisphosphonate is ibandronate; and
(b) about or 0.5 milligrams (mg), about or 1 mg, about or 1.5 mg, about or 2 mg, about or 2.5 mg, about or 3 mg, about or 3.5 mg, about or 4 mg, about or 4.5 mg, about or 5 mg, about or 5.5 mg, about or 6 mg, about or 6.5 mg, about or 7 mg, about or 7.5 mg, about or 8 mg, about or 8.5 mg, about or 9 mg, about or 9.5 mg, or about or 10 mg of ibandronate is administered.

44. The method of claim 43, wherein the amount of ibandronate administered subcutaneously is at or about 3 mg and the amount is administered once every three months.

45. The method of claim 43, wherein the amount of ibandronate administered subcutaneously is or is about 2 mg to 5 mg in a liquid formulation wherein the volume of the formulation is or is about 1 milliliter to 5 milliliters.

46. The method of claim 43, wherein the amount of soluble hyaluronidase administered is at or about 100 Units to 100,000 Units; at or about 1000 Units to 100,000 Units; at or about 3000 Units to 100,000 Units; at or about 5000 Units to 100,000 Units; at or about 10,000 Units to 100,000 Units; at or about 1000 Units to 50,000 Units; at or about 1000 Units to 24,000 Units; at or about 1000 Units to 10,000 Units; or at or about 3000 Units to 10,000 Units.

47. The method of claim 1, wherein:
(a) the bisphosphonate is pamidronate; and
(b) about or 10 mg, about or 20 mg, about or 30 mg, about or 40 mg, about or 50 mg, about or 60 mg, about or 70 mg, about or 80 mg, about or 90 mg, or about or 100 mg of pamidronate is administered.

48. The method of claim 47, wherein the amount of pamidronate administered subcutaneously is or is about 90 mg.

49. The method of claim 47, wherein the amount of pamidronate in the composition is or is about 90 mg in a liquid formulation, wherein the volume of the formulation is or is about 100 milliliters to 200 milliliters.

50. The method of claim 47, wherein the amount of soluble hyaluronidase administered is at or about 100 Units to 100,000 Units; at or about 1000 Units to 100,000 Units; at or about 3000 Units to 100,000 Units; at or about 5000 Units to 100,000 Units; at or about 10,000 Units to 100,000 Units; at or about 1000 Units to 50,000 Units; at or about 1000 Units to 24,000 Units; at or about 1000 Units to 10,000 Units; or at or about 3000 Units to 10,000 Units.

51. The method of claim 1, wherein the hyaluronidase is administered at a ratio (Units (U) hyaluronidase/milligrams (mg) of bisphosphonate) at or about 10 U/mg; at or about 25 U/mg; at or about 100 U/mg; at or about 1000 U/mg; at or about 2500 U/mg; at or about 5000 U/mg; at or about 10,000 U/mg; at or about 20,000 U/mg; at or about 100,000 U/mg; at or about 200,000 U/mg; at or about 1,000,000 U/mg; or at or about 2,000,000 U/mg.

52. The method of claim 51, wherein the hyaluronidase is administered at a ratio (Units hyaluronidase/milligrams of bisphosphonate) at or about 200 U/mg; or at or about 25,000 U/mg.

53. The method of claim 1, wherein the bisphosphonate-treatable or preventable disease or condition is selected from among osteoporosis, Paget's Disease, abnormally increased bone turnover, periodontal disease, tooth loss, bone fractures, rheumatoid arthritis, periprosthetic osteolysis, osteogenesis imperfecta, metastatic bone disease, bone metastases, hypercalcemia of malignancy and multiple myeloma.

54. The method of claim 1, wherein administration of soluble hyaluronidase and bisphosphonate results in an increase in bone density in the subject or a decrease in the rate of bone degradation in the subject following treatment.

55. A combination for treating a bisphosphonate treatable or preventable disease or condition in a human subject in need thereof, comprising:
(a) a first composition comprising a bisphosphonate formulated for single dosage subcutaneous administration at a dosage frequency of no greater than once per week in an amount sufficient for treating the disease or condition; and
(b) a second composition comprising an amount of a soluble hyaluronidase formulated for single dosage subcutaneous administration at a dosage frequency of no greater than once per week, wherein the amount of soluble hyaluronidase is at or about 100 Units to 100,000 Units.

56. The combination claim 55, wherein the amount of soluble hyaluronidase administered is at or about 100 Units to 100,000 Units; at or about 1000 Units to 100,000 Units; at or about 3000 Units to 100,000 Units; at or about 5000 Units to 100,000 Units; at or about 10,000 Units to 100,000 Units; at or about 1000 Units to 50,000 Units; at or about 1000 Units to 24,000 Units; at or about 1000 Units to 10,000 Units; or at or about 3000 Units to 10,000 Units.

57. The combination claim 55, wherein the first composition and second composition are formulated in a single composition for subcutaneous administration.

58. The combination claim 55, wherein:
(a) the amount of bisphosphonate in the first composition is sufficient to treat the disease or condition for a period of at least one week, two weeks, three weeks, four weeks, one month, two months, three months, four months, five months, six months, seven months, eight months, nine months, ten months, eleven months, twelve months, eighteen months or twenty four months without need for additional bisphosphonate administration to the subject during the period; and
(b) the amount of soluble hyaluronidase supplied in the preparation is such that, following subcutaneous administration of the bisphosphonate and hyaluronidase dosages over a desired length of time for completing such administration, the incidence of injection site reactions is eliminated or substantially reduced compared to subcutaneous administration of the same amount of bisphosphonate administered in the absence of the hyaluronidase over the same length of time.

59. The combination claim 55, wherein:
(a) the amount of bisphosphonate supplied in the first composition is sufficient to treat the disease or condition for a period of at least one week, two weeks, three weeks, four weeks, one month, two months, three months, four months, five months, six months, seven months, eight months, nine months, ten months, eleven months, twelve months, eighteen months or twenty four months without need for additional bisphosphonate administration to the subject during the period; and
(b) the amount of bisphosphonate in the first composition is such that, following subcutaneous administration, the bisphosphonate causes the same or substantially no greater degree or severity of injection site reactions compared to subcutaneous administration of about one third to one fifth the amount of bisphosphonate, administered at the same rate, in the absence of hyaluronidase.

60. The combination claim 55, wherein the first composition comprises one or more different bisphosphonates.

61. The combination claim 55, wherein the soluble hyaluronidase comprises a PH20 or a truncated form thereof.

62. The combination of claim 61, wherein the soluble hyaluronidase selected from an ovine, mouse, monkey, bovine or human PH20.

63. The combination of claim 61, wherein the soluble hyaluronidase is a soluble PH20 that lacks a C-terminal glycosylphosphatidylinositol attachment site.

64. The combination of claim 55, wherein the hyaluronidase is selected from among polypeptides containing a sequence of amino acids set forth in any of SEQ ID NOS: 4-9 and 48, and allelic variants, species variants and other variants thereof that retain hyaluronidase activity.

65. The combination of claim 64, wherein the soluble hyaluronidase variant is selected from among polypeptides having at least 60, 65, 70, 75, 80, 85, 88, 90, 91, 92, 93, 94, 95, 96, 97, 98, 99% or more sequence identity along their full length to a contiguous sequence of amino acids set forth in SEQ ID NO:1.

66. The combination of claim 55, wherein the soluble hyaluronidase comprises a polypeptide encoded by a sequence of nucleic acids that encodes a sequence of amino acids set forth in SEQ ID NO:3 or 4, or comprises a polypeptide encoded by a sequence of nucleic acids set forth in SEQ ID NO:49.

67. The combination claim 55, wherein the soluble hyaluronidase comprises rHuPH20.

68. The combination claim 55, wherein the soluble hyaluronidase comprises one or more soluble hyaluronidases.

69. The combination claim 55, wherein the soluble hyaluronidase is glycosylated, pegylated, or sialylated.

70. The combination claim 55, wherein the bisphosphonate is an N-bisphosphonate or a pharmaceutically acceptable salt or ester thereof or any hydrate thereof.

71. The combination claim 55, wherein the bisphosphonate is selected from among alendronate, cimadronate, clodronate, tiludronate, etidronate, ibandronate, neridronate, olpandronate, risedronate, piridronate, pamidronate, zoledronate, pharmaceutically acceptable salts or esters thereof and combinations thereof.

72. The combination claim 55, wherein the bisphosphonate is a nitrogenous bisphosphonate.

73. The combination claim 55, wherein the bisphosphonate is zoledronate, ibandronate or pamidronate.

74. The combination claim 55, wherein the bisphosphonate is provided in the form of a dry powder or a liquid and/or the soluble hyaluronidase is provided in the form of a dry powder or a liquid.

75. The combination of claim 74, wherein the volume of liquid is or is about 1 ml, 5 ml, 10 ml, 25 ml, 50 ml, 100 ml, 150 ml, 200 ml, 300 ml, 400 ml, 500 ml, 600 ml or 700 ml.

76. The combination claim 55, wherein the bisphosphonate in the first composition is or is about 0.5 milligrams (mg), about or 1 mg, about or 3 mg, about or 5 mg, about or 10 mg, about or 20 mg, about or 30 mg, about or 40 mg, about or 50 mg, about or 60 mg, about or 70 mg, about or 80 mg, about or 90 mg, about or 100 mg.

77. The combination claim 55, wherein:
(a) the bisphosphonate is zoledronate; and
(b) the amount of zoledronate in the first composition is or is about 0.5 milligrams (mg), about or 1 mg, about or 1.5 mg, about or 2 mg, about or 2.5 mg, about or 3 mg, about or 3.5 mg, about or 4 mg, about or 4.5 mg, about or 5 mg, about or 5.5 mg, about or 6 mg, about or 6.5 mg, about or 7 mg, about or 7.5 mg, about or 8 mg, about or 8.5 mg, about or 9 mg, about or 9.5 mg, or about or 10 mg.

78. The combination of claim 77, wherein the amount of zoledronate in the first composition is or is about 5 milligrams.

79. The combination of claim 77, wherein the amount of zoledronate in the first composition is or is about 5 milligrams in a liquid formulation, wherein the volume of the formulation is or is about 25 milliliters to 400 milliliters.

80. The combination claim 55, wherein:
(a) the bisphosphonate is ibandronate; and
(b) the amount of ibandronate in the first composition is or is about 0.5 milligrams (mg), about or 1 mg, about or 1.5 mg, about or 2 mg, about or 2.5 mg, about or 3 mg, about or 3.5 mg, about or 4 mg, about or 4.5 mg, about or 5 mg, about or 5.5 mg, about or 6 mg, about or 6.5 mg, about or 7 mg, about or 7.5 mg, about or 8 mg, about or 8.5 mg, about or 9 mg, about or 9.5 mg, or about or 10 mg.

81. The combination of claim 80, wherein the amount of ibandronate in the first composition is or is about 3 milligrams.

82. The combination of claim 80, wherein the amount of ibandronate in the first composition is or is about 3 milligrams in a liquid formulation, wherein the volume of the formulation is or is about 1 milliliter to 5 milliliters.

83. The combination of claim 55, wherein:
(a) the bisphosphonate is pamidronate; and
(b) the amount of pamidronate in the first composition is or is about 10 mg, about or 20 mg, about or 30 mg, about or 40 mg, about or 50 mg, about or 60 mg, about or 70 mg, about or 80 mg, about or 90 mg, or about or 100 mg.

84. The combination of claim 83, wherein the amount of pamidronate in the first composition is or is about 90 mg.

85. The combination of claim 83, wherein the amount of pamidronate in the first composition is or is about 90 milligrams in a liquid formulation, wherein the volume of the formulation is or is about 100 milliliter to 200 milliliters.

86. The combination of claim 55, wherein the second composition is a liquid.

87. The combination of claim 86, wherein the volume of liquid is or is about 1 milliliter (ml), is or is about 5 ml, is or is about 10 ml, is or is about 25 ml, is or is about 50 ml, is or is about 100 ml, is or is about 150 ml, is or is about 200 ml, is or is about 300 ml, is or is about 400 ml, is or is about 500 ml, is or is about 600 ml or is or is about 700 ml.

88. The combination of claim 55, wherein the second composition is a liquid formulation and the concentration of soluble hyaluronidase in the liquid formulation is at or about 10 Units/ml to 5,000,000 Units/ml, 500,000 Units/ml, 100 Units/ml to 100,000 Units/ml, 500 Units/ml to 50,000 Units/ml, 1000 Units/ml to 10,000 Units/ml, 5000 Units/ml to 7500 Units/ml, 5000 Units/ml to 50,000 Units/ml, 1,000 Units/ml to 10,000 Units/ml, or 100 Units/ml to 1000 Units/ml.

89. A pharmaceutical composition, comprising the combination of claim 55.

90. A kit comprising the combination of claim 55, and optionally instructions
